(19) United States
(12) Patent Application Publication
(10) Pub. No.: US 2022/0389515 A1
(43) Pub. Date: Dec. 8, 2022

(54) USE OF BIOMARKERS TO PREDICT CLINICAL SENSITIVITY TO 2-(4-CHLOROPHENYL)-N-((2-(2,6-DIOXOPIPERIDIN-3-YL)-1-OXOISOINDOLIN-5-YL)METHYL)-2,2-DIFLUOROACETAMIDE

(71) Applicant: CELGENE CORPORATION, Summit, NJ (US)

(72) Inventors: Gang LU, San Diego, CA (US); Christine SURKA, San Diego, CA (US)

(73) Assignee: CELGENE CORPORATION, Summit, NJ (US)

(21) Appl. No.: 17/772,101

(22) PCT Filed: Oct. 27, 2020
(86) PCT No.: PCT/US2020/057486

§ 371 Date: Apr. 26, 2022

Related U.S. Application Data
(60) Provisional application No. 62/926,878, filed on Oct. 28, 2019.

(51) Int. Cl. C12Q 1/6886; A61K 31/454; A61P 35/00

(52) U.S. Cl. CPC C12Q 1/6886; A61K 31/454; A61P 35/00; C12Q 2600/106; C12Q 2600/158; C12Q 2600/156

(57) ABSTRACT

A method of identifying a subject having cancer who is likely to be responsive to a treatment comprising a compound or predicting the responsiveness of a subject having or suspected of having cancer to a treatment comprising the compound, comprising: providing a sample from the subject; measuring gene expression level of one or more genes in the sample; and identifying the subject as being likely to be responsive to the treatment comprising the compound if the expression level of the gene is different from a reference level, and wherein the gene is a gene involved in mTOR signaling, or the gene is ILF 2 or ILFS.

### CROSS-REFERENCE TO RELATED APPLICATIONS

This application claims the benefit of U.S. Provisional Patent Application No. 62/926,878, filed Oct. 28, 2019, which is incorporated by reference herein in its entirety.

### 1. FIELD

Provided herein, in some embodiments, are methods of using certain biomarkers in predicting and monitoring clinical sensitivity and therapeutic response to certain compounds in patients having various diseases and disorders, such as cancer (e.g., lymphoma, multiple myeloma (MM), and leukemia, such as acute myeloid leukemia (AML)). Also provided herein, in certain embodiments, are methods of treating diseases using the treatment compounds.

### 2. BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). In general, cancer is divided into solid cancer and hematologic cancer. Examples of solid cancer include, but are not limited to, melanoma, adrenal carcinoma, breast carcinoma, renal cell cancer, pancreatic carcinoma, and small-cell lung carcinoma (SCLC), etc.

Blood cancer generally includes three main types: lymphoma, leukemia, and myeloma. Lymphoma refers to cancers that originate in the lymphatic system. Lymphoma includes, but is not limited to, Hodgkin's lymphoma, non-Hodgkin's lymphoma (NHL), diffuse large B-cell lymphoma (DLBCL), and peripheral T-cell lymphomas (PTCL), etc. Leukemia refers to malignant neoplasms of the blood-forming tissues. Acute leukemia involves predominantly undifferentiated cell populations, whereas chronic leukemia involves more mature cell forms. Acute leukemia is divided into acute lymphoblastic leukemia (ALL) and acute myeloblastic leukemia (AML) types. Chronic leukemia is divided into chronic lymphocytic leukemia (CLL) or chronic myelocytic leukemia (CML). Myeloma is a cancer of plasma cells in the bone marrow. Because myeloma frequently occurs at many sites in the bone marrow, it is often referred to as multiple myeloma (MM).

A tremendous demand therefore for new methods, treatments and compositions that can be used to treat patients with cancer including but not limited to, lymphoma (e.g., NHL), MM, leukemia (e.g., AML), and solid cancer. A number of studies have been conducted with the aim of providing compounds that can safely and effectively be used to treat cancers. For example, we have recently identified certain compounds (e.g., Compound D) useful to treat cancer including but not limited to, leukemia (e.g., AML). However, there is a need to develop efficient, sensitive, and accurate methods to detect, quantify, and characterize the pharmacodynamic activity of these compounds. The present invention satisfies these and other needs.

### 3. SUMMARY OF THE INVENTION

In one aspect, provided herein is a method of identifying a subject having cancer who is likely to be responsive to a treatment comprising a compound or predicting the responsiveness of a subject having or suspected of having cancer to a treatment comprising the compound, comprising:
i. providing a sample from the subject;
ii. measuring gene expression level of one or more genes in the sample; and
iii. identifying the subject as being likely to be responsive to the treatment comprising the compound if the expression level of the gene is different from a reference level,

wherein the compound is 2-(4-chlorophenyl)-N-((2-(2,6-dioxopiperidin-3-yl)-1-oxoisoindolin-5-yl)methyl)-2,2-difluoroacetamide (Compound D), which has the following structure:

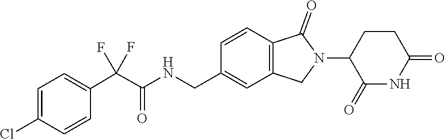 [SMILES: O=C1CCC(N2Cc3cc(CNC(=O)C(F)(F)c4ccc(Cl)cc4)ccc3C2=O)C(=O)N1]

or a stereoisomer or mixture of stereoisomers, isotopologue, pharmaceutically acceptable salt, tautomer, solvate, hydrate, co-crystal, clathrate, or polymorph thereof, and

wherein the gene is a gene involved in mTOR signaling, or the gene is ILF2 or ILF3.

In one aspect, provided herein is a method of treating a subject having cancer with a compound, comprising:

(a) identifying the subject having cancer that may be responsive to the treatment comprising the compound, comprising:
i. providing a sample from the subject;
ii. measuring gene expression level of one or more genes in the sample; and
iii. identifying the subject as being likely to be responsive to the treatment comprising the compound if the expression level of the gene is different from a reference level,

(b) administering the subject a therapeutically effective amount of the compound if the subject is identified as being likely to be responsive to the treatment comprising the compound,

wherein the compound is Compound D, or a stereoisomer or mixture of stereoisomers, isotopologue, pharmaceutically acceptable salt, tautomer, solvate, hydrate, co-crystal, clathrate, or polymorph thereof and

wherein the gene is a gene involved in mTOR signaling, or the gene is ILF2 or ILF3.

In some embodiments, the gene is involved in mTOR signaling. In some embodiments the gene is a positive regulator of mTOR. In some embodiments, the gene is mTOR. In other embodiments, the gene is Raptor. In other embodiments, the gene is Rictor.

In some embodiments of the various methods provided herein, the method comprises identifying the subject as being likely to be responsive to the treatment comprising the compound if the expression level of the gene is lower than a reference level.

In some embodiments, the reference level is the expression level of the gene in a subject resistant to Compound D. In other embodiments, the reference level is the expression level of the gene in a subject without the cancer. In other embodiments, the reference level is a pre-determined level.

In another aspect, the gene is a negative regulator of mTOR signaling. In some embodiments, the gene is TSC1. In other embodiments, the gene is TSC2. In other embodiments, the gene is GCN1. In other embodiments, the gene is GCN2. In other embodiments, the gene is DDIT4. In other embodiments, the gene is ATF4.

In some embodiments of the various methods provided herein, the method comprises identifying the subject as being likely to be responsive to the treatment comprising the compound if the expression level of the gene is higher than a reference level.

In some embodiments, the reference level is the expression level of the gene in a subject responsive to Compound D. In other embodiments, the reference level is the expression level of the gene in a subject without the cancer. In other embodiments, the reference level is a pre-determined level.

In some embodiments, the gene is ILF2. In other embodiments, the gene is ILF3.

In some embodiments of the various methods provided herein, the method comprises identifying the subject as being likely to be responsive to the treatment comprising the compound if the expression level of the gene is higher than a reference level.

In some embodiments, the reference level is the expression level of the gene in a subject responsive to Compound D. In other embodiments, the reference level is the expression level of the gene in a subject without the cancer. In other embodiments, the reference level is a pre-determined level.

In some embodiments of the various methods provided herein, the cancer is a hematological cancer. In another embodiment, the cancer is a lymphoma. In other embodiments the cancer is a leukemia. In other embodiments, the cancer is AML.

In another aspect, provided herein is a method of identifying a subject having cancer who is likely to be responsive to a treatment comprising a compound or predicting the responsiveness of a subject having or suspected of having cancer to a treatment comprising the compound, comprising i. providing a sample from the subject; ii. determining a sequence of a biomarker in the sample; and iii. identifying the subject as being unlikely to be responsive to the treatment comprising the compound if a mutation is identified in the biomarker, and/or identifying the subject as being likely to be responsive to the treatment comprising the compound if the mutation is not identified in the biomarker; wherein the compound is Compound D, or a stereoisomer or mixture of stereoisomers, isotopologue, pharmaceutically acceptable salt, tautomer, solvate, hydrate, co-crystal, clathrate, or polymorph thereof, and wherein the biomarker is CRBN or GSPT1.

In some embodiments, the biomarker is GSPT1, and wherein the mutation is a mutation of amino acid residue C568, L569, V570, D571, K572, K573, S574, G575, or E576 of GSPT1. In some embodiments, the mutation is selected from a group consisting of K572, K573, S574, G575, and combinations thereof. In some embodiments, the mutation comprises G575N.

In other embodiments, the biomarker is CRBN, and wherein the mutation is a mutation of amino acid residue N351, H357, W380, Y384, W386, or W400. In some embodiments, the mutation is Y384A or W386A.

In another aspect, provided herein is a method of treating a subject having cancer comprising administering to the subject a compound, wherein the subject has been determined to be likely to be responsive to the compound according a method comprising i. providing a sample from the subject; ii. determining a sequence of a biomarker in the sample; and iii. identifying the subject as being likely to be responsive to the compound if a mutation is not identified in the biomarker; wherein the compound is Compound D, or a stereoisomer or mixture of stereoisomers, isotopologue, pharmaceutically acceptable salt, tautomer, solvate, hydrate, co-crystal, clathrate, or polymorph thereof, and wherein the biomarker is CRBN or GSPT1.

In yet another aspect, provided herein is a method of treating a subject having cancer comprising administering to the subject a second compound, wherein the subject has been determined to be unlikely to be responsive to a first compound according a method comprising i. providing a sample from the subject; ii. determining a sequence of a biomarker in the sample; and iii. identifying the subject as being unlikely to be responsive to the compound if a mutation is identified in the biomarker; wherein the first compound is Compound D, or a stereoisomer or mixture of stereoisomers, isotopologue, pharmaceutically acceptable salt, tautomer, solvate, hydrate, co-crystal, clathrate, or polymorph thereof, wherein the second compound is not Compound D, or a stereoisomer or mixture of stereoisomers, isotopologue, pharmaceutically acceptable salt, tautomer, solvate, hydrate, co-crystal, clathrate, or polymorph thereof, and wherein the biomarker is CRBN or GSPT1.

In some embodiments, the biomarker is GSPT1, and wherein the mutation is a mutation of amino acid residue C568, L569, V570, D571, K572, K573, S574, G575, or E576 of GSPT1. In some embodiments, the mutation is selected from a group consisting of K572, K573, S574, G575, and combinations thereof. In some embodiments, the mutation comprises G575N.

In other embodiments, the biomarker is CRBN, and wherein the mutation is a mutation of amino acid residue N351, H357, W380, Y384, W386, or W400. In some embodiments, the mutation is Y384A or W386A.

In some embodiments of the various methods provided herein, the cancer is a hematological cancer. In another embodiment, the cancer is a lymphoma. In other embodiments the cancer is a leukemia. In other embodiments, the cancer is AML.

### 4. BRIEF DESCRIPTION OF THE FIGURES

FIG. 1A illustrates the chemical structures of Compound D, Control Compound and lenalidomide (LEN) with the glutarimide ring shown in red. FIG. 1B shows the antiproliferative effect of Compound D in AML cell lines. Cells were incubated with DMSO or Compound D at the indicated concentrations. On Day 3, cell proliferation was assessed by the Cell-Titer Glo (CTG) assay.

FIG. 2A illustrates the volcano plot of differentially abundant proteins in response to Compound D treatment relative to the DMSO control. KG1 cells were treated with DMSO or 100 nM Compound D for 4 hours and subjected to TMT proteomics analysis. The X-axis denotes the log 2-fold change of Compound D versus the DMSO control for each protein. P-values were corrected for multiple hypothesis testing using the Benjamini-Hoschberg method to arrive at an adjusted-P-value (adj-P, also known as a false discovery rate). The y-axis denotes the −log 10 (adj-P) values indicating statistical significance such that proteins lying above the dotted red line are statistically significant findings with adjusted P-values<0.05. FIG. 2B illustrates the immunoblot analysis of KG1 and U937 cells treated with DMSO or Compound D for 4 hours. Where indicated, cells were pretreated with Bortezomib or MLN4924 for 30 minutes. FIGS. 2C and 2D illustrate the immunoblot analysis (FIG. 2C) and cell proliferation (FIG. 2D) of U937-Cas9 parental cells or cells stably transduced with lentiviral vectors expressing a non-targeting sgRNA (sgNT-1), a sgRNA targeting a non-coding region (sgNC-8), or a sgRNA targeting CRBN(sgCRBN-8). Cells were treated with DMSO or Compound D at indicated concentrations. FIGS. 2E and 2F illustrate the immunoblot analysis (left panel) and cell proliferation (right panel) of MOLM-13 (FIG. 2E) and OCI-AML2 (FIG. 2F) parental and CRBN−/− cells. Cells were treated with DMSO or Compound D at indicated concentrations. Results shown in all figure panels are representative of three biological replicates. Data in FIGS. 2D, 2E (right panel), and 2F (right panel) are shown as mean±SD, n=3 technical replicates.

FIG. 3A illustrates FLAG-HA tagged cereblon wild-type (FLAG-HA-CRBN) or Y384A/W386A mutant (FLAG-HA-YWAA) produced in 293FT CRBN−/− cells was used to capture V5 tagged GSPT1 (GSPT1-V5) and V5 tagged IKZF1 (IKZF1-V5) transiently expressed in 293FT CRBN−/− cells. DMSO, lenalidomide (LEN) or Compound D was added into the binding assay. The left panel shows the immunoblot analysis of anti-HA immunoprecipitates, and the right panel shows the immunoblot analysis of 293FT CRBN−/− cells transiently transfected to produce FLAG-HA tagged cereblon (wild-type or YWAA mutant), or V5-tagged GSPT1 or IKZF1. FIG. 3B illustrates the surface representation of GSPT1 in complex with cereblon, DDB1 and Compound D, with DDB1 shown in purple, cereblon in blue, and GSPT1 in orange. The position of Compound D is shown with a white arrow. FIG. 3C illustrates the Fo-Fc omit electron density (green mesh) for Compound D (yellow sticks) contoured at 3.06. FIG. 3D illustrates GSPT1's interaction with cereblon is mediated by a β-hairpin loop. Hydrogen bond interactions between cereblon and the GPST1 β-hairpin are shown as yellow dashes. FIG. 3E illustrates details of the binding interface between cereblon and GSPT1. Compound D is represented as yellow sticks. Predicted polar interactions between Compound D and cereblon are shown as yellow dashes. FIG. 3F illustrates the immunoblot analysis of U937 parental cells or cells stably expressing HA-GSPT1-G575N. Cells were treated with DMSO or Compound D at the indicated concentrations. FIGS. 3G and 311 illustrate the immunoblot analysis (left panel) and cell proliferation (right panel) of MOLM-13 (FIG. 3G) and OCI-AML2 (FIG. 311) parental cells and cells stably expressing HA tagged GSPT1-G575N. Cells were treated with DMSO or Compound D at indicated concentrations.

FIG. 31 illustrates the superimposition of the DDB1-cereblon-GSPT1-Compound D and DDB1-cereblon-GSPT1-Control Compound structures. For the Control Compound complex structure, GSPT1 is shown in light orange, cereblon in light blue, and Control Compound in light yellow. FIGS. 3J and 3K illustrates the growth curve (FIG. 3J) and immunoblot analysis (FIG. 3K) of U937 parental cells or cells transiently transduced with lentiviral vectors expressing control shRNA (shCNTL) or GSPT1 specific shRNAs (shGSPT1-1 to 4). Cell proliferation was quantified with CTG at Days 4, 6, and 8 after transduction. Result shown in all figure panels is representative of three biological replicates. Data in FIG. 3J are shown as mean±SD, n=3 technical replicates.

FIG. 4A illustrates the schematic showing the design of the genome-wide CRISPR screen to identify molecular determinants of Compound D response. FIG. 4B illustrates the cell proliferation curve of U937-Cas9 cells transduced with the lentiviral sgRNA library and treated with DMSO or Compound D. Three days post-transduction, cells were treated with DMSO or 10 μM Compound D for 9 days. FIG. 4C illustrates normalized sgRNA read count comparison of different treatment conditions and technical replicates on day 3 and day 12 post-transduction of the lentiviral sgRNA library. 150 k sgRNAs are used in the scatter plots. Numbers in the upper right boxes indicate the Pearson correlation coefficient between samples. “***” indicates the correlation p-value is <0.001. sgRNAs targeting UBE2G1 or CRBN were shown in green and red, respectively. FIG. 4D illustrates pathway enrichment analysis of genes enriched by Compound D treatment with log 2 fold change (log 2FC)>2 and false discovery rate (FDR)<0.05 relative to the DMSO control. The color and size of the dots represent adjusted significance level and gene ratio respectively. Gene ratios refer to the number of input genes annotated to an individual pathway as a ratio of all input genes annotated to any Reactome pathway. FIG. 4E illustrates the scatter plot of 78 genes significantly enriched by Compound D (log 2FC>2 and FDR<0.05). The X axis denotes the Compound D enrichment score shown as log 2FC; the Y axis denotes the gene essentiality score shown as log 2FC (T12_DMSO vs T3_DMSO). Some of these genes were grouped into 10 functional modules with different color coding. FIG. 4F shows the network graph of enriched pathways among 78 top-ranked genes enriched by Compound D treatment in U937 cells. Enriched pathways from the Reactome database were identified using Fisher's exact test and were selected by adjusted p-value (FDR)<0.05. Pathway nodes are color-coded with different shades of red according to their statistical significance. The grey nodes in the graph depict pathway genes that were enriched by Compound D treatment. The core enriched pathways modulating the response to Compound D are highlighted with green circles. FIGS. 4G and 4H show the log 2FC values of sgRNAs targeting Compound D enriched genes in the functional modules as indicated. Background shown in dark blue represents the log 2FC values of all sgRNAs in the library. Each solid line with a color representing a functional module indicates the log 2FC value of an individual sgRNA. FIG. 4G shows are well-characterized genes known to be essential for the activity of the cereblon E3 ligase complex; FIG. 4H shows novel genes that regulate the response to Compound D with no clear understanding of the underlying mechanisms.

FIG. 5A illustrates the genome-wide CRISPR screen method for identifying genes that confer sensitivity and resistance to Compound D treatment. FIG. 5B shows the genes that drop-out or enrich upon treatment with Compound D in the genome-wide CRISPR screen.

FIG. 6A illustrates the CRISPR competition assay. FIG. 6B depicts the results of knockout of mTOR, Raptor or Rictor gene in U937 cells. FIG. 6C depicts the results of the CRISPR competition assay performed in mTOR knockout cells. FIG. 6D depicts the results of the CRISPR competition assay performed in Raptor knockout cells. FIG. 6E depicts the results of the CRISPR competition assay performed in Rictor knockout cells.

FIG. 7A shows the results of knockout of the TSC1 or TSC2 gene in U937 cells. FIG. 7B shows the results of knockout of the TSC1 or TSC2 gene in OCI-AML2 cells. FIG. 7C shows the results of cell proliferation assays in TSC1 knockout or TSC2 knockout U937 cells. FIG. 7D shows the results of cell proliferation assays in TSC1 knockout or TSC2 knockout OCI-AML2 cells. FIG. 7E shows the results of TSC1 or TSC2 gene knockout in U937 cells. FIG. 7F depicts the results of the CRISPR competition assay performed in TSC1 knockout or TSC2 knockout U937 cells. FIG. 7G depicts the results of the CRISPR competition assay performed in TSC1 knockout or TSC2 knockout OCI-AML2 cells. FIGS. 7H and 7I show the RFP+/GFP+ ratios of U937-Cas9 cells co-expressing RFP and sgNT-1, sgNC-8, or one of three sgRNAs targeting TSC1 (FIG. 7H) or TSC2 (FIG. 7I) mixed with cells co-expressing GFP and sgNT-1 at each indicated timepoint were normalized to the RFP+/GFP+ ratio of the cell mixtures on “Day 0.” FIG. 7J shows the evaluation of the effect of TSC1 or TSC2 knockout on Compound D response in OCI-AML2 cells using a flow cytometry-based CRISPR competition assay. The RFP+/GFP+ ratios of OCI-AML2-Cas9 cells co-expressing RFP and the indicated sgRNAs mixed with cells co-expressing GFP and sgNT-1 at each indicated timepoint were normalized to the RFP+/GFP+ ratio of the cell mixtures on “Day 0.” FIG. 7K shows the results of the DEU analysis, which revealed significant differential splicing of individual CRBN exons (red bars) with knockout of ILF3. An exon, which annotates to the truncated transcript, CRBN.213 (exon bin No. 13), is significantly elevated (FDR=0.02) with ILF3 knockout relative to NT controls. Conversely, exons downstream of this isoform are significantly under-represented (FDR=0.05; exon bin No. 14) in the ILF3 knockout cells relative to parental.

FIG. 8A shows the effects of TSC1 knockout or TSC2 knockout on Compound D induced GSPT1 degradation in U937 cells. FIG. 8B shows the effects of TSC1 knockout or TSC2 knockout on Compound D induced GSPT1 degradation in OCI-AML2 cells. FIG. 8C shows the immunoblot analysis of U937-Cas9 parental cells or cells stably expressing sgRNAs as indicated. Cells were treated with DMSO or Compound D in the presence of cycloheximide. FIGS. 8D and 8E show the immunoblot analysis of U937-Cas9 cells stably expressing HA-tagged GSPT1 and the indicated sgRNAs. Cells were treated with DMSO, Compound D (FIG. 8D) or pomalidomide (POM; FIG. 8E) at the indicated concentrations for 18 hours. FIG. 8F shows the immunoblot analysis of anti-HA immunoprecipitates (top) or whole cell extracts (bottom) of U937-Cas9 parental cells or cells stably expressing the indicated sgRNAs. Cells were treated with MLN4924 and DMSO or Compound D. Result shown in all figure panels is representative of three biological replicates.

FIG. 9A shows the results of knockout of GCN2. FIG. 9B depicts the results of the CRISPR competition assay performed in GCN2 knockout U937 cells. FIG. 9C shows the results of knockout of TSC1, TSC2, GCN1, GCN2, DDIT4, ATF4, or CRBN in U937 cells. FIG. 9D depicts the results of the cell proliferation assays performed in GCN2 knockout, ATF4 knockout, GCN1 knockout, and CRBN knockout U937 cells. FIG. 9E depicts the results of the cell proliferation assays performed in DDIT4 knockout, TSC1 knockout, TSC2 knockout and CRBN knockout U937 cells.

FIG. 10A shows the results of ILF3 knockout in U937 cells. FIG. 10B shows the results of the CRISPR competition assay in the ILF3 knockout U937 cells. FIG. 10C shows the results of ILF2 or ILF3 knockout on Compound D induced GSPT1 degradation in U937 cells and the expression of CRBN in these cells. FIG. 10D shows the results of the CRISPR competition assay in ILF2 knockout U937 cells. FIG. 10E shows the results of ILF2 or ILF3 knockout in OCI-AML2 cells and the expression of CRBN in these cells. FIG. 10F shows the results of the CRISPR competition assay in ILF2 knockout or ILF3 knockout OCI-AML2 cells. FIG. 10G shows the effects of ILF3 knockout on Compound D induced GSPT1 degradation in U937 cells and the expression of CRBN in these cells. FIG. 10H shows the results of qPCR quantitation of full length and alternatively spliced CRBN mRNA transcripts in ILF3 knockout U937 cells.

FIG. 10I shows the schematic design of the flow-cytometry based CRISPR competition assay. FIG. 10J shows the immunoblot analysis of U937-Cas9 cells inducibly expressing sgNT-1, sgNC-1, sgILF3-2 or sgILF3-4. Cells were treated with doxycycline (DOX) for 6 days. FIG. 10K shows the immunoblot analysis of U937-Cas9 parental cells or cells expressing control sgRNAs (sgNT-1 or sgNC-8) or ILF2-specific sgRNAs (sgILF2-1 or sgILF2-6).

FIGS. 11A and 11B show the RNAseq analysis of U937-Cas9 cells with inducible expression sgNT-1 or sgILF3-2 for 7 days. Evidence of differential splicing was observed in a total of 967 unique genes by up- and/or down-regulated exon usage with ILF3 knockout in U937 cells, reaching a corrected significance level (FDR)<0.05. At the gene-level, 791 genes were found to be significantly (FDR<0.05) up- or down-regulated with ILF3 knockout. FIG. 11A shows the venn diagram showing the overlap of genes with significant Differential Exon Usage (DEU; LHS) and genes with Differential Expression at the Gene-level (DEG; RHS). FIG. 11B shows the pathway enrichment analysis of DEU and DEG genes. The color and size of the dots represent adjusted significance level and gene ratio respectively. Gene ratio refers to the number of input genes annotated to an individual pathway as a ratio of all input genes annotated to any Reactome pathway. FIG. 11C shows the schematic diagram adopted from Ensembl showing the genomic locus of CRBN and the gene structures of 15 CRBNmRNA transcripts. Boxes denote exons; solid lines denote introns; shaded areas in each box denote protein coding regions; and unfilled areas in each box represent untranslated regions. CRBNtranscripts 201 and 203 encode two full-length cereblon proteins with one amino acid difference in the N-terminus. CRBN transcript 213 containing a cryptic exon 5 with a premature stop codon encodes a truncated cereblon protein lacking most of its functional domain.

FIGS. 12A-12D show characterization of the role of GCN2 (FIG. 12A), GCN1 (FIG. 12B), ATF4 (FIG. 12C) and DDIT4 (FIG. 12D) in mediating Compound D response using a flow-cytometry based CRISPR competition assay. U937 cells stably expressing Cas9 were infected with a lentiviral vector constitutively co-expressing GFP and sgNT-1, or with lentiviral vectors constitutively co-expressing RFP and sgNT-1, sgNC-8, or one of the gene-specific sgRNAs as indicated. Three days after infection, RFP and GFP cells were mixed at a 1:1 ratio and treated with DMSO or 10 μM Compound D. The change of RFP+/GFP+ ratio was monitored by flow cytometry every 2 days thereafter. The RFP+/GFP+ ratios of U937-Cas9 cells co-expressing RFP and sgNT-1, sgNC-8, or one of three sgRNAs targeting GCN2 (FIG. 12A), GCN1 (FIG. 12B), ATF4 (FIG. 12C) or DDIT4 (FIG. 12D) mixed with cells co-expressing GFP and sgNT-1 at each indicated timepoint were normalized to the RFP+/GFP+ ratio of the cell mixtures on “Day 0.” FIG. 12E shows the schematic diagram showing the design of the flow cytometry-based CRISPR competition assay as shown in FIGS. 12A-12D. FIG. 12F shows the immunoblotting analysis of U937-Cas9 parental cell or cells expressing the indicated sgRNAs used in the CRISPR competition assay as shown in FIGS. 12A-12D. FIGS. 12F-12K Evaluation of the effect of GCN1, GCN2, ATF4 or DDIT4 knockout on Compound D response in OCI-AML2 cells using a flow cytometry-based CRISPR competition assay. FIG. 12G shows a schematic diagram showing the design of the CRISPR competition assay. FIGS. 12H and 12J shows the immunoblot analysis of OCI-AML2-Cas9 parental cells or cells stably expressing the indicated sgRNAs.

FIGS. 12I and 12K shows the RFP+/GFP+ ratios of OCI-AML2-Cas9 cells co-expressing RFP and the indicated sgRNAs mixed with cells co-expressing GFP and sgNT-1 at each indicated timepoint were normalized to the RFP+/GFP+ ratio of the cell mixtures on “Day 0.”

FIG. 13A shows the immunoblot analysis of U937 parental and GCN2−/− cells treated with DMSO or Compound D at the indicated concentrations for 24 hours. The U937 GCN2−/− cell line is derived from a single clone of U937 parental cells stably infected with a lentiviral CRISPR vector targeting GCN2. FIG. 13B shows the immunoblot analysis of whole cell extracts of KG-1 cells. Cells were incubated with DMSO or 200 nM Compound D and lysed at the indicated time points. Arrows pointing to bands in the blot on the right designate the cleaved forms of caspase-3. FIG. 13C shows the quantitative RT-PCR analysis of U937 parental and GCN2−/− cells treated with DMSO or Compound D at the indicated concentrations for 24 hours. The U937 GCN2−/− cell line is derived from a single clone of U937 parental cells stably infected with a lentiviral CRISPR vector targeting GCN2. FIG. 13D shows the quantitative RT-PCR analysis of indicated mRNA transcript in KG-1 cells incubated with DMSO or 200 nM Compound D for 2, 4 or 6 hours. FIG. 13E shows the immunoblot analysis of U937 parental cells and GCN2−/− cells with or without a stably transduced lentiviral vector expressing HA-tagged GCN2 wild-type or mutants as indicated. Cells were treated with DMSO or Compound D for 24 hours. FIG. 13F shows the effect of Compound D on proliferation of cells shown in FIG. 13E. On day 3 after Compound D treatment, cell proliferation was assessed by CTG. Result shown in all figure panels is representative of at least three biological replicates. Data in FIGS. 13C, 13D and 13E are shown as mean±SD, n=3 technical replicates.

### 5. DETAILED DESCRIPTION OF THE INVENTION

The present disclosure is based in part on the surprising findings that responsiveness or resistance to treatment with a compound provided herein, e.g., 2-(4-chlorophenyl)-N-((2-(2,6-dioxopiperidin-3-yl)-1-oxoisoindolin-5-yl)methyl)-2,2-difluoroacetamide (Compound D) correlate with expression levels of certain genes. For example, as shown in Section 6 below, genes involved in mTOR signaling are found by the present disclosure to correlate with Compound D responsiveness or resistance. The present disclosure also identifies other markers such as ILF2 and ILF3 as predictors for response to treatment with Compound D.

### 5.1. Definitions

As used herein, the term “cancer” includes, but is not limited to, solid cancer and hematological cancer. The term “cancer” refers to disease of tissues or organs, including but not limited to, cancers of the bladder, bone, blood, brain, breast, cervix, chest, colon, endometrium, esophagus, eye, head, kidney, liver, lymph nodes, lung, mouth, neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastases, glioblastoma multiforme, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

As used herein, “hematological cancer” includes myeloma, lymphoma, and leukemia. In one embodiment, the myeloma is multiple myeloma. In some embodiments, the leukemia is, for example, acute myelogenous leukemia (AML), acute lymphocytic leukemia (ALL), adult T-cell leukemia, chronic lymphocytic leukemia (CLL), hairy cell leukemia, myelodysplasia, myeloproliferative disorders, chronic myelogenous leukemia (CML), myelodysplastic syndrome (MDS), human lymphotropic virus-type 1 (HTLV-1) leukemia, mastocytosis, or B-cell acute lymphoblastic leukemia. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma.

The term “prognosis risk,” when used in connection with cancer, refers to the possible outcomes of the cancer, including responsiveness to certain treatments, duration or extent of remission, potential survival rate, probability of relapse, etc. Factors that affect a patient's prognosis risk include, but are not limited to, demographic (e.g., age, race, sex, etc.), disease-specific (e.g., cancer stage), genetic (e.g., risk gene), co-morbid (e.g., other conditions accompanying the cancer), etc. A good “prognosis risk” means that the patient is likely to be responsive to certain treatments, is likely to survive, and/or is unlikely to relapse, etc. A poor “prognosis risk” means that the patient is unlikely to be responsive to certain treatments, is unlikely to survive, and/or is likely to relapse, etc.

As used herein, and unless otherwise specified, the terms “treat,” “treating,” and “treatment” refer to an action that occurs while a patient is suffering from the specified cancer, which reduces the severity of the cancer or retards or slows the progression of the cancer.

The term “sensitivity” or “sensitive” when made in reference to treatment with compound is a relative term which refers to the degree of effectiveness of the compound in lessening or decreasing the progress of a tumor or the disease being treated. For example, the term “increased sensitivity” when used in reference to treatment of a cell or tumor in connection with a compound refers to an increase of, at least about 5%, or more, in the effectiveness of the tumor treatment.

As used herein, the terms “compound” and “treatment compound” are used interchangeably, and include the non-limiting examples of compounds disclosed in Section 5.5 below.

As used herein, and unless otherwise specified, the term “therapeutically effective amount” of a compound is an amount sufficient to provide a therapeutic benefit in the treatment or management of a cancer, or to delay or minimize one or more symptoms associated with the presence of the cancer. A therapeutically effective amount of a compound means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent. The term also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “responsiveness” or “responsive” when used in reference to a treatment refers to the degree of effectiveness of the treatment in lessening or decreasing the symptoms of a disease, e.g., cancer, such as MM or AML, being treated. For example, the term “increased responsiveness” when used in reference to a treatment of a cell or a subject refers to an increase in the effectiveness in lessening or decreasing the symptoms of the disease compared to a reference treatment (e.g., of the same cell or subject, or of a different cell or subject) when measured using any methods known in the art. In certain embodiments, the increase in the effectiveness is at least about 5%, at least about 10%, at least about 20%, at least about 30%, at least about 40%, or at least about 50%.

An improvement in the cancer or cancer-related disease can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about 10%, about 20%, about 30%, about 40%, about 50%, about 60%, about 70%, about 80%, or about 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

The term “likelihood” generally refers to an increase in the probability of an event. The term “likelihood” when used in reference to the effectiveness of a patient tumor response generally contemplates an increased probability that the rate of tumor progress or tumor cell growth will decrease. The term “likelihood” when used in reference to the effectiveness of a patient tumor response can also generally mean the increase of indicators, such as mRNA or protein expression, that may evidence an increase in the progress in treating the tumor.

The term “predict” generally means to determine or tell in advance. When used to “predict” the effectiveness of a cancer treatment, for example, the term “predict” can mean that the likelihood of the outcome of the cancer treatment can be determined at the outset, before the treatment has begun, or before the treatment period has progressed substantially.

The term “monitor,” as used herein, generally refers to the overseeing, supervision, regulation, watching, tracking, or surveillance of an activity. For example, the term “monitoring the effectiveness of a compound” refers to tracking the effectiveness in treating cancer in a patient or in a tumor cell culture. Similarly, the term “monitoring,” when used in connection with patient compliance, either individually, or in a clinical trial, refers to the tracking or confirming that the patient is actually taking a drug being tested as prescribed. The monitoring can be performed, for example, by following the expression of mRNA or protein biomarkers.

The term “regulate” as used herein refers to controlling the activity of a molecule or biological function, such as enhancing or diminishing the activity or function.

The term “refractory” or “resistant” refers to a circumstance where patients, even after intensive treatment, have residual cancer cells (e.g., hematological cancer cells, for example, leukemia, lymphoma or multiple myeloma cells) in for example, their lymphatic system, blood, and/or blood forming tissues (e.g., marrow).

A “biological marker” or “biomarker” is a substance whose detection indicates a particular biological state, such as, for example, the presence of cancer. In some embodiments, biomarkers can be determined individually. In other embodiments, several biomarkers can be measured simultaneously. In some embodiments, a “biomarker” indicates a change in the level of mRNA expression that may correlate with the risk or progression of a disease, or with the susceptibility of the disease to a given treatment. In some embodiments, the biomarker is a nucleic acid, such as mRNA or cDNA. In additional embodiments, a “biomarker” indicates a change in the level of polypeptide or protein expression that may correlate with the risk or progression of a disease, or patient's susceptibility to treatment. In some embodiments, the biomarker can be a polypeptide or protein, or a fragment thereof. The relative level of specific proteins can be determined by methods known in the art. For example, antibody based methods, such as an immunoblot, enzyme-linked immunosorbent assay (ELISA), or other methods can be used.

The terms “polypeptide” and “protein,” as used interchangeably herein, refer to a polymer of three or more amino acids in a serial array, linked through peptide bonds. The term “polypeptide” includes proteins, protein fragments, protein analogues, oligopeptides, and the like. The term “polypeptide” as used herein can also refer to a peptide. The amino acids making up the polypeptide may be naturally derived, or may be synthetic. The polypeptide can be purified from a biological sample. The polypeptide, protein, or peptide also encompasses modified polypeptides, proteins, and peptides, e.g., glycopolypeptides, glycoproteins, or glycopeptides; or lipopolypeptides, lipoproteins, or lipopeptides.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from the RNA molecule to give a protein, a polypeptide, or a portion thereof.

The term “expression level” refers to the amount, accumulation, or rate of a biomarker molecule or a gene set. An expression level can be represented, for example, by the amount or the rate of synthesis of a messenger RNA (mRNA) encoded by a gene, the amount or the rate of synthesis of a polypeptide or protein encoded by a gene, or the amount or the rate of synthesis of a biological molecule accumulated in a cell or biological fluid. The term “expression level” refers to an absolute amount of a molecule in a sample or a relative amount of the molecule, determined under steady-state or non-steady-state conditions.

An mRNA that is “upregulated” is generally increased upon a given treatment or condition, or in certain patient groups. An mRNA that is “downregulated” generally refers to a decrease in the level of expression of the mRNA in response to a given treatment or condition, or in certain patient groups. In some situations, the mRNA level can remain unchanged upon a given treatment or condition. An mRNA from a patient sample can be “upregulated” when treated with a drug, as compared to a non-treated control. This upregulation can be, for example, an increase of about 5%, about 10%, about 20%, about 30%, about 40%, about 50%, about 60%, about 70%, about 80%, about 90%, about 100%, about 200%, about 300%, about 500%, about 1,000%, about 5,000%, or more of the comparative control mRNA level. Alternatively, an mRNA can be “downregulated”, or expressed at a lower level, in response to administration of certain compounds or other agents. A downregulated mRNA can be, for example, present at a level of about 99%, about 95%, about 90%, about 80%, about 70%, about 60%, about 50%, about 40%, about 30%, about 20%, about 10%, about 1%, or less of the comparative control mRNA level.

Similarly, the level of a polypeptide or protein biomarker from a patient sample can be increased when treated with a drug, as compared to a non-treated control. This increase can be about 5%, about 10%, about 20%, about 30%, about 40%, about 50%, about 60%, about 70%, about 80%, about 90%, about 100%, about 200%, about 300%, about 500%, about 1,000%, about 5,000%, or more of the comparative control protein level. Alternatively, the level of a protein biomarker can be decreased in response to administration of certain compounds or other agents. This decrease can be, for example, present at a level of about 99%, about 95%, about 90%, about 80%, about 70%, about 60%, about 50%, about 40%, about 30%, about 20%, about 10%, about 1%, or less of the comparative control protein level.

The terms “determining,” “measuring,” “evaluating,” “assessing,” and “assaying” as used herein generally refer to any form of measurement, and include determining whether an element is present or not. These terms include quantitative and/or qualitative determinations. Assessing may be relative or absolute. “Assessing the presence of” can include determining the amount of something present, as well as determining whether it is present or absent.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present.

The terms “isolated” and “purified” refer to isolation of a substance (such as mRNA, DNA, or protein) such that the substance comprises a substantial portion of the sample in which it resides, i.e., greater than the portion of the substance that is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. For example, a sample of isolated mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

As used herein, the term “bound” indicates direct or indirect attachment. In the context of chemical structures, “bound” (or “bonded”) may refer to the existence of a chemical bond directly joining two moieties or indirectly joining two moieties (e.g., via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and embodiments where the attachment is indirect.

The term “sample” as used herein relates to a material or mixture of materials, typically, although not necessarily, in fluid form, containing one or more components of interest.

“Biological sample” as used herein refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, and cells isolated from a mammal. Exemplary biological samples include but are not limited to cell lysate, cells, tissues, organs, organelles, a biological fluid, a blood sample, a urine sample, a skin sample, and the like. Preferred biological samples include, but are not limited to, whole blood, partially purified blood, PBMC, tissue biopsies (including tumor biopsies), circulating tumor cells, and the like.

The term “polymerase chain reaction” or “PCR” as used herein generally refers to a procedure wherein small amounts of a nucleic acid, RNA and/or DNA, are amplified as described, for example, in U.S. Pat. No. 4,683,195. Generally, sequence information from the ends or beyond of the region of interest needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 1987, 51:263-273; PCR Technology (Stockton Press, NY, Erlich, ed., 1989).

“Tautomer” as used herein refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

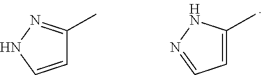

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids known in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like. Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts (calcium, magnesium, sodium, or potassium salts in particular). Suitable organic bases include, but are not limited to, N,N-dibenzylethylenediamine, chloroprocaine, choline, diethanolamine, ethylenediamine, meglumaine (N-methylglucamine), lysine, and procaine.

As used herein and unless otherwise indicated, the term “solvate” means a compound provided herein or a salt thereof that further includes a stoichiometric or non-stoichiometric amount of solvent bound by non-covalent intermolecular forces. Where the solvent is water, the solvate is a hydrate.

As used herein and unless otherwise indicated, the term “co-crystal” means a crystalline form that contains more than one compound in a crystal lattice. Co-crystals include crystalline molecular complexes of two or more non-volatile compounds bound together in a crystal lattice through non-ionic interactions. As used herein, co-crystals include pharmaceutical co-crystals wherein the crystalline molecular complexes containing a therapeutic compound and one or more additional non-volatile compound(s) (referred to herein as counter-molecule(s)). A counter-molecule in a pharmaceutical co-crystal is typically a non-toxic pharmaceutically acceptable molecule, such as, for example, food additives, preservatives, pharmaceutical excipients, or other active pharmaceutical ingredients (API). In some embodiments, pharmaceutical co-crystals enhance certain physicochemical properties of drug products (e.g., solubility, dissolution rate, bioavailability, and/or stability) without compromising the chemical structural integrity of the API. See, e.g., Jones et al., MRS Bulletin 2006, 31, 875-879; Trask, Mol. Pharmaceutics 2007, 4(3):301-309; Schultheiss & Newman, Crystal Growth & Design 2009, 9(6):2950-2967; Shan & Zaworotko, Drug Discovery Today 2008, 13(9/10):440-446; and Vishweshwar et al., J. Pharm. Sci. 2006, 95(3):499-516.

As used herein, and unless otherwise specified, the term “stereoisomer” encompasses all enantiomerically/stereoisomerically pure and enantiomerically/stereoisomerically enriched compounds of this invention.

As used herein and unless otherwise indicated, the term “stereoisomerically pure” means a composition that comprises one stereoisomer of a compound and is substantially free of other stereoisomers of that compound. For example, a stereoisomerically pure composition of a compound having one chiral center will be substantially free of the opposite enantiomer of the compound. A stereoisomerically pure composition of a compound having two chiral centers will be substantially free of other diastereomers of the compound. A typical stereoisomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “stereoisomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereoisomerically pure composition of a compound having one chiral center. Similarly, the term “stereoisomerically enriched” means a stereoisomerically enriched composition of a compound having one chiral center.

As used herein and unless otherwise indicated, the term “prodrug” means a derivative of a compound that can hydrolyze, oxidize, or otherwise react under biological conditions (in-vitro or in-vivo) to provide the compound. Examples of prodrugs include, but are not limited to, derivatives of compounds described herein (e.g., Compound 1) that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues.

It should also be noted compounds can contain unnatural proportions of atomic isotopes at one or more of the atoms. For example, the compounds may be radiolabeled with radioactive isotopes, such as for example tritium (H), iodine-125 (¹²⁵I), sulfur-35 (³⁵S), or carbon-14 (¹⁴C), or may be isotopically enriched, such as with deuterium (2H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds. In some embodiments, isotopologues provided herein are deuterium enriched compounds. In some embodiments, isotopologues provided herein are deuterium enriched compounds, where the deuteration occurs on the chiral center. In some embodiments, provided herein are isotopologues of the compounds provided herein, where deuteration occurs on the chiral center. In some embodiments, provided herein are isotopologues of Compound D, where deuteration occurs on the chiral center.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

It should be noted that if there is a discrepancy between a depicted structure and a name given to that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual (4th ed. 2014); Glover, ed., DNA Cloning, Volumes I and II (2^{nd} ed. 1995); Immunochemical Methods in Cell and Molecular Biology (Academic Press, London); Scopes, Protein Purification: Principles and Practice (Springer Verlag, N.Y., 3rd ed. 1993); and Weir & Blackwell, eds., Handbook of Experimental Immunology, Volumes I-IV (5^{th} ed. 1996).

5.2. Biomarkers and Methods of Use Thereof

5.2.1 Genes (Biomarkers)

In one aspect, provided herein is a method of identifying a subject having cancer who is likely to be responsive to a treatment comprising a compound or predicting the responsiveness of a subject having or suspected of having cancer to a treatment comprising the compound, comprising: i. providing a sample from the subject; ii. measuring gene expression level of one or more gene in the sample; and iii. identifying the subject as being likely to be responsive to the treatment comprising the compound if the expression level of the gene is different from a reference level, wherein the compound is 2-(4-chlorophenyl)-N-((2-(2,6-dioxopiperidin-3-yl)-1-oxoisoindolin-5-yl)methyl)-2,2-difluoroacetamide (Compound D), or a stereoisomer or mixture of stereoisomers, isotopologue, pharmaceutically acceptable salt, tautomer, solvate, hydrate, co-crystal, clathrate, or polymorph thereof, and wherein the gene is a gene involved in mTOR signaling.

In another aspect, provided herein is a method of treating a subject having cancer with a compound, comprising identifying the subject having cancer that may be responsive to the treatment comprising the compound as described above and administering the subject a therapeutically effective amount of the compound if the subject is identified as being likely to be responsive to the treatment comprising the compound, wherein the compound is Compound D, or a stereoisomer or mixture of stereoisomers, isotopologue, pharmaceutically acceptable salt, tautomer, solvate, hydrate, co-crystal, clathrate, or polymorph thereof and wherein the gene is a gene involved in mTOR signaling.

The mammalian target of rapamycin (mTOR), also known as the mechanistic target of rapamycin, is a protein with serine-threonine kinase activity. mTOR belongs to the phospho-inositide 3-kinase (PI3K)-related kinase family. mTOR exists in two functionally distinct complexes: mTOR complex 1 (mTORC1) and mTOR complex 2 (mTORC2).

mTORC1 is comprised of five proteins. In addition to mTOR, complex 1 includes regulatory-associated protein of mTOR (Raptor), mammalian lethal with Sec13 protein 8 (mLST8, also known as GPL), proline-rich AKT substrate 40 kDa (PRAS40), and DEP-domain-containing mTOR-interacting protein (Deptor). Raptor is the major distinguishing protein of mTORC1. Raptor is an adaptor protein that positively regulates mTOR activity. mLST8 is a scaffolding protein that binds to and stabilizes the kinase domain of mTOR. Unlike Raptor and mLST8, PRAS40 and Deptor are negative regulators of mTOR. Thus, during mTOR activation and signaling, PRAS40 and Deptor are not physically associated with mTORC1. When mTOR activation and signaling is reduced, PRAS40 and Deptor are recruited to the complex.

mTORC2 is comprised of six proteins. In addition to mTOR, complex 2 includes rapamycin-insensitive companion of mTOR (Rictor), mammalian stress-activated protein kinase interacting protein (mSIN1), protein observed with Rictor-1 (Protor-1), mLST8, and Deptor. Both Rictor and mSIN1 are scaffolding proteins that stabilize interaction between each other and mTOR. The function of Protor-1, which binds Rictor, is less understood. Similar to mTORC1, mLST8 stabilizes and promotes the kinase activity of mTOR and Deptor negatively regulates mTORC2 activity.

mTOR signaling refers to a series or cascade of events (e.g., phosphorylation) that begin typically upstream of mTOR, leading to its activation. mTOR activation is the result of the formation of the mTOR complex or phosphorylation of mTOR and its associated proteins in the mTOR complex. For example, mTORC1 can be activated by phosphorylation of Raptor, which promotes mTORC1 assembly, or mTOR can be phosphorylated at known serine and threonine residues. Once the mTOR complex has been activated through assembly and phosphorylation, mTOR will phosphorylate downstream proteins and other cell components to promote metabolic changes or growth within the cell. Specifically, mTORC1 signaling activates downstream proteins and components important for lipid and nucleotide synthesis and protein synthesis via ribosome biogenesis and mRNA translation. mTORC2 signaling activates downstream proteins important for lipid and glucose metabolism, and cell survival.

Many intracellular and extracellular signals converge on mTOR to regulate mTOR signaling. These signals include growth factors, energy status, oxygen and amino acids.

Regulation of mTOR signaling can occur upstream of mTOR, it can be intrinsic to the mTOR complex, or regulation can occur downstream of mTOR signaling, e.g., via feedback loops. In normal cells, these signals can positively or negatively regulate mTOR activity. The positive and negative regulators of mTOR can affect mTOR activity in a number of ways. Exemplary positive regulators of mTORC1 include Raptor, mLST8 and Ras homolog enriched in brain (Rheb). Raptor and mLST8 are important scaffolding proteins required for mTOR activity. Knockout studies have confirmed that loss of Raptor or mLST8 impairs mTORC1 formation and kinase activity. Rheb is a GTPase that has been shown to promote mTORC1 activity by directly binding mTOR and mLST8 or by causing mTORC1 to be phosphorylated. It should be noted that while the above mentioned positive regulators are immediately proximate to mTOR, there are multiple upstream cell signals, including those interact directly or indirectly with these regulators. As an example, when a growth factor binds its corresponding receptor, a series of phosphorylation events initiated by PI3K will phosphorylate AKT, which will then lead to the phosphorylation and inactivation of the TSC1/TSC2, a negative regulator of mTOR activity.

Exemplary negative regulators of mTORC1 include Deptor, PRAS40, the tuberous sclerosis complex (TSC1/TSC2), DNA damage inducible transcript 4 (DDIT4), Activating Transcription Factor 4 (ATF4), general control nonderepressible 2 (GCN2), and 5′ adenosine monophosphate-activated protein kinase (AMPK). Deptor binds mTOR to inhibit its kinase activity while PRAS40 inhibits mTOR activity by binding Raptor. TSC1/TSC2 is a heterodimeric complex that inhibits mTORC1 activity by hydrolyzing the Rheb-GTP to its inactive GDP state. Rheb in its GDP state is unable to bind or phosphorylate mTOR. DDIT4 is a protein that is upregulated in the cell in response to stress. DDIT4 can negatively regulate mTOR by increasing the activity of TSC1 and TSC2 proteins. One understanding is that DDIT4 can compete to bind 14-3-3 protein, a protein that binds to and inhibits TSC2. Once DDIT4 binds 14-3-3 protein, the TSC1/TSC2 complex is free to inhibit mTOR activity. ATF4 is a transcription factor that, under stress conditions, will increase the expression of DDIT4. GCN2 is a serine-threonine kinase that can inhibit mTOR through the phosphorylation and inactivation of the translation initiation factor eIF2α, although the mechanism underlying this is not fully understood. Finally, AMPK can suppress mTOR activity in two ways. AMPK can phosphorylate and activate TSC2 or AMPK can phosphorylate Raptor, causing 14-3-3 protein to sequester it from the complex.

Studies have shown that mTORC2 is regulated in part through the Rictor component. Loss of Rictor in mice significantly hinders mTORC2 activity. Additionally, Ribosomal protein S6 kinase beta-1 (p70S6K), which is responsible for ribosome biogenesis and protein translation, can inhibit mTORC2 through phosphorylation of Rictor.

The term “positive regulator of mTOR signaling” as used herein refers to a factor that positively regulates mTOR signaling, including but not limited to by promoting or stimulating mTOR activation or promoting or stimulating the downstream events caused by mTOR activation. The term “negative regulator of mTOR signaling” as used herein refers to a factor that negatively regulates mTOR signaling, including but not limited to by inhibiting or decreasing mTOR activation or inhibiting or decreasing the downstream events caused by mTOR activation.

In some embodiments, the gene used in the present methods is a positive regulator of mTOR signaling, such as mTOR, Raptor, and Rictor. As shown in Section 6 below, knockout of any of these positive regulators of mTOR signaling enhances sensitivity of cancer cells to treatment with Compound D, indicating that lower level expression of such a gene can increase the responsiveness to Compound D treatment. Therefore, when the gene is a positive regulator of mTOR signaling, the method comprises identifying the subject as being likely to be responsive to the treatment comprising the compound if the expression level of the gene is lower than a reference level. In some embodiments, the reference level is the expression level of the gene in a subject resistant to Compound D. In other embodiments, the reference level is the expression level of the gene in a subject without the cancer. In yet other embodiments, the reference level is a pre-determined level, e.g., determined based on a population of subjects.

In other embodiments, the gene used in the present methods is a negative regulator of mTOR signaling, such as TSC1, TSC2, GCN1, GCN2, DDIT4, and ATF4. As shown in Section 6 below, knockout of any of these negative regulators of mTOR signaling confer resistance of cancer cells to treatment with Compound D, indicating that the presence or a higher level expression of such a gene can increase the responsiveness to Compound D treatment. Therefore, when the gene is a negative regulator of mTOR signaling, the method comprises identifying the subject as being likely to be responsive to the treatment comprising the compound if the expression level of the gene is higher than a reference level. In some embodiments, the reference level is the expression level of the gene in a subject resistant to Compound D. In some embodiments, the reference level is the expression level of the gene in a subject responsive to Compound D. In some embodiments, the reference level is the expression level of the gene in a subject without the cancer. In yet other embodiments, the reference level is a pre-determined level, e.g., determined based on a population of subjects.

In one aspect, provided herein is a method of identifying a subject having cancer who is likely to be responsive to a treatment comprising a compound or predicting the responsiveness of a subject having or suspected of having cancer to a treatment comprising the compound, comprising: i. providing a sample from the subject; ii. measuring gene expression level of one or more gene in the sample; and iii. identifying the subject as being likely to be responsive to the treatment comprising the compound if the expression level of the gene is different from a reference level, wherein the compound is 2-(4-chlorophenyl)-N-((2-(2,6-dioxopiperidin-3-yl)-1-oxoisoindolin-5-yl)methyl)-2,2-difluoroacetamide (Compound D), or a stereoisomer or mixture of stereoisomers, isotopologue, pharmaceutically acceptable salt, tautomer, solvate, hydrate, co-crystal, clathrate, or polymorph thereof, and wherein the gene is ILF2 or ILF3. In some embodiments, the gene is ILF2. In some embodiments, the gene is ILF3.

In another aspect, provided herein is a method of treating a subject having cancer with a compound, comprising identifying the subject having cancer that may be responsive to the treatment comprising the compound as described above and administering the subject a therapeutically effective amount of the compound if the subject is identified as being likely to be responsive to the treatment comprising the compound, wherein the compound is Compound D, or a stereoisomer or mixture of stereoisomers, isotopologue, pharmaceutically acceptable salt, tautomer, solvate, hydrate, co-crystal, clathrate, or polymorph thereof and wherein the gene is ILF2 or ILF3. In some embodiments, the gene is ILF2. In some embodiments, the gene is ILF3.

In some embodiments, the method comprises identifying the subject as being likely to be responsive to the treatment comprising the compound if the expression level of ILF2 or ILF3 is higher than a reference level. In some embodiments, the reference level is the expression level of the gene in a subject resistant to Compound D. In some embodiments, the reference level is the expression level of the gene in a subject responsive to Compound D. In some embodiments, the reference level is the expression level of the gene in a subject without the cancer. In yet other embodiments, the reference level is a pre-determined level, e.g., determined based on a population of subjects.

In some embodiments of the various methods provided herein, the cancer is a lymphoma. In other embodiments of the various methods provided herein, the cancer is a leukemia. In a specific embodiment, the cancer is AML.

5.2.2 Selective Treatments

In some embodiments of various methods provided herein (including those described above), a compound provided herein is administered to a patient that has been determined likely to be responsive to the compound. So in one aspect, provided herein is a selective treatment method comprising administering a compound to a patient that has been determined likely to be responsive to the compound based on the methods described here (including those described above).

In another particular embodiment, the compound is Compound D or a stereoisomer or mixture of stereoisomers, isotopologue, pharmaceutically acceptable salt, tautomer, solvate, hydrate, co-crystal, clathrate, or polymorph thereof.

In some embodiments of the various methods provided herein, a treatment compound is administered to a patient likely to be responsive to the treatment compound. Also provided herein are methods of treating patients who have been previously treated for cancer but are non-responsive to standard therapies, as well as those who have not previously been treated. The invention also encompasses methods of treating patients regardless of patient's age, although some diseases or disorders are more common in certain age groups. The invention further encompasses methods of treating patients who have undergone surgery in an attempt to treat the disease or condition at issue, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer.

Dosing and Administration

In certain embodiments, a therapeutically or prophylactically effective amount of the compound provided herein. In certain embodiments, a therapeutically or prophylactically effective amount of Compound D is from about 0.005 to about 20 mg per day, from about 0.05 to 20 mg per day, from about 0.01 to about 10 mg per day, from about 0.01 to about 7 mg per day, from about 0.01 to about 5 mg per day, from about 0.01 to about 3 mg per day, from about 0.05 to about 10 mg per day, from about 0.05 to about 7 mg per day, from about 0.05 to about 5 mg per day, from about 0.05 to about 3 mg per day, from about 0.1 to about 15 mg per day, from about 0.1 to about 10 mg per day, from about 0.1 to about 7 mg per day, from about 0.1 to about 5 mg per day, from about 0.1 to about 3 mg per day, from about 0.5 to about 10 mg per day, from about 0.05 to about 5 mg per day, from about 0.5 to about 3 mg per day, from about 0.5 to about 2 mg per day, from about 0.3 to about 10 mg per day, from about 0.3 to about 8.5 mg per day, from about 0.3 to about 8.1 mg per day, from about 0.6 to about 10 mg per day or from about 0.6 to about 5 mg per day. In one embodiment, a therapeutically or prophylactically effective amount of Compound D is from about 0.005 to about 20 mg per day. In one embodiment, a therapeutically or prophylactically effective amount of Compound D is, from about 0.05 to 20 mg per day. In one embodiment, a therapeutically or prophylactically effective amount of Compound D is from about 0.01 to about 10 mg per day. In one embodiment, a therapeutically or prophylactically effective amount of Compound D is from about 0.01 to about 7 mg per day. In one embodiment, a therapeutically or prophylactically effective amount of Compound D is from about 0.01 to about 5 mg per day. In one embodiment, a therapeutically or prophylactically effective amount of Compound D is from about 0.01 to about 3 mg per day. In one embodiment, a therapeutically or prophylactically effective amount of Compound D is from about 0.05 to about 10 mg per day. In one embodiment, a therapeutically or prophylactically effective amount of Compound D is from about 0.05 to about 7 mg per day. In one embodiment, a therapeutically or prophylactically effective amount of Compound D is from about 0.05 to about 5 mg per day. In one embodiment, a therapeutically or prophylactically effective amount of Compound D is from about 0.05 to about 3 mg per day. In one embodiment, a therapeutically or prophylactically effective amount of Compound D is from about 0.1 to about 15 mg per day. In one embodiment, a therapeutically or prophylactically effective amount of Compound D is from about 0.1 to about 10 mg per day. In one embodiment, a therapeutically or prophylactically effective amount of Compound D is from about 0.1 to about 7 mg per day. In one embodiment, a therapeutically or prophylactically effective amount of Compound D is from about 0.1 to about 5 mg per day. In one embodiment, a therapeutically or prophylactically effective amount of Compound D is from about 0.1 to about 3 mg per day. In one embodiment, a therapeutically or prophylactically effective amount of Compound D is from about 0.5 to about 10 mg per day. In one embodiment, a therapeutically or prophylactically effective amount of Compound D is from about 0.5 to about 5 mg per day. In one embodiment, a therapeutically or prophylactically effective amount of Compound D is from about 0.5 to about 3 mg per day. In one embodiment, a therapeutically or prophylactically effective amount of Compound D is from about 0.5 to about 2 mg per day. In one embodiment, a therapeutically or prophylactically effective amount of Compound D is from about 0.3 to about 10 mg per day. In one embodiment, a therapeutically or prophylactically effective amount of Compound D is from about 0.3 to about 8.5 mg per day. In one embodiment, a therapeutically or prophylactically effective amount of Compound D is from about 0.3 to about 8.1 mg per day. In one embodiment, a therapeutically or prophylactically effective amount of Compound D is from about 0.6 to about 10 mg per day or from about 0.6 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, or about 10 mg per day. In some such embodiments, the therapeutically or prophylactically effective amount is about 0.5, about 0.6, about 0.75, about 1, about 2, about 3, about 4, about 5, about 6 or about 7 mg per day. In some such embodiments, the therapeutically or prophylactically effective amount is about 0.6, about 1.2, about 1.8, about 2.4, or about 3.6 mg per day. In certain embodiments, the therapeutically or prophylactically effective amount is about 0.1 mg per day. In certain embodiments, the therapeutically or prophylactically effective amount is about 0.2 mg per day. In certain embodiments, the therapeutically or prophylactically effective amount is about 0.5 mg per day. In certain embodiments, the therapeutically or prophylactically effective amount is about 1 mg per day. In certain embodiments, the therapeutically or prophylactically effective amount is about 2 mg per day. In certain embodiments, the therapeutically or prophylactically effective amount is about 3 mg per day. In certain embodiments, the therapeutically or prophylactically effective amount is about 4 mg per day. In certain embodiments, the therapeutically or prophylactically effective amount is about 5 mg per day. In certain embodiments, the therapeutically or prophylactically effective amount is about 6 mg per day. In certain embodiments, the therapeutically or prophylactically effective amount is about 7 mg per day. In certain embodiments, the therapeutically or prophylactically effective amount is about 8 mg per day. In certain embodiments, the therapeutically or prophylactically effective amount is about 9 mg per day. In certain embodiments, the therapeutically or prophylactically effective amount is about 10 mg per day.

In one embodiment, the recommended daily dose range of Compound D, for the conditions described herein lie within the range of from about 0.01 mg to about 20 mg per day, preferably given as a single once-a-day dose, or in divided doses throughout a day. In one embodiment, the recommended daily dose range of Compound D, for the conditions described herein lie within the range of from about 0.01 mg to about 15 mg per day, preferably given as a single once-a-day dose, or in divided doses throughout a day. In one embodiment, the recommended daily dose range of Compound D, for the conditions described herein lie within the range of from about 0.01 mg to about 12 mg per day, preferably given as a single once-a-day dose, or in divided doses throughout a day. In some embodiments, the dosage ranges from about 0.1 mg to about 10 mg per day. In other embodiments, the dosage ranges from about 0.5 to about 5 mg per day. Specific doses per day include 0.1, 0.2, 0.5, 0.6, 1, 1.2, 1.5, 1.8, 2, 2.4, 2.5, 3, 3.5, 3.6, 4, 4.5, 5, 5.5, 6, 6.5, 7, 7.2, 7.5, 8, 8.5, 9, 9.5, 10, 10.5, 11, 11.5, 12, 12.5, 13, 13.5, 14, 14.4, 14.5 or 15 mg per day. In other embodiments, the dosage ranges from about 0.5 to about 5 mg per day. Specific doses per day include 0.1, 0.2, 0.5, 0.6, 1, 1.2, 1.5, 1.8, 2, 2.4, 2.5, 3, 3.5, 3.6, 4, 4.5, 5, 5.5, 6, 6.5, 7, 7.5, 8, 8.5, 9, 9.5 or 10 mg per day. In one embodiment, the dose per day is 0.1 mg per day. In one embodiment, the dose per day is 0.2 mg per day. In one embodiment, the dose per day is 0.5 mg per day. In one embodiment, the dose per day is 0.6 mg per day. In one embodiment, the dose per day is 1 mg per day. In one embodiment, the dose per day is 1.2 mg per day. In one embodiment, the dose per day is 1.5 mg per day. In one embodiment, the dose per day is 1.8 mg per day. In one embodiment, the dose per day is 2 mg per day. In one embodiment, the dose per day is 2.4 mg per day. In one embodiment, the dose per day is 2.5 mg per day. In one embodiment, the dose per day is 3 mg per day. In one embodiment, the dose per day is 3.5 mg per day. In one embodiment, the dose per day is 3.6 mg per day. In one embodiment, the dose per day is 4 mg per day. In one embodiment, the dose per day is 4.5 mg per day. In one embodiment, the dose per day is 5 mg per day. In one embodiment, the dose per day is 5.5 mg per day. In one embodiment, the dose per day is 6 mg per day. In one embodiment, the dose per day is 6.5 mg per day. In one embodiment, the dose per day is 7 mg per day. In one embodiment, the dose per day is 7.2 mg per day. In one embodiment, the dose per day is 7.5 mg per day. In one embodiment, the dose per day is 8 mg per day. In one embodiment, the dose per day is 8.5 mg per day. In one embodiment, the dose per day is 9 mg per day. In one embodiment, the dose per day is 9.5 mg per day. In one embodiment, the dose per day is 10 mg per day. In one embodiment, the dose per day is 12 mg per day. In one embodiment, the dose per day is 10 mg per day. In one embodiment, the dose per day is 12 mg per day. In one embodiment, the dose per day is 14.4 mg per day. In one embodiment, the dose per day is 15 mg per day.

In a specific embodiment, the recommended starting dosage may be 0.1, 0.5, 0.6, 0.7, 1, 1.2, 1.5, 1.8, 2, 2.4, 2.5, 3, 3.5, 3.6, 4, 4.5, 5, 5.5, 6, 6.5 or 7 mg per day. In another embodiment, the recommended starting dosage may be 0.1, 0.5, 0.6, 1, 1.2, 1.8, 2, 2.4, 3, 3.6, 4, or 5 mg per day. In one embodiment, the dose may be escalated to 7, 8, 9 10, 12, or 15 mg/day. In one embodiment, the dose may be escalated to 7, 8, 9 or 10 mg/day.

In a specific embodiment, Compound D can be administered in an amount of about 0.1 mg/day to patients with leukemia, including AML. In a particular embodiment, Compound D can be administered in an amount of about 1 mg/day to patients with leukemia, including AML. In a particular embodiment, Compound D can be administered in an amount of about 3 mg/day to patients with leukemia, including AML. In a particular embodiment, Compound D can be administered in an amount of about 4 mg/day to patients with leukemia, including AML. In a particular embodiment, Compound D provided herein can be administered in an amount of about 5 mg/day to patients with leukemia, including AML. In a particular embodiment, Compound D provided herein can be administered in an amount of about 6 mg/day to patients with leukemia, including AML. In a particular embodiment, Compound D provided herein can be administered in an amount of about 7 mg/day to patients with leukemia, including AML. In a particular embodiment, Compound D provided herein can be administered in an amount of about 10 mg/day to patients with leukemia, including AML. In a particular embodiment, Compound D provided herein can be administered in an amount of about 12 mg/day to patients with leukemia, including AML. In a particular embodiment, Compound D provided herein can be administered in an amount of about 15 mg/day to patients with leukemia, including AML.

In a specific embodiment, Compound D can be administered in an amount of about 0.1 mg/day to patients with MDS. In a particular embodiment, Compound D can be administered in an amount of about 1 mg/day to patients with MDS. In a particular embodiment, Compound D can be administered in an amount of about 3 mg/day to patients with MDS. In a particular embodiment, Compound D can be administered in an amount of about 4 mg/day to patients with MDS. In a particular embodiment, Compound D provided herein can be administered in an amount of about 5 mg/day to patients with MDS. In a particular embodiment, Compound D provided herein can be administered in an amount of about 6 mg/day to patients with MDS. In a particular embodiment, Compound D provided herein can be administered in an amount of about 7 mg/day to patients with MDS. In a particular embodiment, Compound D provided herein can be administered in an amount of about 10 mg/day to patients with MDS. In a particular embodiment, Compound D provided herein can be administered in an amount of about 12 mg/day to patients with MDS. In a particular embodiment, Compound D provided herein can be administered in an amount of about 15 mg/day to patients with MDS.

In certain embodiments, the therapeutically or prophylactically effective amount is from about 0.001 to about 20 mg/kg/day, from about 0.01 to about 15 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day. In certain embodiments, the therapeutically or prophylactically effective amount is from about 0.001 to about 20 mg/kg/day. In certain embodiments, the therapeutically or prophylactically effective amount is from about 0.01 to about 15 mg/kg/day. In certain embodiments, the therapeutically or prophylactically effective amount is from about 0.01 to about 10 mg/kg/day. In certain embodiments, the therapeutically or prophylactically effective amount is from about 0.01 to about 9 mg/kg/day. In certain embodiments, the therapeutically or prophylactically effective amount is 0.01 to about 8 mg/kg/day. In certain embodiments, the therapeutically or prophylactically effective amount is from about 0.01 to about 7 mg/kg/day. In certain embodiments, the therapeutically or prophylactically effective amount is from about 0.01 to about 6 mg/kg/day. In certain embodiments, the therapeutically or prophylactically effective amount is from about 0.01 to about 5 mg/kg/day. In certain embodiments, the therapeutically or prophylactically effective amount is from about 0.01 to about 4 mg/kg/day. In certain embodiments, the therapeutically or prophylactically effective amount is from about 0.01 to about 3 mg/kg/day. In certain embodiments, the therapeutically or prophylactically effective amount is from about 0.01 to about 2 mg/kg/day. In certain embodiments, the therapeutically or prophylactically effective amount is from about 0.01 to about 1 mg/kg/day. In certain embodiments, the therapeutically or prophylactically effective amount is from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of Compound D administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM. In certain embodiments, the amount of Compound D administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of a formulation of Compound D administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM. In other embodiments, the amount of a formulation of Compound D administered is sufficient to provide a plasma concentration of the compound at steady state, ranging from about 5 to about 100 nM. In other embodiments, the amount of a formulation of Compound D administered is sufficient to provide a plasma concentration of the compound at steady state, ranging from about 5 to about 50 nM. In other embodiments, the amount of a formulation of Compound D administered is sufficient to provide a plasma concentration of the compound at steady state, ranging from about 10 to about 100 nM. In other embodiments, the amount of a formulation of Compound D administered is sufficient to provide a plasma concentration of the compound at steady state, ranging from about 10 to about 50 nM. In other embodiments, the amount of a formulation of Compound D administered is sufficient to provide a plasma concentration of the compound at steady state, ranging from about 50 to about 100 nM.

As used herein, the term “plasma concentration at steady state” is the concentration reached after a period of administration of a formulation provided herein. Once steady state is reached, there are minor peaks and troughs on the time dependent curve of the plasma concentration of the solid form.

In certain embodiments, the amount of a formulation of Compound D administered is sufficient to provide a maximum plasma concentration (peak concentration) of the compound, ranging from about 0.001 to about 500 μM, about 0.002 to about 200 PM, about 0.005 to about 100 μM, about 0.01 to about 50 μM, from about 1 to about 50 PM, about 0.02 to about 25 μM, from about 0.05 to about 20 μM, from about 0.1 to about 20 μM, from about 0.5 to about 20 μM, or from about 1 to about 20 μM. In certain embodiments, the amount of a formulation of Compound D administered is sufficient to provide a maximum plasma concentration (peak concentration) of the compound, ranging from about 0.001 to about 500 μM. In certain embodiments, the amount of a formulation of Compound D administered is sufficient to provide a maximum plasma concentration (peak concentration) of the compound, ranging from about 0.002 to about 200 μM. In certain embodiments, the amount of a formulation of Compound D administered is sufficient to provide a maximum plasma concentration (peak concentration) of the compound, ranging from about 0.005 to about 100 μM. In certain embodiments, the amount of a formulation of Compound D administered is sufficient to provide a maximum plasma concentration (peak concentration) of the compound, ranging from about 0.01 to about 50 μM. In certain embodiments, the amount of a formulation of Compound D administered is sufficient to provide a maximum plasma concentration (peak concentration) of the compound, ranging from about 1 to about 50 μM. In certain embodiments, the amount of a formulation of Compound D administered is sufficient to provide a maximum plasma concentration (peak concentration) of the compound, ranging from about 0.02 to about 25 μM. In certain embodiments, the amount of a formulation of Compound D administered is sufficient to provide a maximum plasma concentration (peak concentration) of the compound, ranging from about 0.05 to about 20 μM. In certain embodiments, the amount of a formulation of Compound D administered is sufficient to provide a maximum plasma concentration (peak concentration) of the compound, ranging from about 0.1 to about 20 μM. In certain embodiments, the amount of a formulation of Compound D administered is sufficient to provide a maximum plasma concentration (peak concentration) of the compound, ranging from about 0.5 to about 20 μM. In certain embodiments, the amount of a formulation of Compound D administered is sufficient to provide a maximum plasma concentration (peak concentration) of the compound, ranging from about 1 to about 20 μM.

In certain embodiments, the amount of a formulation of Compound D administered is sufficient to provide a minimum plasma concentration (trough concentration) of the compound, ranging from about 0.001 to about 500 μM, about 0.002 to about 200 PM, about 0.005 to about 100 μM, about 0.01 to about 50 μM, from about 1 to about 50 PM, about 0.01 to about 25 μM, from about 0.01 to about 20 μM, from about 0.02 to about 20 μM, from about 0.02 to about 20 M, or from about 0.01 to about 20 μM. In certain embodiments, the amount of a formulation of Compound D administered is sufficient to provide a minimum plasma concentration (trough concentration) of the compound, ranging from about 0.001 to about 500 μM. In certain embodiments, the amount of a formulation of Compound D administered is sufficient to provide a minimum plasma concentration (trough concentration) of the compound, ranging from about 0.002 to about 200 μM. In certain embodiments, the amount of a formulation of Compound D administered is sufficient to provide a minimum plasma concentration (trough concentration) of the compound, ranging from about 0.005 to about 100 μM. In certain embodiments, the amount of a formulation of Compound D administered is sufficient to provide a minimum plasma concentration (trough concentration) of the compound, ranging from about 0.01 to about 50 PM. In certain embodiments, the amount of a formulation of Compound D administered is sufficient to provide a minimum plasma concentration (trough concentration) of the compound, ranging from about 1 to about 50 μM, about 0.01 to about 25 μM. In certain embodiments, the amount of a formulation of Compound D administered is sufficient to provide a minimum plasma concentration (trough concentration) of the compound, ranging from about 0.01 to about 20 PM. In certain embodiments, the amount of a formulation of Compound D administered is sufficient to provide a minimum plasma concentration (trough concentration) of the compound, ranging from about 0.02 to about 20 μM. In certain embodiments, the amount of a formulation of Compound D administered is sufficient to provide a minimum plasma concentration (trough concentration) of the compound, ranging from about 0.02 to about 20 μM. In certain embodiments, the amount of a formulation of Compound D administered is sufficient to provide a minimum plasma concentration (trough concentration) of the compound, ranging from about 0.01 to about 20 μM.

In certain embodiments, the amount of a formulation of Compound D administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL. In certain embodiments, the amount of a formulation of Compound D administered is sufficient to provide an area under the curve (AUC) of the compound, ranging from about 100 to about 100,000 ng*hr/mL. In certain embodiments, the amount of a formulation of Compound D administered is sufficient to provide an area under the curve (AUC) of the compound, ranging from about 1,000 to about 50,000 ng*hr/mL. In certain embodiments, the amount of a formulation of Compound D administered is sufficient to provide an area under the curve (AUC) of the compound, ranging from about 5,000 to about 25,000 ng*hr/mL. In certain embodiments, the amount of a formulation of Compound D administered is sufficient to provide an area under the curve (AUC) of the compound, ranging from about 5,000 to about 10,000 ng*hr/mL.

In certain embodiments, the patient to be treated with one of the methods provided herein has not been treated with anti-cancer therapy prior to the administration of a formulation of Compound D provided herein. In certain embodiments, the patient to be treated with one of the methods provided herein has been treated with anti-cancer therapy prior to the administration of a formulation of Compound D provided herein. In certain embodiments, the patient to be treated with one of the methods provided herein has developed drug resistance to the anti-cancer therapy.

The methods provided herein encompass treating a patient regardless of patient's age, although some diseases or disorders are more common in certain age groups.

The formulation of Compound D provided herein can be delivered as a single dose such as, e.g., a single bolus injection, or over time, such as, e.g., continuous infusion over time or divided bolus doses over time. The formulation of Compound D can be administered repeatedly if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The formulation of Compound D provided herein can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound is administered once or more than once each day, for example, for a period of time. The term “continuous” is intended to mean that a therapeutic compound is administered daily for an uninterrupted period of at least 10 days to 52 weeks. The term “intermittent” or “intermittently” as used herein is intended to mean stopping and starting at either regular or irregular intervals. For example, intermittent administration of the formulation of Compound D is administration for one to six days per week, administration in cycles (e.g., daily administration for one to ten consecutive days of a 28 day cycle, then a rest period with no administration for rest of the 28 day cycle; or daily administration for two to eight consecutive weeks, then a rest period with no administration for up to one week), or administration on alternate days. Cycling therapy with Compound D is discussed elsewhere herein.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, Compound D is administered once a day. In another embodiment, Compound D is administered twice a day. In yet another embodiment, Compound D provided herein is administered three times a day. In still another embodiment, Compound D provided herein is administered four times a day. In still another embodiment, Compound D provided herein is administered once every other day. In still another embodiment, Compound D provided herein is administered twice a week. In still another embodiment, Compound D provided herein is administered once every week. In still another embodiment, Compound D provided herein is administered once every two weeks. In still another embodiment, Compound D provided herein is administered once every three weeks. In still another embodiment, Compound D provided herein is administered once every four weeks.

In certain embodiments, a formulation of Compound D provided herein is administered once per day from one day to six months, from one week to three months, from one week to four weeks, from one week to three weeks, or from one week to two weeks. In certain embodiments, a formulation of Compound D provided herein is administered once per day for one week, two weeks, three weeks, or four weeks. In one embodiment, a formulation of Compound D provided herein is administered once per day for 1 day. In one embodiment, a formulation of Compound D provided herein is administered once per day for 2 days. In one embodiment, a formulation of Compound D provided herein is administered once per day for 3 days. In one embodiment, a formulation of Compound D provided herein is administered once per day for 4 days. In one embodiment, a formulation of Compound D provided herein is administered once per day for 5 days. In one embodiment, a formulation of Compound D provided herein is administered once per day for 6 days. In one embodiment, a formulation of Compound D provided herein is administered once per day for one week. In one embodiment, a formulation of Compound D provided herein is administered once per day for up to 10 days. In another embodiment, a formulation of Compound D provided herein is administered once per day for two weeks. In yet another embodiment, a formulation of Compound D provided herein is administered once per day for three weeks. In still another embodiment, a formulation of Compound D provided herein is administered once per day for four weeks.

Combination Therapy

In one embodiment, provided herein is a method of treating, preventing, and/or managing cancer, comprising administering to a patient Compound D in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors, and optionally in combination with radiation therapy, blood transfusions, or surgery. Examples of second active agents are disclosed herein.

In one embodiment, provided herein is a method of treating, preventing, and/or managing cancer, comprising administering to a patient a formulation of Compound D provided herein in combination with one or more second active agents, and optionally in combination with radiation therapy, blood transfusions, or surgery. Examples of second active agents are disclosed herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. E.g., “in combination” may include administration as a mixture, simultaneous administration using separate formulations, and consecutive administration in any order. “Consecutive” means that a specific time has passed between the administration of the active agents. For example, “consecutive” may be that more than 10 minutes have passed between the administration of the separate active agents. The time period can then be more than 10 min, more than 30 minutes, more than 1 hour, more than 3 hours, more than 6 hours or more than 12 hours. E.g., a first therapy (e.g., a prophylactic or therapeutic agent such as a formulation of Compound D provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

In one embodiment, administration of Compound D, including a formulation of Compound D provided herein, and one or more second active agents to a patient can occur simultaneously or sequentially by the same or different routes of administration. In one embodiment, administration of Compound D, including a formulation of Compound D provided herein, and one or more second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the cancer being treated.

The route of administration of Compound D, including a formulation of Compound D provided herein, is independent of the route of administration of a second therapy. Thus, in one embodiment, Compound D, including a formulation of Compound D provided herein, is administered intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, Compound D, including a formulation of Compound D provided herein, and a second therapy are administered by the same mode of administration, by IV. In another embodiment, Compound D, including a formulation of Compound D provided herein, is administered by one mode of administration, e.g., by IV, whereas the second agent (an anti-cancer agent) is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated and/or managed, the severity and stage of disease, and the amount of Compound D and any optional additional active agents concurrently administered to the patient.

One or more second active ingredients or agents can be used together with Compound D in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, particularly, therapeutic antibodies to cancer antigens. Typical large molecule active agents are biological molecules, such as naturally occurring or synthetic or recombinant proteins. Proteins that are particularly useful in the methods and compositions provided herein include proteins that stimulate the survival and/or proliferation of hematopoietic precursor cells and immunologically active poietic cells in vitro or in vivo. Other useful proteins stimulate the division and differentiation of committed erythroid progenitors in cells in vitro or in vivo. Particular proteins include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; GM-CF and GM-CSF; and EPO.

In certain embodiments, GM-CSF, G-CSF, SCF or EPO is administered subcutaneously during about five days in a four or six week cycle in an amount ranging from about 1 to about 750 mg/m²/day, from about 25 to about 500 mg/m²/day, from about 50 to about 250 mg/m²/day, or from about 50 to about 200 mg/m²/day. In certain embodiments, GM-CSF may be administered in an amount of from about 60 to about 500 mcg/m² intravenously over 2 hours or from about 5 to about 12 mcg/m²/day subcutaneously. In certain embodiments, G-CSF may be administered subcutaneously in an amount of about 1 mcg/kg/day initially and can be adjusted depending on rise of total granulocyte counts. The maintenance dose of G-CSF may be administered in an amount of about 300 (in smaller patients) or 480 mcg subcutaneously. In certain embodiments, EPO may be administered subcutaneously in an amount of 10,000 Unit 3 times per week.

Particular proteins that can be used in the methods and compositions include, but are not limited to: filgrastim, which is sold in the United States under the trade name Neupogen® (Amgen, Thousand Oaks, Calif.); sargramostim, which is sold in the United States under the trade name Leukine® (Immunex, Seattle, Wash.); and recombinant EPO, which is sold in the United States under the trade name Epogen® (Amgen, Thousand Oaks, Calif.).

Recombinant and mutated forms of GM-CSF can be prepared as described in U.S. Pat. Nos. 5,391,485; 5,393,870; and 5,229,496; all of which are incorporated herein by reference. Recombinant and mutated forms of G-CSF can be prepared as described in U.S. Pat. Nos. 4,810,643; 4,999,291; 5,528,823; and 5,580,755; the entireties of which are incorporated herein by reference.

Also provided for use in combination with Compound D, including a formulation of Compound D, are native, naturally occurring, and recombinant proteins. Further encompassed are mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet, M. L. and Morrison, S. L., J. Immunol. Methods 248:91-101 (2001).

Antibodies that can be used in combination with Compound D, including a formulation of Compound D provided herein, include monoclonal and polyclonal antibodies. Examples of antibodies include, but are not limited to, trastuzumab (Herceptin®), rituximab (Rituxan®), bevacizumab (Avastin™), pertuzumab (Omnitarg™), tositumomab (Bexxar®), edrecolomab (Panorex®), and G250. The formulation of Compound D can also be combined with, or used in combination with, anti-TNF-α antibodies, and/or anti-EGFR antibodies, such as, for example, Erbitux® or panitumumab.

Large molecule active agents may be administered in the form of anti-cancer vaccines. For example, vaccines that secrete, or cause the secretion of, cytokines such as IL-2, G-CSF, and GM-CSF can be used in the methods and pharmaceutical compositions provided. See, e.g., Emens, L. A., et al., Curr. Opinion Mol. Ther. 3(1):77-84 (2001).

Second active agents that are small molecules can also be used to alleviate adverse effects associated with the administration of a formulation of Compound D provided herein. However, like some large molecules, many are believed to be capable of providing a synergistic effect when administered with (e.g., before, after, or simultaneously) Compound D, including a formulation of Compound D provided herein. Examples of small molecule second active agents include, but are not limited to, anti-cancer agents, antibiotics, immunosuppressive agents, and steroids.

In certain embodiments, the second agent is an HSP inhibitor, a proteasome inhibitor, a FLT3 inhibitor or an mTOR inhibitor. In some embodiments, the mTOR inhibitor is an mTOR kinase inhibitor.

Examples of anti-cancer agents to be used within the methods or compositions described herein include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; clofarabine; crisnatol mesylate; cyclophosphamide; Ara-C; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; omacetaxine; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sorafenib; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs to be included within the methods herein include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; Ara-C ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®); imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosane polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In certain embodiments, the second agent is selected from one or more checkpoint inhibitors. In one embodiment, one checkpoint inhibitor is used in combination with Compound D or a formulation of Compound D in the methods provided herein. In another embodiment, two checkpoint inhibitors are used in combination with Compound D or a formulation of Compound D in connection with the methods provided herein. In yet another embodiment, three or more checkpoint inhibitors are used in combination with Compound D or a formulation of Compound D in connection with the methods provided herein.

As used herein, the term “immune checkpoint inhibitor” or “checkpoint inhibitor” refers to molecules that totally or partially reduce, inhibit, interfere with or modulate one or more checkpoint proteins. Without being limited by a particular theory, checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In one embodiment, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™

In one embodiment, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In one embodiment, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity.

In one embodiment, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®).

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitor is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitor is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is an IDO inhibitor. In one embodiment, the IDO inhibitor is INCB024360. In another embodiment, the IDO inhibitor is indoximod.

In certain embodiments, the combination therapies provided herein include two or more of the checkpoint inhibitors described herein (including checkpoint inhibitors of the same or different class). Moreover, the combination therapies described herein can be used in combination with second active agents as described herein where appropriate for treating diseases described herein and understood in the art.

In certain embodiments, Compound D can be used in combination with one or more immune cells expressing one or more chimeric antigen receptors (CARs) on their surface (e.g., a modified immune cell). Generally, CARs comprise an extracellular domain from a first protein e.g., an antigen-binding protein), a transmembrane domain, and an intracellular signaling domain. In certain embodiments, once the extracellular domain binds to a target protein such as a tumor-associated antigen (TAA) or tumor-specific antigen (TSA), a signal is generated via the intracellular signaling domain that activates the immune cell, e.g., to target and kill a cell expressing the target protein.

Extracellular domains: The extracellular domains of the CARs bind to an antigen of interest. In certain embodiments, the extracellular domain of the CAR comprises a receptor, or a portion of a receptor, that binds to said antigen. In certain embodiments, the extracellular domain comprises, or is, an antibody or an antigen-binding portion thereof. In specific embodiments, the extracellular domain comprises, or is, a single chain Fv (scFv) domain. The single-chain Fv domain can comprise, for example, a VL linked to VH by a flexible linker, wherein said VL and VH are from an antibody that binds said antigen.

In certain embodiments, the antigen recognized by the extracellular domain of a polypeptide described herein is a tumor-associated antigen (TAA) or a tumor-specific antigen (TSA). In various specific embodiments, the tumor-associated antigen or tumor-specific antigen is, without limitation, Her2, prostate stem cell antigen (PSCA), alpha-fetoprotein (AFP), carcinoembryonic antigen (CEA), cancer antigen-125 (CA-125), CA19-9, calretinin, MUC-1, B cell maturation antigen (BCMA), epithelial membrane protein (EMA), epithelial tumor antigen (ETA), tyrosinase, melanoma-24 associated antigen (MAGE), CD19, CD22, CD27, CD30, CD34, CD45, CD70, CD99, CD 117, EGFRvIII (epidermal growth factor variant III), mesothelin, PAP (prostatic acid phosphatase), prostein, TARP (T cell receptor gamma alternate reading frame protein), Trp-p8, STEAPI (six-transmembrane epithelial antigen of the prostate 1), chromogranin, cytokeratin, desmin, glial fibrillary acidic protein (GFAP), gross cystic disease fluid protein (GCDFP-15), HMIB-45 antigen, protein melan-A (melanoma antigen recognized by T lymphocytes; MART-I), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein. In certain other embodiments, the TAA or TSA recognized by the extracellular domain of a CAR is integrin avP3 (CD61), galactin, or Ral-B.

In certain embodiments, the TAA or TSA recognized by the extracellular domain of a CAR is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXBI, SPA17, SSX, SYCPI, or TPTE.

In certain other embodiments, the TAA or TSA recognized by the extracellular domain of a CAR is a carbohydrate or ganglioside, e.g., fuc-GMI, GM2 (oncofetal antigen-immunogenic-1; OFA-I-1); GD2 (OFA-I-2), GM3, GD3, and the like.

In certain other embodiments, the TAA or TSA recognized by the extracellular domain of a CAR is alpha-actinin-4, Bage-1, BCR-ABL, Bcr-Abl fusion protein, beta-catenin, CA 125, CA 15-3 (CA 27.29BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-All, hsp70-2, KIAA0205, Mart2, Mum-1, 2, and 3, neo-PAP, myosin class I, OS-9, pml-RARa fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel17), tyrosinase, TRP-1, TRP-2, MAGE-1, MAGE-3, RAGE, GAGE-1, GAGE-2, p15(58), RAGE, SCP-1, Hom/Mel-40, PRAME, p53, HRas, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, or TPS.

In various specific embodiments, the tumor-associated antigen or tumor-specific antigen is an AML-related tumor antigen, as described in S. Anguille et al, Leukemia (2012), 26, 2186-2196.

Other tumor-associated and tumor-specific antigens are known to those in the art.

Receptors, antibodies, and scFvs that bind to TSAs and TAAs, useful in constructing chimeric antigen receptors, are known in the art, as are nucleotide sequences that encode them.

In certain specific embodiments, the antigen recognized by the extracellular domain of a chimeric antigen receptor is an antigen not generally considered to be a TSA or a TAA, but which is nevertheless associated with tumor cells, or damage caused by a tumor. In certain embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the antigen is a DAMP, e.g., a heat shock protein, chromatin-associated protein high mobility group box 1 (HMGB 1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

Transmembrane domain: In certain embodiments, the extracellular domain of the CAR is joined to the transmembrane domain of the polypeptide by a linker, spacer or hinge polypeptide sequence, e.g., a sequence from CD28 or a sequence from CTLA4. The transmembrane domain can be obtained or derived from the transmembrane domain of any transmembrane protein, and can include all or a portion of such transmembrane domain. In specific embodiments, the transmembrane domain can be obtained or derived from, e.g., CD8, CD16, a cytokine receptor, and interleukin receptor, or a growth factor receptor, or the like.

Intracellular signaling domains: In certain embodiments, the intracellular domain of a CAR is or comprises an intracellular domain or motif of a protein that is expressed on the surface of T cells and triggers activation and/or proliferation of said T cells. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3( ) or ITAM-containing portions thereof. In a specific embodiment, the intracellular domain is a CD3(intracellular signaling domain. In other specific embodiments, the intracellular domain is from a lymphocyte receptor chain, a TCR/CD3 complex protein, an Fe receptor subunit or an IL-2 receptor subunit. In certain embodiments, the CAR additionally comprises one or more co-stimulatory domains or motifs, e.g., as part of the intracellular domain of the polypeptide. The one or more co-stimulatory domains or motifs can be, or can comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif, or any combination thereof.

The CAR may also comprise a T cell survival motif. The T cell survival motif can be any polypeptide sequence or motif that facilitates the survival of the T lymphocyte after stimulation by an antigen. In certain embodiments, the T cell survival motif is, or is derived from, CD3, CD28, an intracellular signaling domain of IL-7 receptor (IL-7R), an intracellular signaling domain of IL-12 receptor, an intracellular signaling domain of IL-15 receptor, an intracellular signaling domain of IL-21 receptor, or an intracellular signaling domain of transforming growth factor β (TGFβ) receptor.

The modified immune cells expressing the CARs can be, e.g., T lymphocytes (T cells, e.g., CD4+ T cells or CD8+ T cells), cytotoxic lymphocytes (CTLs) or natural killer (NK) cells. T lymphocytes used in the compositions and methods provided herein may be naive T lymphocytes or MHC-restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T cells have been isolated from, or are expanded from T lymphocytes isolated from, peripheral blood, cord blood, or lymph. Immune cells to be used to generate modified immune cells expressing a CAR can be isolated using art-accepted, routine methods, e.g., blood collection followed by apheresis and optionally antibody-mediated cell isolation or sorting.

The modified immune cells are preferably autologous to an individual to whom the modified immune cells are to be administered. In certain other embodiments, the modified immune cells are allogeneic to an individual to whom the modified immune cells are to be administered. Where allogeneic T lymphocytes or NK cells are used to prepare modified T lymphocytes, it is preferable to select T lymphocytes or NK cells that will reduce the possibility of graft-versus-host disease (GVHD) in the individual. For example, in certain embodiments, virus-specific T lymphocytes are selected for preparation of modified T lymphocytes; such lymphocytes will be expected to have a greatly reduced native capacity to bind to, and thus become activated by, any recipient antigens. In certain embodiments, recipient-mediated rejection of allogeneic T lymphocytes can be reduced by co-administration to the host of one or more immunosuppressive agents, e.g., cyclosporine, tacrolimus, sirolimus, cyclophosphamide, or the like.

T lymphocytes, e.g., unmodified T lymphocytes, or T lymphocytes expressing CD3 and CD28, or comprising a polypeptide comprising a CD3(signaling domain and a CD28 co-stimulatory domain, can be expanded using antibodies to CD3 and CD28, e.g., antibodies attached to beads; see, e.g., U.S. Pat. Nos. 5,948,893; 6,534,055; 6,352,694; 6,692,964; 6,887,466; and 6,905,681.

The modified immune cells, e.g., modified T lymphocytes, can optionally comprise a “suicide gene” or “safety switch” that enables killing of substantially all of the modified immune cells when desired. For example, the modified T lymphocytes, in certain embodiments, can comprise an HSV thymidine kinase gene (HSV-TK), which causes death of the modified T lymphocytes upon contact with gancyclovir. In another embodiment, the modified T lymphocytes comprise an inducible caspase, e.g., an inducible caspase 9 (icaspase9), e.g., a fusion protein between caspase 9 and human FK506 binding protein allowing for dimerization using a specific small molecule pharmaceutical. See Straathof et al., Blood 105(11):4247-4254 (2005).

Specific second active agents useful in the methods or compositions include, but are not limited to, rituximab, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, interferon alpha, pegylated interferon alpha (e.g., PEG INTRON-A), capecitabine, cisplatin, thiotepa, fludarabine, carboplatin, liposomal daunorubicin, Ara-C, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

In certain embodiments of the methods provided herein, use of a second active agent in combination with Compound D, including a formulation of Compound D provided herein, may be modified or delayed during or shortly following administration of Compound D, including a formulation of Compound D provided herein, as deemed appropriate by the practitioner of skill in the art. In certain embodiments, subjects being administered Compound D, including a formulation of Compound D provided herein, alone or in combination with other therapies may receive supportive care including antiemetics, myeloid growth factors, and transfusions of platelets, when appropriate. In some embodiments, subjects being administered Compound D, including a formulation of Compound D provided herein, may be administered a growth factor as a second active agent according to the judgment of the practitioner of skill in the art. In some embodiments, provided is administration of Compound D, including a formulation of Compound D provided herein, in combination with erythropoietin or darbepoetin (Aranesp).

In one aspect, provided herein is a method of treating, preventing, managing, and/or ameliorating locally advanced or metastatic transitional cell bladder cancer comprising administering a formulation of Compound D with gemcitabine, cisplatinum, 5-fluorouracil, mitomycin, methotrexate, vinblastine, doxorubicin, carboplatin, thiotepa, paclitaxel, docetaxel, atezolizumab, avelumab, durvalumab, keytruda (pembrolizumab) and/or nivolumab.

In one aspect, methods of treating, preventing, managing, and/or ameliorating a cancer provided herein comprise administering a formulation of Compound D in combination with a second active ingredient as follows: temozolomide to pediatric patients with relapsed or progressive brain tumors or recurrent neuroblastoma; celecoxib, etoposide and cyclophosphamide for relapsed or progressive CNS cancer; temodar to patients with recurrent or progressive meningioma, malignant meningioma, hemangiopericytoma, multiple brain metastases, relapsed brain tumors, or newly diagnosed glioblastoma multiforme; irinotecan to patients with recurrent glioblastoma; carboplatin to pediatric patients with brain stem glioma; procarbazine to pediatric patients with progressive malignant gliomas; cyclophosphamide to patients with poor prognosis malignant brain tumors, newly diagnosed or recurrent glioblastoma multiforme; Gliadel® for high grade recurrent malignant gliomas; temozolomide and tamoxifen for anaplastic astrocytoma; or topotecan for gliomas, glioblastoma, anaplastic astrocytoma or anaplastic oligodendroglioma.

In one aspect, methods of treating, preventing, managing, and/or ameliorating a metastatic breast cancer provided herein comprise administering a formulation of Compound D with methotrexate, cyclophosphamide, capecitabine, 5-fluorouracil, taxane, temsirolimus, ABRAXANE® (paclitaxel protein-bound particles for injectable suspension) (albumin-bound), lapatinib, herceptin, pamidronate disodium, eribulin mesylate, everolimus, gemcitabine, palbociclib, ixabepilone, kadcyla, pertuzumab, theotepa, anastrozole, docetaxel, doxorubicin hydrochloride, epirubicin hydrochloride, toremifene, fulvestrant, goserelin acetate, ribociclib, megestrol acetate, vinblastin, aromatase inhibitors, such as letrozole, exemestane, selective estrogen modulators, estrogen receptor antagonists, anthracyclines, emtansine, and/or pexidartinib to patients with metastatic breast cancer.

In one aspect, methods of treating, preventing, managing, and/or ameliorating neuroendocrine tumors provided herein comprise administering a formulation of Compound D with at least one of everolimus, avelumab, sunitinib, nexavar, leucovorin, oxaliplatin, temozolomide, capecitabine, bevacizumab, doxorubicin (Adriamycin), fluorouracil (Adrucil, 5-fluorouracil), streptozocin (Zanosar), dacarbazine, sandostatin, lanreotide, and/or pasireotide to patients with neuroendocrine tumors.

In one aspect, methods of treating, preventing, managing, and/or ameliorating a metastatic breast cancer provided herein comprise administering a formulation of Compound D with methotrexate, gemcitabine, cisplatin, cetuximab, 5-fluorouracil, bleomycin, docetaxel, carboplatin, hydroxyurea, pembrolizumab and/or nivolumab to patients with recurrent or metastatic head or neck cancer.

In one aspect, methods of treating, preventing, managing, and/or ameliorating a pancreatic cancer provided herein comprise administering a formulation of Compound D with gemcitabine, ABRAXANE®, 5-fluorouracil, afinitor, irinotecan, mitomycin C, sunitinib, sunitinibmalate, and/or tarceva to patients with pancreatic cancer.

In one aspect, methods of treating, preventing, managing, and/or ameliorating a colon or rectal cancer provided herein comprise administering a formulation of Compound D with ARISA®, avastatin, oxaliplatin, 5-fluorouracil, irinotecan, capecitabine, cetuximab, ramucirumab, panitumumab, bevacizumab, leucovorin calcium, lonsurf, regorafenib, ziv-aflibercept, taxol, and/or taxotere.

In one aspect, methods of treating, preventing, managing, and/or ameliorating a refractory colorectal cancer provided herein comprise administering a formulation of Compound D with capecitabine and/or vemurafenib to patients with refractory colorectal cancer, or patients who fail first line therapy or have poor performance in colon or rectal adenocarcinoma.

In one aspect, methods of treating, preventing, managing, and/or ameliorating a colorectal cancer provided herein comprise administering a formulation of Compound D with fluorouracil, leucovorin, and/or irinotecan to patients with colorectal cancer, including stage 3 and stage 4, or to patients who have been previously treated for metastatic colorectal cancer.

In certain embodiments, a formulation of Compound D provided herein is administered to patients with refractory colorectal cancer in combination with capecitabine, xeloda, and/or irinotecan.

In certain embodiments, a formulation of Compound D provided herein is administered with capecitabine and irinotecan to patients with refractory colorectal cancer or to patients with unresectable or metastatic colorectal carcinoma.

In one aspect, the methods provided herein comprise administering a formulation of Compound D with interferon alpha or capecitabine to patients with unresectable or metastatic hepatocellular carcinoma; or with cisplatin and thiotepa, or with sorafenib tosylate to patients with primary or metastatic liver cancer.

In one aspect, the methods provided herein comprise administering a formulation of Compound D with doxorubicin, paclitaxel, vinblastine, pegylated interferon alpha and/or recombinant interferon alpha-2b to patients with Kaposi's sarcoma.

In one aspect, the methods provided herein comprise administering a formulation of Compound D with at least one of enasidenib, arsenic trioxide, fludarabine, carboplatin, daunorubicin, cyclophosphamide, cytarabine, doxorubicin, idarubicin, mitoxantrone hydrochloride, thioguanine, vincristine, midostaurin and/or topotecan to patients with acute myeloid leukemia, including refractory or relapsed or high-risk acute myeloid leukemia.

In one aspect, the methods provided herein comprise administering a formulation of Compound D with at least one of enasidenib, liposomal daunorubicin, topotecan and/or cytarabine to patients with unfavorable karyotype acute myeloblastic leukemia.

In one aspect, the methods provided herein comprise administering Compound D with an IDH2 inhibitor to a patient having leukemia, wherein the leukemia is characterized by the presence of a mutant allele of IDH2. Exemplary IDH2 inhibitors are disclosed in U.S. Pat. Nos. 9,732,062; 9,724,350; 9,738,625; and 9,579,324; and US Publication Nos. 2016-0159771and US 2016-0158230 A1. In one aspect, the methods provided herein comprise administering Compound D with enasidenib to a patient having leukemia, wherein the leukemia is characterized by the presence of a mutant allele of IDH2. In certain embodiments, the combination of Compound D and an IDH2 inhibitor increases differentiated cells (CD34−/CD38) and erythroblasts in a patient having acute myeloid leukemia, wherein the acute myeloid leukemia is characterized by the presence of IDH2 R140Q. In certain embodiments, the combination of Compound D and an IDH2 inhibitor reduces progenitor cells (CD34+/CD38+) and HSC in a patient having acute myeloid leukemia, wherein the acute myeloid leukemia is characterized by the presence of IDH2 R140Q.

In one aspect, the methods provided herein comprise administering Compound D with enasidenib to a patient having acute myeloid leukemia, wherein the acute myeloid leukemia is characterized by the presence of a mutant allele of IDH2. In one embodiment, the mutant allele of IDH2 is IDH2 R140Q or R172K.

In one aspect, the methods provided herein comprise administering a formulation of Compound D with enasidenib to a patient having leukemia, wherein the leukemia is characterized by the presence of a mutant allele of IDH2. In one aspect, the methods provided herein comprise administering a formulation of Compound D with enasidenib to a patient having acute myeloid leukemia, wherein the acute myeloid leukemia is characterized by the presence of a mutant allele of IDH2. In one embodiment, the mutant allele of IDH2 is IDH2 R140Q or R172K.

In one aspect, the methods provided herein comprise administering Compound D with 6-(6-(trifluoromethyl)pyridin-2-yl)-N2-(2-(trifluoromethyl)pyridin-4-yl)-1,3,5-triazine-2,4-diamine (Compound 2) to a patient having leukemia, wherein the leukemia is characterized by the presence of a mutant allele of IDH2. In one aspect, the methods provided herein comprise administering Compound D with Compound 2 to a patient having acute myeloid leukemia, wherein the acute myeloid leukemia is characterized by the presence of a mutant allele of IDH2. In one embodiment, the mutant allele of IDH2 is IDH2 R140Q or R172K.

In one aspect, the methods provided herein comprise administering a formulation of Compound D with Compound 2 to a patient having leukemia, wherein the leukemia is characterized by the presence of a mutant allele of IDH2. In one aspect, the methods provided herein comprise administering a formulation of Compound D with Compound 2 to a patient having acute myeloid leukemia, wherein the acute myeloid leukemia is characterized by the presence of a mutant allele of IDH2. In one embodiment, the mutant allele of IDH2 is IDH2 R140Q or R172K.

In one aspect, the methods provided herein comprise administering a formulation of Compound D with methotrexate, mechlorethamine hydrochloride, afatinib dimaleate, pemetrexed, bevacizumab, carboplatin, cisplatin, ceritinib, crizotinib, ramucirumab, pembrolizumab, docetaxel, vinorelbine tartrate, gemcitabine, ABRAXANE®, erlotinib, geftinib, irinotecan, everolimus, alectinib, brigatinib, nivolumab, osimertinib, atezolizumab, necitumumab and/or to patients with non-small cell lung cancer.

In one aspect, the methods provided herein comprise administering a formulation of Compound D with carboplatin and irinotecan to patients with non-small cell lung cancer.

In one aspect, the methods provided herein comprise administering a formulation of Compound D with doxetaxol to patients with non-small cell lung cancer who have been previously treated with carbo/etoposide and radiotherapy.

In one aspect, the methods provided herein comprise administering a formulation of Compound D with carboplatin and/or taxotere, or in combination with carboplatin, pacilitaxel and/or thoracic radiotherapy to patients with non-small cell lung cancer.

In one aspect, the methods provided herein comprise administering a formulation of Compound D with taxotere to patients with stage IIIB or IV non-small cell lung cancer.

In one aspect, the methods provided herein comprise administering a formulation of Compound D with oblimersen (Genasense®), methotrexate, mechlorethamine hydrochloride, etoposide, topotecan and/or doxorubicin to patients with small cell lung cancer.

In one aspect, the methods provided herein comprise administering a formulation of Compound D with Venetoclax, ABT-737 (Abbott Laboratories) and/or obatoclax (GX15-070) to patients with lymphoma and other blood cancers.

In one aspect, the methods provided herein comprise administering a formulation of Compound D with a second active ingredient such as vinblastine or fludarabine adcetris, ambochlorin, becenum, bleomycin, brentuximab vedotin, carmustinem chlorambucil, cyclophosphamide, dacarbazine, doxorubicin, lomustine, matulane, mechlorethamine hydrochloride, prednisone, procarbazine hydrochloride, vincristine, methotrexate, nelarabin, belinostat, bendamustine HCl, tositumomab, and iodine 131 tositumomab, denileukin diftitox, dexamethasone, pralatrexate, prelixafor, obinutuzumab, ibritumomab, tiuxefan, ibritinib, idelasib, intron A, romidepsin, lenalidomide, rituximab, and/or vorinostat to patients with various types of lymphoma, including, but not limited to, Hodgkin's lymphoma, non-Hodgkin's lymphoma, cutaneous T-Cell lymphoma, cutaneous B-Cell lymphoma, diffuse large B-Cell lymphoma or relapsed or refractory low grade follicular lymphoma.

In one aspect, the methods provided herein comprise administering a formulation of Compound D with taxotere, dabrafenib, imlygic, ipilimumab, pembrolizumab, nivolumab, trametinib, vemurafenib, talimogene laherparepvec, IL-2, IFN, GM-CSF, and/or dacarbazine, aldesleukin, cobimetinib, Intron A®, peginterferon Alfa-2b, and/or trametinib to patients with various types or stages of melanoma.

In one aspect, the methods provided herein comprise administering a formulation of Compound D with vinorelbine or pemetrexed disodium to patients with malignant mesothelioma, or stage IIIB non-small cell lung cancer with pleural implants or malignant pleural effusion mesothelioma syndrome.

In one aspect, the methods of treating patients with various types or stages of multiple myeloma provided herein comprise administering a formulation of Compound D with dexamethasone, zoledronic acid, palmitronate, GM-CSF, biaxin, vinblastine, melphalan, busulphan, cyclophosphamide, IFN, prednisone, bisphosphonate, celecoxib, arsenic trioxide, PEG INTRON-A, vincristine, becenum, bortezomib, carfilzomib, doxorubicin, panobinostat, lenalidomide, pomalidomide, thalidomide, mozobil, carmustine, daratumumab, elotuzumab, ixazomib citrate, plerixafor or a combination thereof.

In certain embodiments, a formulation of Compound D provided herein is administered to patients with various types or stages of multiple myeloma in combination with chimeric antigen receptor (CAR) T-cells. In certain embodiments the CAR T cell in the combination targets B cell maturation antigen (BCMA), and in more specific embodiments, the CAR T cell is bb2121 or bb21217. In some embodiments, the CAR T cell is JCARH125.

In certain embodiments, a formulation of Compound D provided herein is administered to patients with relapsed or refractory multiple myeloma in combination with doxorubicin (Doxil®), vincristine and/or dexamethasone (Decadron®).

In certain embodiments, the methods provided herein comprise administering a formulation of Compound D to patients with various types or stages of ovarian cancer such as peritoneal carcinoma, papillary serous carcinoma, refractory ovarian cancer or recurrent ovarian cancer, in combination with taxol, carboplatin, doxorubicin, gemcitabine, cisplatin, xeloda, paclitaxel, dexamethasone, avastin, cyclophosphamide, topotecan, olaparib, thiotepa, melphalan, niraparib tosylate monohydrate, rubraca or a combination thereof.

In certain embodiments, the methods provided herein comprise administering a formulation of Compound D to patients with various types or stages of prostate cancer, in combination with xeloda, 5 FU/LV, gemcitabine, irinotecan plus gemcitabine, cyclophosphamide, vincristine, dexamethasone, GM-CSF, celecoxib, taxotere, ganciclovir, paclitaxel, adriamycin, docetaxel, estramustine, Emcyt, denderon, zytiga, bicalutamide, cabazitaxel, degarelix, enzalutamide, zoladex, leuprolide acetate, mitoxantrone hydrochloride, prednisone, sipuleucel-T, radium 223 dichloride, or a combination thereof.

In certain embodiments, the methods provided herein comprise administering a formulation of Compound D to patients with various types or stages of renal cell cancer, in combination with capecitabine, IFN, tamoxifen, IL-2, GM-CSF, Celebrex®, flutamide, goserelin acetate, nilutamide or a combination thereof.

In certain embodiments, the methods provided herein comprise administering a formulation of Compound D to patients with various types or stages of gynecologic, uterus or soft tissue sarcoma cancer in combination with IFN, dactinomycin, doxorubicin, imatinib mesylate, pazopanib, hydrochloride, trabectedin, eribulin mesylate, olaratumab, a COX-2 inhibitor such as celecoxib, and/or sulindac.

In one aspect, the methods provided herein comprise administering a formulation of Compound D to patients with various types or stages of solid tumors in combination with celecoxib, etoposide, cyclophosphamide, docetaxel, apecitabine, IFN, tamoxifen, IL-2, GM-CSF, or a combination thereof.

In one aspect, the methods provided herein comprise administering a formulation of Compound D to patients with scleroderma or cutaneous vasculitis in combination with celebrex, etoposide, cyclophosphamide, docetaxel, apecitabine, IFN, tamoxifen, IL-2, GM-CSF, or a combination thereof.

In one aspect, the methods provided herein comprise administering a formulation of Compound D to patients with MDS in combination with azacitidine, cytarabine, daunorubicin, decitabine, idarubicin, lenalidomide, enasidenib, or a combination thereof.

In one aspect, the methods provided herein comprise administering Compound D to patients with hematological cancer in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors. In one aspect, the methods provided herein comprise administering a formulation of Compound D to patients with a hematological cancer in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one aspect, the methods provided herein comprise administering Compound D to patients with leukemia in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors. In certain embodiments, a formulation of Compound D provided herein is administered to patients with leukemia in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one aspect, the methods provided herein comprise administering Compound D to patients with AML in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors. In certain embodiments, a formulation of Compound D provided herein is administered to patients with AML in combination with one or more second agents selected from JAK inhibitors, FLT3 inhibitors, mTOR inhibitors, spliceosome inhibitors, BET inhibitors, SMG1 inhibitors, ERK inhibitors, LSD1 inhibitors, BH3 mimetics, topoisomerase inhibitors, and RTK inhibitors.

In one aspect, the methods provided herein comprise administering Compound D to patients with leukemia in combination with an mTOR inhibitor. In certain embodiments, a formulation of Compound D provided herein is administered to patients with leukemia in combination with an mTOR inhibitor. In certain embodiments, the mTOR inhibitor is selected from everolimus, MLN-0128 and AZD8055. In some embodiments, the mTOR inhibitor is an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, Compound D is administered to patients with leukemia in combination with 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223). In certain embodiments, Compound D is administered to patients with leukemia in combination with 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, Compound D is administered to patients with leukemia in combination with everolimus. In certain embodiments, Compound D is administered to patients with leukemia in combination with MLN-0128. In certain embodiments, Compound D is administered to patients with leukemia in combination with AZD8055.

In one aspect, the methods provided herein comprise administering Compound D to patients with AML in combination with an mTOR inhibitor. In certain embodiments, a formulation of Compound D provided herein is administered to patients with AML in combination with an mTOR inhibitor. In certain embodiments, the mTOR inhibitor is selected from everolimus, MLN-0128 and AZD8055. In some embodiments, the mTOR inhibitor is an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In certain embodiments, Compound D is administered to patients with AML in combination with 1-ethyl-7-(2-methyl-6-(1H-1,2,4-triazol-3-yl)pyridin-3-yl)-3,4-dihydropyrazino[2,3-b]pyrazin-2(1H)-one. In certain embodiments, Compound D is administered to patients with AML in combination with everolimus. In certain embodiments, everolimus is administered to patients with AML prior to administration of Compound D. In certain embodiments, Compound D is administered to patients with AML in combination with MLN-0128. In certain embodiments, Compound D is administered to patients with AML in combination with AZD8055.

In one aspect, the methods provided herein comprise administering Compound D to patients with MPN in combination with a JAK inhibitor. In certain embodiments, a formulation of Compound D provided herein is administered to patients with MPN in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, filgotinib, decernotinib, barcitinib, ruxolitinib, fedratinib, NS-018 and pacritinib. In certain embodiments, the JAK inhibitor is selected from tofacitinib, momelotinib, ruxolitinib, fedratinib, NS-018 and pacritinib. In certain embodiments, Compound D is administered to patients with MPN in combination with tofacitinib. In certain embodiments, Compound D is administered to patients with MPN in combination with momelotinib. In certain embodiments, Compound D is administered to patients with MPN in combination with filgotinib. In certain embodiments, Compound D is administered to patients with MPN in combination with decernotinib. In certain embodiments, Compound D is administered to patients with MPN in combination with barcitinib. In certain embodiments, Compound D is administered to patients with MPN in combination with ruxolitinib. In certain embodiments, Compound D is administered to patients with MPN in combination with fedratinib. In certain embodiments, Compound D is administered to patients with MPN in combination with NS-018. In certain embodiments, Compound D is administered to patients with MPN in combination with pacritinib. In certain embodiments, the MPN is IL-3 independent. In certain embodiments, the MPN is characterized by a JAK 2 mutation, for example, a JAK2V617F mutation.

In one aspect, the methods provided herein comprise administering Compound D to patients with myelofibrosis in combination with a JAK inhibitor. In certain embodiments, a formulation of Compound D provided herein is administered to patients with myelofibrosis in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, ruxolitinib, fedratinib, NS-018 and pacritinib. In certain embodiments, Compound D is administered to patients with myelofibrosis in combination with tofacitinib. In certain embodiments, Compound D is administered to patients with myelofibrosis in combination with momelotinib. In certain embodiments, Compound D is administered to patients with myelofibrosis in combination with ruxolitinib. In certain embodiments, Compound D is administered to patients with myelofibrosis in combination with fedratinib. In certain embodiments, Compound D is administered to patients with myelofibrosis in combination with NS-018. In certain embodiments, Compound D is administered to patients with myelofibrosis in combination with pacritinib. In certain embodiments, the myeolofibrosis is characterized by a JAK 2 mutation, for example, a JAK2V617F mutation. In some embodiments, the myelofibrosis is primary myelofibrosis. In other embodiments, the myelofibrosis is secondary myelofibrosis. In some such embodiments, the secondary myelofibrosis is post polycythemia vera myelofibrosis. In other embodiments, the secondary myelofibrosis is post essential thrombocythemia myelofibrosis.

In one aspect, the methods provided herein comprise administering Compound D to patients with leukemia in combination with a JAK inhibitor. In certain embodiments, a formulation of Compound D provided herein is administered to patients with leukemia in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, filgotinib, decernotinib, barcitinib, ruxolitinib, fedratinib, NS-018 and pacritinib. In certain embodiments, the JAK inhibitor is selected from momelotinib, ruxolitinib, fedratinib, NS-018 and pacritinib. In certain embodiments, Compound D is administered to patients with leukemia in combination with tofacitinib. In certain embodiments, Compound D is administered to patients with leukemia in combination with momelotinib. In certain embodiments, Compound D is administered to patients with leukemia in combination with filgotinib. In certain embodiments, Compound D is administered to patients with leukemia in combination with decernotinib. In certain embodiments, Compound D is administered to patients with leukemia in combination with barcitinib. In certain embodiments, Compound D is administered to patients with leukemia in combination with ruxolitinib. In certain embodiments, Compound D is administered to patients with leukemia in combination with fedratinib. In certain embodiments, Compound D is administered to patients with leukemia in combination with NS-018. In certain embodiments, Compound D is administered to patients with leukemia in combination with pacritinib. In certain embodiments, the MPN is characterized by a JAK 2 mutation, for example, a JAK2V617F mutation.

In one aspect, the methods provided herein comprise administering Compound D to patients with AML in combination with a JAK inhibitor. In certain embodiments, a formulation of Compound D provided herein is administered to patients with AML in combination with a JAK inhibitor. In one aspect the JAK inhibitor is selected from a JAK1 inhibitor, a JAK2 inhibitor and a JAK3 inhibitor. In certain embodiments, the JAK inhibitor is selected from tofacitinib, momelotinib, filgotinib, decernotinib, barcitinib, ruxolitinib, fedratinib, NS-018 and pacritinib. In certain embodiments, the JAK inhibitor is selected from momelotinib, ruxolitinib, fedratinib, NS-018 and pacritinib. In certain embodiments, Compound D is administered to patients with AML in combination with tofacitinib. In certain embodiments, Compound D is administered to patients with AML in combination with momelotinib. In certain embodiments, Compound D is administered to patients with AML in combination with filgotinib. In certain embodiments, Compound D is administered to patients with AML in combination with decernotinib. In certain embodiments, Compound D is administered to patients with AML in combination with barcitinib. In certain embodiments, Compound D is administered to patients with AML in combination with ruxolitinib. In certain embodiments, Compound D is administered to patients with AML in combination with fedratinib. In certain embodiments, Compound D is administered to patients with AML in combination with NS-018. In certain embodiments, Compound D is administered to patients with AML in combination with pacritinib. In certain embodiments, the MPN is characterized by a JAK 2 mutation, for example, a JAK2V617F mutation.

In one aspect, the methods provided herein comprise administering Compound D to patients with leukemia in combination with a FLT3 kinase inhibitor. In certain embodiments, a formulation of Compound D provided herein is administered to patients with leukemia in combination with a FLT3 kinase inhibitor. In certain embodiments, the FLT3 kinase inhibitor is selected from quizartinib, sunitinib, sunitinib malate, midostaurin, pexidartinib, lestaurtinib, tandutinib, and crenolanib. In certain embodiments, Compound D is administered to patients with leukemia in combination with quizartinib. In certain embodiments, Compound D is administered to patients with leukemia in combination with sunitinib. In certain embodiments, Compound D is administered to patients with leukemia in combination with midostaurin. In certain embodiments, Compound D is administered to patients with leukemia in combination with pexidartinib. In certain embodiments, Compound D is administered to patients with leukemia in combination with lestaurtinib. In certain embodiments, Compound D is administered to patients with leukemia in combination with tandutinib. In certain embodiments, Compound D is administered to patients with leukemia in combination with crenolanib. In certain embodiments, the patient carries a FLT3-ITD mutation.

In one aspect, the methods provided herein comprise administering Compound D to patients with AML in combination with a FLT3 kinase inhibitor. In certain embodiments, a formulation of Compound D provided herein is administered to patients with AML in combination with a FLT3 kinase inhibitor. In certain embodiments, the FLT3 kinase inhibitor is selected from quizartinib, sunitinib, sunitinib malate, midostaurin, pexidartinib, lestaurtinib, tandutinib, quizartinib and crenolanib. In certain embodiments, Compound D is administered to patients with AML in combination with quizartinib. In certain embodiments, Compound D is administered to patients with AML in combination with sunitinib. In certain embodiments, Compound D is administered to patients with AML in combination with midostaurin. In certain embodiments, Compound D is administered to patients with AML in combination with pexidartinib. In certain embodiments, Compound D is administered to patients with AML in combination with lestaurtinib. In certain embodiments, Compound D is administered to patients with AML in combination with tandutinib. In certain embodiments, Compound D is administered to patients with AML in combination with crenolanib. In certain embodiments, the patient carries a FLT3-ITD mutation.

In certain embodiments, Compound D is administered to patients with leukemia in combination with a spliceosome inhibitor. In certain embodiments, Compound D is administered to patients with AML in combination with a spliceosome inhibitor. In certain embodiments, the spliceosome inhibitor is pladienolide B, 6-deoxypladienolide D, or H3B-8800.

In one aspect, the methods provided herein comprise administering Compound D to patients with leukemia in combination with an SMG1 kinase inhibitor. In certain embodiments, a formulation of Compound D provided herein is administered to patients with leukemia in combination with an SMG1 kinase inhibitor. In one aspect, the methods provided herein comprise administering Compound D to patients with AML in combination with an SMG1 kinase inhibitor. In certain embodiments, a formulation of Compound D provided herein is administered to patients with AML in combination with an SMG1 kinase inhibitor. In certain embodiments, the SMG1 inhibitor is 1-ethyl-7-(2-methyl-6-(1H-1,2,4-triazol-3-yl)pyridin-3-yl)-3,4-dihydropyrazino[2,3-b]pyrazin-2(1H)-one, chloro-N,N-diethyl-5-((4-(2-(4-(3-methylureido)phenyl)pyridin-4-yl)pyrimidin-2-yl)amino)benzenesulfonamide (compound Ii), or a compound disclosed in A. Gopalsamy et al, Bioorg. Med Chem Lett. 2012, 22:6636-66412 (for example, chloro-N,N-diethyl-5-((4-(2-(4-(3-methylureido)phenyl)pyridin-4-yl)pyrimidin-2-yl)amino)benzenesulfonamide.

In one aspect, the methods provided herein comprise administering Compound D to patients with leukemia in combination with a BCL2 inhibitor. In certain embodiments, a formulation of Compound D provided herein is administered to patients with leukemia in combination with a BCL2 inhibitor. In certain embodiments, Compound D is administered to patients with AML in combination with a BCL2 inhibitor. In certain embodiments, a formulation of Compound D provided herein is administered to patients with AML in combination with a BCL2 inhibitor, for example, venetoclax or navitoclax. In certain embodiments, the BCL2 inhibitor is venetoclax.

In one embodiment, provided herein is a method for treating of AML that is resistant to treatment with a BCL2 inhibitor, comprising administering Compound D. In one embodiment, provided herein is a method for treating of AML that has acquired resistance to venetoclax treatment, comprising administering Compound D. In one embodiment, provided herein is a method for treating of AML that has acquired resistance to venetoclax treatment, comprising administering a combination of Compound D and a BCL2 inhibitor. In one embodiment, provided herein is a method for treating of AML that has acquired resistance to venetoclax treatment, comprising administering a combination of Compound D and venetoclax.

In one aspect, the methods provided herein comprise administering Compound D to patients with leukemia in combination with a topoisomerase inhibitor. In certain embodiments, a formulation of Compound D provided herein is administered to patients with leukemia in combination with a topoisomerase inhibitor. In certain embodiments, Compound D is administered to patients with AML in combination with a topoisomerase inhibitor. In certain embodiments, a formulation of Compound D provided herein is administered to patients with AML in combination with a topoisomerase inhibitor, for example, irinotecan, topotecan, camptothecin, lamellarin D, etoposide, teniposide, doxorubicin, daunorubicin, mitoxantrone, amsacrine, ellipticines, aurintricarboxylic acid, or HU-331. In certain embodiments, the topoisomerase inhibitor is topotecan.

In certain embodiments, Compound D is administered to patients with leukemia in combination with a BET inhibitor. In certain embodiments, Compound D is administered to patients with AML in combination with a BET inhibitor. In certain embodiments, the BET inhibitor is selected from GSK525762A, OTX015, BMS-986158, TEN-010, CPI-0610, INCB54329, BAY1238097, FT-1101, C90010, ABBV-075, BI 894999, GS-5829, GSK1210151A (I-BET-151), CPI-203, RVX 208, XD46, MS436, PFI-1, RVX2135, ZEN3365, XD14, ARV-771, MZ-1, PLX5117, 4-[2-(cyclopropylmethoxy)-5-(methanesulfonyl)phenyl]-2-methylisoquinolin-1(2H)-one (Compound A), EP11313 and EP11336.

In certain embodiments, Compound D is administered to patients with leukemia in combination with an LSD1 inhibitor. In certain embodiments, Compound D is administered to patients with AML in combination with an LSD1 inhibitor. In certain embodiments, the LSD1 inhibitor is selected from ORY-1001, ORY-2001, INCB-59872, IMG-7289, TAK 418, GSK-2879552, and 4-[2-(4-amino-piperidin-1-yl)-5-(3-fluoro-4-methoxy-phenyl)-1-methyl-6-oxo-1,6-dihydropyrimidin-4-yl]-2-fluoro-benzonitrile or a salt thereof (e.g. besylate salt, Compound B).

In one aspect, the methods provided herein comprise administering Compound D to patients with leukemia in combination with triptolide, retaspimycin, alvespimycin, 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223), 1-ethyl-7-(2-methyl-6-(1H-1,2,4-triazol-3-yl)pyridin-3-yl)-3,4-dihydropyrazino[2,3-b]pyrazin-2(1H)-one (CC-115), rapamycin, MLN-0128, everolimus, AZD8055, pladienolide B, topotecan, thioguanine, mitoxantrone, etoposide, decitabine, daunorubicin, clofarabine, cladribine, 6-mercaptopurine, chloro-N,N-diethyl-5-((4-(2-(4-(3-methylureido)phenyl)pyridin-4-yl)pyrimidin-2-yl)amino)benzenesulfonamide (compound Ii), fedratinib, sunitinib, pexidartinib, midostaurin, lestaurtinib, momelotinib, quizartinib, and crenolanib.

In one aspect, the methods provided herein comprise administering Compound D to patients with AML in combination with triptolide, retaspimycin, alvespimycin, 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223), 1-ethyl-7-(2-methyl-6-(1H-1,2,4-triazol-3-yl)pyridin-3-yl)-3,4-dihydropyrazino[2,3-b]pyrazin-2(1H)-one (CC-115), rapamycin, MLN-0128, everolimus, AZD8055, pladienolide B, topotecan, thioguanine, mitoxantrone, etoposide, decitabine, daunorubicin, clofarabine, cladribine, 6-mercaptopurine, chloro-N,N-diethyl-5-((4-(2-(4-(3-methylureido)phenyl)pyridin-4-yl)pyrimidin-2-yl)amino)benzenesulfonamide (compound Ii), fedratinib, sunitinib, pexidartinib, midostaurin, lestaurtinib, momelotinib, quizartinib, and crenolanib.

In one aspect, the methods provided herein comprise administering Compound D to patients with cancer in combination with an mTOR inhibitor, wherein the cancer is selected from breast cancer, kidney cancer, pancreatic cancer, gastrointestinal cancer, lung cancer, neuroendocrine tumor (NET), and renal cell carcinoma (RCC). In certain embodiments, a formulation of Compound D provided herein is administered to patients with cancer in combination with a topoisomerase inhibitor. In certain embodiments, a formulation of Compound D provided herein is administered to cancer patients in combination with an mTOR inhibitor, wherein the cancer is selected from breast cancer, kidney cancer, pancreatic cancer, gastrointestinal cancer, lung cancer, neuroendocrine tumor (NET), and renal cell carcinoma. In certain embodiments, the mTOR inhibitor is selected from everolimus, MLN-0128 and AZD8055. In some embodiments, the mTOR inhibitor is an mTOR kinase inhibitor. In certain embodiments, the mTOR kinase inhibitor is selected from 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223) and 1-ethyl-7-(2-methyl-6-(1H-1,2,4-triazol-3-yl)pyridin-3-yl)-3,4-dihydropyrazino[2,3-b]pyrazin-2(1H)-one (CC-115). In one embodiment, the mTOR kinase inhibitor is 7-(6-(2-hydroxypropan-2-yl)pyridin-3-yl)-1-((trans)-4-methoxycyclohexyl)-3,4-dihydropyrazino[2,3-b]pyrazin-2(1H)-one (CC-223). In one embodiment, the mTOR kinase inhibitor is 1-ethyl-7-(2-methyl-6-(1H-1,2,4-triazol-3-yl)pyridin-3-yl)-3,4-dihydropyrazino[2,3-b]pyrazin-2(1H)-one (CC-115). In one embodiment, the mTOR inhibitor is everolimus. In one embodiment, the mTOR inhibitor is temsirolimus. In one embodiment, the mTOR inhibitor is MLN-0128. In one embodiment, the mTOR inhibitor is AZD8055.

In certain embodiments, Compound D is administered to breast cancer patients in combination with everolimus. In certain embodiments, a formulation of Compound D provided herein is administered to breast cancer patients in combination with everolimus.

In certain embodiments, Compound D is administered to kidney cancer patients in combination with everolimus. In certain embodiments, a formulation of Compound D provided herein is administered to kidney cancer patients in combination with everolimus.

In certain embodiments, Compound D is administered to pancreatic cancer patients in combination with everolimus. In certain embodiments, a formulation of Compound D provided herein is administered to pancreatic cancer patients in combination with everolimus.

In certain embodiments, Compound D is administered to gastrointestinal cancer patients in combination with everolimus. In certain embodiments, a formulation of Compound D provided herein is administered to gastrointestinal cancer patients in combination with everolimus.

In certain embodiments, Compound D is administered to lung cancer patients in combination with everolimus. In certain embodiments, a formulation of Compound D provided herein is administered to lung cancer patients in combination with everolimus.

In certain embodiments, Compound D is administered to neuroendocrine tumor patients in combination with everolimus. In certain embodiments, a formulation of Compound D provided herein is administered to neuroendocrine tumor patients in combination with everolimus.

In certain embodiments, Compound D is administered to renal cell carcinoma patients in combination with everolimus. In certain embodiments, a formulation of Compound D provided herein is administered to renal cell carcinoma patients in combination with everolimus.

Also encompassed herein is a method of increasing the dosage of an anti-cancer drug or agent that can be safely and effectively administered to a patient, which comprises administering to the patient (e.g., a human) Compound D, for example, a formulation of Compound D provided herein in combination with the second anti-cancer drug. Patients that can benefit by this method are those likely to suffer from an adverse effect associated with anti-cancer drugs for treating a specific cancer of the skin, subcutaneous tissue, lymph nodes, brain, lung, liver, bone, intestine, colon, heart, pancreas, adrenal, kidney, prostate, breast, colorectal, or combinations thereof. The administration of Compound D, for example, a formulation of Compound D provided herein, alleviates or reduces adverse effects which are of such severity that it would otherwise limit the amount of anti-cancer drug.

Also encompassed herein is a method of decreasing the dosage of an anti-cancer drug or agent that can be safely and effectively administered to a patient, which comprises administering to the patient (e.g., a human) Compound D, for example, a formulation of Compound D provided herein in combination with the second anti-cancer drug. Patients that can benefit by this method are those likely to suffer from an adverse effect associated with anti-cancer drugs for treating a specific cancer of the skin, subcutaneous tissue, lymph nodes, brain, lung, liver, bone, intestine, colon, heart, pancreas, adrenal, kidney, prostate, breast, colorectal, or combinations thereof. The administration of Compound D, for example, a formulation of Compound D provided herein, potentiates the activity of the anti-cancer drug, which allows for a reduction in dose of the anti-cancer drug while maintaining efficacy, which in turn can alleviate or reduce the adverse effects which are of such severity that it limited the amount of anti-cancer drug.

In one embodiment, Compound D is administered daily in an amount ranging from about 0.1 to about 20 mg, from about 1 to about 15 mg, from about 1 to about 10 mg, or from about 1 to about 15 mg prior to, during, or after the occurrence of the adverse effect associated with the administration of an anti-cancer drug to a patient. In certain embodiments, Compound D is administered in combination with specific agents such as heparin, aspirin, coumadin, or G-CSF to avoid adverse effects that are associated with anti-cancer drugs such as but not limited to neutropenia or thrombocytopenia.

In one embodiment, Compound D, for example, a formulation of Compound D provided herein, is administered to patients with diseases and disorders associated with or characterized by, undesired angiogenesis in combination with additional active ingredients, including, but not limited to, anti-cancer drugs, anti-inflammatories, antihistamines, antibiotics, and steroids.

In another embodiment, encompassed herein is a method of treating, preventing and/or managing cancer, which comprises administering Compound D, for example, a formulation of Compound D provided herein, in conjunction with (e.g. before, during, or after) at least one anti-cancer therapy including, but not limited to, surgery, immunotherapy, biological therapy, radiation therapy, or other non-drug based therapy presently used to treat, prevent and/or manage cancer. The combined use of the compound provided herein and other anti-cancer therapy may provide a unique treatment regimen that is unexpectedly effective in certain patients. Without being limited by theory, it is believed that Compound D may provide additive or synergistic effects when given concurrently with at least one anti-cancer therapy.

As discussed elsewhere herein, encompassed herein is a method of reducing, treating and/or preventing adverse or undesired effects associated with other anti-cancer therapy including, but not limited to, surgery, chemotherapy, radiation therapy, hormonal therapy, biological therapy and immunotherapy. Compound D, for example, a formulation of Compound D provided herein, and other active ingredient can be administered to a patient prior to, during, or after the occurrence of the adverse effect associated with other anti-cancer therapy.

In certain embodiments, the methods provided herein comprise administration of one or more of calcium, calcitriol, or vitamin D supplementation with Compound D. In certain embodiments, the methods provided herein comprise administration of calcium, calcitriol, and vitamin D supplementation prior to the treatment with Compound D. In certain embodiments, the methods provided herein comprise administration of calcium, calcitriol, and vitamin D supplementation prior to the administration of first dose of Compound D in each cycle. In certain embodiments, the methods provided herein comprise administration of calcium, calcitriol, and vitamin D supplementation at least up to 3 days prior to the treatment with Compound D. In certain embodiments, the methods provided herein comprise administration of calcium, calcitriol, and vitamin D supplementation prior to the administration of first dose of Compound D in each cycle. In certain embodiments, the methods provided herein comprise administration of calcium, calcitriol, and vitamin D supplementation at least up to 3 days prior to the administration of first dose of Compound D in each cycle. In certain embodiments, the methods provided herein comprise administration of calcium, calcitriol, and vitamin D supplementation prior to administration of first dose of Compound D in each cycle and continues after administration of the last dose of Compound D in each cycle. In certain embodiments, the methods provided herein comprise administration of calcium, calcitriol, and vitamin D supplementation at least up to 3 days prior to administration of first dose of Compound D in each cycle and continues until at least up to 3 days after administration of the last dose of Compound D in each cycle (e.g., at least up to day 8 when Compound D is administered on Days 1-5). In one embodiment, the methods provided herein comprise administration of calcium, calcitriol, and vitamin D supplementation at least up to 3 days prior to administration of day 1 of each cycle and continue until ≥3 days after the last dose of Compound D in each cycle (eg, ≥Day 8 when Compound D is administered on Days 1-5, ≥Day 13 when Compound D is administered on Days 1-3 and Days 8-10).

In certain embodiments, calcium supplementation is administered to deliver at least 1200 mg of elemental calcium per day given in divided doses. In certain embodiments, calcium supplementation is administered as calcium carbonate in a dose of 500 mg administered three times a day per orally (PO).

In certain embodiments, calcitriol supplementation is administered to deliver 0.25 μg calcitriol (PO) once daily.

In certain embodiments, vitamin D supplementation is administered to deliver about 500 IU to about 50,000 IU vitamin D once daily. In certain embodiments, vitamin D supplementation is administered to deliver about 1000 IU vitamin D once daily. In certain embodiments, vitamin D supplementation is administered to deliver about 50,000 IU vitamin D weekly. In certain embodiments, vitamin D supplementation is administered to deliver about 1000 IU vitamin D2 or D3 once daily. In certain embodiments, vitamin D supplementation is administered to deliver about 500 IU vitamin D once daily. In certain embodiments, vitamin D supplementation is administered to deliver about 50,000 IU vitamin D weekly. In certain embodiments, vitamin D supplementation is administered to deliver about 20,000 IU vitamin D weekly. In certain embodiments, vitamin D supplementation is administered to deliver about 1000 IU vitamin D2 or D3 once daily. In certain embodiments, vitamin D supplementation is administered to deliver about 50,000 IU vitamin D2 or D3 weekly. In certain embodiments, vitamin D supplementation is administered to deliver about 20,000 IU vitamin D2 or D3 weekly.

In certain embodiments, a formulation of Compound D provided herein and doxetaxol are administered to patients with non-small cell lung cancer who were previously treated with carbo/VP 16 and radiotherapy.

Use With Transplantation Therapy

Compound D, for example, a formulation of Compound D provided herein, can be used to reduce the risk of Graft Versus Host Disease (GVHD). Therefore, encompassed herein is a method of treating, preventing and/or managing cancer, which comprises administering Compound D, for example, a formulation of Compound D provided herein, in conjunction with transplantation therapy.

As those of ordinary skill in the art are aware, the treatment of cancer is often based on the stages and mechanism of the disease. For example, as inevitable leukemic transformation develops in certain stages of cancer, transplantation of peripheral blood stem cells, hematopoietic stem cell preparation or bone marrow may be necessary. The combined use of Compound D, for example, a formulation of Compound D provided herein, and transplantation therapy provides a unique and unexpected synergism. In particular, a formulation of Compound D provided herein exhibits immunomodulatory activity that may provide additive or synergistic effects when given concurrently with transplantation therapy in patients with cancer.

Compound D, for example, a formulation of Compound D provided herein, can work in combination with transplantation therapy reducing complications associated with the invasive procedure of transplantation and risk of GVHD. Encompassed herein is a method of treating, preventing and/or managing cancer which comprises administering to a patient (e.g., a human) formulation of Compound D provided herein before, during, or after the transplantation of umbilical cord blood, placental blood, peripheral blood stem cell, hematopoietic stem cell preparation, or bone marrow. Some examples of stem cells suitable for use in the methods provided herein are disclosed in U.S. Pat. No. 7,498,171, the disclosure of which is incorporated herein by reference in its entirety.

In one embodiment, Compound D, for example, a formulation of Compound D provided herein, is administered to patients with acute myeloid leukemia before, during, or after transplantation.

In one embodiment, Compound D, for example, a formulation of Compound D provided herein, is administered to patients with multiple myeloma before, during, or after the transplantation of autologous peripheral blood progenitor cells.

In one embodiment, Compound D, for example, a formulation of Compound D provided herein, is administered to patients with NHL (e.g., DLBCL) before, during, or after the transplantation of autologous peripheral blood progenitor cells.

Cycling Therapy

In certain embodiments, Compound D, for example, a formulation of Compound D provided herein, are cyclically administered to a patient independent of the cancer treated. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improve the efficacy of the treatment.

In certain embodiments, Compound D, for example, a formulation of Compound D provided herein, is administered daily in a single or divided dose in a four to six week cycle with a rest period of about a week or two weeks. In certain embodiments, Compound D, for example, a formulation of Compound D provided herein, is administered daily in a single or divided doses for one to ten consecutive days of a 28 day cycle, then a rest period with no administration for rest of the 28 day cycle. The cycling method further allows the frequency, number, and length of dosing cycles to be increased. Thus, encompassed herein in certain embodiments is the administration of Compound D, for example, a formulation of Compound D provided herein, for more cycles than are typical when it is administered alone. In certain embodiments, Compound D, for example, a formulation of Compound D provided herein, is administered for a greater number of cycles that would typically cause dose-limiting toxicity in a patient to whom a second active ingredient is not also being administered.

In one embodiment, Compound D, for example, a formulation of Compound D provided herein, is administered daily and continuously for three or four weeks to administer a dose of Compound D from about 0.1 to about 20 mg/d followed by a break of one or two weeks.

In another embodiment, Compound D, for example, a formulation of Compound D provided herein, is administered intravenously and a second active ingredient is administered orally, with administration of Compound D, for example, a formulation of Compound D provided herein, occurring 30 to 60 minutes prior to a second active ingredient, during a cycle of four to six weeks. In certain embodiments, the combination of Compound D, for example, a formulation of Compound D provided herein, and a second active ingredient is administered by intravenous infusion over about 90 minutes every cycle. In certain embodiments, one cycle comprises the administration from about 0.1 to about 150 mg/day of Compound D, for example, a formulation of Compound D provided herein, and from about 50 to about 200 mg/m2/day of a second active ingredient daily for three to four weeks and then one or two weeks of rest. In certain embodiments, the number of cycles during which the combinatorial treatment is administered to a patient is ranging from about one to about 24 cycles, from about two to about 16 cycles, or from about four to about three cycles.

In one embodiment, a cycling therapy provided herein comprises administering Compound D, for example, a formulation of Compound D provided herein, in a treatment cycle which includes an administration period of up to 5 days followed by a rest period. In one embodiment, the treatment cycle includes an administration period of 5 days followed by a rest period. In one embodiment, the treatment cycle includes an administration period of up to 10 days followed by a rest period. In one embodiment, the rest period is from about 10 days up to about 40 days. In one embodiment, the treatment cycle includes an administration period of up to 10 days followed by a rest period from about 10 days up to about 40 days. In one embodiment, the treatment cycle includes an administration period of up to 10 days followed by a rest period from about 23 days up to about 37 days. In one embodiment, the rest period is from about 23 days up to about 37 days. In one embodiment, the rest period is 23 days. In one embodiment, the treatment cycle includes an administration period of up to 10 days followed by a rest period of 23 days. In one embodiment, the rest period is 37 days. In one embodiment, the treatment cycle includes an administration period of up to 10 days followed by a rest period of 37 days.

In one embodiment, the treatment cycle includes an administration of Compound D, for example, a formulation of Compound D provided herein, on days 1 to 5 of a 28 day cycle. In another embodiment, the treatment cycle includes an administration of Compound D, for example, a formulation of Compound D provided herein, on days 1-10 of a 28 day cycle. In one embodiment, the treatment cycle includes an administration on days 1 to 5 of a 42 day cycle. In another embodiment, the treatment cycle includes an administration on days 1-10 of a 42 day cycle. In another embodiment, the treatment cycle includes an administration on days 1-5 and 15-19 of a 28 day cycle. In another embodiment, the treatment cycle includes an administration on days 1-3 and 8-10 of a 28 day cycle.

In one embodiment, the treatment cycle includes an administration of Compound D, for example, a formulation of Compound D provided herein, on days 1 to 21 of a 28 day cycle. In another embodiment, the treatment cycle includes an administration on days 1 to 5 of a 7 day cycle. In another embodiment, the treatment cycle includes an administration on days 1 to 7 of a 7 day cycle.

Any treatment cycle described herein can be repeated for at least 2, 3, 4, 5, 6, 7, 8, or more cycles. In certain instances, the treatment cycle as described herein includes from 1 to about 24 cycles, from about 2 to about 16 cycles, or from about 2 to about 4 cycles. In certain instances a treatment cycle as described herein includes from 1 to about 4 cycles. In certain embodiments, cycle 1 to 4 are all 28 day cycles. In certain embodiments, cycle 1 is a 42 day cycle and cycles 2 to 4 are 28 day cycles. In some embodiments, Compound D, for example, a formulation of Compound D provided herein, is administered for 1 to 13 cycles of 28 days (e.g. about 1 year). In certain instances, the cycling therapy is not limited to the number of cycles, and the therapy is continued until disease progression. Cycles, can in certain instances, include varying the duration of administration periods and/or rest periods described herein.

In one embodiment the treatment cycle includes administering Compound D at a dosage amount of about 0.3 mg/day, 0.6 mg/day, 1.2 mg/day, 1.8 mg/day, 2.4 mg/day, 3.6 mg/day, 5.4 mg/day, 7.2 mg/day, 8.1 mg/day, 9.0 mg/day, 10.0 mg/day, 10.8 mg/day, or 12.2 mg/day administered once per day. In one embodiment the treatment cycle includes administering Compound D at a dosage amount of about 0.3 mg/day, 0.6 mg/day, 1.2 mg/day, 1.8 mg/day, 2.4 mg/day, 3.6 mg/day, 5.4 mg/day, 7.2 mg/day, 8.1 mg/day, 9.0 mg/day, 10.0 mg/day, 10.8 mg/day, 12.2 mg/day, or 20 mg/day administered once per day. In one embodiment the treatment cycle includes administering Compound D at a dosage amount of about 0.6 mg/day, 1.2 mg/day, 1.8 mg/day, 2.4 mg/day, or 3.6 mg/day, administered once per day. In some such embodiments, the treatment cycle includes administering Compound D at a dosage amount of about 0.6 mg, 1.2 mg, 1.8 mg, 2.4 mg, or 3.6 mg on days 1 to 3 of a 28 day cycle. In other embodiments, the treatment cycle includes administering Compound D at a dosage amount of about 0.6 mg, 1.2 mg, 1.8 mg, 2.4 mg, or 3.6 mg on days 1 to 5 and 15 to 19 of a 28 day cycle. In other embodiments, the treatment cycle includes administering Compound D at a dosage amount of about 0.6 mg, 1.2 mg, 1.8 mg, 2.4 mg, 3.6 mg, 5.4 mg/day, 7.2 mg/day, 8.1 mg/day, 9.0 mg/day, or 10.0 mg/day, on days 1 to 5 and 15 to 19 of a 28 day cycle.

Compound D, for example, a formulation of Compound D provided herein, can be administered at the same amount for all administration periods in a treatment cycle. Alternatively, in one embodiment, the compound is administered at different doses in the administration periods.

In one embodiment, a formulation of Compound D provided herein is administered to a subject in a cycle, wherein the cycle comprises administering the formulation for at least 5 days in a 28 day cycle. In one embodiment, a formulation of Compound D provided herein is administered to a subject in a cycle, wherein the cycle comprises administering the formulation on days 1 to 5 of a 28 day cycle. In one embodiment, the formulation is administered to deliver Compound D in a dose of about 0.1 mg to about 20 mg on days 1 to 5 of a 28 day cycle. In one embodiment, the formulation is administered to deliver Compound D in a dose of about 0.5 mg to about 5 mg on days 1 to 5 of a 28 day cycle. In one embodiment, the formulation is administered to deliver Compound D in a dose of about 0.5 mg to about 10 mg on days 1 to 5 of a 28 day cycle. In one embodiment, a formulation of Compound D provided herein is administered to a subject in a cycle, wherein the cycle comprises administering the formulation on days 1 to 5 and 15 to 19 of a 28 day cycle. In one embodiment, the formulation is administered to deliver Compound D in a dose of about 0.1 mg to about 20 mg on days 1 to 5 and 15 to 19 of a 28 day cycle. In one embodiment, the formulation is administered to deliver Compound D in a dose of about 0.5 mg to about 5 mg on days 1 to 5 and 15 to 19 of a 28 day cycle. In one embodiment, the formulation is administered to deliver Compound D in a dose of about 0.5 mg to about 10 mg on days 1 to 5 and 15 to 19 of a 28 day cycle.

In one embodiment, provided herein is a method of treating of AML by administering to a subject a formulation of Compound D provided herein in a cycle, wherein the cycle comprises administering the formulation to deliver Compound D in a dose of about 0.1 mg to about 20 mg for at least 5 days in a 28 day cycle. In one embodiment, provided herein is a method of treating of AML by administering to a subject a formulation of Compound D provided herein in a cycle, wherein the cycle comprises administering the formulation to deliver Compound D in a dose of about 0.1 mg to about 20 mg on days 1 to 5 of a 28 day cycle. In one embodiment, provided herein is a method of treating of AML by administering to a subject a formulation of Compound D provided herein in a cycle, wherein the cycle comprises administering the formulation to deliver Compound D in a dose of about 0.1 mg to about 5 mg on days 1 to 5 of a 28 day cycle. In one embodiment, provided herein is a method of treating of AML by administering to a subject a formulation of Compound D provided herein in a cycle, wherein the cycle comprises administering the formulation to deliver Compound D in a dose of about 0.5 mg to about 5 mg on days 1 to 5 of a 28 day cycle. In another embodiment, provided herein is a method of treating of AML by administering to a subject a formulation of Compound D provided herein in a cycle, wherein the cycle comprises administering the formulation to deliver Compound D in a dose of about 0.1 mg to about 20 mg on days 1 to 5 and 15 to 19 of a 28 day cycle. In one embodiment, provided herein is a method of treating of AML by administering to a subject a formulation of Compound D provided herein in a cycle, wherein the cycle comprises administering the formulation to deliver Compound D in a dose of about 0.1 mg to about 5 mg on days 1 to 5 and 15 to 19 of a 28 day cycle. In one embodiment, provided herein is a method of treating of AML by administering to a subject a formulation of Compound D provided herein in a cycle, wherein the cycle comprises administering the formulation to deliver Compound D in a dose of about 0.5 mg to about 5 mg on days 1 to 5 and 15 to 19 of a 28 day cycle.

In one embodiment, provided herein is a method of treating of MDS by administering to a subject a formulation of Compound D provided herein in a cycle, wherein the cycle comprises administering the formulation to deliver Compound D in a dose of about 0.1 mg to about 20 mg for at least 5 days in a 28 day cycle. In one embodiment, provided herein is a method of treating of MDS by administering to a subject a formulation of Compound D provided herein in a cycle, wherein the cycle comprises administering the formulation to deliver Compound D in a dose of about 0.1 mg to about 20 mg on days 1 to 5 of a 28 day cycle. In one embodiment, provided herein is a method of treating of MDS by administering to a subject a formulation of Compound D provided herein in a cycle, wherein the cycle comprises administering the formulation to deliver Compound D in a dose of about 0.1 mg to about 5 mg on days 1 to 5 of a 28 day cycle. In one embodiment, provided herein is a method of treating of MDS by administering to a subject a formulation of Compound D provided herein in a cycle, wherein the cycle comprises administering the formulation to deliver Compound D in a dose of about 0.5 mg to about 5 mg on days 1 to 5 of a 28 day cycle. In another embodiment, provided herein is a method of treating of MDS by administering to a subject a formulation of Compound D provided herein in a cycle, wherein the cycle comprises administering the formulation to deliver Compound D in a dose of about 0.1 mg to about 20 mg on days 1 to 5 and 15 to 19 of a 28 day cycle. In one embodiment, provided herein is a method of treating of MDS by administering to a subject a formulation of Compound D provided herein in a cycle, wherein the cycle comprises administering the formulation to deliver Compound D in a dose of about 0.1 mg to about 5 mg on days 1 to 5 and 15 to 19 of a 28 day cycle. In one embodiment, provided herein is a method of treating of MDS by administering to a subject a formulation of Compound D provided herein in a cycle, wherein the cycle comprises administering the formulation to deliver Compound D in a dose of about 0.5 mg to about 5 mg on days 1 to 5 and 15 to 19 of a 28 day cycle.

5.3. Methods of Detecting and Quantifying Biomarkers

In certain embodiments, the biomarkers provide herein can be measured by the protein level, RNA level, DNA level, or cDNA level of the biomarker. In certain embodiments of the various methods provided herein, the two or more of the steps are performed sequentially. In other embodiments of the methods provided herein, two or more of steps are performed in parallel (e.g., at the same time).

Several protein detection and quantization methods can be used to measure the level of a biomarker. Any suitable protein quantization method can be used. In some embodiments, antibody-based methods are used. Exemplary methods that can be used include, but are not limited to, immunoblotting (Western blot), ELISA, immunohistochemistry, flow cytometry, cytometry bead array, mass spectroscopy, and the like. Several types of ELISA are commonly used, including direct ELISA, indirect ELISA, and sandwich ELISA.

In certain embodiments, provided herein are methods of detecting and quantifying the protein level of biomarker (e.g., a gene product) from a biological sample, comprising contacting proteins within the sample with a first antibody that immunospecifically binds to the biomarker protein. In some embodiments, the methods provided herein further comprise (i) contacting the biomarker protein bound to the first antibody with a second antibody with a detectable label, wherein the second antibody immunospecifically binds to the biomarker protein, and wherein the second antibody immunospecifically binds to a different epitope on the biomarker protein than the first antibody; (ii) detecting the presence of the second antibody bound to the biomarker protein; and (iii) determining the amount of the biomarker protein based on the amount of detectable label in the second antibody. In other embodiments, the methods provided herein further comprise (i) contacting the biomarker protein bound to the first antibody with a second antibody with a detectable label, wherein the second antibody immunospecifically binds to the first antibody; (ii) detecting the presence of the second antibody bound to the first antibody; and (iii) determining the amount of the biomarker protein based on the amount of detectable label in the second antibody.

In some embodiments of the various methods provided herein, the method comprises using dual staining immunohistochemistry to determine the level of a biomarker. In a dual staining immunohistochemistry assay, a biomarker provided herein and another cancer biomarker are simultaneously detected using a first labeled antibody targeting a biomarker provided herein and a second labeled antibody targeting a cancer biomarker. Such assay can improve the specificity, accuracy, and sensitivity for detecting and measuring a biomarker provided herein. In some embodiments, the cancer biomarker is a lymphoma biomarker. In other embodiments, the cancer biomarker is an NHL biomarker. In certain embodiments, the cancer biomarker is a DLBCL biomarker. In some embodiments, the cancer biomarker is an MM biomarker. In other embodiments, the cancer biomarker is a leukemia biomarker. In yet other embodiments, the cancer biomarker is an AML biomarker.

Thus, in some embodiments, the method provided herein comprises (i) contacting proteins within a sample with a first antibody that immunospecifically binds to a biomarker provided herein, the first antibody being coupled with a first detectable label; (ii) contacting the proteins within the sample with a second antibody that immunospecifically binds to a cancer biomarker, the second antibody being coupled with a second detectable label; (iii) detecting the presence of the first antibody and the second antibody bound to the proteins; and (iv) determining the level of the biomarker provided herein based on the amount of detectable label in the first antibody, and determining the level of the cancer biomarker based on the amount of detectable label in the second antibody. In some embodiments, the cancer biomarker is a lymphoma biomarker. In other embodiments, the cancer biomarker is an NHL biomarker. In certain embodiments, the cancer biomarker is a DLBCL biomarker. In some embodiments, the cancer biomarker is an MM biomarker. In other embodiments, the cancer biomarker is a leukemia biomarker. In yet other embodiments, the cancer biomarker is an AML biomarker.

Several methods of detecting or quantitating mRNA levels are known in the art.

Exemplary methods include, but are not limited to, northern blots, ribonuclease protection assays, PCR-based methods, and the like. The mRNA sequence of a biomarker can be used to prepare a probe that is at least partially complementary to the mRNA sequence. The probe can then be used to detect the mRNA in a sample, using any suitable assay, such as PCR-based methods, northern blotting, a dipstick assay, and the like.

In other embodiments, a nucleic acid assay for testing for compound activity in a biological sample can be prepared. An assay typically contains a solid support and at least one nucleic acid contacting the support, where the nucleic acid corresponds to at least a portion of an mRNA that has altered expression during a compound treatment in a patient, such as the mRNA of a biomarker. The assay can also have a means for detecting the altered expression of the mRNA in the sample.

The assay method can be varied depending on the type of mRNA information desired. Exemplary methods include but are not limited to Northern blots and PCR-based methods (e.g., qRT-PCR). Methods such as qRT-PCR can also accurately quantitate the amount of the mRNA in a sample.

Any suitable assay platform can be used to determine the presence of mRNA in a sample. For example, an assay may be in the form of a dipstick, a membrane, a chip, a disk, a test strip, a filter, a microsphere, a slide, a multi-well plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film, a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.). See, e.g., Ausubel et al., Short Protocols in Molecular Biology (Wiley & Sons, 3rd ed. 1995); Sambrook et al., Molecular Cloning: A Laboratory Manual (Cold Spring Harbor, N.Y., 3rd ed. 2001). In some embodiments, the sample is labeled with fluorescent label. Exemplary fluorescent dyes include, but are not limited to, xanthene dyes, fluorescein dyes (e.g., fluorescein isothiocyanate (FITC), 6-carboxyfluorescein (FAM), 6 carboxy-2′,4′,7′,4,7-hexachlorofluorescein (HEX), 6-carboxy-4′,5′-dichloro-2′,7′-dimethoxyfluorescein (JOE)), rhodamine dyes (e.g., rhodamine 110 (R110), N,N,N′,N′-tetramethyl-6-carboxyrhodamine (TAMRA), 6-carboxy-X-rhodamine (ROX), 5-carboxyrhodamine 6G (R6G5 or G5), 6-carboxyrhodamine 6G (R6G6 or G6)), cyanine dyes (e.g., Cy3, Cy5 and Cy7), Alexa dyes (e.g., Alexa-fluor-555), coumarin, Diethylaminocoumarin, umbelliferone, benzimide dyes (e.g., Hoechst 33258), phenanthridine dyes (e.g., Texas Red), ethidium dyes, acridine dyes, carbazole dyes, phenoxazine dyes, porphyrin dyes, polymethine dyes, BODIPY dyes, quinoline dyes, Pyrene, Fluorescein Chlorotriazinyl, eosin dyes, Tetramethylrhodamine, Lissamine, Napthofluorescein, and the like.

In some embodiments, the mRNA sequences comprise at least one mRNA of a biomarker provided herein.

The nucleic acids may be present in specific, addressable locations on a solid support, each corresponding to at least a portion of mRNA sequences that are differentially expressed upon treatment of a compound in a cell or a patient.

A typical mRNA assay method can contain the steps of 1) obtaining surface-bound subject probes; 2) hybridizing a population of mRNAs to the surface-bound probes under conditions sufficient to provide for specific binding; (3) post-hybridization washing to remove nucleic acids not specifically bound to the surface-bound probes; and (4) detecting the hybridized mRNAs. The reagents used in each of these steps and their conditions for use may vary depending on the particular application.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions may be employed.

Hybridization is typically performed under stringent hybridization conditions. Standard hybridization techniques (e.g., under conditions sufficient to provide for specific binding of target mRNAs in the sample to the probes) are described in Kallioniemi et al., Science 1992, 258:818-821 and International Patent Application Publication No. WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al., Meth. Enzymol. 1981, 21:470-480; Angerer et al., Genetic Engineering: Principles and Methods, Vol 7, pgs 43-65 (Plenum Press, New York, Setlow and Hollaender, eds. 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art.

Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

Other methods, such as PCR-based methods, can also be used to detect the expression of a biomarker provided herein. Examples of PCR methods can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, quantitative Reverse Transcription-PCR (qRT-PCR) can be used for both the detection and quantification of RNA targets (Bustin et al., Clin. Sci. 2005, 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663, which is incorporated by reference herein in its entirety.

In contrast to regular reverse transcriptase-PCR and analysis by agarose gels, qRT-PCR gives quantitative results. An additional advantage of qRT-PCR is the relative ease and convenience of use. Instruments for qRT-PCR, such as the Applied Biosystems 7500, are available commercially, so are the reagents, such as TaqMan® Sequence Detection Chemistry. For example, TaqMan® Gene Expression Assays can be used, following the manufacturer's instructions. These kits are pre-formulated gene expression assays for rapid, reliable detection and quantification of human, mouse, and rat mRNA transcripts. An exemplary qRT-PCR program, for example, is 50° C. for 2 minutes, 95° C. for 10 minutes, 40 cycles of 95° C. for 15 seconds, then 60° C. for 1 minute.

To determine the cycle number at which the fluorescence signal associated with a particular amplicon accumulation crosses the threshold (referred to as the CT), the data can be analyzed, for example, using 7500 Real-Time PCR System Sequence Detection software vs. using the comparative CT relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

5.4. Subjects, Samples, and Types of Cells

In certain embodiments, the various methods provided herein use samples (e.g., biological samples) from subjects or individuals (e.g., patients). The subject can be a patient, such as, a patient with a cancer (e.g., lymphoma, MM, or leukemia). The subject can be a mammal, for example, a human. The subject can be male or female, and can be an adult, a child, or an infant. Samples can be analyzed at a time during an active phase of a cancer (e.g., lymphoma, MM, or leukemia), or when the cancer (e.g., lymphoma, MM, or leukemia) is inactive. In certain embodiments, more than one sample from a subject can be obtained.

In certain embodiments, the sample used in the methods provided herein comprises body fluids from a subject. Non-limiting examples of body fluids include blood (e.g., whole blood), blood plasma, amniotic fluid, aqueous humor, bile, cerumen, cowper's fluid, pre-ejaculatory fluid, chyle, chyme, female ejaculate, interstitial fluid, lymph, menses, breast milk, mucus, pleural fluid, pus, saliva, sebum, semen, serum, sweat, tears, urine, vaginal lubrication, vomit, water, feces, internal body fluids (including cerebrospinal fluid surrounding the brain and the spinal cord), synovial fluid, intracellular fluid (the fluid inside cells), and vitreous humour (the fluid in the eyeball). In some embodiments, the sample is a blood sample. The blood sample can be obtained using conventional techniques as described in, e.g., Innis et al, eds., PCR Protocols (Academic Press, 1990). White blood cells can be separated from blood samples using conventional techniques or commercially available kits, e.g., RosetteSep kit (Stein Cell Technologies, Vancouver, Canada). Sub-populations of white blood cells, e.g., mononuclear cells, B cells, T cells, monocytes, granulocytes, or lymphocytes, can be further isolated using conventional techniques, e.g., magnetically activated cell sorting (MACS) (Miltenyi Biotec, Auburn, Calif.) or fluorescently activated cell sorting (FACS) (Becton Dickinson, San Jose, Calif.).

In one embodiment, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3 mL. In another embodiment, the blood sample is about 0.3, about 0.4, about 0.5, about 0.6, about 0.7, about 0.8, about 0.9, about 1.0, about 1.5, about 2.0, about 2.5, about 3.0, about 3.5, about 4.0, about 4.5, about 5.0, about 6.0, about 7.0, about 8.0, about 9.0, or about 10.0 mL.

In some embodiments, the sample used in the present methods comprises a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy.

In one embodiment, the sample used in the methods provided herein is obtained from the subject prior to the subject receiving a treatment for the disease or disorder. In another embodiment, the sample is obtained from the subject during the subject receiving a treatment for the disease or disorder. In another embodiment, the sample is obtained from the subject after the subject receiving a treatment for the disease or disorder. In various embodiments, the treatment comprises administering a compound (e.g., a compound provided in Section 5.5 below) to the subject.

In certain embodiments, the sample used in the methods provided herein comprises a plurality of cells, such as cancer (e.g., lymphoma, MM, or leukemia) cells. Such cells can include any type of cells, e.g., stem cells, blood cells (e.g., peripheral blood mononuclear cells (PBMC)), lymphocytes, B cells, T cells, monocytes, granulocytes, immune cells, or cancer cells.

B cells (B lymphocytes) include, for example, plasma B cells, memory B cells, B1 cells, B2 cells, marginal-zone B cells, and follicular B cells. B cells can express immunoglobulins (antibodies) and B cell receptor.

Specific cell populations can be obtained using a combination of commercially available antibodies (e.g., antibodies from Quest Diagnostic (San Juan Capistrano, Calif.) or Dako (Denmark)).

In certain embodiments, the cells in the methods provided herein are PBMC. In certain embodiments, the sample used in the methods provided herein is from a disease tissue, e.g., from an individual having cancer (e.g., lymphoma, MM, or leukemia).

In certain embodiments, cell lines are used as disease models for evaluating effects of compounds, studying mechanisms of action, or establishing reference levels of biomarkers, etc. In some embodiments, the cells used in the methods provided herein are from a cancer (e.g., AML) cell line. In certain embodiments, the cells are from a lymphoma cell line. In other embodiments, the cells are from an MM cell line. In other embodiments, the cells are from a leukemia cell line. In some embodiments, the leukemia cell line is a CLL cell line. In other embodiments, the leukemia cell line is an ALL cell line. In yet other embodiments, the leukemia cell line is a CML cell line. In yet other embodiments, the leukemia cell line is an AML cell line. In one embodiment, the AML cell line is KG-1 cell line. In another embodiment, the AML cell line is KG-1a cell line. In yet another embodiment, the AML cell line is KASUMI-1 cell line. In still another embodiment, the AML cell line is NB4 cell line. In one embodiment, the AML cell line is MV-4-11 cell line. In another embodiment, the AML cell line is MOLM-13 cell line. In yet another embodiment, the AML cell line is HL-60 cell line. In still another embodiment, the AML cell line is U-937 cell line. In one embodiment, the AML cell line is OCI-AML2 cell line. In another embodiment, the AML cell line is OCI-AML3 cell line. In yet another embodiment, the AML cell line is HNT-34 cell line. In still another embodiment, the AML cell line is ML-2 cell line. In one embodiment, the AML cell line is AML-193 cell line. In another embodiment, the AML cell line is F36-P cell line. In yet another embodiment, the AML cell line is KASUMI-3 cell line. In still another embodiment, the AML cell line is MUTZ-8 cell line. In one embodiment, the AML cell line is GDM-1 cell line. In another embodiment, the AML cell line is SIG-M5 cell line. In yet another embodiment, the AML cell line is TF-1 cell line. In still another embodiment, the AML cell line is Nomo-1 cell line. In one embodiment, the AML cell line is UT-7 cell line. In another embodiment, the AML cell line is THP-1 cell line.

In certain embodiments, the methods provided herein are useful for detecting gene rearrangement in cells from a healthy individual. In certain embodiments, the number of cells used in the methods provided herein can range from a single cell to about 10⁹ cells. In some embodiments, the number of cells used in the methods provided herein is about 1×10⁴, about 5×10⁴, about 1×10⁵, about 5×10⁵, about 1×10⁶, about 5×10⁶, about 1×10⁷, about 5×10⁷, about 1×10⁸, about 5×10⁸, or about 1×10⁹.

The number and type of cells collected from a subject can be monitored, for example, by measuring changes in cell surface markers using standard cell detection techniques such as flow cytometry, cell sorting, immunocytochemistry (e.g., staining with tissue specific or cell-marker specific antibodies), fluorescence activated cell sorting (FACS), magnetic activated cell sorting (MACS), by examining the morphology of cells using light or confocal microscopy, and/or by measuring changes in gene expression using techniques well known in the art, such as PCR and gene expression profiling. These techniques can be used, too, to identify cells that are positive for one or more particular markers.

In certain embodiments, subsets of cells are used in the methods provided herein. Methods of sorting and isolating specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc. FACS is a well-known method for separating particles, including cells, based on the fluorescent properties of the particles (Kamarch, Methods Enzymol. 1987, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one embodiment, RNA (e.g., mRNA) or protein is purified from a tumor, and the level of a gene set is measured by mRNA or protein expression analysis. In certain embodiments, the level of a gene set is measured by transcriptomic profiling, qRT-PCR, microarray, high throughput sequencing, or other similar methods known in the art. In other embodiments, the level of a gene set is measured by ELISA, flow cytometry, immunofluorescence, or other similar methods known in the art.

5.5. Compounds

The compound suitable for use in the methods and formulations provided herein is Compound D: 2-(4-chlorophenyl)-N-((2-(2,6-dioxopiperidin-3-yl)-1-oxoisoindolin-5-yl)methyl)-2,2-difluoroacetamide having the structure:

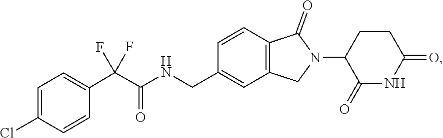 [SMILES: O=C1CCC(N2Cc3cc(CNC(=O)C(F)(F)c4ccc(Cl)cc4)ccc3C2=O)C(=O)N1]

or its stereoisomers or mixture of stereoisomers, isotopologues, pharmaceutically acceptable salts, tautomers, solvates, hydrates, co-crystals, clathrates, or polymorphs thereof. In certain embodiments, Compound D refers to 2-(4-chlorophenyl)-N-((2-(2,6-dioxopiperidin-3-yl)-1-oxoisoindolin-5-yl)methyl)-2,2-difluoroacetamide.

Compound D can be prepared according to the methods described in the Examples provided herein or as described in U.S. Pat. No. 9,499,514, the disclosure of which is incorporated herein by reference in its entirety. The compound can also be synthesized according to other methods apparent to those of skill in the art based upon the teaching herein.

In certain embodiments, Compound D is a solid. In certain embodiments, Compound D is a hydrate. In certain embodiments, Compound D is solvated. In certain embodiments, Compound D is anhydrous.

In certain embodiments, Compound D is amorphous. In certain embodiments, Compound D is crystalline. In certain embodiments, Compound D is in a crystalline form described in U.S. Publication No. 2017-0197934 filed on Jan. 6, 2017, which is incorporated herein by reference in its entirety.

The solid forms of Compound D can be prepared according to the methods described in the disclosure of U.S. Publication No. 2017-0197934 filed on Jan. 6, 2017. The solid forms can also be prepared according to other methods apparent to those of skill in the art.

In one embodiment, Compound D is polymorph Form A, Form B, Form C, Form D, Form E or an amorphous form of 2-(4-chlorophenyl)-N-((2-(2,6-dioxopiperidin-3-yl)-1-oxoisoindolin-5-yl)methyl)-2,2-difluoroacetamide. Polymorphs of 2-(4-chlorophenyl)-N-((2-(2,6-dioxopiperidin-3-yl)-1-oxoisoindolin-5-yl)methyl)-2,2-difluoroacetamide are briefly described herein. In certain embodiments, Compound D has a polymorph form as described in US Publication No. 2019/0030018, the disclosure of which is incorporated herein by reference in its entirety, and portion of which is described in more detail below.

### Form A of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein are prepared from Form A of Compound D.

In one embodiment, Form A is an anhydrous form of Compound D. In another embodiment, Form A of Compound D is crystalline.

In certain embodiments, Form A is obtained by crystallization from certain solvent systems, for example, solvent systems comprising one or more of the following solvents: acetone and the solvent mixture of isopropanol and water at room temperature. In certain embodiments, Form A is obtained as an intermediate solid form from slurries at elevated temperature, for example about 50° C., in ethanol/water (1:1), acetone or acetonitrile.

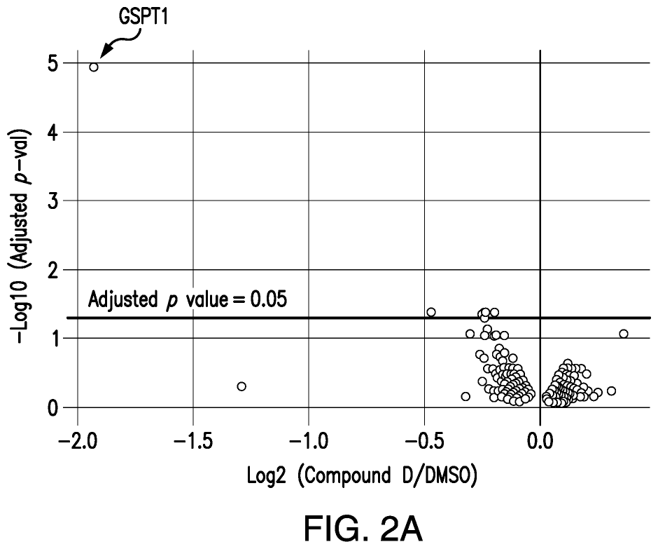

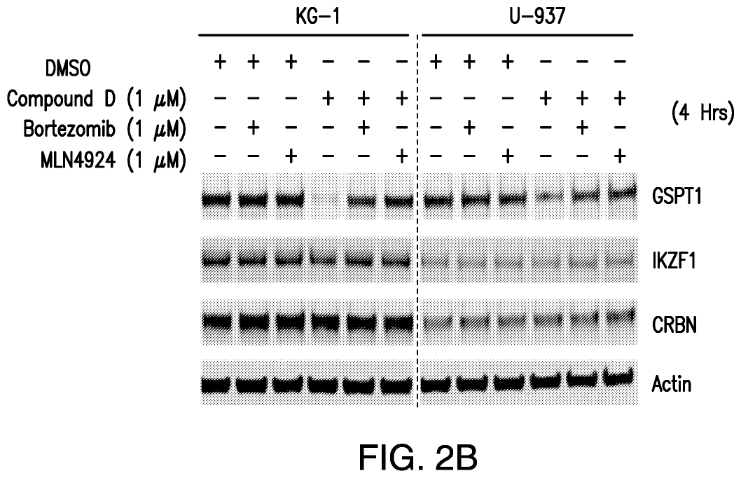

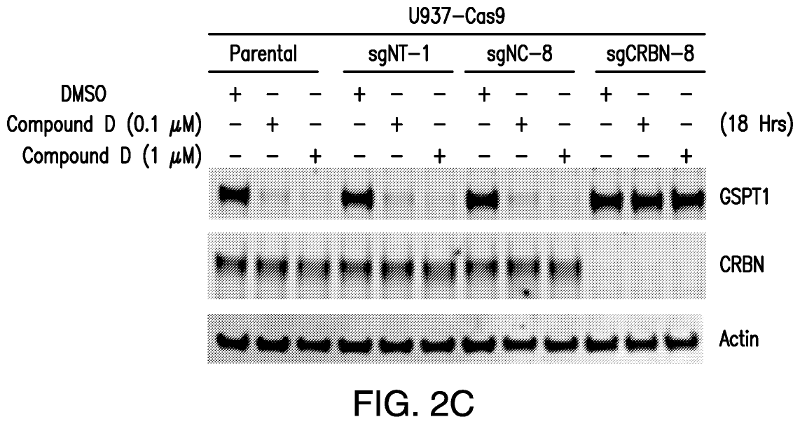

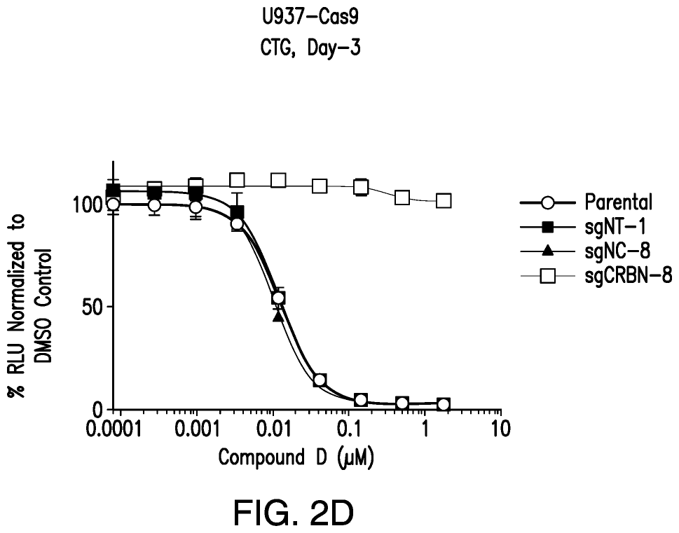

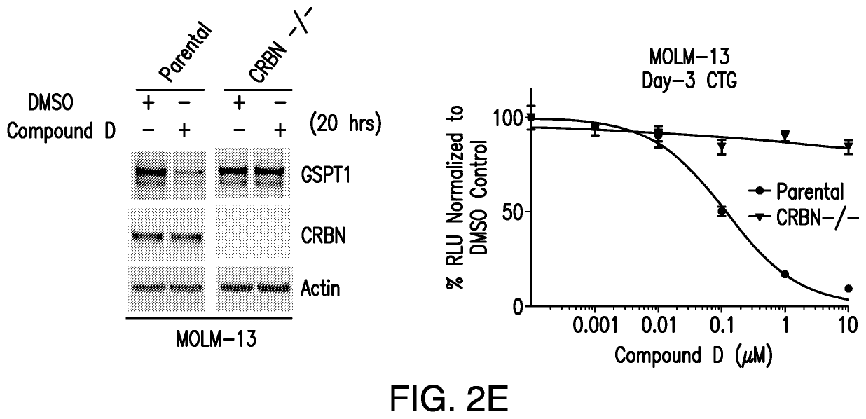

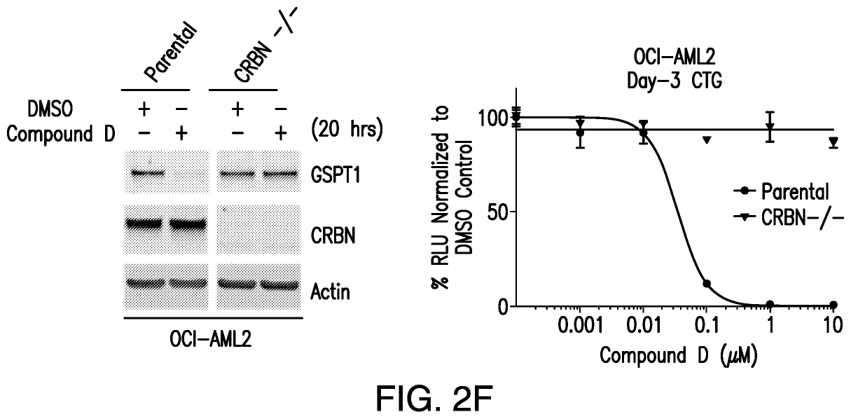

In certain embodiments, Form A is substantially crystalline, as indicated by, e.g., X-ray powder diffraction measurements. In one embodiment, Form A of Compound D has an X-ray powder diffraction pattern substantially as shown in FIG. 2 of US Publication No. 2019/0030018.

In one embodiment, Form A of Compound D has one or more characteristic X-ray powder diffraction peaks at a two-theta angle of approximately 11.5, 15.6, 16.6, 17.2, 18.1, 19.0, 19.6, 21.1, 23.2 or 24.8 degrees 2θ as depicted in FIG. 2 of US Publication No. 2019/0030018. In another embodiment, Form A of Compound D has one, two, three or four characteristic X-ray powder diffraction peaks at a two-theta angle of approximately 15.6, 16.6, 17.2 or 24.8 degrees 2θ. In another embodiment, Form A of Compound D has one, two, three, four, five, six or seven characteristic X-ray powder diffraction peaks as set forth in Table A. In another embodiment, Form A of Compound D has one, two, or three characteristic X-ray powder diffraction peaks as set forth in Table A.

TABLE A

| | Pos. | d-spacing | Rel. Int. |
|---|---|---|---|
| No. | [°2 Th.] | [Å] | [%] |
| 1 | 7.23 | 12.2187 | 17.6 |
| 2 | 11.52 | 7.6789 | 29.7 |
| 3 | 15.22 | 5.8209 | 7.5 |
| 4 | 15.62 | 5.6720 | 31.2 |
| 5 | 16.58 | 5.3466 | 40.3 |
| 6 | 17.19 | 5.1576 | 100.0 |
| 7 | 18.08 | 4.9056 | 22.3 |
| 8 | 19.00 | 4.6702 | 19.6 |
| 9 | 19.60 | 4.5302 | 22.1 |
| 10 | 21.05 | 4.2197 | 29.2 |
| 11 | 21.74 | 4.0884 | 8.3 |
| 12 | 22.01 | 4.0388 | 7.1 |
| 13 | 22.47 | 3.9576 | 6.0 |
| 14 | 23.22 | 3.8312 | 28.6 |
| 15 | 24.17 | 3.6825 | 5.6 |
| 16 | 24.77 | 3.5945 | 57.2 |
| 17 | 25.59 | 3.4813 | 14.6 |
| 18 | 25.94 | 3.4356 | 10.5 |
| 19 | 26.63 | 3.3470 | 17.4 |
| 20 | 27.73 | 3.2172 | 10.0 |
| 21 | 28.51 | 3.1307 | 7.1 |
| 22 | 29.88 | 2.9906 | 19.3 |
| 23 | 30.76 | 2.9065 | 7.1 |
| 24 | 31.59 | 2.8327 | 11.1 |
| 25 | 34.82 | 2.5766 | 4.8 |
| 26 | 36.05 | 2.4913 | 4.3 |

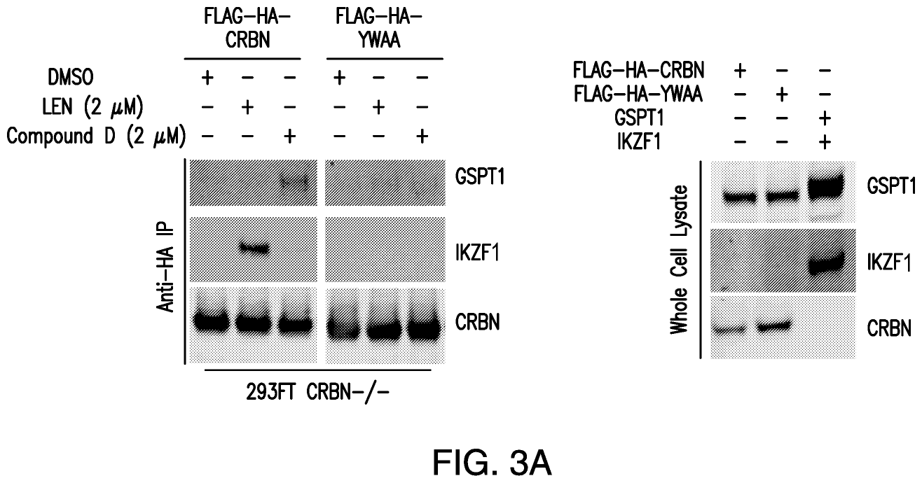

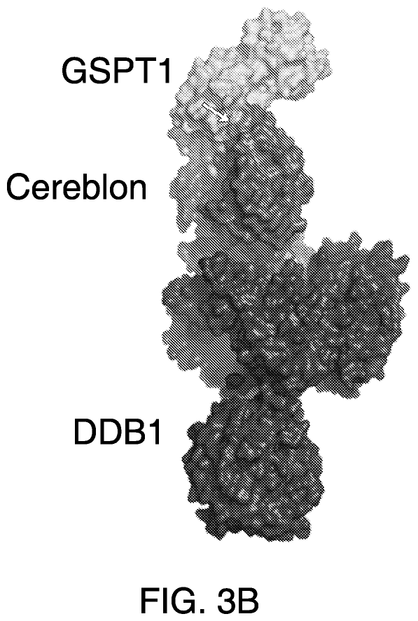

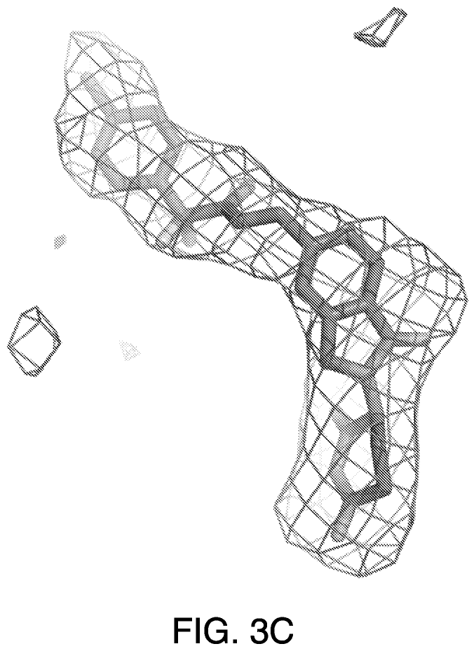

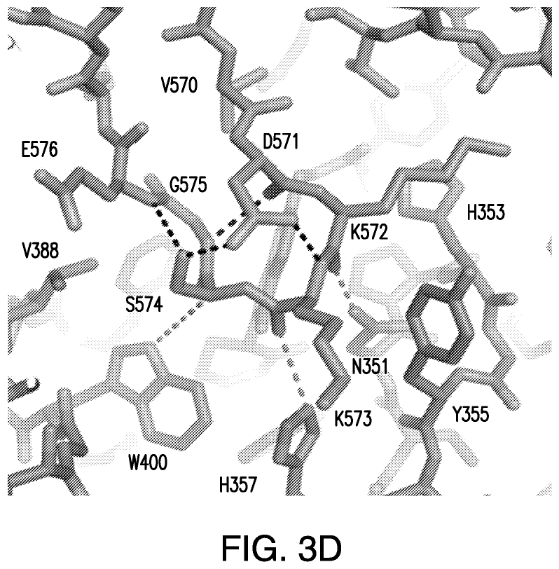

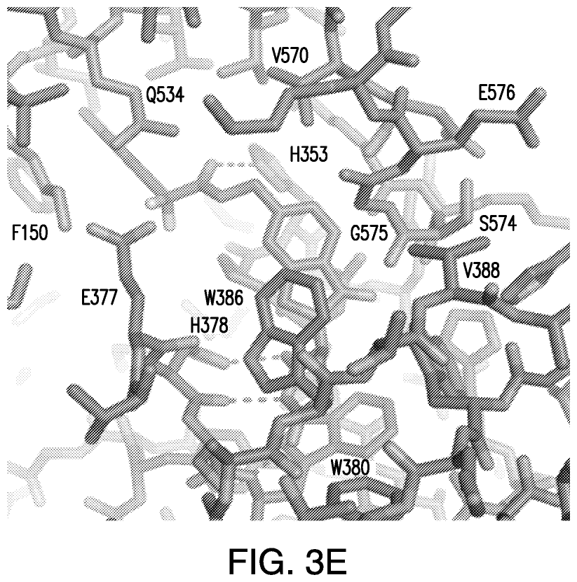

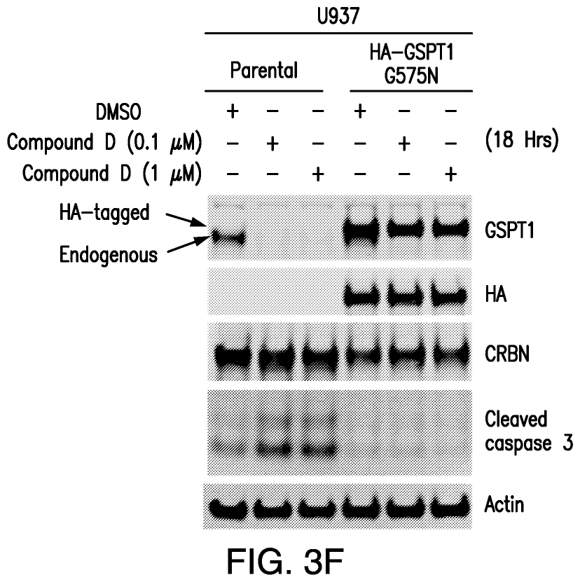

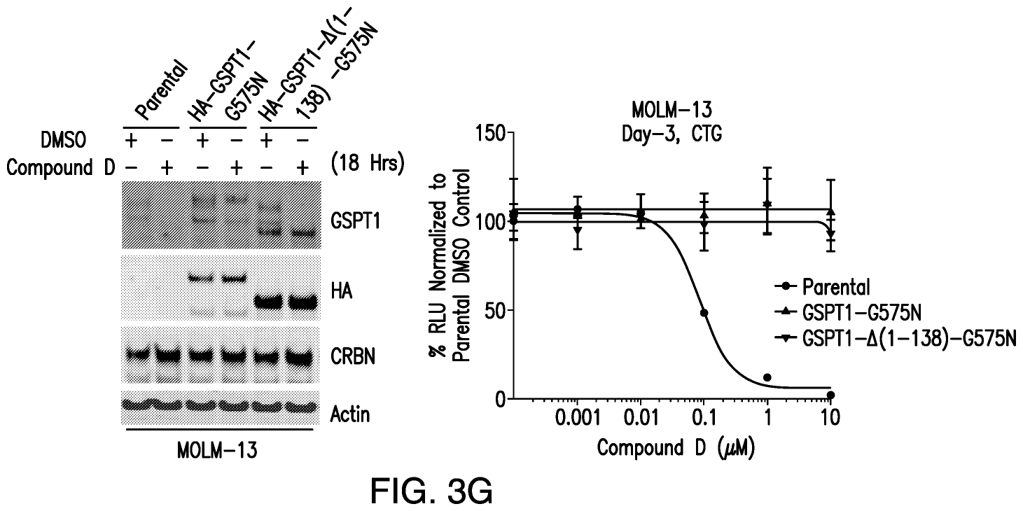

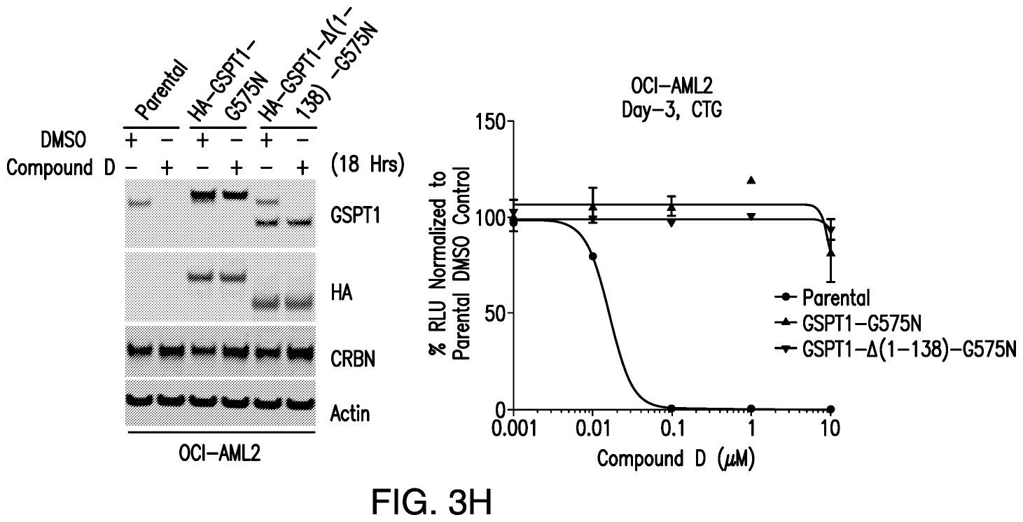

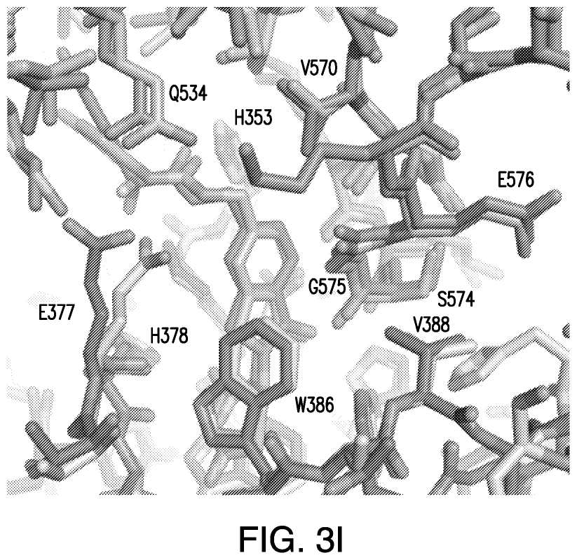

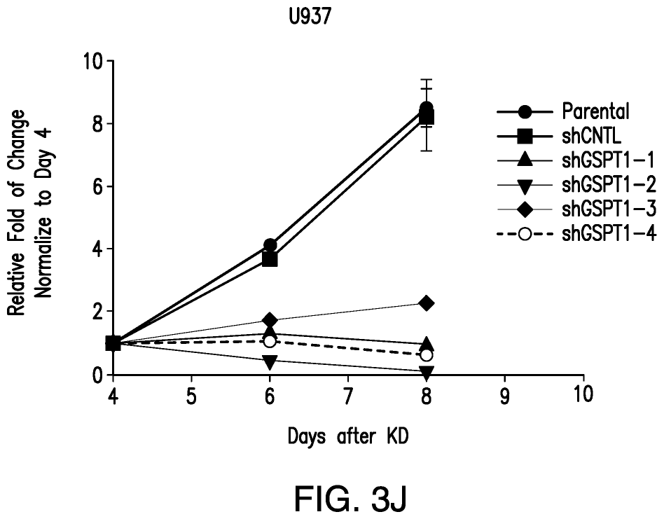

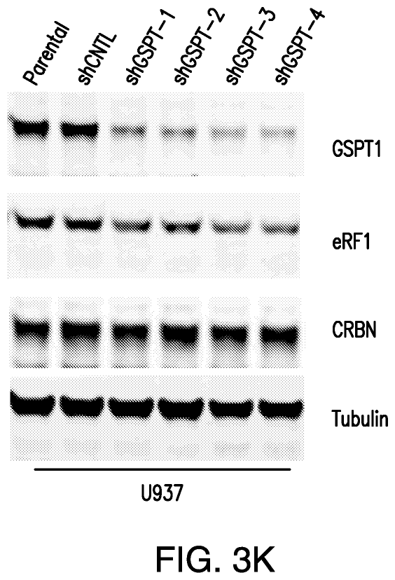

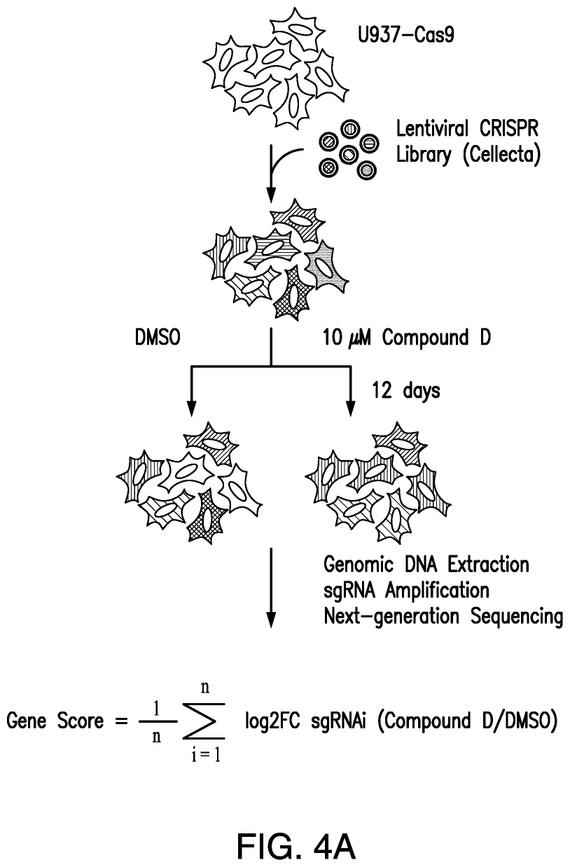

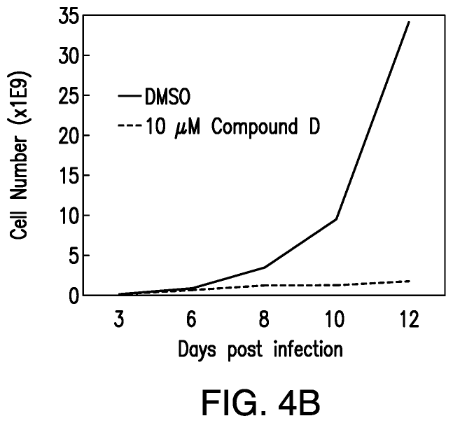

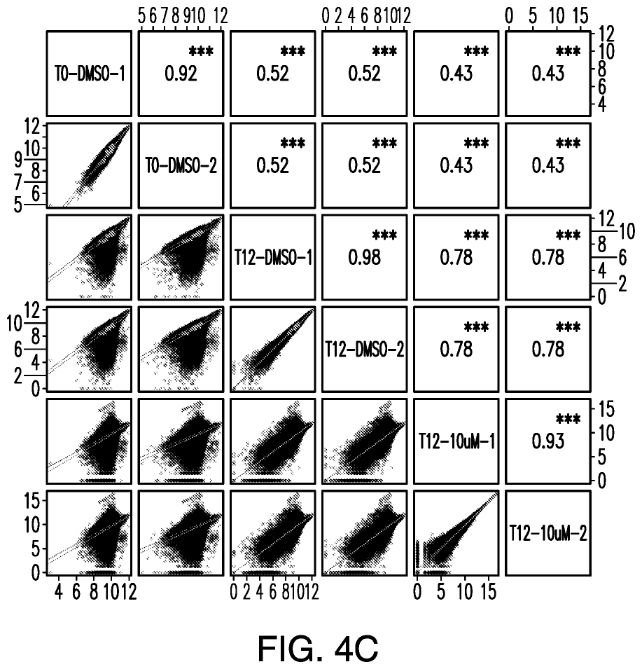

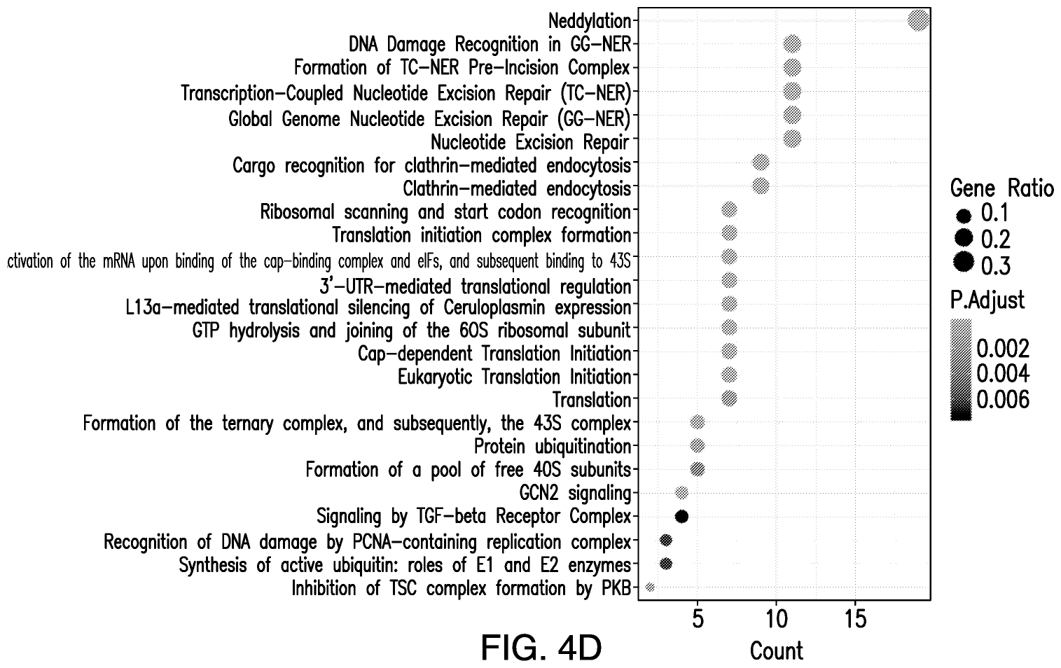

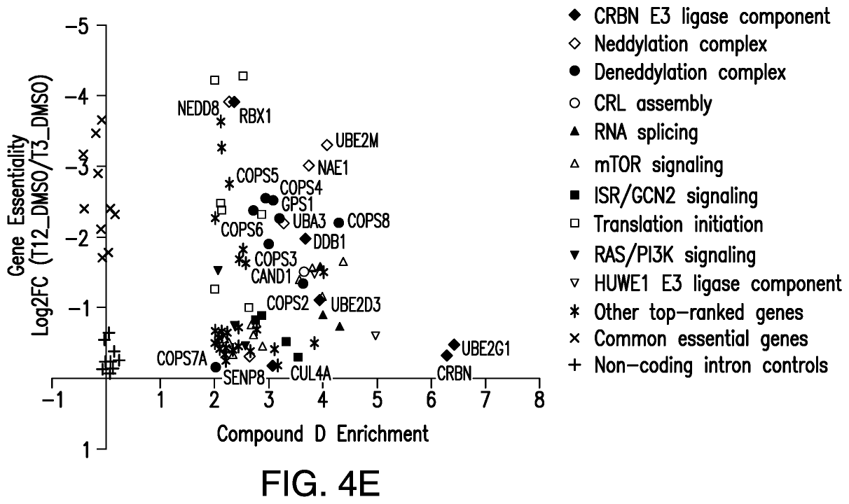

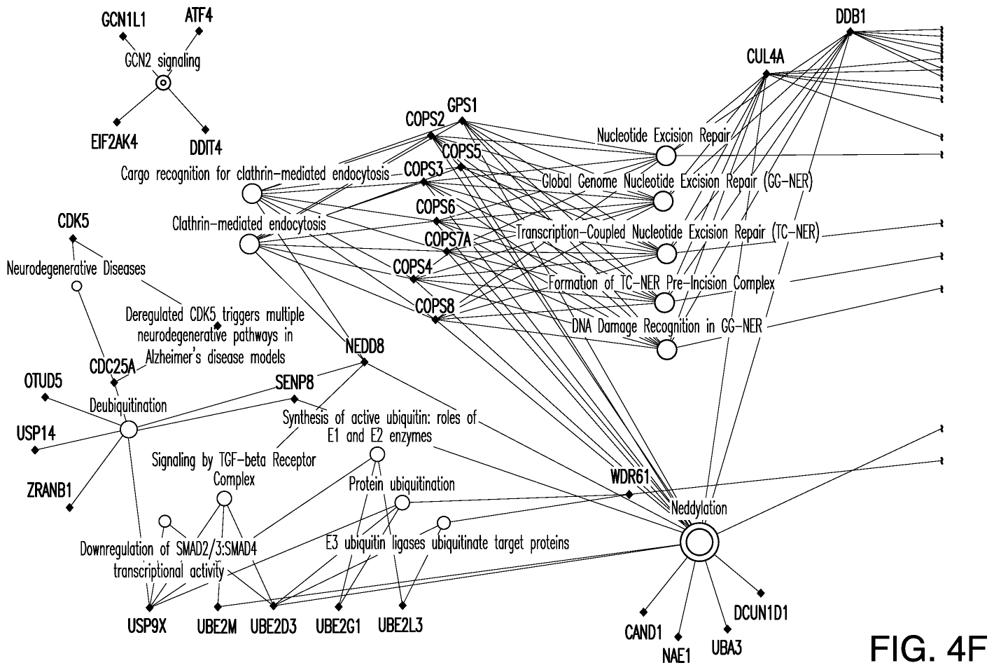

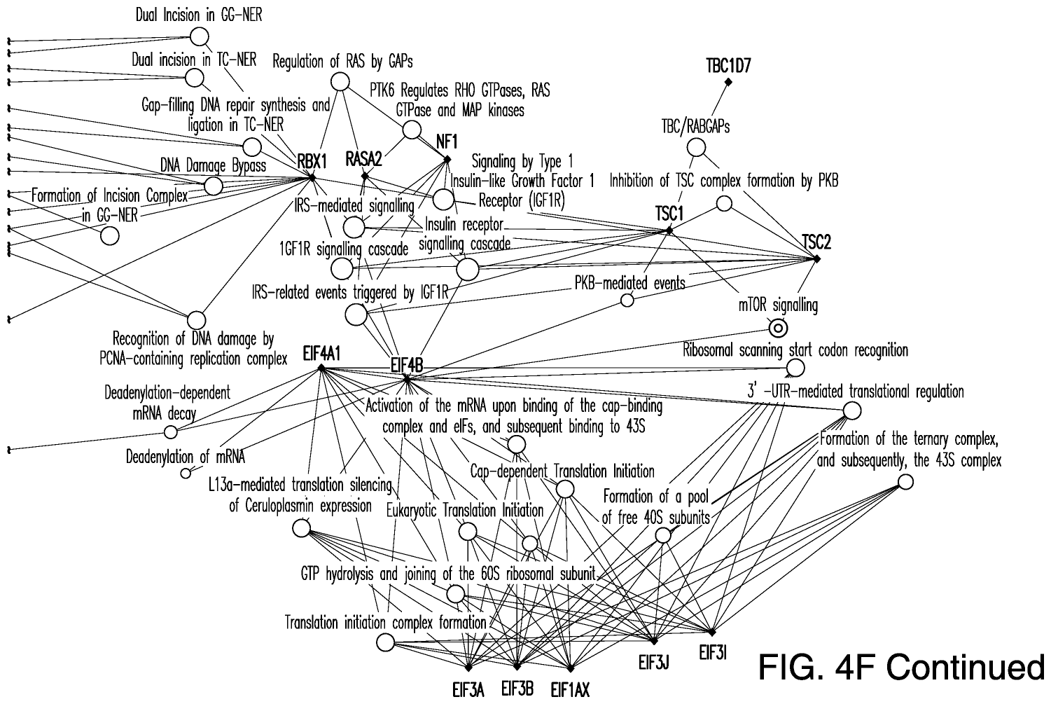

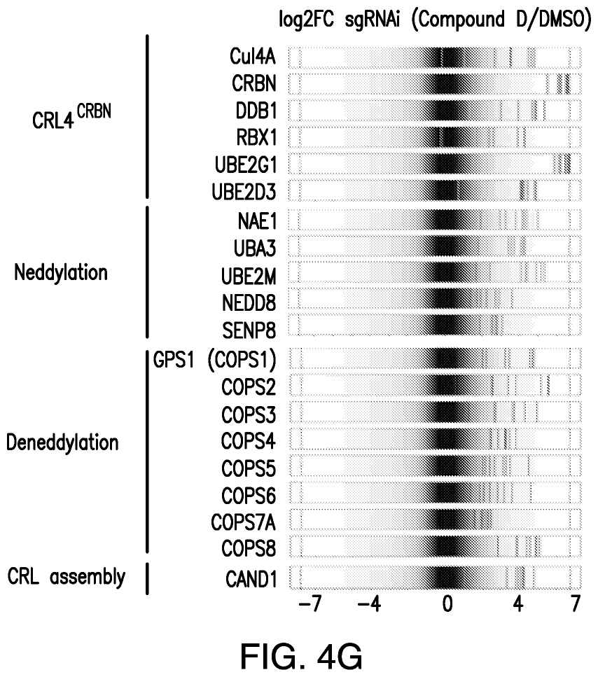

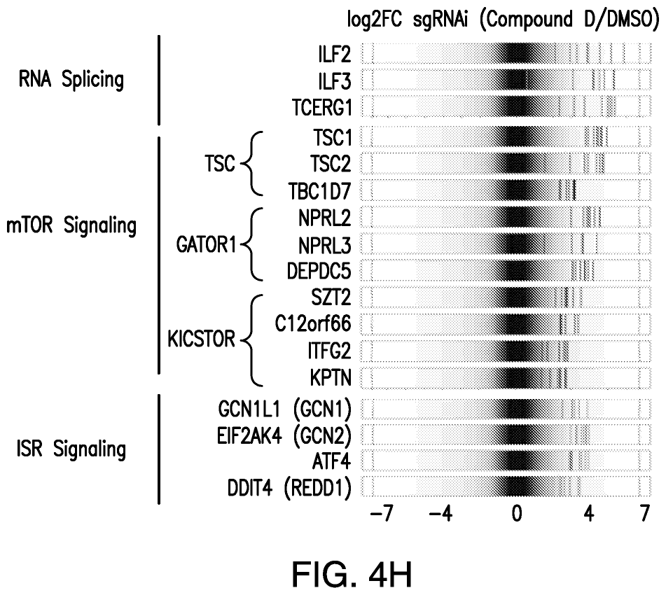

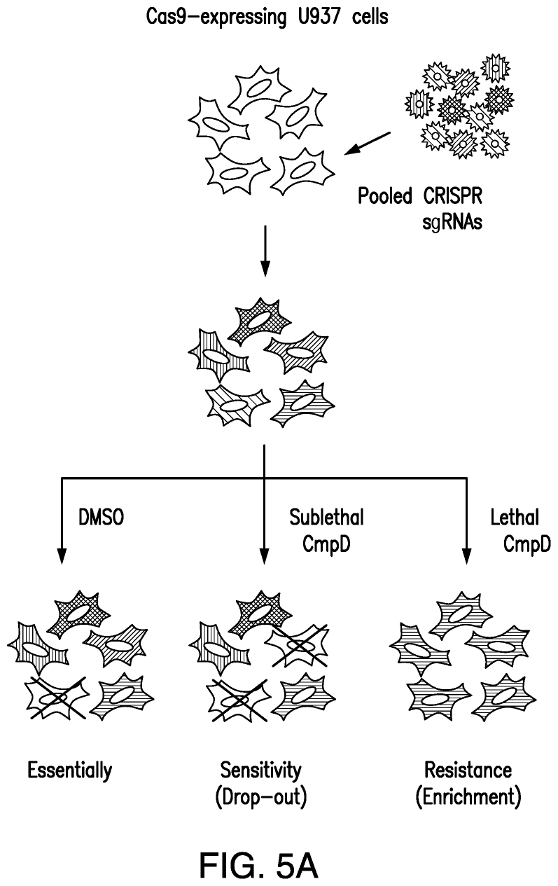

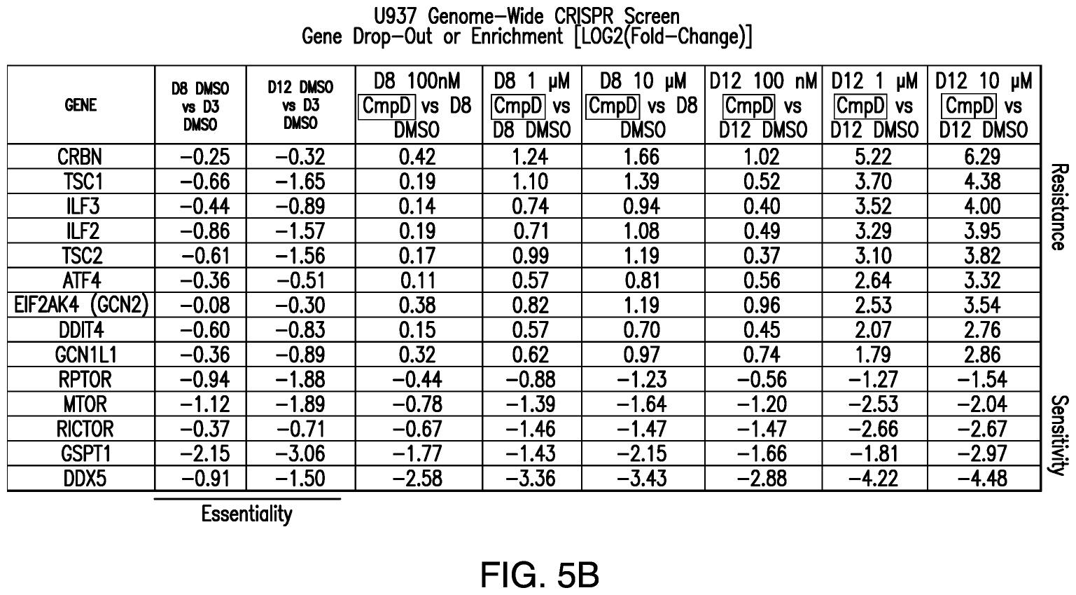

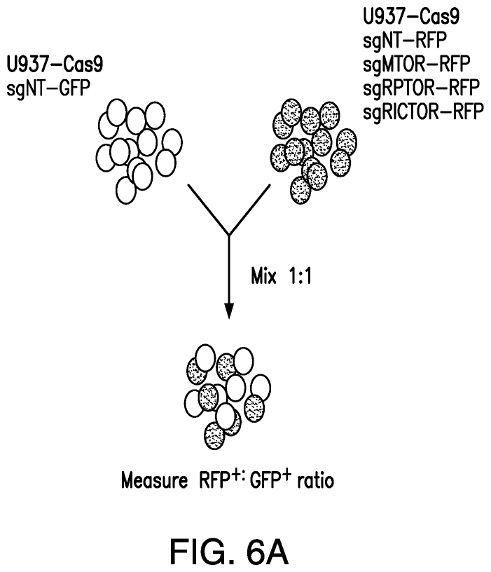

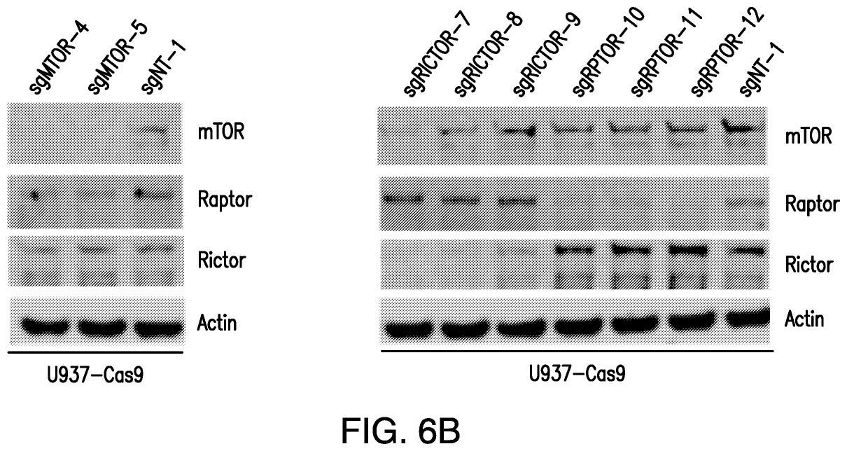

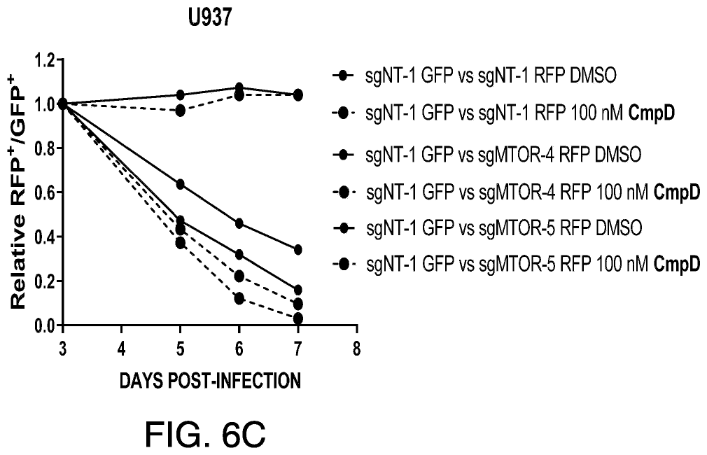

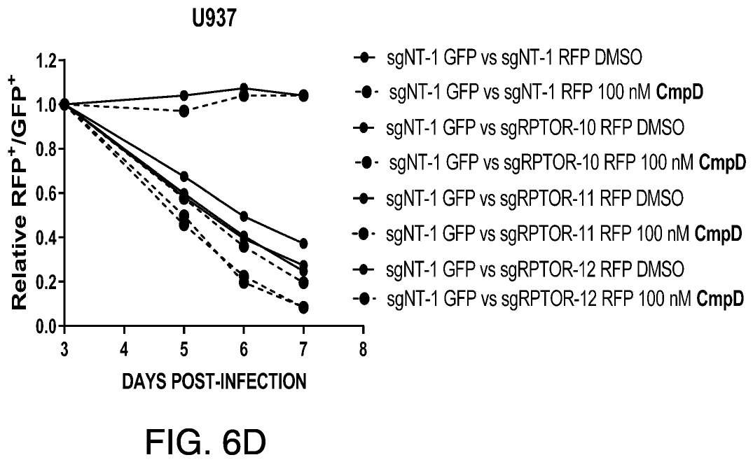

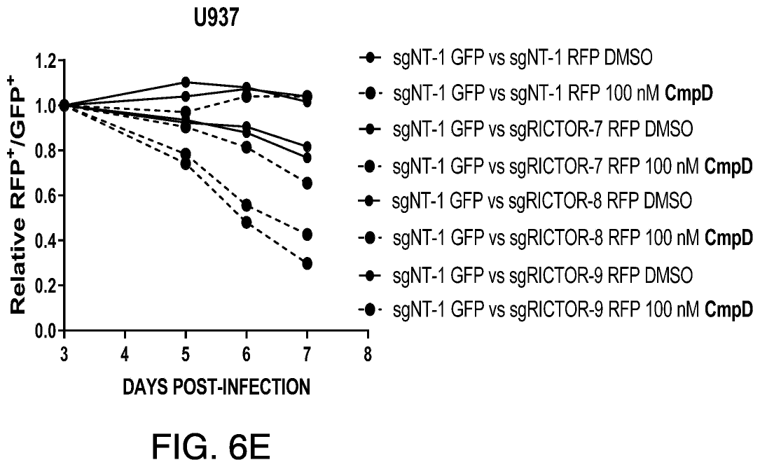

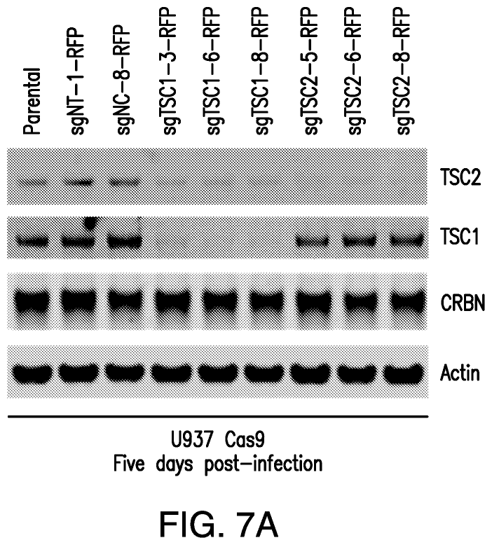

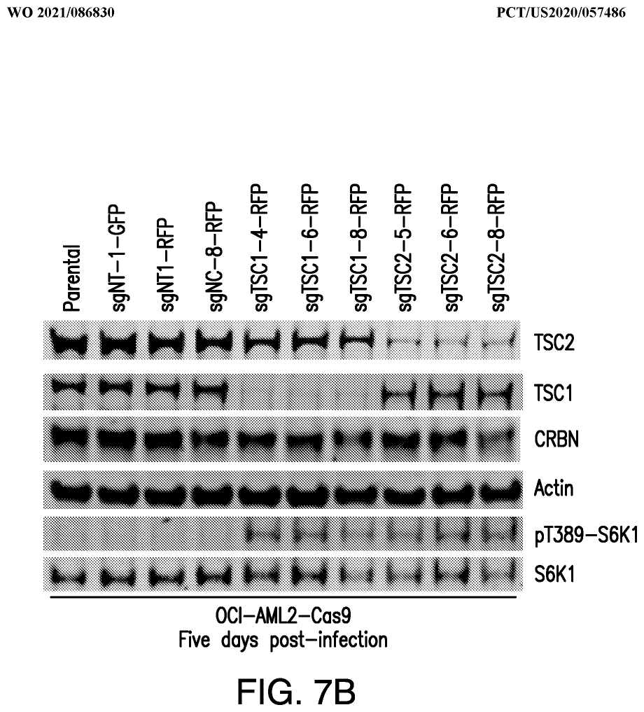

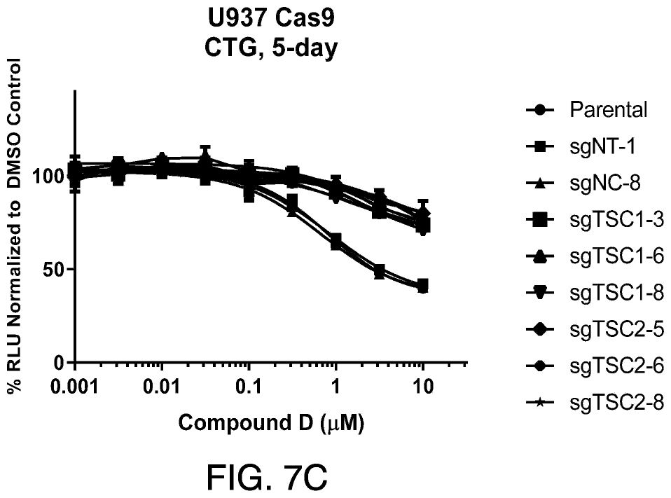

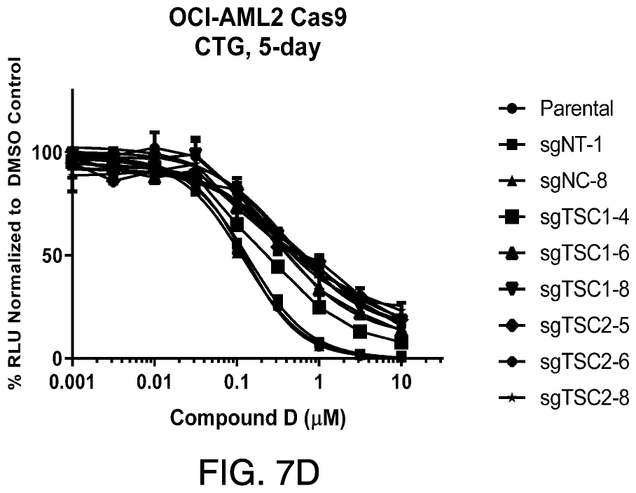

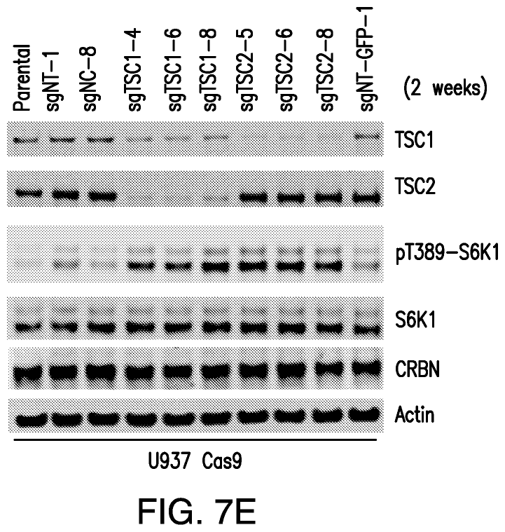

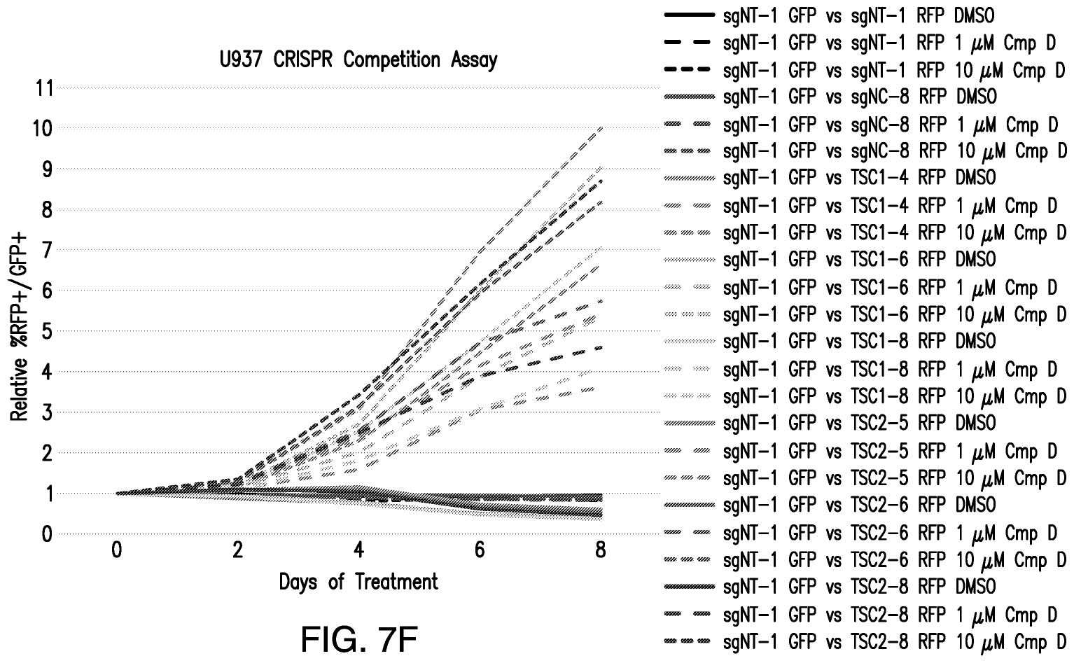

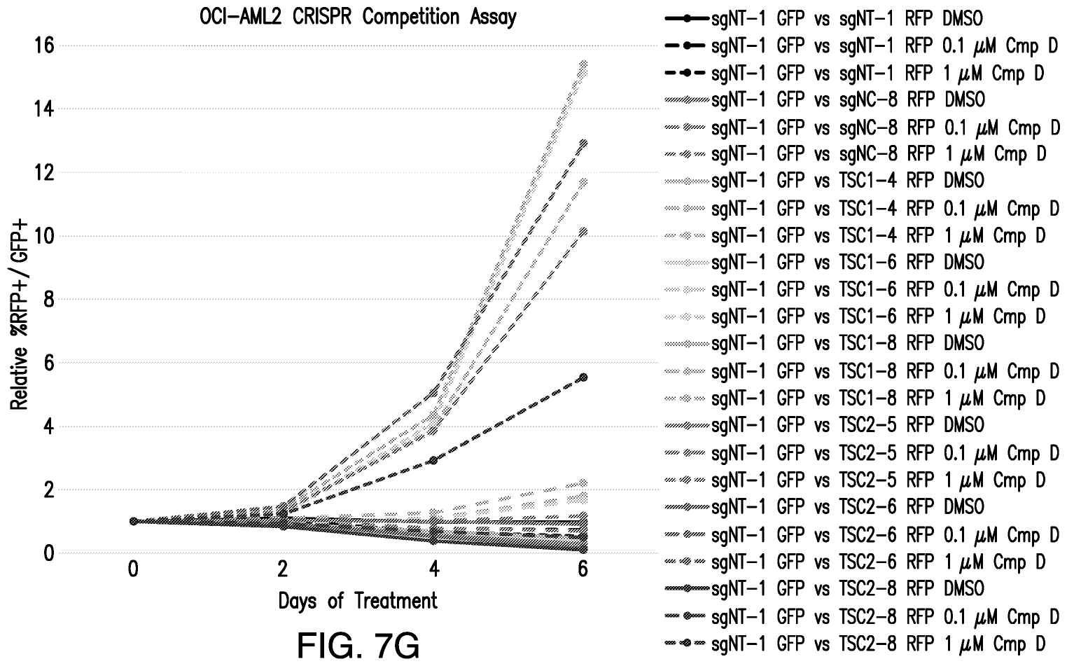

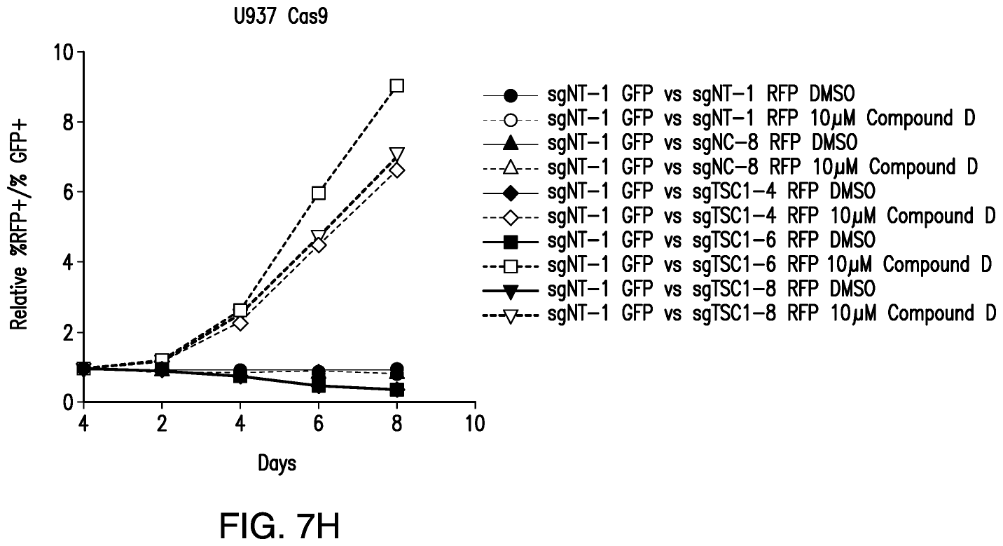

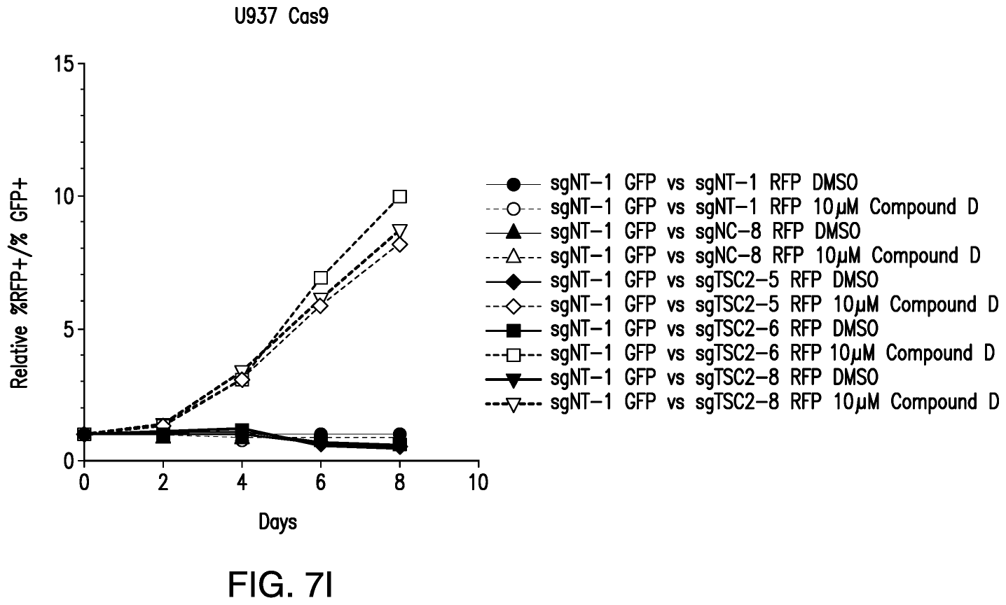

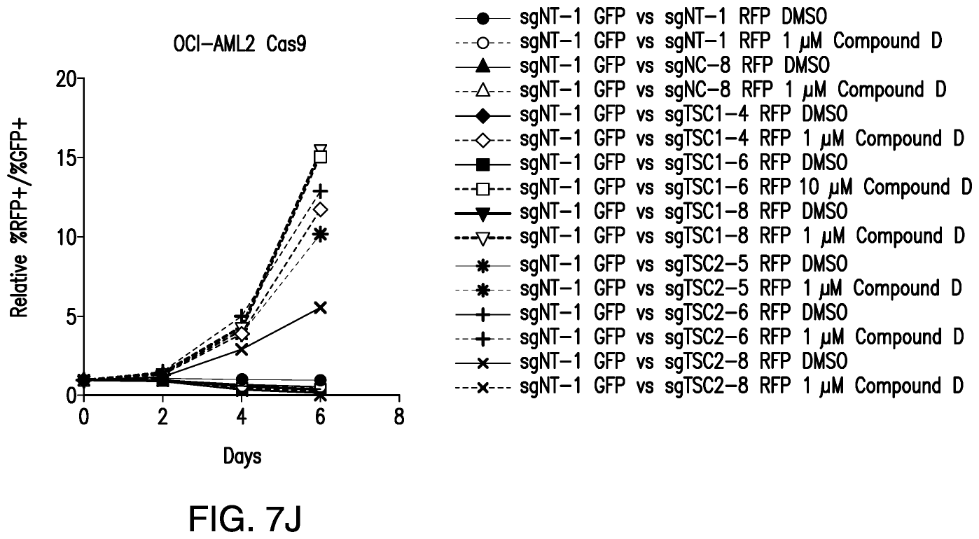

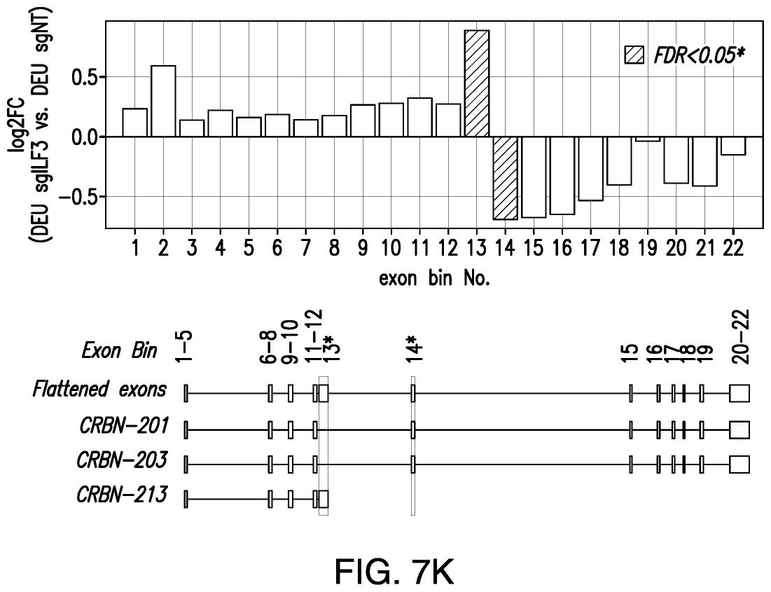

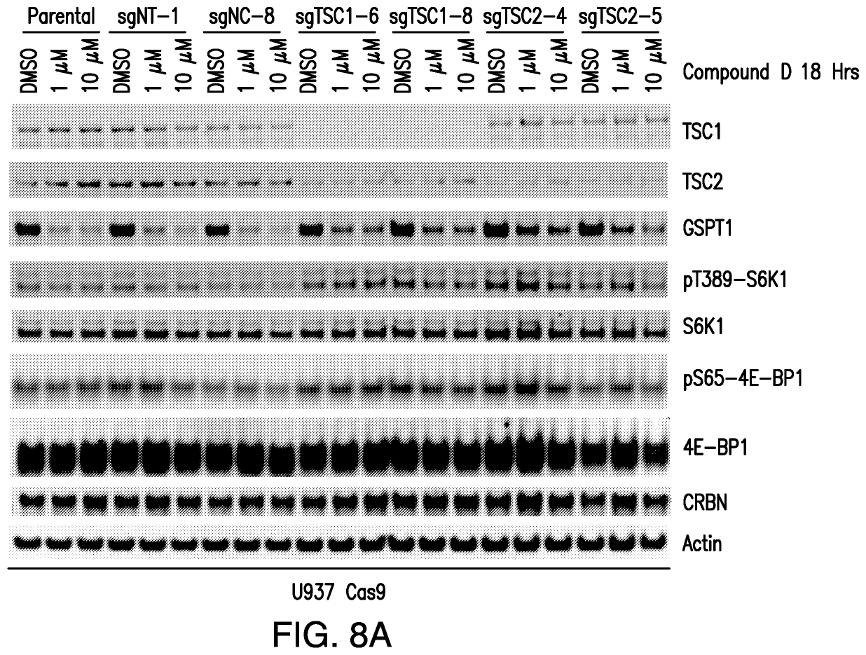

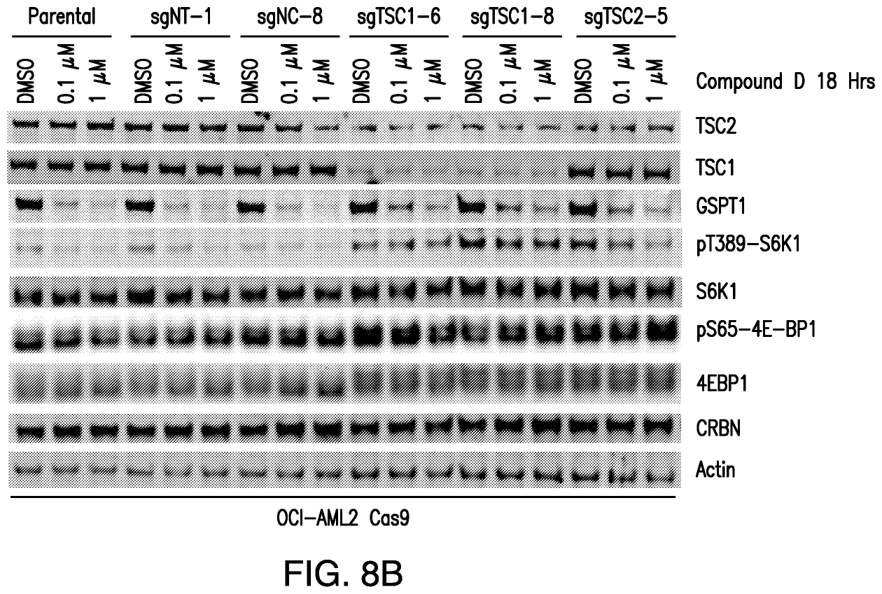

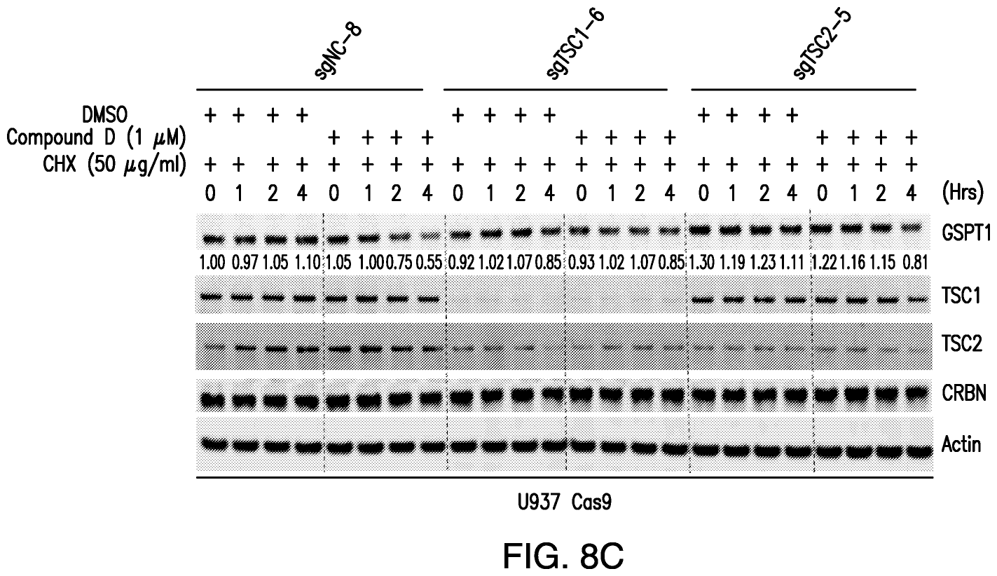

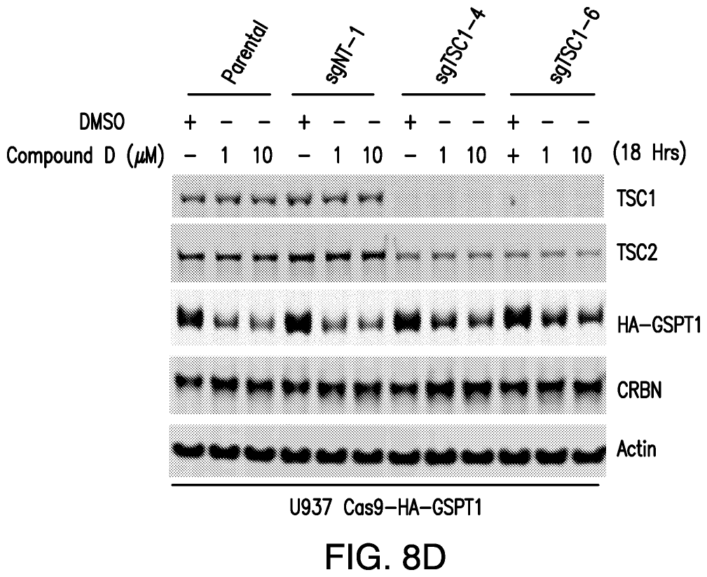

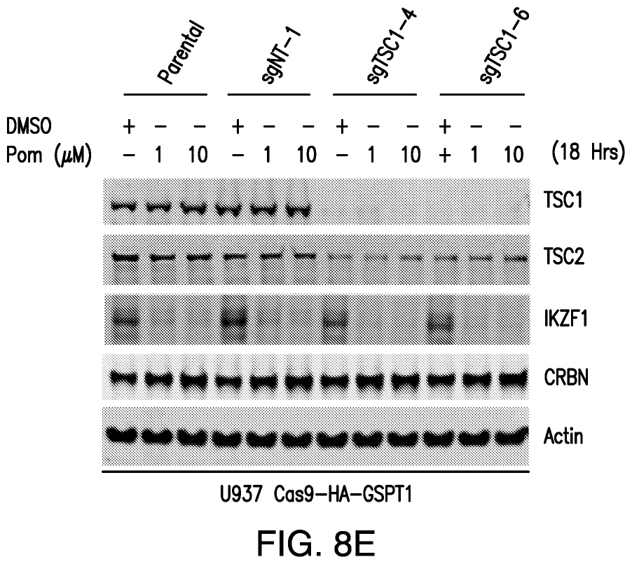

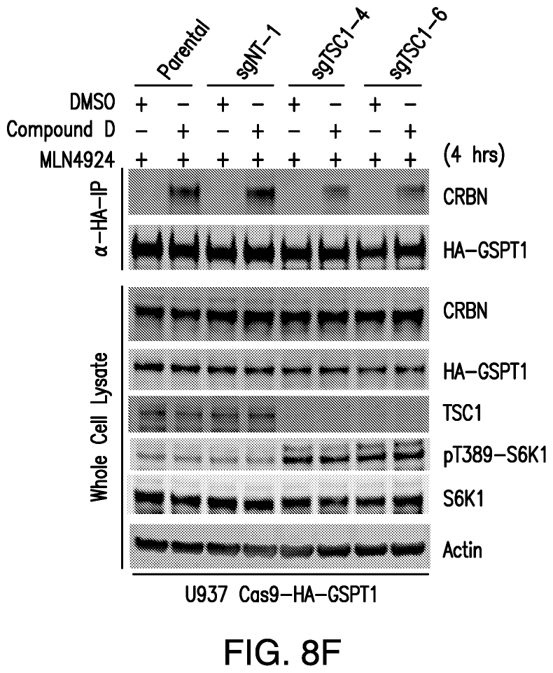

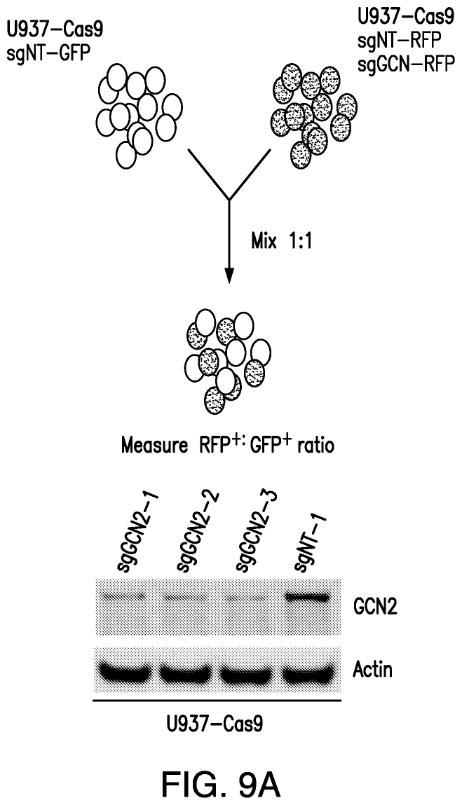

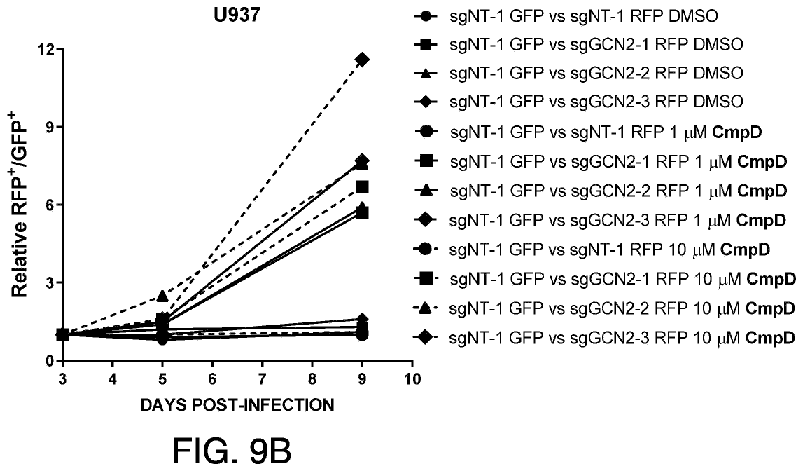

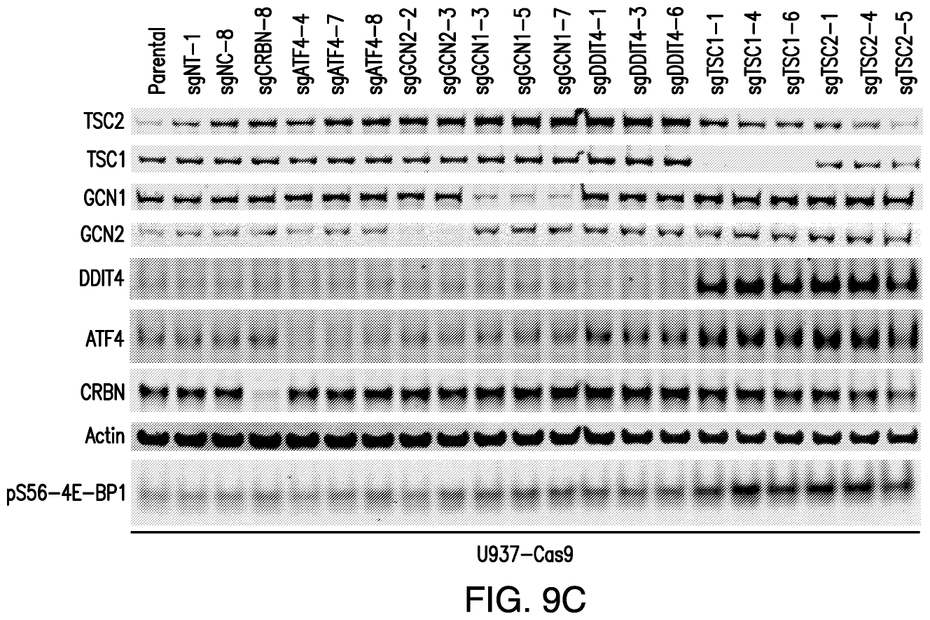

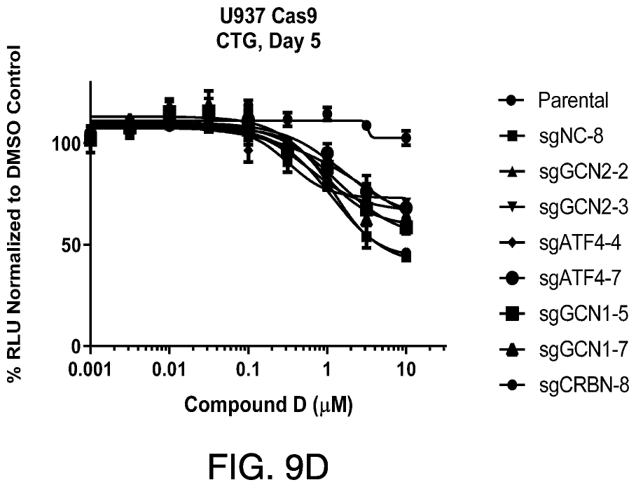

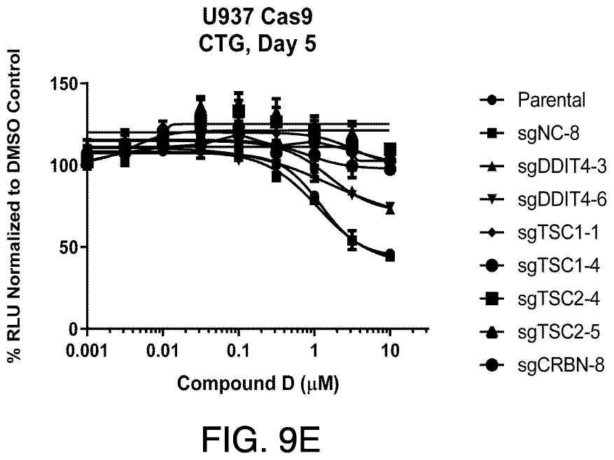

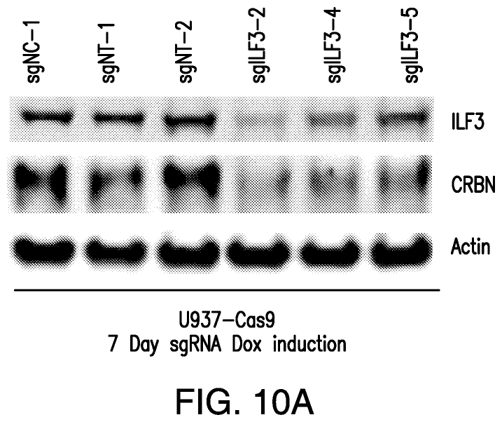

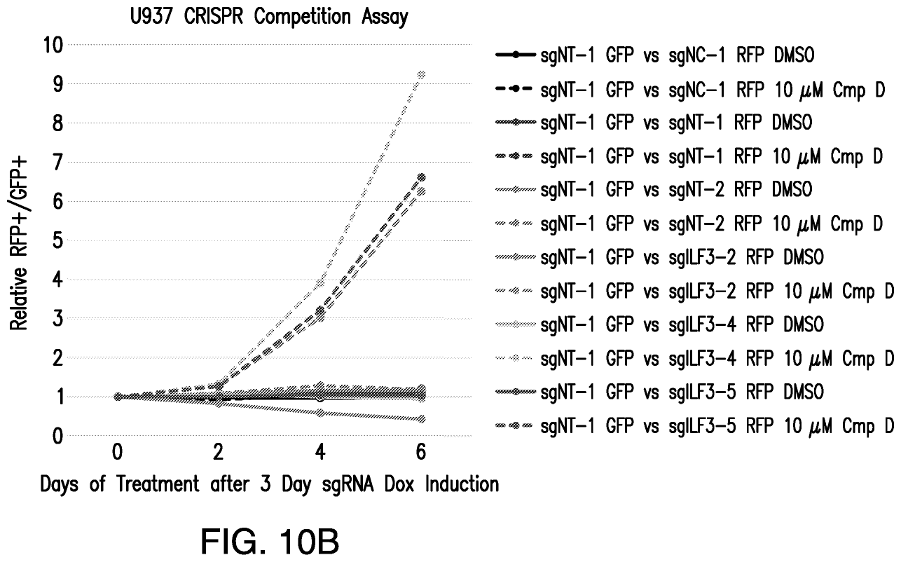

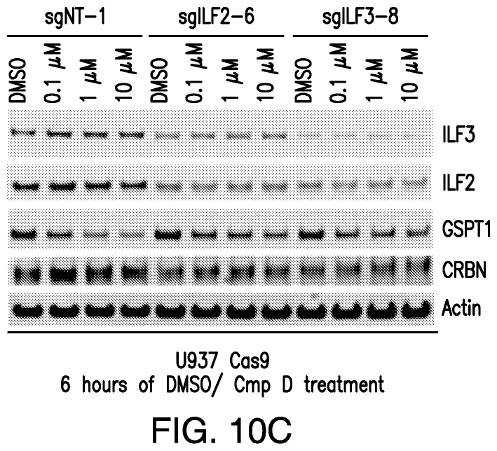

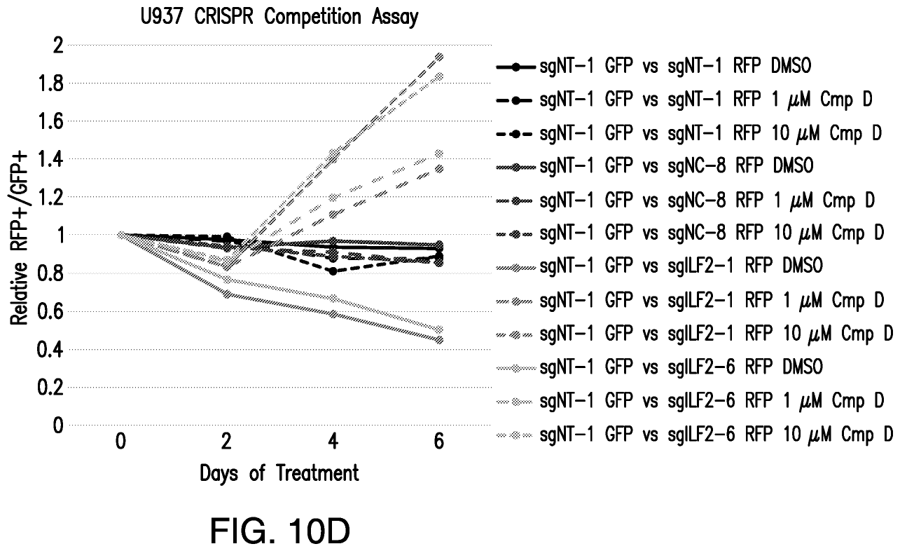

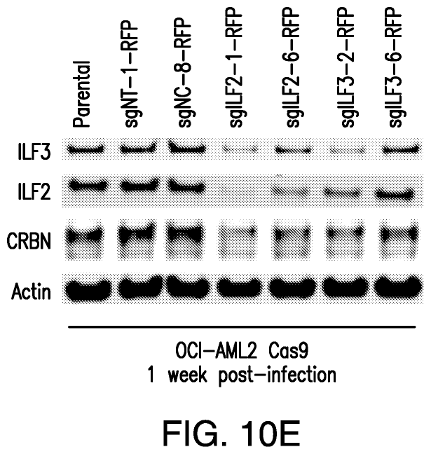

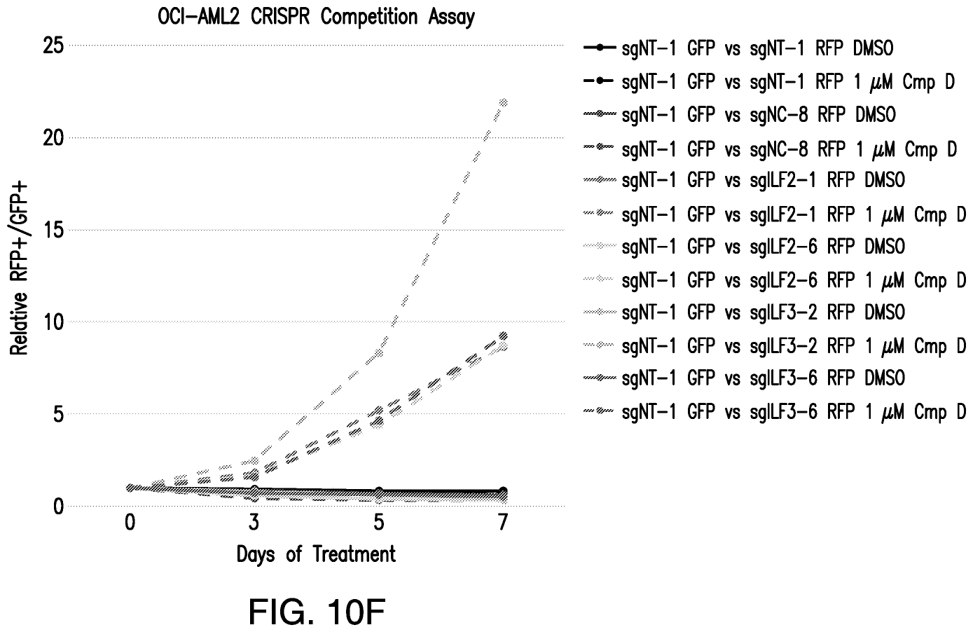

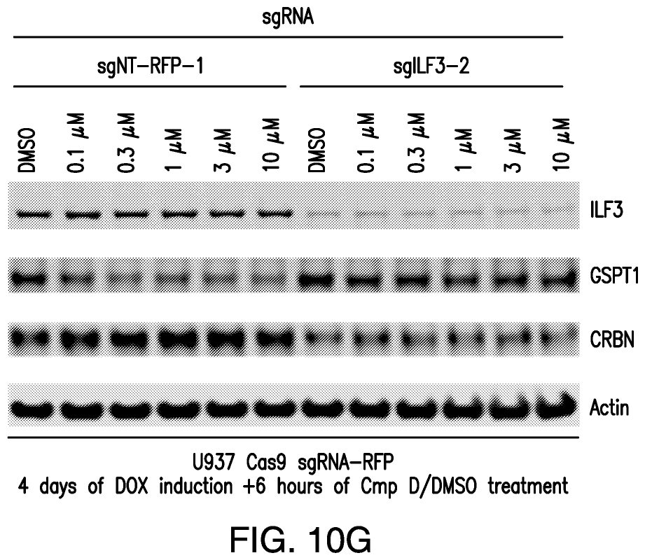

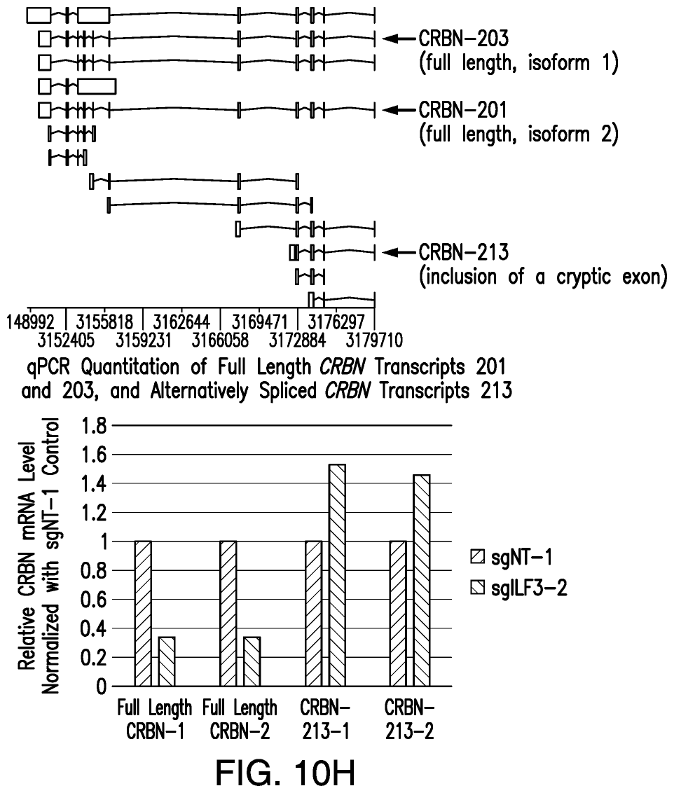

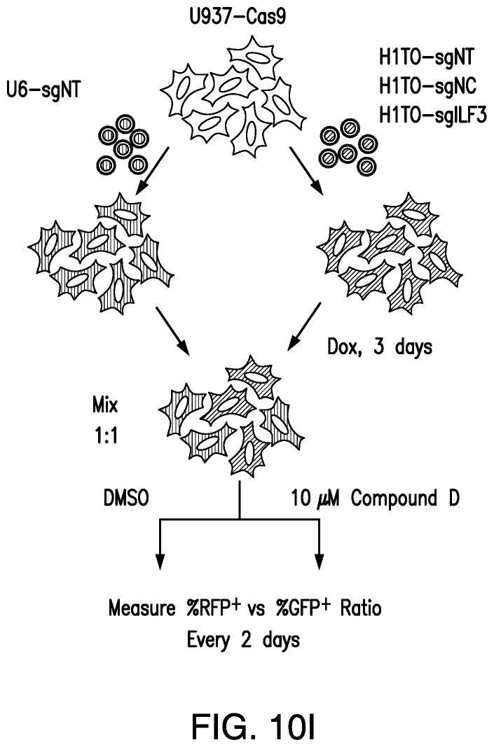

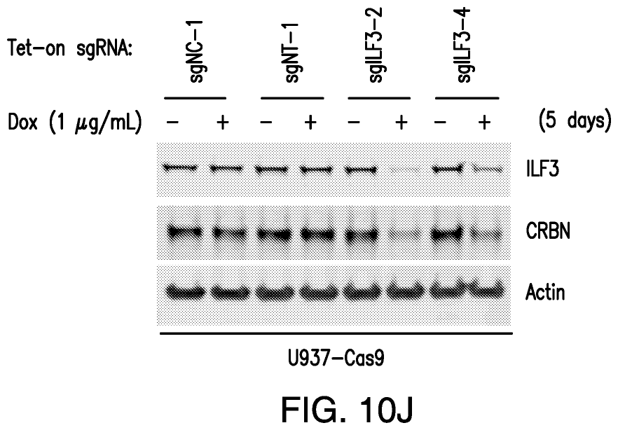

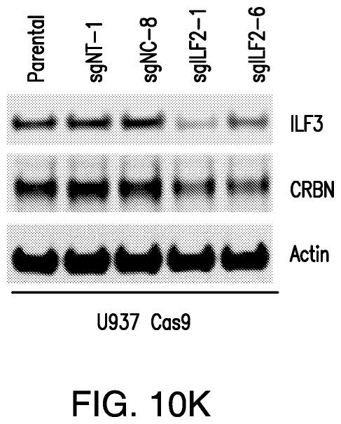

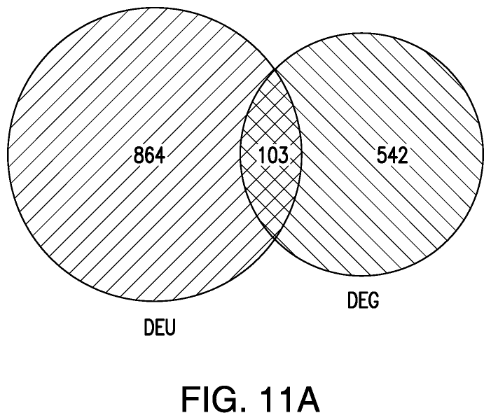

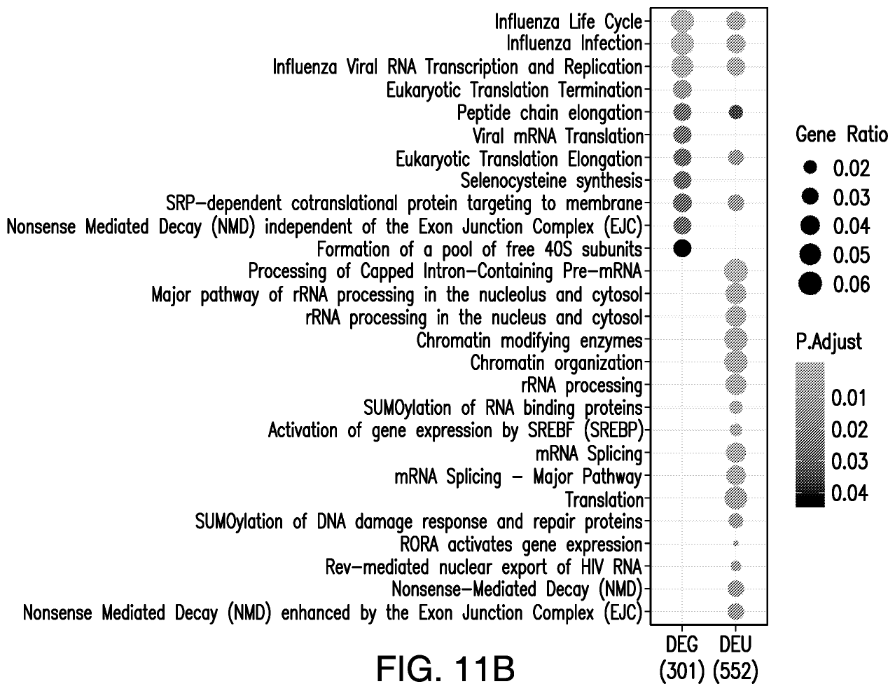

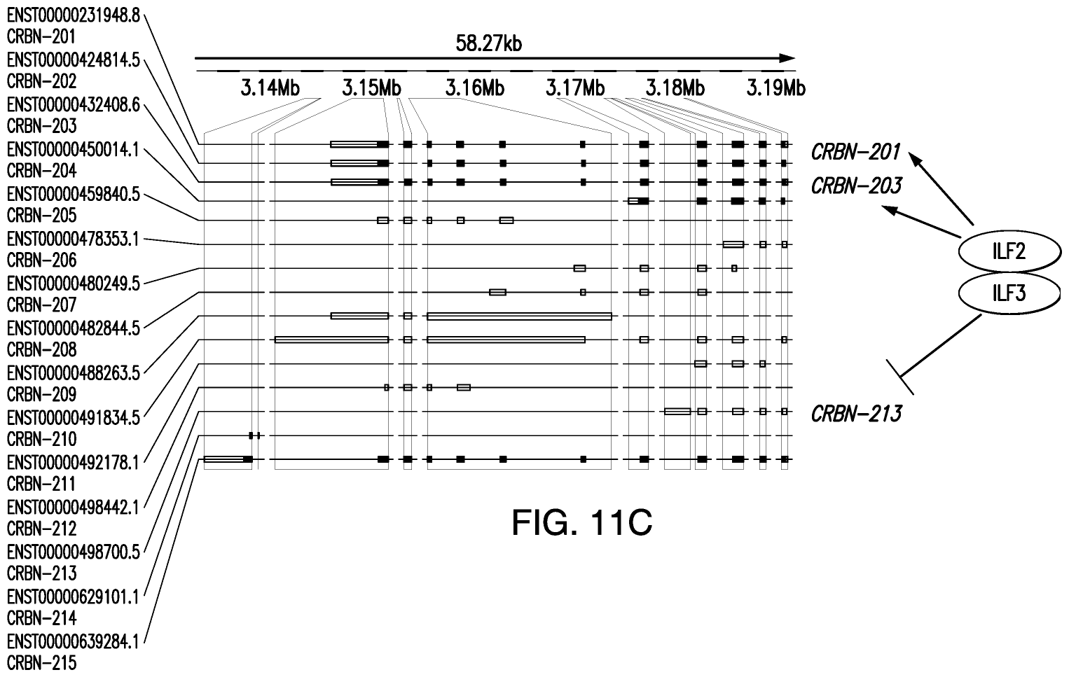

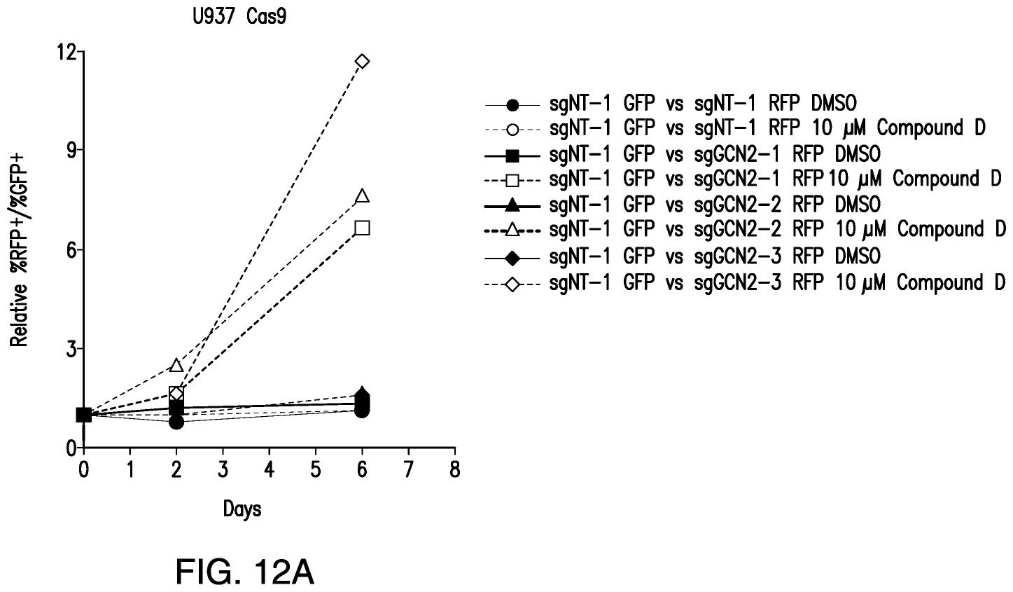

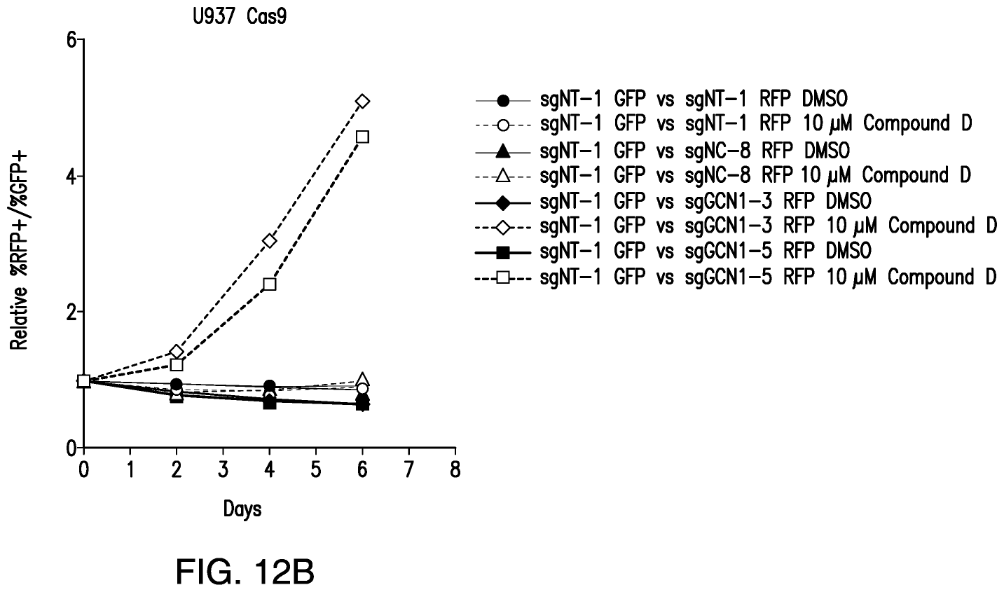

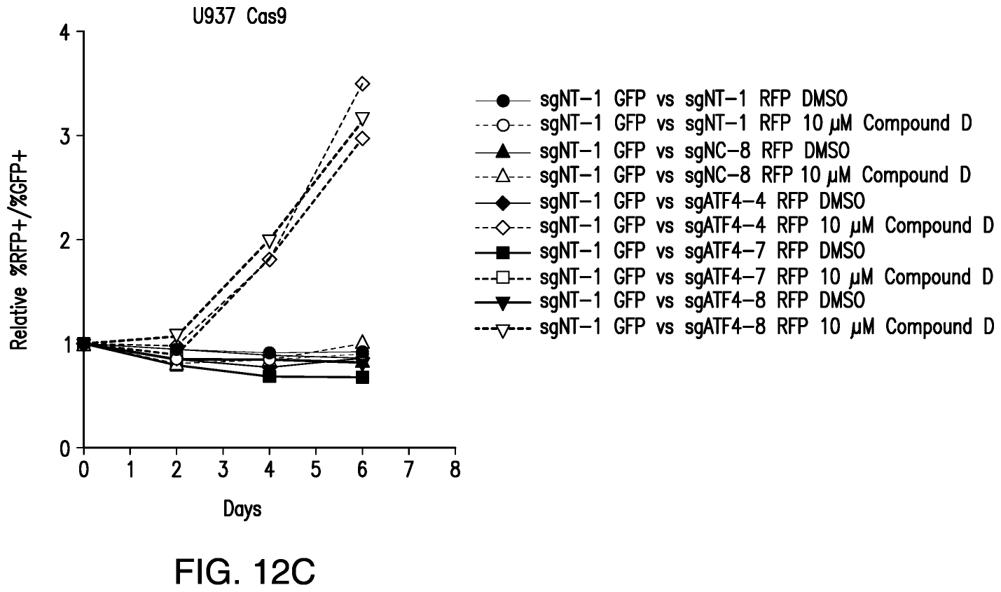

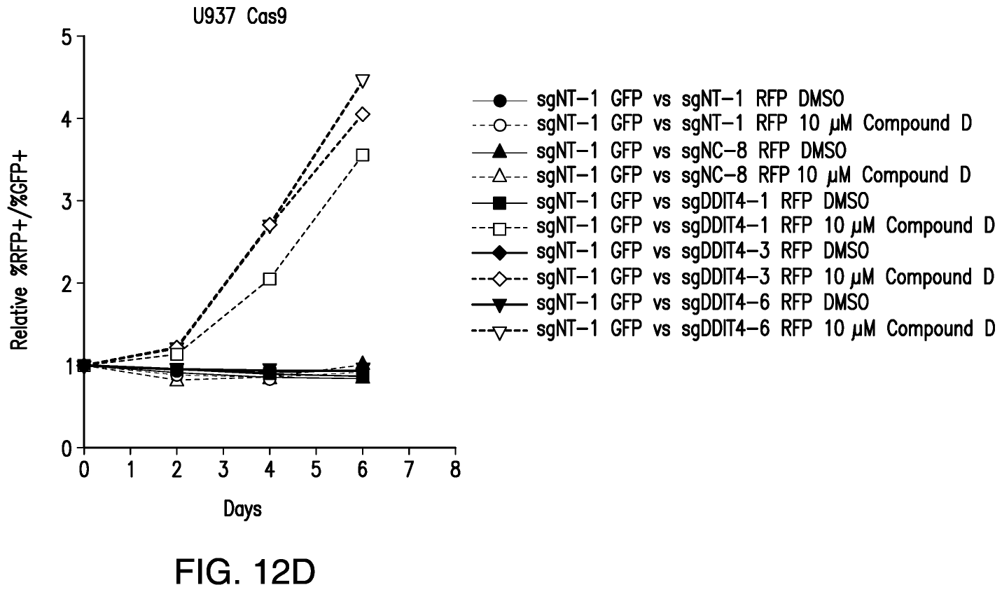

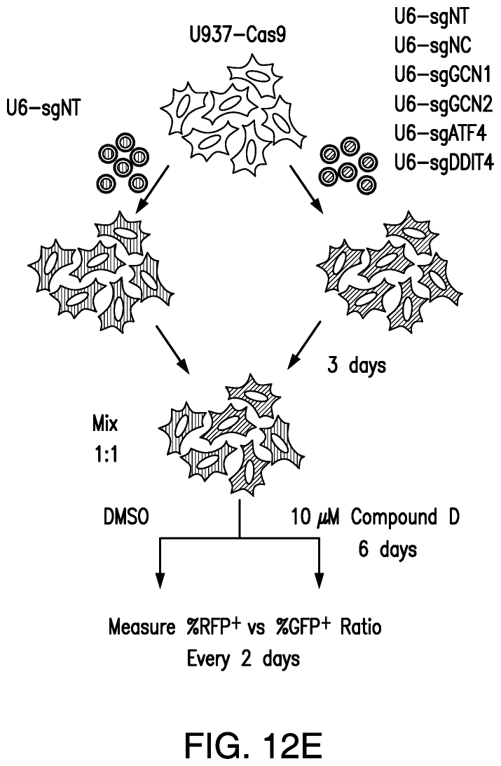

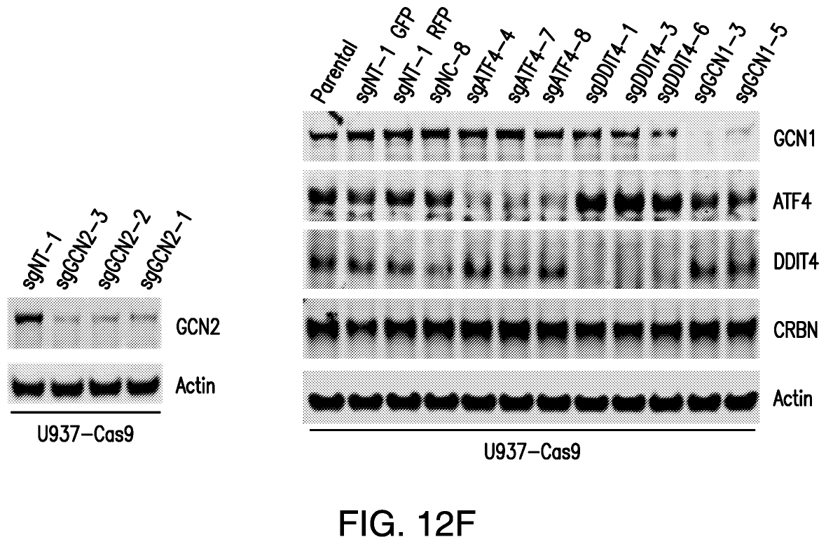

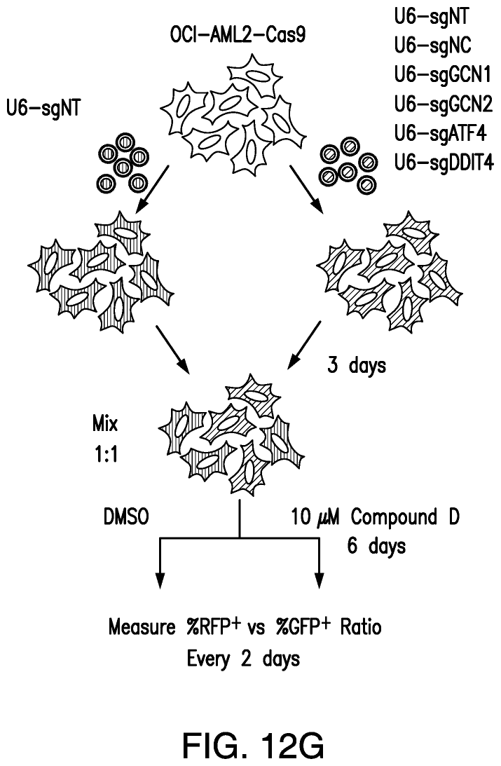

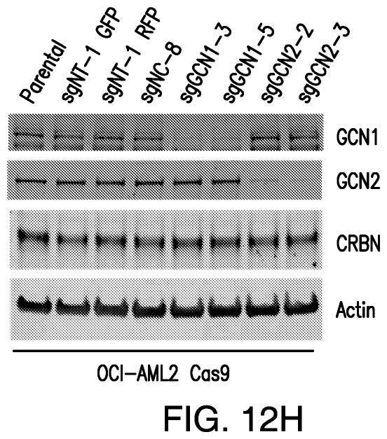

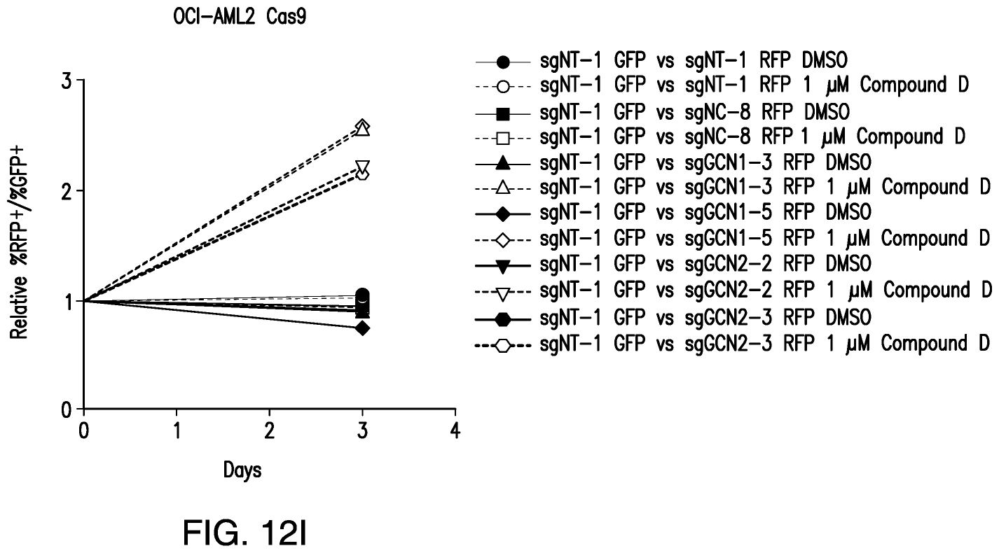

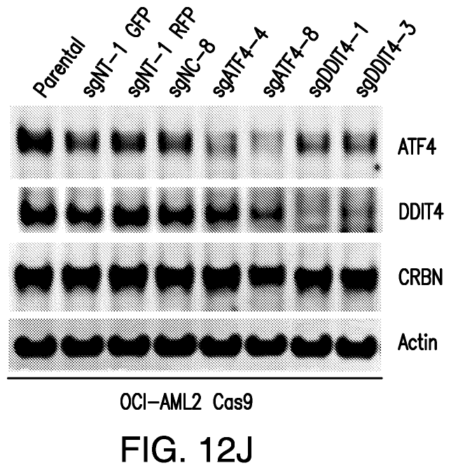

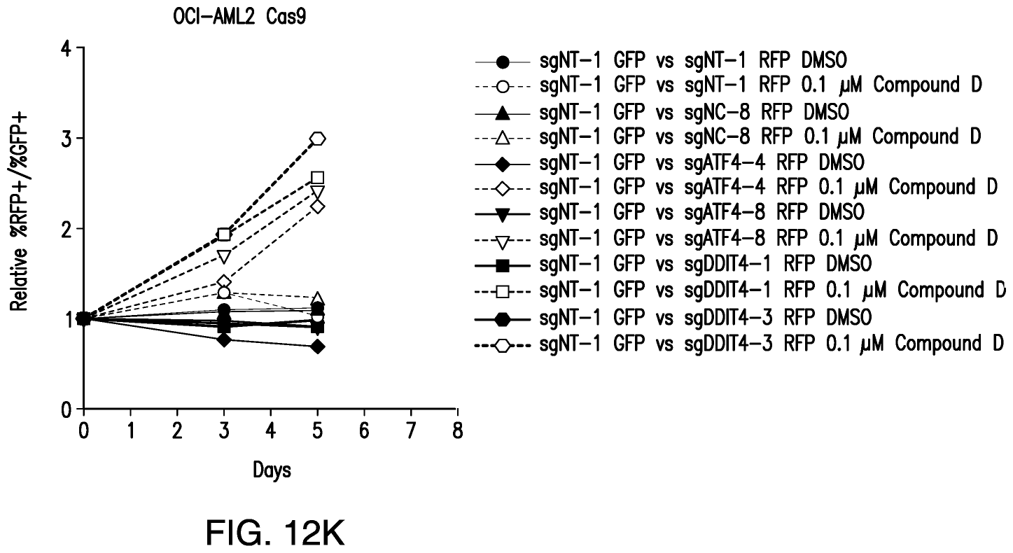

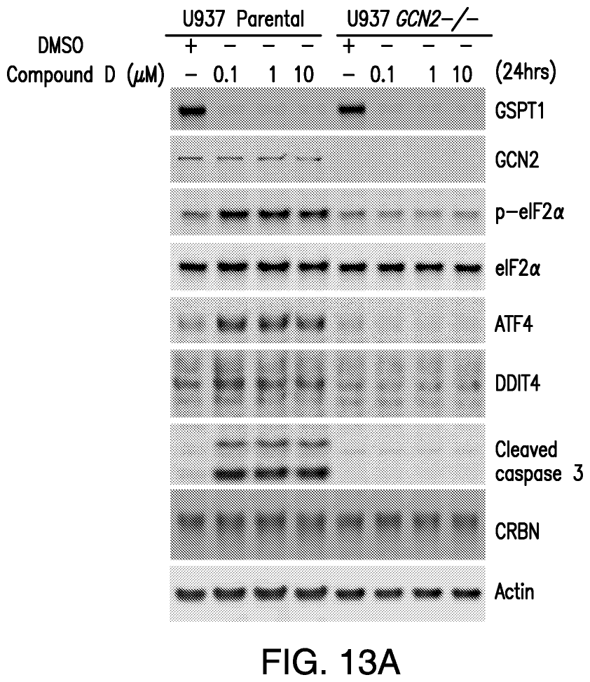

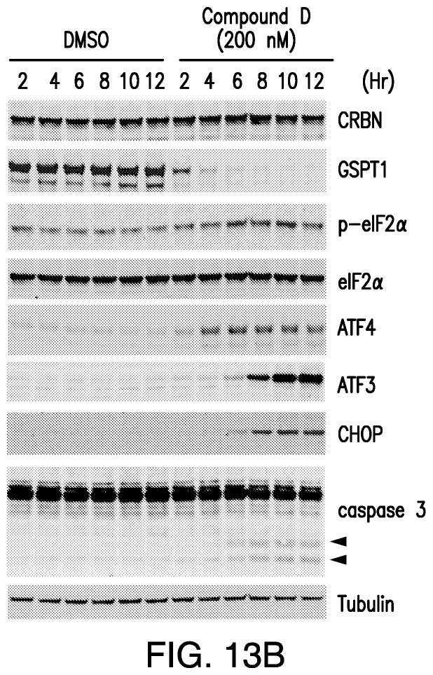

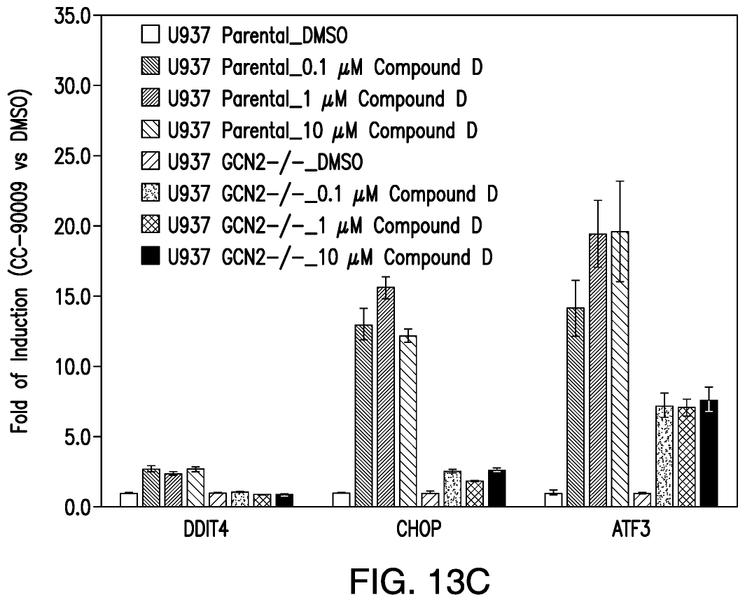

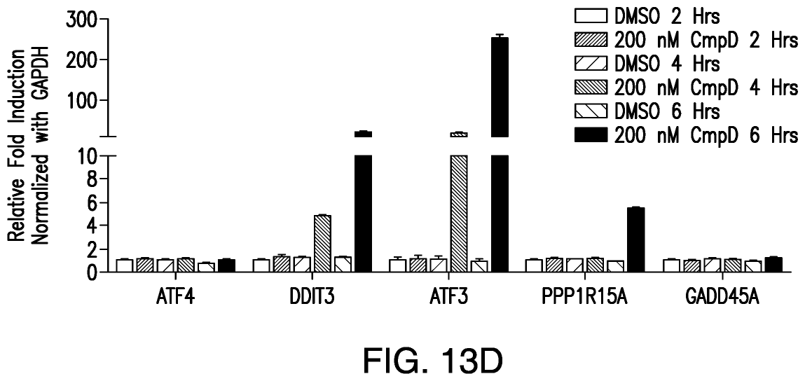

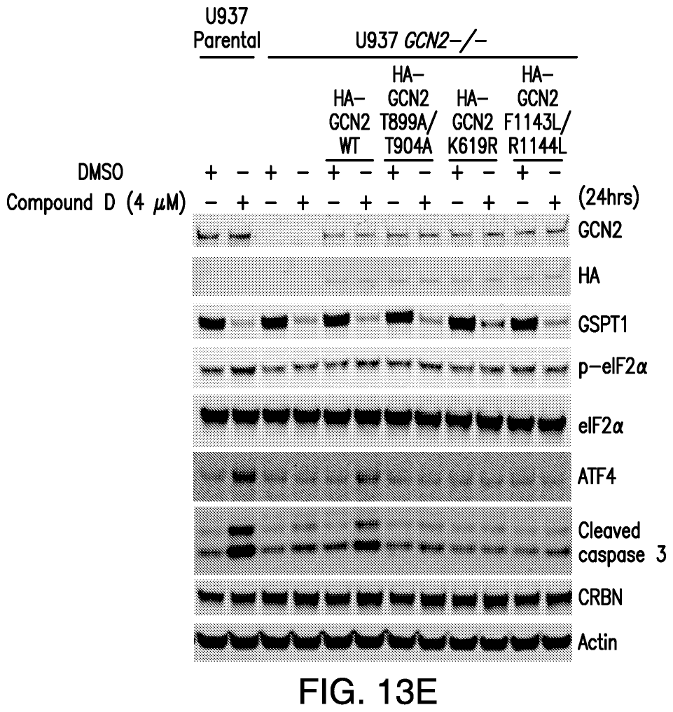

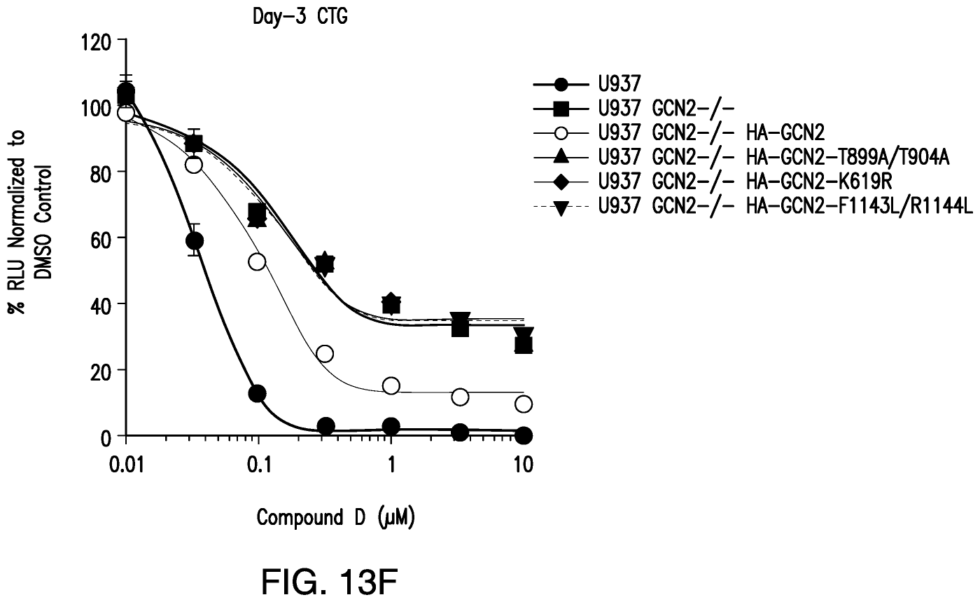

In one embodiment, Form A of Compound D has the SEM picture as shown in FIG. 3 of US Publication No. 2019/0030018.

In one embodiment, the crystalline form of Compound D has a thermogravimetric (TGA) thermograph corresponding substantially to the representative TGA thermogram as depicted in FIG. 4 of US Publication No. 2019/0030018. In certain embodiments, no TGA weight loss is observed for Form A.

In one embodiment, crystalline form A of Compound D has a DSC thermogram corresponding substantially as depicted in FIG. 5 of US Publication No. 2019/0030018. In certain embodiments, Form A is characterized by a DSC plot comprising a melting event with an onset temperature of 229° C. and heat of fusion of 118 J/g.

In certain embodiments, Form A is characterized by dynamic vapor sorption analysis. A representative dynamic vapor sorption (DVS) isotherm plot is shown in FIG. 6 of US Publication No. 2019/0030018. In certain embodiments, when the relative humidity (“RH”) is increased from about 0% to about 90% RH, Form A exhibits less than 1.5%, less than 1.2% or about 1.2% w/w water uptake. In certain embodiments, Form A comprises less than 0.1% water as determined in a coulometric Karl Fischer (KF) titrator equipped with an oven sample processor set at 225° C.

In certain embodiments, no significant degradation or residual solvent for Form A is observed by ¹H NMR (see FIG. 7 of US Publication No. 2019/0030018).

In certain embodiments, Form A of Compound D is characterized by its stability profile upon compression. In certain embodiments, Form A is stable, e.g., its XRPD pattern remains substantially unchanged with broader diffraction peaks, upon application of 2000-psi pressure for about 1 minute (see FIG. 8 of US Publication No. 2019/0030018).

In still another embodiment, Form A of Compound D is substantially pure. In certain embodiments, the substantially pure Form A of Compound D is substantially free of other solid forms, e.g., amorphous form. In certain embodiments, the purity of the substantially pure Form A of Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

Certain embodiments Form A of Compound D is substantially pure. In certain embodiments herein Form A of Compound D is substantially free of other solid forms comprising Compound D including, e.g., Forms B, C, D, E and/or an amorphous solid form comprising Compound D. In certain embodiments, Form A is a mixture of solid forms comprising Compound D, including, e.g., a mixture comprising one or more of the following: Forms B, C, D, E and an amorphous solid form comprising Compound D.

### Form B of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein are prepared from anhydrous Form B of Compound D.

In certain embodiments, Form B is obtained by anti-solvent recrystallization from certain solvent systems, for example, solvent systems comprising one or more of the following solvents: methanol/water, DMSO/isopropanol, DMSO/toluene, and DMSO/water. In certain embodiments, Form B is obtained by cooling recrystallization from THF/water (1:1).

In certain embodiments, Form B is crystalline, as indicated by, e.g., X-ray powder diffraction measurements. In one embodiment, Form B of Compound D has an X-ray powder diffraction pattern substantially as shown in FIG. 9 of US Publication No. 2019/0030018.

In one embodiment, Form B of Compound D has one or more characteristic X-ray powder diffraction peaks at a two-theta angle of approximately 15.4, 16.3, 16.7, 17.7, 20.4, 25.6 or 27.5, degrees 2θ as depicted in FIG. 9 of US Publication No. 2019/0030018. In another embodiment, Form B of Compound D has one, two, three or four characteristic X-ray powder diffraction peaks at a two-theta angle of approximately 16.7, 25.6, 15.4 or 16.3 degrees 2θ. In another embodiment, Form B of Compound D has one, two, three, four, five, six or seven characteristic X-ray powder diffraction peaks as set forth in Table B. In another embodiment, Form B of Compound D has one, two, or three characteristic X-ray powder diffraction peaks as set forth in Table B.

TABLE B

| | Pos. | d-spacing | Rel. Int. |
|---|---|---|---|
| No. | [°2 Th.] | [Å] | [%] |
| 1 | 7.01 | 12.6035 | 9.3 |
| 2 | 11.58 | 7.6444 | 8.3 |
| 3 | 11.80 | 7.5027 | 6.8 |
| 4 | 12.73 | 6.9551 | 18.4 |
| 5 | 15.38 | 5.7601 | 34.8 |
| 6 | 16.32 | 5.4330 | 31.4 |
| 7 | 16.72 | 5.3012 | 100.0 |
| 8 | 17.72 | 5.0046 | 26.6 |
| 9 | 18.13 | 4.8930 | 19.8 |
| 10 | 18.77 | 4.7271 | 7.5 |
| 11 | 20.41 | 4.3516 | 22.0 |
| 12 | 21.02 | 4.2258 | 15.9 |
| 13 | 21.21 | 4.1881 | 13.5 |
| 14 | 21.93 | 4.0529 | 3.4 |
| 15 | 23.68 | 3.7581 | 14.2 |
| 16 | 25.01 | 3.5601 | 10.4 |
| 17 | 25.63 | 3.4755 | 37.3 |
| 18 | 26.19 | 3.4030 | 9.8 |
| 19 | 26.73 | 3.3349 | 8.5 |
| 20 | 27.45 | 3.2499 | 20.9 |
| 21 | 27.71 | 3.2193 | 9.4 |
| 22 | 28.22 | 3.1623 | 11.8 |
| 23 | 29.48 | 3.0296 | 4.7 |
| 24 | 30.10 | 2.9692 | 15.0 |
| 25 | 31.08 | 2.8775 | 18.3 |
| 26 | 31.65 | 2.8272 | 6.2 |
| 27 | 34.29 | 2.6150 | 3.4 |

In one embodiment, Form B of Compound D has the SEM picture as shown in FIG. 10 of US Publication No. 2019/0030018. In one embodiment, a crystalline form of Compound D has a thermogravimetric (TGA) thermograph corresponding substantially to the representative TGA thermogram as depicted in FIG. 11 of US Publication No. 2019/0030018. In certain embodiments, Form B shows no TGA weight loss below 170° C. In certain embodiments, Form B shows a TGA weight loss of 0.4% between 170-230° C.

In one embodiment, crystalline Form B of Compound D has a DSC thermogram corresponding substantially as depicted in FIG. 12 of US Publication No. 2019/0030018. In certain embodiments, Form B is characterized by a DSC plot comprising a melt/recrystallization event at 219˜224° C. and a major melting event with a peak temperature of 231° C.

In certain embodiments, Form B is characterized by dynamic vapor sorption analysis. A representative dynamic vapor sorption (DVS) isotherm plot is shown in FIG. 13 of US Publication No. 2019/0030018. In certain embodiments, when the relative humidity (“RH”) is increased from about 0% to about 90% RH, Form B exhibits about 1.4% w/w water uptake. In certain embodiments, Form B comprises less than 0.1% water as determined in a coulometric Karl Fischer (KF) titrator equipped with an oven sample processor set at 225° C.

In certain embodiments, Form B shows no significant degradation or residual solvent by ¹H NMR (see FIG. 14 of US Publication No. 2019/0030018).

In certain embodiments, Form B of Compound D is characterized by its stability profile upon compression. In certain embodiments, Form B is stable, e.g., its XRPD pattern remains substantially unchanged with broader diffraction peaks, upon application of 2000-psi pressure for about 1 minute (see FIG. 15 of US Publication No. 2019/0030018).

In still another embodiment, Form B of Compound D is substantially pure. In certain embodiments, the substantially pure Form B of Compound D is substantially free of other solid forms, e.g., amorphous form. In certain embodiments, the purity of the substantially pure Form B of Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

Certain embodiments, Form B of Compound D is substantially pure. In certain embodiments, Form B of Compound D is substantially free of other solid forms comprising Compound D including, e.g., Forms A, C, D, E, and/or an amorphous solid form comprising Compound D. In certain embodiments, Form B is a mixture of solid forms comprising Compound D, including, e.g., a mixture comprising one or more of the following: Forms A, C, D, E, and an amorphous solid form comprising Compound D.

### Form C of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein are prepared from anhydrous Form C of Compound D. In certain embodiments, Form C is the most thermodynamically stable anhydrate among the crystal forms of Compound D.

In certain embodiments, Form C is obtained by slurrying Compound D in certain solvent systems, for example, solvent systems comprising one or more of the following solvents: acetonitril/water, acetone, or ethanol/water for extended period of time.

In certain aspects, Form C is obtained by slurrying Form B (1×wt) in acetone (30×vol) at an elevated temperature, for example, from 60-80° C. or 70-75° C. for at least 24 hours, and cooling the mixture to room temperature. In one aspect, the slurrying is conducted at a temperature of 70-75° C. under nitrogen pressure of 50-55-psi. In one aspect, the mixture is cooled to room temperature over at least 6 hours.

In certain embodiments, Form C is crystalline, as indicated by, e.g., X-ray powder diffraction measurements. In one embodiment, Form C of Compound D has an X-ray powder diffraction pattern substantially as shown in FIG. 16 of US Publication No. 2019/0030018.

In one embodiment, Form C of Compound D has one or more characteristic X-ray powder diffraction peaks at a two-theta angle of approximately 7.4, 11.5, 15.8, 16.7, 16.9, 17.7, 18.4, 19.2, 19.5, 21.1, 23.4, 24.7, or 29.9, degrees 2θ as depicted in FIG. 16 of US Publication No. 2019/0030018. In another embodiment, Form C of Compound D has one, two, three or four characteristic X-ray powder diffraction peaks at a two-theta angle of approximately 16.7, 16.9, 17.7 or 24.7 degrees 2θ. In another embodiment, Form C of Compound D has one, two, three, four, five, six or seven characteristic X-ray powder diffraction peaks as set forth in Table C. In another embodiment, Form C of Compound D has one, two, or three characteristic X-ray powder diffraction peaks as set forth in Table C.

TABLE C

| | Pos. | d-spacing | Rel. Int. |
|---|---|---|---|
| No. | [°2 Th.] | [Å] | [%] |
| 1 | 7.36 | 12.0091 | 32.0 |
| 2 | 9.14 | 9.6750 | 8.3 |
| 3 | 11.51 | 7.6855 | 44.7 |
| 4 | 12.22 | 7.2420 | 4.9 |
| 5 | 15.17 | 5.8398 | 8.4 |
| 6 | 15.82 | 5.6011 | 31.8 |
| 7 | 16.68 | 5.3140 | 57.1 |
| 8 | 16.92 | 5.2392 | 86.8 |
| 9 | 17.72 | 5.0057 | 100.0 |
| 10 | 18.39 | 4.8242 | 21.9 |
| 11 | 19.18 | 4.6268 | 36.4 |
| 12 | 19.45 | 4.5649 | 27.1 |
| 13 | 21.11 | 4.2077 | 40.4 |
| 14 | 21.82 | 4.0724 | 12.4 |
| 15 | 22.28 | 3.9902 | 12.0 |
| 16 | 22.57 | 3.9398 | 17.6 |
| 17 | 23.36 | 3.8082 | 24.7 |
| 18 | 24.26 | 3.6695 | 7.1 |
| 19 | 24.71 | 3.6026 | 72.5 |
| 20 | 25.74 | 3.4615 | 16.9 |
| 21 | 26.03 | 3.4231 | 9.7 |
| 22 | 26.51 | 3.3627 | 17.7 |
| 23 | 27.88 | 3.1998 | 18.0 |
| 24 | 28.70 | 3.1104 | 6.9 |
| 25 | 29.91 | 2.9871 | 30.5 |
| 26 | 30.43 | 2.9375 | 10.7 |
| 27 | 30.83 | 2.9006 | 5.8 |
| 28 | 32.01 | 2.7960 | 16.6 |
| 29 | 37.94 | 2.3718 | 5.5 |

In one embodiment, Form C of Compound D has the SEM picture as shown in FIG. 17 of US Publication No. 2019/0030018. In one embodiment, a crystalline form of Compound D has a thermogravimetric (TGA) thermograph corresponding substantially to the representative TGA thermogram as depicted in FIG. 18 of US Publication No. 2019/0030018. In certain embodiments, Form C shows no TGA weight loss.

In one embodiment, crystalline Form C of Compound D has a DSC thermogram corresponding substantially as depicted in FIG. 19 of US Publication No. 2019/0030018. In certain embodiments, Form C is characterized by a DSC plot comprising melting event with an onset temperature of 232° C. and heat of fusion of 126 J/g.

In certain embodiments, Form C is characterized by dynamic vapor sorption analysis. A representative dynamic vapor sorption (DVS) isotherm plot is shown in FIG. 20 of US Publication No. 2019/0030018. In certain embodiments, when the relative humidity (“RH”) is increased from about 0% to about 90% RH, Form C exhibits about 0.6% w/w water uptake. In certain embodiments, Form C comprises less than 0.1% water as determined in a coulometric Karl Fischer (KF) titrator equipped with an oven sample processor set at 225° C.

In certain embodiments, Form C shows no significant degradation or residual solvent by ¹H NMR (see FIG. 21 of US Publication No. 2019/0030018).

In certain embodiments, Form C of Compound D is characterized by its stability profile upon compression. In certain embodiments, Form C is stable, e.g., its XRPD pattern remains substantially unchanged with broader diffraction peaks, upon application of 2000-psi pressure for about 1 minute (see FIG. 22 of US Publication No. 2019/0030018).

In still another embodiment, Form C of Compound D is substantially pure. In certain embodiments, the substantially pure Form C of Compound D is substantially free of other solid forms, e.g., amorphous form. In certain embodiments, the purity of the substantially pure Form C of Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

In certain embodiments, Form C of Compound D is substantially pure. In certain embodiments, Form C of Compound D is substantially free of other solid forms comprising Compound D including, e.g., Forms A, B, D, E, and/or an amorphous solid form comprising Compound D. In certain embodiments, Form C is a mixture of solid forms comprising Compound D, including, e.g., a mixture comprising one or more of the following: Forms A, B, D, E, and an amorphous solid form comprising Compound D.

### Form D of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein are prepared from Form D of Compound D. In certain embodiments, Form D of Compound D is a DMSO solvate.

In certain embodiments, Form D is obtained by heating Form B in DMSO/methyl isobutyl ketone and cooling the solution.

In certain embodiments, Form D is crystalline, as indicated by, e.g., X-ray powder diffraction measurements. In one embodiment, Form D of Compound D has an X-ray powder diffraction pattern substantially as shown in FIG. 23 of US Publication No. 2019/0030018.

In one embodiment, Form D of Compound D has one or more characteristic X-ray powder diffraction peaks at a two-theta angle of approximately 14.1, 14.3, 18.8, 19.1, 23.6 or 24.0 degrees 2θ as depicted in FIG. 23 of US Publication No. 2019/0030018. In another embodiment, Form D of Compound D has one, two, three or four characteristic X-ray powder diffraction peaks at a two-theta angle of approximately 14.1, 14.3, 18.8 or 19.1 degrees 2θ. In another embodiment, Form D of Compound D has one, two, three, four, five, six or seven characteristic X-ray powder diffraction peaks as set forth in Table D. In another embodiment, Form D of Compound D has one, two, or three characteristic X-ray powder diffraction peaks as set forth in Table D.

TABLE D

| | Pos. | d-spacing | Rel. |
|---|---|---|---|
| No. | [°2 Th.] | [Å] | Int. [%] |
| 1 | 4.77 | 18.5435 | 3.0 |
| 2 | 9.57 | 9.2399 | 7.0 |
| 3 | 10.55 | 8.3876 | 3.1 |
| 4 | 11.95 | 7.4070 | 3.7 |
| 5 | 12.50 | 7.0808 | 3.5 |
| 6 | 14.06 | 6.2990 | 100.0 |
| 7 | 14.30 | 6.1927 | 92.9 |
| 8 | 16.13 | 5.4943 | 3.8 |
| 9 | 17.02 | 5.2097 | 8.4 |
| 10 | 17.50 | 5.0676 | 19.8 |
| 11 | 17.78 | 4.9881 | 8.0 |
| 12 | 18.09 | 4.9049 | 7.7 |
| 13 | 18.27 | 4.8561 | 9.0 |
| 14 | 18.75 | 4.7326 | 58.5 |
| 15 | 19.09 | 4.6482 | 63.5 |
| 16 | 21.04 | 4.2228 | 7.3 |
| 17 | 22.77 | 3.9053 | 10.9 |
| 18 | 23.58 | 3.7738 | 53.6 |
| 19 | 24.02 | 3.7045 | 24.6 |
| 20 | 24.90 | 3.5756 | 8.4 |
| 21 | 25.22 | 3.5310 | 10.0 |
| 22 | 26.37 | 3.3796 | 9.4 |
| 23 | 26.63 | 3.3470 | 7.9 |
| 24 | 28.21 | 3.1640 | 5.8 |
| 25 | 29.82 | 2.9958 | 3.0 |
| 26 | 30.16 | 2.9629 | 5.0 |
| 27 | 30.45 | 2.9361 | 6.7 |
| 28 | 32.48 | 2.7566 | 3.3 |
| 29 | 33.03 | 2.7120 | 8.1 |
| 30 | 33.69 | 2.6604 | 3.4 |
| 31 | 35.32 | 2.5413 | 3.0 |
| 32 | 37.96 | 2.3702 | 3.2 |
| 33 | 38.70 | 2.3269 | 3.0 |

In one embodiment, provided herein is a crystalline form of Compound D having a thermogravimetric (TGA) thermograph corresponding substantially to the representative TGA thermogram as depicted in FIG. 24 of US Publication No. 2019/0030018. In certain embodiments, Form D shows TGA weight loss of about 14.1% up to 140° C.

In certain embodiments, Form D comprises DMSO in about 14.3 wt % as measured by gas chromatography.

In still another embodiment, Form D of Compound D is substantially pure. In certain embodiments, the substantially pure Form D of Compound D is substantially free of other solid forms, e.g., amorphous form. In certain embodiments, the purity of the substantially pure Form D of Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

In certain embodiments Form D of Compound D is substantially pure. In certain embodiments, Form D of Compound D is substantially free of other solid forms comprising Compound D including, e.g., Forms A, B, C, E, and/or an amorphous solid form comprising Compound D as provided herein. In certain embodiments, Form D is a mixture of solid forms comprising Compound D, including, e.g., a mixture comprising one or more of the following: Forms A, B, C, E, and an amorphous solid form comprising Compound D.

### Form E of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein are prepared from Form E of Compound D. In certain embodiments, Form E of Compound D is a DMSO solvate.

In certain embodiments, Form E is obtained from Form C in DMSO/MIBK or DMSO/IPA or DMSO/anisole at room temperature.

In certain embodiments, Form E is crystalline, as indicated by, e.g., X-ray powder diffraction measurements. In one embodiment, Form E of Compound D has an X-ray powder diffraction pattern substantially as shown in FIG. 25 of US Publication No. 2019/0030018.

In one embodiment, Form E of Compound D has one or more characteristic X-ray powder diffraction peaks at a two-theta angle of approximately 10.5, 12.5, 16.1, 17.0, 18.5, 21.2, 21.7, 22.6, 22.9, 23.4, 23.8, 24.1, 25.1 or 26.7, degrees 2θ as depicted in FIG. 25 of US Publication No. 2019/0030018. In another embodiment, Form E of Compound D has one, two, three or four characteristic X-ray powder diffraction peaks at a two-theta angle of approximately 16.1, 17.0, 21.2 or 22.9 degrees 2θ. In another embodiment, Form E of Compound D has one, two, three, four, five, six or seven characteristic X-ray powder diffraction peaks as set forth in Table E. In another embodiment, Form E of Compound D has one, two, or three characteristic X-ray powder diffraction peaks as set forth in Table E.

TABLE E

| | Pos. | d-spacing | Rel. |
|---|---|---|---|
| No. | [°2 Th.] | [Å] | Int. [%] |
| 1 | 4.20 | 21.0329 | 9.6 |
| 2 | 10.48 | 8.4394 | 32.0 |
| 3 | 12.54 | 7.0591 | 28.4 |
| 4 | 14.52 | 6.1023 | 9.9 |
| 5 | 15.51 | 5.7131 | 17.7 |
| 6 | 16.08 | 5.5121 | 100.0 |
| 7 | 16.97 | 5.2256 | 94.5 |
| 8 | 17.77 | 4.9908 | 17.1 |
| 9 | 18.48 | 4.8001 | 20.5 |
| 10 | 19.54 | 4.5422 | 14.7 |
| 11 | 21.15 | 4.2007 | 62.8 |
| 12 | 21.72 | 4.0924 | 20.8 |
| 13 | 22.64 | 3.9270 | 57.4 |
| 14 | 22.91 | 3.8826 | 59.9 |
| 15 | 23.43 | 3.7977 | 23.6 |
| 16 | 23.83 | 3.7348 | 23.2 |
| 17 | 24.13 | 3.6881 | 29.5 |
| 18 | 25.14 | 3.5421 | 35.2 |
| 19 | 26.72 | 3.3362 | 49.5 |
| 20 | 27.68 | 3.2232 | 14.6 |
| 21 | 27.93 | 3.1949 | 15.3 |
| 22 | 28.86 | 3.0942 | 15.6 |
| 23 | 29.08 | 3.0703 | 18.3 |
| 24 | 30.12 | 2.9671 | 7.1 |
| 25 | 30.92 | 2.8923 | 12.8 |
| 26 | 32.35 | 2.7672 | 5.0 |
| 27 | 33.21 | 2.6979 | 6.9 |

In one embodiment, provided herein is a crystalline form of Compound D having a thermogravimetric (TGA) thermograph corresponding substantially to the representative TGA thermogram as depicted in FIG. 26 of US Publication No. 2019/0030018. In certain embodiments, Form E shows TGA weight loss of about 19.4% up to 120° C. In certain embodiments, Form E shows additional weight loss of 24.9% between 120 and 220° C.

In one embodiment, Form E of Compound D is substantially pure. In certain embodiments, the substantially pure Form E of Compound D is substantially free of other solid forms, e.g., amorphous form. In certain embodiments, the purity of the substantially pure Form E of Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

In certain embodiments, Form E of Compound D is substantially pure. In certain embodiments herein, Form E of Compound D is substantially free of other solid forms comprising Compound D including, e.g., Forms A, B, C, D and/or an amorphous solid form comprising Compound D. In certain embodiments, Form E is a mixture of solid forms comprising Compound D, including, e.g., a mixture comprising one or more of the following: Forms A, B, C, D and an amorphous solid form comprising Compound D.

### Amorphous Form of 2-(4-chlorophenyl)-N-((2-(2,6-dioxopiperidin-3-yl)-1-oxoisoindolin-5-yl)methyl)-2,2-difluoroacetamide

In certain embodiments, the formulations provided herein comprise amorphous Compound D.

In certain embodiments, provided herein are methods for making the amorphous form by heating Compound D in THE and water and cooling the solution.

In one embodiment, provided herein is an amorphous solid form of Compound D having a modulated DSC thermogram as depicted in FIG. 27 of US Publication No. 2019/0030018.

In one embodiment, amorphous Compound D has an X-ray powder diffraction pattern substantially as shown in FIG. 28 of US Publication No. 2019/0030018.

In one embodiment, amorphous Compound D has a ¹H NMR spectrum substantially as shown in FIG. 29 of US Publication No. 2019/0030018.

In still another embodiment, amorphous Compound D is substantially pure. In certain embodiments, the substantially pure amorphous Compound D is substantially free of other solid forms, e.g., Form A, Form B, Form C, Form D or Form E. In certain embodiments, the purity of the substantially pure amorphous Compound D is no less than about 95% pure, no less than about 96% pure, no less than about 97% pure, no less than about 98% pure, no less than about 98.5% pure, no less than about 99% pure, no less than about 99.5% pure, or no less than about 99.8% pure.

### Isotopologues of 2-(4-chlorophenyl)-N-((2-(2,6-dioxopiperidin-3-yl)-1-oxoisoindolin-5-yl)methyl)-2,2-difluoroacetamide

Also provided herein are isotopically enriched analogs of 2-(4-chlorophenyl)-N-((2-(2,6-dioxopiperidin-3-yl)-1-oxoisoindolin-5-yl)methyl)-2,2-difluoroacetamide (“isotopologues”) provided herein. Isotopic enrichment (for example, deuteration) of pharmaceuticals to improve pharmacokinetics (“PK”), pharmacodynamics (“PD”), and toxicity profiles, has been demonstrated previously with some classes of drugs. See, for example, Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D, Chem. Biol. Interact. 117: 191 (1999).

Without being limited by any particular theory, isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen will cause a decrease in the reaction rate and the process will slow down. This phenomenon is known as the Deuterium Kinetic Isotope Effect (“DKIE”). (See, e.g, Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. Without being limited by a particular theory, high DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects.

Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, will provide a similar kinetic isotope effects.

In certain embodiments, the compound provided herein is a prodrug of a compound provided herein (e.g., a prodrug of Compound D). Exemplary compounds include those disclosed in US Publication No. 2017/0197933, the disclosure of which is incorporated herein by reference in its entirety.

5.6. Pharmaceutical Compositions

In some embodiments, the compound provided herein is formulated in a pharmaceutical composition. In some embodiments, Compound D is provided in stable formulations of Compound D. In one embodiment, the formulations of Compound D comprise a solid form of 2-(4-chlorophenyl)-N-((2-(2,6-dioxopiperidin-3-yl)-1-oxoisoindolin-5-yl)methyl)-2,2-difluoroacetamide. In one embodiment, the formulations of Compound D comprise an amorphous form of 2-(4-chlorophenyl)-N-((2-(2,6-dioxopiperidin-3-yl)-1-oxoisoindolin-5-yl)methyl)-2,2-difluoroacetamide.

In certain embodiments, the formulations are prepared with dimethylsulfoxide as a co-solvent or a processing aid. In certain embodiments, the formulations are prepared with formic acid as co-solvent or a processing aid. In certain embodiments, the formulations are prepared without any co-solvent or processing aid.

In certain embodiments, the formulations comprise dimethylsulfoxide as a co-solvent or a processing aid. In certain embodiments, the formulations comprise formic acid as a co-solvent or a processing aid. In certain embodiments, the formulations do not comprise any co-solvent or processing aid.

In certain embodiments, the formulations provided herein are lyophilized formulations. In certain embodiments, the formulations provided herein are reconstituted formulations obtained in a pharmaceutically acceptable solvent to produce a pharmaceutically acceptable solution.

Formulation Ia

In one embodiment, provided herein are formulations comprising Compound D in an amount of about 0.05-0.2%, a citrate buffer in an amount of about 3%-6%, and hydroxypropyl O-cyclodextrin (HPBCD) in an amount of about 92-98% based on total weight of the formulation.

In one embodiment, provided herein are formulations comprising Compound D in an amount of about 0.05-0.2%, a citrate buffer in an amount of about 3%-6%, and sulfobutyl ether-beta-cyclodextrin in an amount of about 92-98% based on total weight of the formulation.

In one embodiment, provided herein are formulations comprising Compound D in an amount of about 0.05-0.2%, a citrate buffer in an amount of about 3%-6%, HPBCD in an amount of about 92-98%, and no more than about 1% dimethyl sulfoxide based on total weight of the formulation.

In one embodiment, provided herein are formulations comprising Compound D in an amount of about 0.05-0.2%, a citrate buffer in an amount of about 3%-6%, sulfobutyl ether-beta-cyclodextrin in an amount of about 92-98%, and no more than about 1% dimethyl sulfoxide based on total weight of the formulation.

In one embodiment, provided herein are formulations comprising Compound D in an amount of about 0.08-0.15%, a citrate buffer in an amount of about 3%-6%, and HPBCD in an amount of about 94-96%, based on total weight of the formulation.

In one embodiment, provided herein are formulations comprising Compound D in an amount of about 0.08-0.15%, a citrate buffer in an amount of about 3%-6%, and sulfobutyl ether-beta-cyclodextrin in an amount of about 94-96%, and based on total weight of the formulation.

In one embodiment, provided herein are formulations comprising Compound D in an amount of about 0.08-0.15%, a citrate buffer in an amount of about 3%-6%, HPBCD in an amount of about 94-96%, and no more than about 1% dimethyl sulfoxide based on total weight of the formulation.

In one embodiment, provided herein are formulations comprising Compound D in an amount of about 0.08-0.15%, a citrate buffer in an amount of about 3%-6%, sulfobutyl ether-beta-cyclodextrin in an amount of about 94-96%, and no more than about 1% dimethyl sulfoxide based on total weight of the formulation.

In one aspect, the formulation provided herein comprises Compound D in an amount of about 0.08 to about 0.15% based on the total weight of the formulation. In certain embodiments, the amount of Compound D is from about 0.09% to about 0.15%, about 0.1% to about 0.13% or about 0.11% to about 0.12% based on the total weight of the formulation. In certain embodiments, the amount of Compound D is about 0.05%, 0.07%, 0.09%, 0.11%, 0.12%, 0.13%, or 0.15% based on the total weight of the formulation. In one embodiment, the amount of Compound D in the formulation is about 0.12% based on the total weight of the formulation.

In another aspect, provided herein is a formulation that comprises Compound D in an amount of about 0.5 mg to about 2 mg in a 20 cc vial. In still another aspect is a formulation that comprises Compound D in an amount of about 0.5 mg to about 1.5 mg, about 0.75 mg to about 1.25 mg, or about 0.8 mg to about 1.1 mg in a 20 cc vial. In one aspect Compound D is present in an amount of about 0.7, 0.75, 0.76, 0.8, 0.9, 1.0, 1.05 or 1.2 mg in a 20 cc vial. In one aspect Compound D is present in an amount of about 1.05 mg in a 20 cc vial.

In one aspect, the formulations provided herein contain a citrate buffer. In one aspect, the amount of citrate buffer in the formulations provided herein is from about 3% to about 6% based on total weight of the formulation. In one aspect, the amount of citrate buffer in the formulations provided herein is about 3%, 3.5%, 4%, 4.2%, 4.5% or 5% based on total weight of the formulation. In one aspect, the amount of citrate buffer in the formulations provided herein is about 4.2% based on total weight of the formulation. In one aspect, the amount of citrate buffer in the formulations provided herein is about 37 mg in a 20 cc vial.

In one embodiment, the citrate buffer comprises anhydrous citric acid and anhydrous sodium citrate. In certain embodiments, the amount of anhydrous citric acid is from about 1.5% to about 3%, about 1.75% to about 2.75%, or about 2% to about 2.5% based on total weight of the formulation. In certain embodiments, the amount of anhydrous citric acid in the formulation is about 1.5%, 1.75%, 2%, 2.1%, or 2.5% based on total weight of the formulation. In one embodiment, the amount of anhydrous citric acid in the formulation is about 2%, 2.1%, 2.22% or 2.3% based on total weight of the formulation. In one embodiment, the amount of anhydrous citric acid in the formulation is about 2.10% based on total weight of the formulation.

In still another aspect is a formulation that comprises anhydrous citric acid in an amount of about 16 mg to about 20 mg in a 20 cc vial. In one embodiment, the amount of anhydrous citric acid is about 16, 17, 18, 18.2, 18.4, 18.6, 18.8, 19 or 20 mg in a 20 cc vial. In one embodiment, the amount of anhydrous citric acid is about 18.6 mg in a 20 cc vial.

In certain embodiments, the amount of anhydrous sodium citrate is from about 1.5% to about 3%, about 1.75% to about 2.75%, or about 2% to about 2.5% based on total weight of the formulation. In certain embodiments, the amount of anhydrous sodium citrate in the formulation is about 1.5%, 1.75%, 2%, 2.1%, or 2.5% based on total weight of the formulation. In one embodiment, the amount of anhydrous sodium citrate in the formulation is about 2%, 2.05%, 2.08% or 2.1% based on total weight of the formulation. In one embodiment, the amount of anhydrous sodium citrate in the formulation is about 2.08% based on total weight of the formulation.

In still another aspect is a formulation that comprises anhydrous sodium citrate in an amount of about 16 mg to about 20 mg in a 20 cc vial. In one embodiment, the amount of anhydrous sodium citrate is about 16, 17, 18, 18.2, 18.4, 18.6, 18.8, 19 or 20 mg in a 20 cc vial. In one embodiment, the amount of anhydrous sodium citrate is about 18.4 mg in a 20 cc vial.

In certain embodiments, the amount of HPBCD in the formulations provided herein is about 94 to about 97% based on total weight of the formulation. In one embodiment, the amount of HPBCD in the formulations provided herein is about 94.5%, 95%, 95.5%, or 96% based on total weight of the formulation. In one embodiment, the amount of HPBCD in the formulations provided herein is about 95% based on total weight of the formulation.

In certain embodiments, the amount of sulfobutyl ether-beta-cyclodextrin in the formulations provided herein is about 94 to about 97% based on total weight of the formulation. In one embodiment, the amount of sulfobutyl ether-beta-cyclodextrin in the formulations provided herein is about 94.5%, 95%, ⁹5.5%, or 96% based on total weight of the formulation. In one embodiment, the amount of sulfobutyl ether-beta-cyclodextrin in the formulations provided herein is about 95% based on total weight of the formulation.

In another aspect is a formulation that comprises HPBCD in an amount of about 800-900 mg in a 20 cc vial. In another aspect is a formulation that comprises HPBCD in an amount of about 810-880 mg, 820-860 mg or 830-850 mg in a 20 cc vial. In another aspect is a formulation that comprises HPBCD in an amount of about 840 mg in a 20 cc vial.

In another aspect is a formulation that comprises sulfobutyl ether-beta-cyclodextrin in an amount of about 800-900 mg in a 20 cc vial. In another aspect is a formulation that comprises sulfobutyl ether-beta-cyclodextrin in an amount of about 810-880 mg, 820-860 mg or 830-850 mg in a 20 cc vial. In another aspect is a formulation that comprises sulfobutyl ether-beta-cyclodextrin in an amount of about 840 mg in a 20 cc vial.

In another aspect is a formulation that comprises Kleptose® HPB in an amount of about 840 mg in a 20 cc vial.

In one embodiment, the formulations comprise dimethyl sulfoxide in an amount of no more than about 1.5% based on total weight of the formulation. In one embodiment, the formulations comprise dimethyl sulfoxide in an amount of up to 0.1%, 0.2%, 0.3%, 0.4%, 0.6%, 0.7%, 0.8%, 0.9% or 1% based on total weight of the formulation. In one embodiment, the formulations comprise no more than about 0.1%, 0.2%, 0.3%, 0.4%, 0.6%, 0.7%, 0.8%, 0.9% or 1% dimethyl sulfoxide based on total weight of the formulation. In one embodiment, the formulations comprise dimethyl sulfoxide in an amount of up to about 0.1 to about 1.5% based on total weight of the formulation. In one embodiment, the amount of dimethyl sulfoxide in the formulations provided herein is about 0.1 to about 1.3% based on total weight of the formulation. In one embodiment, the amount of dimethyl sulfoxide in the formulations provided herein is about 0.1%, 0.2%, 0.3%, 0.4%, 0.6%, 0.7%, 0.8%, 0.9% or 1% based on total weight of the formulation. In one embodiment, the formulations provided herein do not contain any dimethyl sulfoxide. In one embodiment, the amount of dimethyl sulfoxide in the formulations provided herein is about 0.4% to 0.8% based on total weight of the formulation.

In another aspect is a formulation that comprises dimethyl sulfoxide in an amount of about 4 to 7 mg in a 20 cc vial. In another aspect is a formulation that comprises dimethyl sulfoxide in an amount of about 4.5-6.5 mg, or 5 to 6 mg in a 20 cc vial.

In certain embodiments, the formulation provided herein is lyophilized, and the lyophilized formulation upon reconstitution has a pH of about 4 to 5. In certain embodiments, the formulation upon reconstitution has a pH of about 4.2 to 4.4. In one embodiment, the lyophilized formulation upon reconstitution has a pH of about 4, 4.1, 4.2, 4.3, 4.4, 4.5, 4.6, 4.7, 4.8, 4.9 or 5.

In certain embodiments, the lyophilized formulation upon reconstitution has an osmolality of about 250-290 mOsm/kg. In certain embodiments, the lyophilized formulation upon reconstitution has an osmolality of about 260-280 mOsm/kg.

In certain embodiments, provided herein is a container comprising a formulation provided herein. In one aspect, the container is a glass vial. In one aspect, the container is a 20 cc glass vial.

In one aspect provided herein is a formulation in a 20 cc vial that comprises: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide and a pharmaceutically acceptable carrier or excipient that includes a bulking agent as described herein. In one embodiment, the formulation further comprises no more than about 7 mg dimethyl sulfoxide as residual solvent. In one embodiment, the formulation comprises no more than about 6 mg dimethyl sulfoxide as residual solvent. In one embodiment, the formulation comprises no more than about 5 mg dimethyl sulfoxide as residual solvent. In one embodiment, the formulation comprises no more than about 4 mg dimethyl sulfoxide as residual solvent. In one embodiment, the formulation comprises from about 3 mg to about 7 mg, about 4 mg to about 6 mg, about 4 mg to about 5 mg or about 5 mg to about 6 mg dimethyl sulfoxide as residual solvent. In one embodiment, the formulation comprises about 4, 4.5, 5, 5.3, 5.5, 5.7, 6 or 6.5 mg dimethyl sulfoxide as residual solvent.

In one embodiment, provided herein are formulations consisting essentially of Compound D in an amount of about 0.05-0.2%, a citrate buffer in an amount of about 3%-6%, and HPBCD in an amount of about 92-98% based on total weight of the formulation.

In one embodiment, provided herein are formulations consisting essentially of Compound D in an amount of about 0.05-0.2%, a citrate buffer in an amount of about 3%-6%, and sulfobutyl ether-beta-cyclodextrin in an amount of about 92-98% based on total weight of the formulation.

In one embodiment, provided herein are formulations consisting essentially of Compound D in an amount of about 0.05-0.2%, a citrate buffer in an amount of about 3%-6%, HPBCD in an amount of about 92-98%, and no more than about 1% dimethyl sulfoxide based on total weight of the formulation.

In one embodiment, provided herein are formulations consisting essentially of Compound D in an amount of about 0.05-0.2%, a citrate buffer in an amount of about 3%-6%, sulfobutyl ether-beta-cyclodextrin in an amount of about 92-98%, and no more than about 1% dimethyl sulfoxide based on total weight of the formulation.

In one aspect provided herein is a formulation in a 20 cc vial that comprises: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, a pharmaceutically acceptable carrier or excipient that includes a buffer and bulking agent as described herein, and about 5 mg to about 6 mg dimethyl sulfoxide as residual solvent. The buffer and bulking agent can be present at an amount as described herein.

In one aspect provided herein is a formulation in a 20 cc vial that comprises: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent as described herein. In one embodiment, the formulation in a 20 cc vial is reconstituted with 3.8 mL sterile water for injection.

In one aspect provided herein is a formulation in a 20 cc vial that consists essentially of: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent as described herein. In one embodiment, the formulation in a 20 cc vial is reconstituted with 3.8 mL sterile water for injection.

In one aspect provided herein is a formulation in a 20 cc vial that consists of: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent as described herein. In one embodiment, the formulation in a 20 cc vial is reconstituted with 3.8 mL sterile water for injection.

In one embodiment, provided herein is an aqueous formulation comprising Compound D in an amount of about 0.05-0.2% based on total weight of the solids, a citrate buffer in an amount of about 3%-6% based on total weight of the solids, HPBCD in an amount of about 92-98% based on total weight of the solids, and a diluent.

In one embodiment, provided herein is an aqueous formulation consisting essentially of Compound D in an amount of about 0.05-0.2% based on total weight of the solids, a citrate buffer in an amount of about 3%-6% based on total weight of the solids, HPBCD in an amount of about 92-98% based on total weight of the solids, and a diluent.

In one aspect provided herein is an aqueous formulation that comprises: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent and about 3.8 mL diluent.

In one aspect provided herein is an aqueous formulation that consists essentially of: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent and about 3.8 mL diluent.

In one aspect provided herein is an aqueous formulation that consists of: Compound D at an amount that provides 1.05 mg 2-(4-chlorophenyl)-N-((2-(2,6-dioxopiperidin-3-yl)-1-oxoisoindolin-5-yl)methyl)-2,2-difluoroacetamide, 18.6 mg anhydrous citric acid, 18.4 mg anhydrous sodium citrate, 840 mg HPBCD, and about 5 mg to about 6 mg dimethyl sulfoxide as residual solvent and about 3.8 mL diluent.

### Formulation Ib

In one embodiment, provided herein are formulations comprising Compound D in an amount of about 0.01-0.15%, hydroxypropyl P-cyclodextrin in an amount of about 99.1-99.99%. In one embodiment, provided herein are formulations comprising Compound D in an amount of about 0.01-0.15%, hydroxypropyl P-cyclodextrin in an amount of about 99.1-99.99%, and no more than about 0.5% formic acid based on total weight of the formulation.

In one embodiment, provided herein are formulations comprising Compound D in an amount of about 0.05-0.25% and HPBCD in an amount of about 99.1-99.9% based on total weight of the formulation.

In one embodiment, provided herein are formulations comprising Compound D in an amount of about 0.05-0.25%, HPBCD in an amount of about 99.1-99.9%, and no more than about 0.5% formic acid based on total weight of the formulation.

In one embodiment, provided herein are formulations comprising Compound D in an amount of about 0.05-0.25% and HPBCD in an amount of about 99.75-99.9% based on total weight of the formulation.

In one embodiment, provided herein are formulations comprising Compound D in an amount of about 0.05-0.25%, HPBCD in an amount of about 99.75-99.9%, and no more than about 0.5% formic acid based on total weight of the formulation.

In one embodiment, provided herein are formulations comprising Compound D in an amount of about 0.05-0.25%, HPBCD in an amount of about 99.75-99.9%, and no more than about 0.2% formic acid based on total weight of the formulation.

In one embodiment, provided herein are formulations comprising Compound D in an amount of about 0.08-0.15% and HPBCD in an amount of about 99.8-99.9% based on total weight of the formulation.

In one embodiment, provided herein are formulations comprising Compound D in an amount of about 0.08-0.15%, HPBCD in an amount of about 99.8-99.9%, and no more than about 0.5% formic acid based on total weight of the formulation.

In one embodiment, provided herein are formulations comprising Compound D in an amount of about 0.08-0.15%, HPBCD in an amount of about 99.8-99.9%, and no more than about 0.12% formic acid based on total weight of the formulation.

In one embodiment, provided herein are formulations comprising Compound D in an amount of about 0.12% and HPBCD in an amount of about 99.88% based on total weight of the formulation.

In one embodiment, provided herein are formulations comprising Compound D in an amount of about 0.05-0.25% and sulfobutyl ether-beta-cyclodextrin in an amount of about 99.1-99.9%, based on total weight of the formulation.

In one embodiment, provided herein are formulations comprising Compound D in an amount of about 0.05-0.25%, sulfobutyl ether-beta-cyclodextrin in an amount of about 99.1-99.9%, and no more than about 0.5% formic acid based on total weight of the formulation.

In one embodiment, provided herein are formulations comprising Compound D in an amount of about 0.05-0.25% and sulfobutyl ether-beta-cyclodextrin in an amount of about 99.75-99.9%, based on total weight of the formulation.

In one embodiment, provided herein are formulations comprising Compound D in an amount of about 0.08-0.15% and sulfobutyl ether-beta-cyclodextrin in an amount of about 99.8-99.9% based on total weight of the formulation.

In one embodiment, provided herein are formulations comprising Compound D in an amount of about 0.08-0.15%, sulfobutyl ether-beta-cyclodextrin in an amount of about 99.8-99.9%, and no more than about 0.5% formic acid based on total weight of the formulation.

In one embodiment, provided herein are formulations comprising Compound D in an amount of about 0.12% and sulfobutyl ether-beta-cyclodextrin in an amount of about 99.88% based on total weight of the formulation.

In one aspect, the formulation provided herein comprises Compound D in an amount of about 0.08 to about 0.15% based on the total weight of the formulation. In certain embodiments, the amount of Compound D is from about 0.09% to about 0.15%, about 0.1% to about 0.13% or about 0.11% to about 0.12% based on the total weight of the formulation. In certain embodiments, the amount of Compound D is about 0.05%, 0.07%, 0.09%, 0.11%, 0.12%, 0.13%, or 0.15% based on the total weight of the formulation. In one embodiment, the amount of Compound D in the formulation is about 0.12% based on the total weight of the formulation.

In another aspect, provided herein is a formulation that comprises Compound D in an amount of about 0.5 mg to about 2 mg in a 20 cc vial. In still another aspect is a formulation that comprises Compound D in an amount of about 0.5 mg to about 1.5 mg, about 0.75 mg to about 1.25 mg, or about 0.8 mg to about 1.1 mg in a 20 cc vial. In one aspect Compound D is present in an amount of about 0.7, 0.75, 0.76, 0.8, 0.9, 1.0, 1.05 or 1.2 mg in a 20 cc vial. In one aspect Compound D is present in an amount of about 1 mg in a 20 cc vial.

In one embodiment, the amount of HPBCD in the formulations provided herein is about 97 to about 99.9% based on total weight of the formulation. In one embodiment, the amount of HPBCD in the formulations provided herein is about 98 to about 99.9% based on total weight of the formulation. In one embodiment, the amount of HPBCD in the formulations provided herein is about 99.1%, 99.3%, 99.5%, 99.7% or 99.9% based on total weight of the formulation. In one embodiment, the amount of HPBCD in the formulations provided herein is about 99.5% based on total weight of the formulation. In another aspect is a formulation that comprises HPBCD in an amount of about 750-850 mg in a 20 cc vial. In another aspect is a formulation that comprises HPBCD in an amount of about 790-840 mg, 780-830 mg or 790-810 mg in a 20 cc vial. In another aspect is a formulation that comprises HPBCD in an amount of about 800 mg in a 20 cc vial.

In another aspect is a formulation that comprises Kleptose® HPB in an amount of about 800 mg in a 20 cc vial.

In one embodiment, the amount of sulfobutyl ether-beta-cyclodextrin in the formulations provided herein is about 97 to about 99.9% based on total weight of the formulation. In one embodiment, the amount of sulfobutyl ether-beta-cyclodextrin in the formulations provided herein is about 98 to about 99.9% based on total weight of the formulation. In one embodiment, the amount of sulfobutyl ether-beta-cyclodextrin in the formulations provided herein is about 99.1%, 99.3%, 99.5%, 99.7% or 99.9% based on total weight of the formulation. In one embodiment, the amount of sulfobutyl ether-beta-cyclodextrin in the formulations provided herein is about 99.5% based on total weight of the formulation.

In another aspect is a formulation that comprises sulfobutyl ether-beta-cyclodextrin in an amount of about 750-850 mg in a 20 cc vial. In another aspect is a formulation that comprises sulfobutyl ether-beta-cyclodextrin in an amount of about 790-840 mg, 780-830 mg or 790-810 mg in a 20 cc vial. In another aspect is a formulation that comprises sulfobutyl ether-beta-cyclodextrin in an amount of about 800 mg in a 20 cc vial.

In another aspect is a formulation that comprises Kleptose® HPB in an amount of about 800 mg in a 20 cc vial.

In one embodiment, the formulations comprise formic acid in no more than about 0.5% based on total weight of the formulation. In one embodiment, the formulations comprise formic acid in an amount of up to about 0.05%, 0.07%, 0.09%, 0.1%, 0.2%, 0.3%, 0.4% or 0.5% based on total weight of the formulation. In one embodiment, the formulations comprise formic acid in no more than about 0.05%, 0.07%, 0.09%, 0.1%, 0.2%, 0.3%, 0.4% or 0.5% based on total weight of the formulation. In one embodiment, the amount of formic acid in the formulations provided herein is about 0.05 to about 0.5% based on total weight of the formulation. In one embodiment, the amount of formic acid in the formulations provided herein is about 0.05 to about 0.1% based on total weight of the formulation. In one embodiment, the amount of formic acid in the formulations provided herein is about 0.05%, 0.07%, 0.09%, 0.1%, 0.2%, 0.3%, 0.4% or 0.5% based on total weight of the formulation. In one embodiment, the formulations provided herein do not contain any formic acid. In one embodiment, the amount of formic acid in the formulations provided herein is about 0.05% to 0.09% based on total weight of the formulation.

In another aspect is a formulation that comprises formic acid in an amount of no more than about 1 mg in a 20 cc vial. In another aspect is a formulation that comprises formic acid in an amount of up to about 0.2, 0 5, 0.7, 0.9 mg or 1 mg in a 20 cc vial. In another aspect is a formulation that comprises formic acid in an amount of about 0.3-0.9 mg, or 0.4 to 0.8 mg in a 20 cc vial.

In another aspect, provided herein is a formulation that comprises Compound D in an amount of about 1 mg and HPBCD in an amount of about 800 mg in a 20 cc vial.

In another aspect, provided herein is a formulation that comprises Compound D in an amount of about 1 mg, HPBCD in an amount of about 800 mg and formic acid in an amount of about 0.9 mg in a 20 cc vial.

### Formulation Ic

In one embodiment, provided herein are formulations comprising Compound D in an amount of about 0.01-0.08% and HPBCD in an amount of about 99.40-99.99% based on total weight of the formulation.

In one embodiment, provided herein are formulations comprising Compound D in an amount of about 0.01-0.08%, HPBCD in an amount of about 99.40-99.99%, and no more than about 0.5% formic acid based on total weight of the formulation.

In one embodiment, provided herein are formulations comprising Compound D in an amount of about 0.03-0.06% and HPBCD in an amount of about 99.60-99.99% based on total weight of the formulation.

In one embodiment, provided herein are formulations comprising Compound D from about 0.01 to about 0.08%, hydroxypropyl P-cyclodextrin from about 99.40% to about 99.99%, and formic acid from about 0.1 to about 0.3% based on total weight of the formulation

In one aspect, the formulation provided herein comprises Compound D in an amount of about 0.02 to about 0.06% based on the total weight of the formulation. In certain embodiments, the amount of Compound D is from about 0.03% to about 0.06%, or about 0.04% to about 0.06% based on the total weight of the formulation. In certain embodiments, the amount of Compound D is about 0.03%, 0.04%, 0.05% or 0.06% based on the total weight of the formulation. In one embodiment, the amount of Compound D in the formulation is about 0.05% based on the total weight of the formulation.

In another aspect, provided herein is a formulation that comprises Compound D in an amount of about 0.75 mg to about 1.5 mg in a 20 cc vial. In still another aspect is a formulation that comprises Compound D in an amount of about 0.75 mg to about 1.25 mg in a 20 cc vial. In one aspect Compound D is present in an amount of about 0.75, 0.8, 0.9, 1.0, 1.05 or 1.2 mg in a 20 cc vial. In one aspect Compound D is present in an amount of about 1 mg in a 20 cc vial.

In one embodiment, the amount of HPBCD in the formulations provided herein is about 99.40 to about 99.99% based on total weight of the formulation. In one embodiment, the amount of HPBCD in the formulations provided herein is about 99.5, 99.6, 99.7, 99.8, 99.9, 99.95, or 99.99% based on total weight of the formulation. In another aspect is a formulation that comprises HPBCD in an amount of about 1800-1900 mg in a 20 cc vial. In another aspect is a formulation that comprises HPBCD in an amount of about 1850-1900 mg in a 20 cc vial. In another aspect is a formulation that comprises HPBCD in an amount of about 1875 mg in a 20 cc vial.

In one embodiment, the formulations comprise formic acid in no more than about 0.5% based on total weight of the formulation. In one embodiment, the formulations comprise formic acid in an amount of up to about 0.05%, 0.07%, 0.09%, 0.1%, 0.2%, 0.3%, 0.4% or 0.5% based on total weight of the formulation. In one embodiment, the formulations comprise formic acid in no more than about 0.05%, 0.07%, 0.09%, 0.1%, 0.2%, 0.3%, 0.4% or 0.5% based on total weight of the formulation. In one embodiment, the amount of formic acid in the formulations provided herein is about 0.05 to about 0.3% based on total weight of the formulation. In one embodiment, the amount of formic acid in the formulations provided herein is about 0.05 to about 0.25% based on total weight of the formulation. In one embodiment, the amount of formic acid in the formulations provided herein is about 0.05%, 0.07%, 0.09%, 0.1%, 0.2%, or 0.3% based on total weight of the formulation. In one embodiment, the formulations provided herein do not contain any formic acid. In one embodiment, the amount of formic acid in the formulations provided herein is about 0.11% to 0.3% based on total weight of the formulation.

In another aspect is a formulation that comprises formic acid in an amount of no more than about 4 mg in a 20 cc vial. In another aspect is a formulation that comprises formic acid in an amount of up to about 1, 1.8, 2, 2.1, 2.5, 3, 3.5, 3.8, 3.9, 4, 4.5, 4.9 mg or 5 mg in a 20 cc vial. In another aspect is a formulation that comprises formic acid in an amount of about 1-1.8 mg, 2.1-3.8 mg, or 3.9-4.9 mg in a 20 cc vial.

In another aspect, provided herein is a formulation that comprises Compound D in an amount of about 1 mg, and HPBCD in an amount of about 1875 mg in a 20 cc vial.

In another aspect, provided herein is a formulation that comprises Compound D in an amount of about 1 mg, HPBCD in an amount of about 1875 mg and formic acid in an amount of about 2.1-3.8 mg in a 20 cc vial.

Formulations without Co-Solvent

In one embodiment, provided herein are formulations comprising Compound D in an amount of about 0.15-0.5%, a citrate buffer in an amount of about 15% to about 35%, and HPBCD in an amount of about 92% to about 98%, based on total weight of the formulation. In one embodiment, the citrate buffer comprises anhydrous citric acid and anhydrous sodium citrate.

In one embodiment, provided herein are formulations comprising Compound D in an amount of about 0.25-0.30%, a citrate buffer in an amount of about 30-32%, and HPBCD in an amount of about 67-69%, based on total weight of the formulation.

In one embodiment, provided herein are formulations comprising Compound D in an amount of about 0.30-0.33%, a citrate buffer in an amount of about 17-18%, and HPBCD in an amount of about 80-85%, based on total weight of the formulation.

Exemplary Formulations

In one embodiment, provided herein are formulations consisting essentially of Compound D in an amount of about 0.05-0.25% and HPBCD in an amount of about 99.75-99.95% based on total weight of the formulation.

In one embodiment, provided herein are formulations consisting essentially of Compound D in an amount of about 0.05-0.25% and HPBCD in an amount of about 99.75-99.99% based on total weight of the formulation.

In one embodiment, provided herein are formulations consisting essentially of Compound D in an amount of about 0.05-0.25% and sulfobutyl ether-beta-cyclodextrin in an amount of about 99.75-99.95%, based on total weight of the formulation.

In one aspect provided herein is a formulation in a 20 cc vial that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, and about 0.6 mg formic acid as described herein. In one embodiment, the formulation in a 20 cc vial is reconstituted with 4.5 mL sterile water for injection.

In one aspect provided herein is a formulation in a 20 cc vial that consists essentially of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, and about 0.6 mg formic acid as described herein. In one embodiment, the formulation in a 20 cc vial is reconstituted with 4.5 mL sterile water for injection.

In one aspect provided herein is a formulation in a 20 cc vial that consists of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, and about 0.6 mg formic acid as described herein. In one embodiment, the formulation in a 20 cc vial is reconstituted with 4.5 mL sterile water for injection.

In one aspect provided herein is a formulation in a 20 cc vial that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg sulfobutyl ether-beta-cyclodextrin, and about 0.6 mg formic acid as described herein. In one embodiment, the formulation in a 20 cc vial is reconstituted with 4.5 mL sterile water for injection.

In one aspect provided herein is a formulation in a 20 cc vial that consists essentially of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg sulfobutyl ether-beta-cyclodextrin, and about 0.6 mg formic acid as described herein. In one embodiment, the formulation in a 20 cc vial is reconstituted with 4.5 mL sterile water for injection.

In one aspect provided herein is a formulation in a 20 cc vial that consists of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg sulfobutyl ether-beta-cyclodextrin, and about 0.6 mg formic acid as described herein. In one embodiment, the formulation in a 20 cc vial is reconstituted with 4.5 mL sterile water for injection.

In one aspect provided herein is a formulation in a 20 cc vial that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 1875 mg HPBCD, and about 2.1-3.8 mg formic acid as described herein. In one embodiment, the formulation in a 20 cc vial is reconstituted with 12.5 ml Normal Saline for injection.

In one aspect provided herein is a formulation in a 20 cc vial that consists essentially of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 1875 mg HPBCD, and about 2.1-3.8 mg formic acid as described herein. In one embodiment, the formulation in a 20 cc vial is reconstituted with 12.5 ml Normal Saline for injection.

In one aspect provided herein is a formulation in a 20 cc vial that consists of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 1875 mg HPBCD, and about 2.1-3.8 mg formic acid as described herein. In one embodiment, the formulation in a 20 cc vial is reconstituted with 12.5 ml Normal Saline for injection.

In one embodiment, provided herein is an aqueous formulation comprising Compound D in an amount of about 0.05-0.25% based on total weight of the solids, and HPBCD in an amount of about 99.1-99.9% based on total weight of the solids, and a diluent.

In one embodiment, provided herein is an aqueous formulation comprising Compound D in an amount of about 0.05-0.25% based on total weight of the solids, and HPBCD in an amount of about 99.75-99.95% based on total weight of the solids, and a diluent.

In one embodiment, provided herein is an aqueous formulation consisting essentially of Compound D in an amount of about 0.05-0.25% based on total weight of the solids, and HPBCD in an amount of about 99.75-99.95% based on total weight of the solids, and a diluent.

In one aspect provided herein is an aqueous formulation that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, about 0.6 mg formic acid and about 4.5 mL diluent.

In one aspect provided herein is an aqueous formulation that consists of. Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, about 0.6 mg formic acid and about 4.5 mL diluent.

In one embodiment, provided herein is an aqueous formulation comprising Compound D in an amount of about 0.01-0.08% based on total weight of the solids, and HPBCD in an amount of about 99.50-99.99% based on total weight of the solids, and a diluent.

In one embodiment, provided herein is an aqueous formulation comprising Compound D in an amount of about 0.01-0.08% based on total weight of the solids, and HPBCD in an amount of about 99.50-99.99% based on total weight of the solids, and a diluent.

In one embodiment, provided herein is an aqueous formulation consisting essentially of Compound D in an amount of about 0.01-0.08% based on total weight of the solids, and HPBCD in an amount of about 99.50-99.99% based on total weight of the solids, and a diluent.

In one aspect provided herein is an aqueous formulation that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, about 0.6 mg formic acid and about 4.5 mL diluent.

In one aspect provided herein is an aqueous formulation that consists of: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, 800 mg HPBCD, about 0.6 mg formic acid and about 4.5 mL diluent.

In certain embodiments, the formulation provided herein is lyophilized, and the lyophilized formulation upon reconstitution has a pH of about 2.5 to 4. In certain embodiments, the lyophilized formulation upon reconstitution has a pH of about 2.5 to 3.5. In certain embodiments, the lyophilized formulation upon reconstitution has a pH of about 3.0 to 3.6. In one embodiment, the lyophilized formulation upon reconstitution has a pH of about 2.5, 3, 3.2, 3.4, 3.6, 3.8 or 4. In one embodiment, the lyophilized formulation upon reconstitution has a pH of about 2.5, 2.8, 3, 3.2, 3.4, 3.6, 3.8 or 4.

In certain embodiments, the lyophilized formulation upon reconstitution has an osmolality of about 260-290 mOsm/kg. In certain embodiments, the lyophilized formulation upon reconstitution has an osmolality of about 280 mOsm/kg. In certain embodiments, the lyophilized formulation upon reconstitution has an osmolality of about 260-370 mOsm/kg. In certain embodiments, the lyophilized formulation upon reconstitution has an osmolality of about 360 mOsm/kg. In certain embodiments, the lyophilized formulation upon reconstitution has an osmolality of about 350-450 mOsm/kg. In certain embodiments, the lyophilized formulation upon reconstitution has an osmolality of about 416 mOsm.

In certain embodiments, the lyophilized formulation is reconstituted with half normal saline (0.45% sodium chloride sterile solution for injection) and has an osmolality of about 280-320 mOsm/kg upon reconstitution. In certain embodiments, the lyophilized formulation is reconstituted with half normal saline (0.45% sodium chloride sterile solution for injection), and has a pH of 3.0-3.2 and an osmolality of about 280-320 mOsm/kg upon reconstitution. In certain embodiments, the lyophilized formulation is reconstituted with 4.5 mL of half normal saline (0.45% sodium chloride sterile solution for injection), and has a pH of 3.0-3.2 and an osmolality of about 280-320 mOsm/kg upon reconstitution. In one embodiment, the reconstituted solution of the required dose is diluted with normal saline (0.9% sodium chloride sterile solution for injection) in an infusion bag to a volume to 50 mL for 30-minute intravenous administration.

In certain embodiments, the lyophilized formulation is reconstituted with normal saline and has an osmolality of about 440 mOsm/kg upon reconstitution. In one embodiment, the reconstituted solution of the required dose is diluted with normal saline to a volume to 50 mL to obtain a dosing solution having an osmolality of about 310-380 mOsm/kg. In one embodiment, the reconstituted solution of the required dose is diluted with normal saline to a volume to 50 mL to obtain a dosing solution having an osmolality of about 310-355 mOsm/kg. In one embodiment, the reconstituted solution of the required dose is diluted with normal saline to a volume to 50 mL to obtain a dosing solution having an osmolality of about 317-371 mOsm/kg. In one embodiment, the reconstituted solution of the required dose is diluted with normal saline to a volume to 50 mL to obtain a dosing solution having an osmolality of about 317 mOsm/kg. In one embodiment, the reconstituted solution of the required dose is diluted with normal saline to a volume to 50 mL to obtain a dosing solution having an osmolality of about 371 mOsm/kg. In one embodiment, the osmolality of the dosing solution is no more than 352 mOsm/kg. In one embodiment, the osmolality of the dosing solution having a dose of 4.8 mg Compound D is 352 mOsm/kg.

In certain embodiments, provided herein is a container comprising a formulation provided herein. In one aspect, the container is a glass vial. In one aspect, the container is a 20 cc glass vial.

In one aspect provided herein is a formulation in a 20 cc vial that comprises: Compound D at an amount that provides 1 mg 2-(4-chlorophenyl)-N-((2-(2,6-dioxopiperidin-3-yl)-1-oxoisoindolin-5-yl)methyl)-2,2-difluoroacetamide, and a bulking agent as described herein. In one embodiment, the formulation further comprises no more than about 5 mg formic acid as residual solvent. In one embodiment, the formulation further comprises no more than about 4 mg formic acid as residual solvent. In one embodiment, the formulation further comprises no more than about 3 mg formic acid as residual solvent. In one embodiment, the formulation further comprises no more than about 2 mg formic acid as residual solvent. In one embodiment, the formulation further comprises no more than about 1.5 mg formic acid as residual solvent. In one embodiment, the formulation further comprises no more than about 1 mg formic acid as residual solvent. In one embodiment, the formulation further comprises no more than about 0.8 mg formic acid as residual solvent. In one embodiment, the formulation comprises from about 0.4 mg to about 1.5 mg, about 0.5 mg to about 1 mg, or about 0.5 mg to about 0.9 mg formic acid as residual solvent. In one embodiment, the formulation comprises about 0.4 mg, about 0.6 mg, about 0.8 mg, about 1 mg or about 1.5 mg formic acid as residual solvent. In one embodiment, the formulation comprises formic acid as residual solvent in an amount from about 1.0 mg/mg of Compound D to about 1.8 mg/mg of Compound D, about 2.1 mg/mg of Compound D to about 3.8 mg/mg of Compound D, or about 3.9 mg/mg of Compound D to about 4.9 mg/mg of Compound D.

The formulations of Compound D provided herein can be administered to a patient in need thereof using standard therapeutic methods for delivering Compound D including, but not limited to, the methods described herein. In one embodiment, the formulations provided herein are reconstituted in a pharmaceutically acceptable solvent to produce a pharmaceutically acceptable solution, wherein the solution is administered (such as by intravenous injection) to the patient.

In one aspect, the formulations provided herein lyophilized, and the lyophilized formulations are suitable for reconstitution with a suitable diluent to the appropriate concentration prior to administration. In one embodiment, the lyophilized formulation is stable at room temperature. In one embodiment, the lyophilized formulation is stable at room temperature for up to about 24 months. In one embodiment, the lyophilized formulation is stable at room temperature for up to about 24 months, up to about 18 months, up to about 12 months, up to about 6 months, up to about 3 months or up to about 1 month. In one embodiment, the lyophilized formulation is stable upon storage under accelerated condition of 40° C./75% RH for up to about 12 months, up to about 6 months or up to about 3 months.

The lyophilized formulation provided herein can be reconstituted for parenteral administration to a patient using any pharmaceutically acceptable diluent. Such diluents include, but are not limited to Sterile Water for Injection (SWFI), Dextrose 5% in Water (D5W), or a cosolvent system. Any quantity of diluent may be used to reconstitute the lyophilized formulation such that a suitable solution for injection is prepared. Accordingly, the quantity of the diluent must be sufficient to dissolve the lyophilized formulation. In one embodiment, 1-5 mL or 1 to 4 mL of a diluent are used to reconstitute the lyophilized formulation to yield a final concentration of, about 0.05-0.3 mg/mL or about 0.15-0.25 mg/mL of Compound D. In certain embodiments, the final concentration of Compound D in the reconstituted solution is about 0.25 mg/mL. In certain embodiments, the final concentration of Compound D in the reconstituted solution is about 0.20 mg/mL. In certain embodiments, the volume of the reconstitution diluent varies between 3 ml and 5 ml to yield a final concentration of 0.15-0.3 mg/mL. In certain embodiments, depending on the required dose, multiple vials may be used for reconstitution.

The reconstituted solutions of lyophilized formulation can be stored and used within up to about 24 hours, about 12 hours or about 8 hours. In one embodiment, the reconstituted aqueous solution is stable at room temperature from about 1-24, 2-20, 2-15, 2-10 hours upon reconsititution. In one embodiment, the reconstituted aqueous solution is stable at room temperature for up to about 20, 15, 12, 10, 8, 6, 4 or 2 hours upon reconsititution. In some embodiments, the solution is used within 8 hour of preparation. In some embodiments, the solution is used within 5 hour of preparation. In some embodiments, the solution is used within 1 hour of preparation.

Process for Making Formulations

The formulations provided herein can be prepared by any of the methods known in the art and as described herein, but all methods include the step of bringing the active ingredient into association with the pharmaceutically acceptable excipient, which constitutes one or more necessary ingredients (such as bulking agent and/or buffer).

In one aspect, the formulations provided herein are prepared by dissolving Compound D, a bulking agent and a citrate buffer in water and dimethyl sulfoxide (DMSO) to obtain a solution, and optionally lyophilizing the solution.

In one embodiment, the process for preparing the formulation comprises: dissolving HPBCD in a citrate buffer to obtain a buffer solution, dissolving Compound D in DMSO to obtain a premix, adding the premix to the buffer solution to obtain a solution; and optionally lyophilizing the solution to produce the lyophilized formulation.

In one embodiment, the process comprises dissolving Kleptose® HPB in a 20 mM, pH 4-4.5 citrate buffer to obtain a buffer solution, dissolving Compound D in DMSO to obtain an active premix, adding the premix to the buffer solution to obtain a mixture, adding water to the mixture to obtain a bulk solution, filtering the bulk solution through one or more 0.45 μm and 0.22 μm filters to obtain a filtered solution, filling the filtered solution into a vial, and lyophilizing the solution. In one embodiment, the solution is filtered through one 0.45 μm and two 0.22 μm filters. In one embodiment, the process comprises dissolving Kleptose® HPB in a 20 mM, pH 4.3 citrate buffer to obtain a buffer solution, dissolving Compound D in DMSO to obtain an active premix, adding the premix to the buffer solution to obtain a mixture, adding water to the mixture to obtain a bulk solution, filtering the bulk solution through one 0.45 μm filter and two 0.22 μm filters to obtain a filtered solution, filling the filtered solution into a 20 cc glass vial, and optionally lyophilizing the solution. In one embodiment, the vial is sealed under nitrogen after lyophilization.

In one aspect, the formulations provided herein are prepared by dissolving Compound D in formic acid to obtain a premix, dissolving HPBCD in water to obtain a solution, adding the premix to the solution to obtain a drug solution; and optionally lyophilizing the drug solution to produce the lyophilized formulation.

In one aspect, the formulations provided herein are prepared by dissolving Compound D in formic acid to obtain an active premix, dissolving Kleptose® HPB in water to obtain a Kleptose solution, adding the premix to the Kleptose solution to obtain a mixture, adding water to the mixture to obtain a bulk solution, filtering the bulk solution through one or more 0.45 μm and 0.22 μm filters to obtain a filtered solution, filling the filtered solution into a vial, and lyophilizing the solution. In one embodiment, the solution is filtered through one 0.45 μm and two 0.22 μm filters. In one embodiment, the process comprises dissolving Compound Din formic acid to obtain an active premix, dissolving Kleptose® HPB in water to obtain a Kleptose solution, adding the premix to the Kleptose solution to obtain a mixture, adding water to the mixture to obtain a bulk solution, filtering the bulk solution through one 0.45 μm and two 0.22 μm filters to obtain a filtered solution, filling the filtered solution into a 20 cc glass vial, and lyophilizing the solution. In one embodiment, the vial is sealed under nitrogen after lyophilization.

In one aspect, the lyophilization process contains three stages: freezing, primary drying, and secondary drying. A liquid formulation is transformed to a lyophilized powder form by going through complete solidification through freezing stage, sublimation of ice and solvents through primary drying, and desorption of residual moisture and solvents through secondary drying. The shelf temperature and chamber pressure in the primary drying and secondary drying are controlled to obtain the desired quality of the finished drug product. In one aspect of the process, the cake appearance and structure was characterized by visual inspection. 5.7. Kits

In one aspect, provided herein is a kit for identifying a subject having cancer who is likely to be responsive to a treatment compound, comprising a means for determining the expression level of one or more genes.

In another aspect, provided herein is a kit for treating cancer, comprising a means for determining the expression level of one or more genes in a sample.

In yet another aspect, provided herein is a kit for monitoring the efficacy of a treatment compound in treating cancer in a subject, comprising a means for determining the expression level of one or more genes in a sample.

In certain embodiments, the cancer is blood cancer. In one embodiment, the blood cancer is lymphoma. In another embodiment, the blood cancer is leukemia. In yet another embodiment, the blood cancer is MM. In a specific embodiment, the leukemia is ALL. In another specific embodiment, the leukemia is AML. In yet another specific embodiment, the leukemia is CLL. In still another embodiment, the leukemia is CML. In some embodiments, the AML is relapsed. In certain embodiments, the AML is refractory. In other embodiments, the AML is resistant to conventional therapy.

In certain embodiments, the kit provided herein measures the expression level of one or more genes provided herein and described above for determining the responsiveness of a subject to treatment with Compound D. The kit may include tools for obtaining a sample from a subject. The kit may include tools or agents or measuring the expression level of one or more genes. The kit may also include instructions on how to interpret the measurement, e.g., by providing a reference level of the gene.

In certain embodiments of various kits provided herein, the sample is obtained from a tumor biopsy, a node biopsy, or a biopsy from the bone marrow, spleen, liver, brain, or breast.

In certain embodiments, provided herein is a kit for detecting the mRNA level of one or more genes. In certain embodiments, the kit comprises one or more probes that bind specifically to the mRNAs of the one or more genes. In certain embodiments, the kit further comprises a washing solution. In certain embodiments, the kit further comprises reagents for performing a hybridization assay, mRNA isolation or purification means, detection means, as well as positive and negative controls. In certain embodiments, the kit further comprises an instruction for using the kit. The kit can be tailored for in-home use, clinical use, or research use.

In certain embodiments, provided herein is a kit for detecting the protein level of one or more genes. In certain embodiments, the kits comprises a dipstick coated with an antibody that recognizes the protein biomarker, washing solutions, reagents for performing the assay, protein isolation or purification means, detection means, as well as positive and negative controls. In certain embodiments, the kit further comprises an instruction for using the kit. The kit can be tailored for in-home use, clinical use, or research use.

Such a kit can employ, for example, a dipstick, a membrane, a chip, a disk, a test strip, a filter, a microsphere, a slide, a multi-well plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an organ, an organelle, a biological fluid, a blood sample, a urine sample, or a skin sample.

In another embodiment, the kit comprises a solid support, nucleic acids attached to the support, where the nucleic acids are complementary to at least 20, 50, 100, 200, 350, or more bases of mRNA, and a means for detecting the expression of the mRNA in a biological sample.

In a specific embodiment, the pharmaceutical or assay kit comprises, in a container, a compound or a pharmaceutical composition thereof, and further comprises, in one or more containers, components for isolating RNA. In another specific embodiment, the pharmaceutical or assay kit comprises, in a container, a compound or a pharmaceutical composition, and further comprises, in one or more containers, components for conducting RT-PCR, qRT-PCR, deep sequencing, or microarray.

In certain embodiments, the kits provided herein employ means for detecting the expression of a biomarker by qRT-PCR, microarray, flow cytometry, or immunofluorescence. In other embodiments, the expression of the biomarker is measured by ELISA-based methodologies or other similar methods known in the art.

In another specific embodiment, the pharmaceutical or assay kit comprises, in a container, a compound or a pharmaceutical composition thereof, and further comprises, in one or more containers, components for isolating protein. In another specific embodiment, the pharmaceutical or assay kit comprises, in a container, a compound or a pharmaceutical composition, and further comprises, in one or more containers, components for conducting flow cytometry or ELISA.

In another aspect, provided herein are kits for determining level of a gene that supply the materials necessary to measure the abundance of one or more gene products (e.g., one, two, three, four, five, or more genes) provided herein. Such kits may comprise materials and reagents required for measuring RNA or protein. In some embodiments, such kits include microarrays, wherein the microarray is comprised of oligonucleotides and/or DNA and/or RNA fragments which hybridize to one or more gene products provided herein, or any combination thereof. In some embodiments, such kits may include primers for PCR of either the RNA product or the cDNA copy of the RNA product of the genes. In some embodiments, such kits may include primers for PCR as well as probes for qPCR. In some embodiments, such kits may include multiple primers and multiple probes, wherein some of the probes have different fluorophores so as to permit simultaneously measuring multiple gene products provided herein. In some embodiments, such kits may further include materials and reagents for creating cDNA from RNA. In some embodiments, such kits may include antibodies specific for the protein products of the gene provided herein. Such kits may additionally comprise materials and reagents for isolating RNA and/or proteins from a biological sample. In addition, such kits may include materials and reagents for synthesizing cDNA from RNA isolated from a biological sample. In some embodiments, such kits may include a computer program product embedded on computer readable media for predicting whether a patient is clinically sensitive to a compound. In some embodiments, the kits may include a computer program product embedded on a computer readable media along with instructions.

In some embodiments, such kits measure the expression of one or more nucleic acid products of the genes provided herein. In accordance with this embodiment, the kits may comprise materials and reagents that are necessary for measuring the expression of particular nucleic acid products of the genes provided herein. For example, a microarray or RT-PCR kit may be produced for a specific condition and contain only those reagents and materials necessary for measuring the levels of specific RNA transcript products of the genes provided herein, to predict whether a hematological cancer in a patient is clinically sensitive to a compound. Alternatively, in some embodiments, the kits can comprise materials and reagents necessary for measuring the expression of particular nucleic acid products of genes other than the genes provided herein. For example, in certain embodiments, the kits comprise materials and reagents necessary for measuring the expression levels of 1, 2, 3, 4, 5, 6, 7, 8, 9, 10, 11, 12, 13, 14, 15, 16, 17, 20, 25, 30, 35, 40, 45, 50, or more of the genes, in addition to reagents and materials necessary for measuring the expression levels of the genes provided herein. In other embodiments, the kits contain reagents and materials necessary for measuring the expression levels of at least 1, at least 2, at least 3, at least 4, at least 5, at least 6, at least 7, at least 8, at least 9, at least 10, or more of the genes provided herein, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not the genes provided herein.

For nucleic acid microarray kits, the kits generally comprise probes attached to a solid support surface. In one such embodiment, probes can be either oligonucleotides or longer probes including probes ranging from 150 nucleotides to 800 nucleotides in length. The probes may be labeled with a detectable label. In a specific embodiment, the probes are specific for one or more of the gene products of the biomarkers provided herein. The microarray kits may comprise instructions for performing the assay and methods for interpreting and analyzing the data resulting from performing the assay. In a specific embodiment, the kits comprise instructions for predicting whether a hematological cancer in a patient is clinically sensitive to a compound. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own suitable container.

In certain embodiments, a nucleic acid microarray kit comprises materials and reagents necessary for measuring the expression levels of 1, 2, 3, 4, 5, 6, 7, 8, 9, 10, 15, 20, 25, 30, 35, 40, 45, 50, or more of the genes provided herein, or a combination thereof, in addition to reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more genes other than those of the genes provided herein. In other embodiments, a nucleic acid microarray kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes provided herein, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not of the genes provided herein. In another embodiment, a nucleic acid microarray kit contains reagents and materials necessary for measuring the expression levels of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, or more of the genes of the genes provided herein, or any combination thereof, and 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000, or 500-1000 genes that are not the genes provided herein.

For quantitative PCR, the kits generally comprise pre-selected primers specific for particular nucleic acid sequences. The quantitative PCR kits may also comprise enzymes suitable for amplifying nucleic acids (e.g., polymerases such as Taq polymerase), deoxynucleotides, and buffers needed for amplification reaction. The quantitative PCR kits may also comprise probes specific for the nucleic acid sequences associated with or indicative of a condition. The probes may or may not be labeled with a fluorophore. The probes may or may not be labeled with a quencher molecule. In some embodiments, the quantitative PCR kits also comprise components suitable for reverse-transcribing RNA, including enzymes (e.g., reverse transcriptases such as AMV, MMLV, and the like) and primers for reverse transcription along with deoxynucleotides and buffers needed for reverse transcription reaction. Each component of the quantitative PCR kit is generally in its own suitable container. Thus, these kits generally comprise distinct containers suitable for each individual reagent, enzyme, primer and probe. Further, the quantitative PCR kits may comprise instructions for performing the reaction and methods for interpreting and analyzing the data resulting from performing the reaction. In a specific embodiment, the kits contain instructions for predicting whether a hematological cancer in a patient is clinically sensitive to a compound.

For antibody-based kits, the kit can comprise, for example: (1) a first antibody (which may or may not be attached to a solid support) that binds to a peptide, polypeptide or protein of interest; and, optionally, (2) a second, different antibody that binds to either the first antibody or the peptide, polypeptide, or protein, and is conjugated to a detectable label (e.g., a fluorescent label, radioactive isotope, or enzyme). In a specific embodiment, the peptide, polypeptide, or protein of interest is associated with or indicative of a condition (e.g., a disease). The antibody-based kits may also comprise beads for conducting immunoprecipitation. Each component of the antibody-based kits is generally in its own suitable container. Thus, these kits generally comprise distinct containers suitable for each antibody and reagent. Further, the antibody-based kits may comprise instructions for performing the assay and methods for interpreting and analyzing the data resulting from performing the assay. In a specific embodiment, the kits contain instructions for predicting whether a hematological cancer in a patient is clinically sensitive to a compound.

In one embodiment, a kit provided herein comprises a compound provided herein, or a pharmaceutically acceptable salt, solvate, stereoisomer, isotopologue, prodrug, hydrate, co-crystal, clathrate, or a polymorph thereof. Kits may further comprise additional active agents, including but not limited to those disclosed herein.

Kits provided herein may further comprise devices that are used to administer the active ingredients. Examples of such devices include, but are not limited to, syringes, drip bags, patches, and inhalers.

Kits may further comprise cells or blood for transplantation, as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to, water for injection USP; aqueous vehicles (such as, but not limited to, sodium chloride injection, Ringer's injection, dextrose injection, dextrose and sodium chloride injection, and lactated Ringer's injection); water-miscible vehicles (such as, but not limited to, ethyl alcohol, polyethylene glycol, and polypropylene glycol); and non-aqueous vehicles (such as, but not limited to, corn oil, cottonseed oil, peanut oil, sesame oil, ethyl oleate, isopropyl myristate, and benzyl benzoate).

In certain embodiments of the methods and kits provided herein, solid phase supports are used for purifying proteins, labeling samples, or carrying out the solid phase assays. Examples of solid phases suitable for carrying out the methods disclosed herein include beads, particles, colloids, single surfaces, tubes, multi-well plates, microtiter plates, slides, membranes, gels, and electrodes. When the solid phase is a particulate material (e.g., a bead), it is, in one embodiment, distributed in the wells of multi-well plates to allow for parallel processing of the solid phase supports.

It is noted that any combination of the above-listed embodiments, for example, with respect to one or more reagents, such as, without limitation, nucleic acid primers, solid support, and the like, are also contemplated in relation to any of the various methods and/or kits provided herein.

Certain embodiments of the invention are illustrated by the following non-limiting examples.

### 6. EXAMPLES

The examples below are carried out using standard techniques, which are well known and routine to those of skill in the art, except where otherwise described in detail. The examples are intended to be merely illustrative.

6.1. The Anti-Proliferative Activity of Compound D

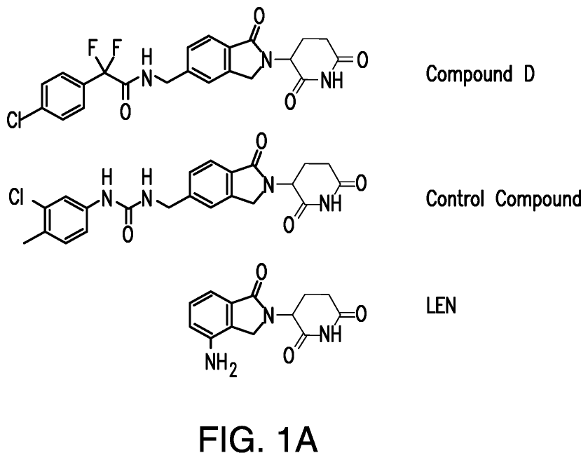

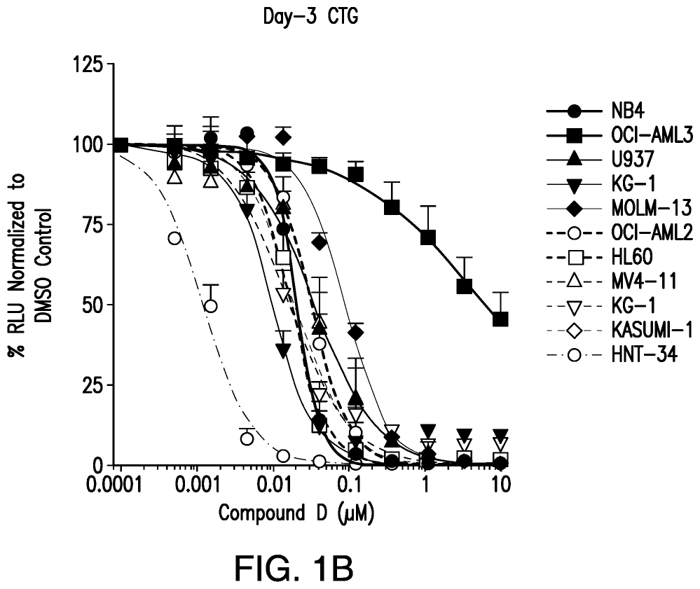

Compound D demonstrated potent anti-proliferative activity in 10 out of 11 AML cell lines (FIGS. 1A and 1B).

For cell proliferation assays, human cancer cell lines cultured in the growth medium recommended by the vendor (as described above) were seeded into black 384-well plates containing DMSO or test compounds. The seeding density of each cell line was optimized to allow for cell growth in the linear growth phase during a 3-day culture period. To test the compounds' effect on cell proliferation in AML cell lines, 5,000 to 10,000 cells per well in 200 L complete culture media were seeded into black 96-well plates containing DMSO or test compounds. After 72 hours, cell proliferation was assessed using the CTG assay according to the manufacturer's instructions. To test the effect of GSPT1 depletion on cell proliferation, U937 cells were infected with lentiviral shRNA vectors for 4 days, and cell proliferation was quantified using CTG every other day thereafter. Relative cell proliferation was normalized against cell growth values on day 4. The growth inhibitory curve was processed and graphed using GraphPad Prism Version 7 (P<0.05, unpaired two-sided t-test, is considered as significant).

Like all other cereblon modulators, Compound D contains a glutarimide ring (FIG. 1A) that can bind cereblon and redirect it to target neosubstrates for degradation. Thus, the effect of Compound D on the global proteome in AML cell line KG-1 was assessed using tandem mass tag (TMT) quantitative mass spectrometry. Treatment with 0.1 μM Compound D for 4 hours triggered significant reduction of GSPT1 protein abundance with minimal effect on the rest of the proteome (FIG. 2A). The downregulation of GSPT1 by Compound D can be completely blocked by proteasomal inhibition with bortezomib, inactivation of the CRL4^{CRBN} E3 ubiquitinligase complex with a NEDD8 E1 inhibitor MLN4924, or knockout of CRBN via CRISPR/Cas9 mediated gene editing (FIGS. 2B, 2C, 2E and 2F).

6.2. Targeted Degradation of GSPT1 by Compound D Via a Glycine-Containing Degron Mediates the Anti-Proliferative Activity of Compound D

Compound D promoted the binding of cereblon to GSPT1 but not IKZF1 when added directly into the binding assays performed with cell extracts, whereas lenalidomide exhibited binding selectivity toward IKZF1 over GSPT1 (FIG. 3A). A cereblon mutant carrying two point mutations Y384A and W386A, which impair the docking of cereblon modulators into the tri-tryptophan pocket of cereblon thalidomide-binding domain, completely abolished the binding of cereblon to GSPT1 or IKZF1 in the presence of Compound D or lenalidomide, respectively (FIG. 3A).

To further define the molecular basis of the selective recruitment of GSPT1 to the cereblon E3 ligase complex by Compound D, GSPT1 was co-crystalized in complex with human DDB1, cereblon and Compound D (FIGS. 3B and 3C).

To express and purify DDB1-cereblon, ZZ-domain-6×His-thrombin-tagged human cereblon (amino acids 40-442) and the full length human DDB1 were co-expressed in SF9 insect cells in ESF921 medium (Expression Systems) with 50 μM zinc acetate. Cells were lysed in 50 mM Tris-HCl pH 7.5, 500 mM NaCl, 10 mM imidazole, 10% glycerol, 5 mM BME, Salt Active Nuclease (Sigma-Aldrich), and Protease Inhibitor XL Capsules, EDTA-free (Pierce) using a handheld homogenizer. The lysate was centrifuged at 38,400×g for 45 minutes, and the clarified lysate was incubated with Ni-NTA affinity resin (Qiagen) with rotation for 1 hour. The complex was eluted with lysis buffer with 250 mM imidazole, and the ZZ-domain-6×His tag was removed by thrombin cleavage (Enzyme Research) overnight, combined with dialysis in lysis buffer. The cleaved protein was run over a HisTrap column (GE Healthcare), and the flow-through and wash was diluted to 150 mM NaCl and run over an ANX HiTrap ion exchange column (GE Healthcare). The ANX column was washed with 50 mM Tris-HCl pH 7.5, 150 mM NaCl, 3 mM TCEP, followed by 50 mM Bis-Tris pH 6.0, 150 mM NaCl, 3 mM TCEP. The CRBN-DDB1 peak was run over a Superdex S200 26/60 column (GE Healthcare) in 10 mM HEPES pH 7.0, 240 mM NaCl, and 3 mM TECP. The cereblon-DDB1 complex was concentrated to 50 mg/mL.

To express and purify GSPT1, GSPT1 domains 2 and 3 (amino acids 437-633) were expressed as an MBP-fusion in Escherichia coli BL21 (DE3) Star cells (Life Technologies) using 2XYT media (Teknova). Cells were induced at a density of OD600 of 0.6 and grown overnight at 16° C. Cells were pelleted, resuspended in 50 mM Tris pH 7.5, 200 mM NaCl, 1 mM TCEP, 10% glycerol, lysozyme (Sigma), Benzonase (Novagen), and Protease Inhibitor XL Capsules, EDTA-free (Pierce). Cells were sonicated and the lysate was centrifuged at 384,000×g for 45 min. The lysate was incubated with amylose resin (NEB) at 4° C. with rotation for 1 hour. The protein was eluted in lysis buffer with 10 mM maltose, and the MBP tag was removed by overnight cleavage with thrombin (Enzyme Research). Cleaved GSPT1 was diluted to 90 mM NaCl and run over a Heparin HiTrap column (GE Healthcare). The GSPT1 peak was run over a Superdex 75 26/600 column (GE Healthcare) in 10 mM HEPES pH 7.0, 240 mM NaCl, and 3 mM TECP. The peak containing GSPT1 was concentrated to 10 mg/mL for crystallization trials.

Crystallization of the complex was achieved by sitting-drop vapour diffusion. Cereblon-DDB1 and GSPT1 were mixed together to equimolar stoichiometry at a final concentration of 150 μM. The solution of cereblon-DDB1-GSPT1 in the presence of 500 M Compound D was mixed 1:1 with, and subsequently equilibrated against, a mother liquor solution of 300 mM sodium citrate, 100 mM Tris-HCl (pH 7.5), 20% PEG 3350 and incubated at 20° C. Crystals were cryoprotected in the reservoir solution supplemented with 20% ethylene glycol and cooled under liquid nitrogen. Data were collected from a single crystal at the Advanced Light Source, beamline 5.0.2. The structure of human cereblon-DDB1-GSPT1-Compound D was solved by molecular replacement using Phaser (McCoy et al., J. Appl. Crystallogr., 2007, 40:658-74), with human cereblon-DDB1-GSPT1-Control Compound (PDB code 5HXB) as a search model. Subsequent manual model building using Coot and refinement were performed using Phenix with non-crystallographic symmetry and external structure restraints (Liebschner et al., Acta Crystallogr. D Struct. Biol., 2019, 75:861-77).

GSPT1 interaction with cereblon and Compound D is mediated by a P-hairpin degron loop formed by GSPT1 residues 568-576 (FIG. 3D). Hydrogen bond interactions are formed between the backbone carbonyls of GSPT1 residues K572, K573, and S574 and cereblon residues N351, H357, and W400, respectively (FIG. 3D). The glutarimide moiety of Compound D binds the cereblon tri-tryptophan pocket formed by residues W380, W386, and W400, presenting the isoindoline ring above the cereblon surface such that it forms Van der Waals and hydrophobic interactions with GSPT1 glycine residue 575 (FIG. 3E). Replacement of glycine 575 with asparagine conferred complete resistance to Compound D-induced degradation (FIGS. 3F-311). Compound D extends from the isoindolinone, making a hydrogen bond interaction between the difluoroacetamide moiety and the side chain of cereblon residues H353 and positioning the chlorophenyl moiety proximal to the j-sheet core of GSPT1 domain 3 (FIG. 3E). A notable difference in the side chain position of cereblon residue E377 is observed relative to the Control Compound structure, suggesting a lack of interaction between E377 and Compound D (FIG. 3I).

While ablation of CRBN completely abolished the anti-proliferative effect of Compound D in U937, OCI-AML2 and MOLM-13 (FIGS. 2D-2F), it is possible that the anti-AML activity of Compound D is mediated through GSPT1 and/or additional substrate(s) that cannot be detected by mass spectrometry owing to technological limitations. To explore this hypothesis, the antiproliferative effect of Compound D in U937, OCI-AML2 and MOLM-13 parental cells and cells stably overexpressing GSPT1-G575N was determined. GSPT1 stabilization completely inhibited the response to Compound D (FIGS. 3F-311), ruling out the participation of other substrates in the antitumor effect of Compound D. Moreover, RNAi-mediated GSPT1 knockdown led to rapid loss of cell fitness in U937 cells (FIGS. 3J and 3K), consistent with our previous observations in other AML cell lines (Matyskiela et al., Nat. Chem. Biol., 2016, 535:252-57). Thus, GSPT1 degradation is both necessary and sufficient to account for the anti-AML activity of Compound D.

6.3. Genes Involved in Response to Compound D in AML Cells

A genome-wide CRISPR screen (see FIG. 5A) was used to determine genes involved in Compound D sensitivity and resistance in an AML cell line. U937 cells stably expressing Cas9 protein were inoculated with a pooled small guide RNA (sgRNA) library. The pooled human sgRNA library expressed 4-8 sgRNAs targeting over 19,000 protein-coding genes for a total of 150,076 unique sgRNAs. The U937 cells were treated with 100 nM, of Compound D for 2 days (D3 post-infection), 7 days (D8 post-infection), or 11 days (D12 post-infection) or with 1 μM or 10 μM of Compound D for 5 days (D8 post-infection) or 9 days (D12 post-infection).

Specifically, a total of 6×10⁸ U937 cells stably expressing Cas9 protein were inoculated with a lentiviral supernatant containing a small guide RNA (sgRNA) library (Cellecta) at a multiplicity of infection (MOI) of 0.3 according to the Cellecta CRISPR pooled sgRNA libraries screening guide to ensure that each cell was transduced with only one sgRNA. The Cellecta pooled human sgRNA library used expressed 4-8 sgRNAs targeting each of over 19,000 protein-coding genes for a total of 150,076 unique sgRNAs. Vectors also expressed a RFP reporter and a puromycin resistance gene. After 24 hours of puromycin selection, cells were split into samples and treated with DMSO or with a sub-lethal Compound D dose of 100 nM for the sensitivity screen. After an additional 48 hours, the DMSO-treated cells were split into three samples that were treated with either DMSO, semi-lethal, or lethal doses of Compound D of 1 μM or 10 μM for the resistance screens. Cells were grown in 2 L flasks with agitation and at least 200 million cells were maintained after each passage to exceed the 150K library complexity by more than 1000-fold, as recommended by the Cellecta CRISPR screening guide for maintaining library representation. Compound D was fully replenished after the first five days of treatment. 9×10⁷ cell pellets were collected in technical duplicates on days 3, 8, and 12 post-infection for genomic DNA isolation and sequencing library preparation. The samples collected on day 3 represented TO controls for comparison to treatment days 8 and 12. To generate sgRNA libraries for Next Generation Sequencing (NGS), genomic DNA was isolated from the pellets and the sgRNA portion of the constructs was amplified. Libraries were sequenced on the Illumina HiSeq 4000. Data were analyzed by comparing sgRNA counts between samples. For the sensitivity screen, sgRNAs that had ‘dropped out’ with Compound D treatment compared to DMSO indicated genes whose knockout enhanced sensitivity. Enriched sgRNAs in samples treated with lethal doses relative to DMSO indicated genes whose knockout conferred resistance (see FIG. 5A).

FIG. 5B shows the genes that dropped-out or enriched upon treatment with Compound D. The results indicate that knockout of RPTOR, MTOR, RICTOR, GSPT1 or DDX5 enhanced sensitivity to lethal doses of Compound D, whereas knockout of CRBN, TSC1, TSC2, ILF2, ILF3, ATF4, GCN2, DDIT4 or GCN1L1 genes conferred resistance to lethal doses of Compound D.

To further elucidate the mechanism of anti-proliferative activity of Compound D in AML, another genome-wide positive-selection CRISPR-Cas9 screen was used to delineate genes and pathways that govern the response to Compound D (FIG. 4A). U937 cells stably expressing Cas9 were transduced with a pooled lentiviral library targeting every protein-encoding gene with 4-8 different sgRNAs at a low multiplicity of infection (MOI) of 0.3. At day 3 post-transduction, cells were treated with 10 μM Compound D or DMSO vehicle control for an additional 9 days, followed by amplification of sgRNA coding regions from genomic DNA in surviving cells, and next generation sequencing to identify and quantify the abundance of sgRNAs.

To generate sgRNA libraries for Next Generation Sequencing (NGS), 9×10⁷ cells per sample were lysed with Qiagen Buffer P1 with added RNase A. Lysates were adjusted to 0.5% SDS and chromatin was sheared into 10-100 kb fragments with a probe sonicator. After treatment with Proteinase K, genomic DNA was extracted with Phenol:Chloroform:Isoamyl Alcohol and precipitated overnight with isopropanol and 10% sodium acetate. The DNA pellet was washed with ethanol and dissolved in water. Total genomic DNA was quantified on the Nanodrop.

To amplify the sgRNA portions of the transduced sgRNA construct in each cell, PCR was performed on all of the genomic DNA isolated per sample with 25 μg DNA amplified per reaction. Twenty-four cycles of PCR were performed with an annealing temperature of 65° C. After visualizing the 477 base-pair PCR products on an agarose gel, PCR reactions from each sample were combined, mixed, and 100 μl per sample was cleaned with 1× volume of SPRIselect beads in a 2-step cleanup protocol to eliminate both primers and genomic DNA carryover. Products were eluted in Qiagen Elution Buffer and measured on the Nanodrop. A second PCR reaction was performed on the first PCR product to incorporate dual-indexed Illumina primers into the final sgRNA libraries. Six cycles of PCR were run with an annealing temperature of 65° C. and four cycles were run with an annealing temperature of 71° C. to reduce non-specific products. After mixing the four reactions per sample and confirming the 339 base-pair libraries on an agarose gel, 100 μl of each library was cleaned with 1× volume of SPRIselect beads in a 2-step cleanup protocol to eliminate both primers and carryover of the first PCR reaction.

Final sgRNA libraries were visualized on the Agilent Bioanalyzer and quantified with the KAPA library quantitation kit. Samples were diluted to 3 nM and 2-3 samples were run per lane on the Illumina HiSeq 4000. Each lane also contained 5% molar ratio spike-in of PhiX to enhance sequence diversity. Data were analyzed by comparing sgRNA counts between samples. Enriched sgRNAs in samples treated with lethal doses relative to the DMSO condition at the same timepoint indicated genes whose knockout conferred resistance.

The log 2 fold change (log 2FC) in sgRNA read count in the Compound D treated sample as compared with DMSO control treated sample was designated as the enrichment score, and the average log 2FC value of all sgRNAs for a gene of interest was used to quantify the effect of gene knockout on Compound D response (FIG. 4A). Compound D treatment led to growth arrest of U937 cells transduced with the lentiviral CRISPR library, and significantly affected the sgRNA distribution as compared to the DMSO control (FIGS. 4B and 4C). A subset of sgRNAs including those targeting CRBN and UBE2G1 clustered separately from the remaining sgRNAs (FIG. 4C).

Pathway enrichment analysis of top-ranked genes with log 2FC>2 and false discovery rate (FDR)<0.05 yielded a few protein complexes and signaling cascades that regulate the response to Compound D (FIGS. 4D-4F). As expected, a significant fraction of Compound D-enriched genes encode proteins known to be essential for the biological activities of all cereblon modulators, including subunits of the cereblon E3 ligase complex and the COP9 signalosome, the ubiquitin conjugation enzymes UBE2D3 and UBE2G1, components of the NEDD8 conjugation pathway, and the Cullin Ring E3 ligase assembly factor CAND1 (FIGS. 4E and 4G). In addition, a number of candidate genes that have not been previously reported to affect the response to other cereblon modulators were uncovered with a well-defined mechanistic basis. These include modulators of RNA alternative splicing such as ILF2 and ILF3, suppressors of mTOR signaling such as TSC1 and TSC2, and key components of the integrated stress response (ISR) pathway such as GCN1 (GCN1L1), GCN2 (EIF2AK4), and ATF4 (FIGS. 4D and 4H). Multiple sgRNAs targeting each of these top-ranked genes were significantly enriched by Compound D, strongly supporting the on-target gene knockout effect on Compound D response (FIGS. 4G and 4H).

To further analyze the underlying mechanism of Compound D sensitivity and resistance, further studies with respect to these genes were performed.

6.4. Knockout of mTOR, Raptor and Rictor Enhances Sensitivity to Compound D

To investigate the role of the mTOR signaling pathway in mediating Compound D sensitivity, a CRISPR competition assay was performed (see FIG. 6A). U937 cells stably expressing Cas9 were infected with a lentiviral vector expressing GFP and non-targeting sgRNA (sgNT), or lentiviral vectors expressing RFP and non-targeting sgRNA (sgNT), or lentiviral vectors expressing RFP and sgRNAs targeting MTOR, RICTOR or RAPTOR. Cells were then treated with DMSO or 100 nM Compound D. The RFP+/GFP+ ratio was monitored by flow cytometry every 2-3 days thereafter.

To confirm knockout of the target genes, one million cells per sample were washed in ice-cold 1×PBS before harvest in Buffer A [50 mM Tris.Cl (pH 7.6), 150 mM NaCl, 1% Triton X-100, 1 mM EDTA, 1 mM EGTA, 1 mM P-glycerophosphate, 2.5 mM sodium pyrophosphate, 1 mM Na3VO4, 1 μg/mL leupeptin, one tablet of Complete ULTRA protease inhibitor cocktail (Roche), and one tablet of PhosSTOP phosphatase inhibitor cocktail (Roche)]. Whole cell extracts were collected after centrifugation at top speed for 10 minutes, resolved by SDS-PAGE gel electrophoresis, transferred onto a nitrocellulose membrane using the Turboblot system (Bio-Rad), and probed with the indicated primary antibodies. Bound antibodies were detected with IRDye-680 or -800 conjugated secondary antibodies using a LI-COR scanner. Confirmation of mTOR, Raptor and Rictor knockout is shown in FIG. 6B.

As shown in FIGS. 6C-6E, knockout of mTOR, Raptor and Rictor enhances U937 sensitivity to Compound D compared to control cells, indicating the mTOR pathway and its components may be involved in Compound D resistance.

6.5. Compound D Resistance Mediated by mTOR

In response to amino acid and glucose stimulation, mTOR is translocated to the lysosomal surface, where it is activated by Rheb (Liu and Sabatini, Nat. Rev. Mol. Cell Biol., 2020, 21:246). The lysosomal translocation of mTOR is negatively regulated by the KICSTOR complex (consisting of SZT2, C12orf66, ITFG2 and KPTN) and the GATORI complex (consisting of NPRL2, NPRL3 and DEPDC5), whereas Rheb is suppressed by the TSC complex (consisting of TSC1, TSC2 and TBC1D7) (Liu and Sabatini, Supra).

To further analyze the role of mTOR in Compound D responsiveness, various regulators (e.g., negative regulators) of mTOR signaling were knocked out.

Loss of TSC1 or TSC2 Conferred Resistance to Compound D

Knockout of TSC1 and TSC2 was accomplished in AML cell lines, U937 and OCI-AML2, by CRISPR-mediated gene editing as described above.

Specifically, U937 cells stably expressing Cas9 were infected with lentiviral vectors constitutively expressing RFP and non-targeting or non-coding sgRNA (sgNT) controls, or sgRNAs targeting TSC1 or TSC2. Infected cells were selected with 1 μg/ml puromycin. Five days after infection, one million cells per sample were washed in ice-cold 1×PBS twice before harvest in Buffer A [50 mM Tris.Cl (pH 7.6), 150 mM NaCl, 1% Triton X-100, 1 mM EDTA, 1 mM EGTA, 1 mM P-glycerophosphate, 2.5 mM sodium pyrophosphate, 1 mM Na3VO4, 1 g/mL leupeptin, one tablet of Complete ULTRA protease inhibitor cocktail (Roche), and one tablet of PhosSTOP phosphatase inhibitor cocktail (Roche)]. Whole cell extracts were collected after centrifugation at top speed for 10 minutes, resolved by SDS-PAGE gel electrophoresis, transferred onto a nitrocellulose membrane using the Turboblot system (Bio-Rad), and probed with the indicated primary antibodies. Bound antibodies were detected with IRDye-680 or -800 conjugated secondary antibodies using a LI-COR scanner.

Similarly, OCI-AML2 cells stably expressing Cas9 were infected with lentiviral vectors constitutively expressing RFP and non-targeting or non-coding sgRNA (sgNT) controls or sgRNAs targeting TSC1 or TSC2. Infected cells were selected with 1 μg/ml puromycin. Five days after infection, one million OCI-AML2 cells per sample were washed with ice-cold 1×PBS and lysed with 100 μl 2×LDS buffer containing 2-mercaptoethanol. Lysates were boiled for 10 minutes at 95° C. Whole cell extracts were resolved by SDS-PAGE gel electrophoresis, transferred onto a nitrocellulose membrane using the Turboblot system (Bio-Rad), and probed with the indicated primary antibodies. Bound antibodies were detected with IRDye-680 or -800 conjugated secondary antibodies using a LI-COR scanner.

Western blot analysis confirmed knockout of these genes in U937 and OCI-AML2 cells, respectively (see FIGS. 7A and 7B).

Then, a cell proliferation assay was performed. Compound D dissolved in DMSO was injected into 96-well cell culture plates using a HP D300 digital dispenser in triplicate Compound D concentrations. One week after infection to knock-out the indicated genes, each of U937 Cas9 cell lines and OCI-AML2 Cas9 cell lines were plated in triplicate with 5000 cells in 100 μL complete culture media per well in the 96-well plates. After 5 days, cell proliferation was assessed using CTG (CellTiter-Glo) according to the manufacturer's instructions to assess the effects of Compound D on proliferation of each cell line. FIGS. 7C and 7D show that Compound D exhibited dose-dependent anti-proliferative effects in parental, non-targeting (sgNT-1) and non-coding (sgNC-8) controls. However, depletion of TSC1 and TSC2 in U937 cells diminished this effect.

Next, CRISPR competition assays were conducted in U937 and OCI-AML2 cells as described above. Specifically, U937 cells stably expressing Cas9 were infected with lentiviral vector constitutively expressing GFP and non-targeting sgRNA (sgNT), or with lentiviral vectors constitutively expressing RFP and non-targeting sgRNA (sgNT) or sgRNAs targeting TSC1 or TSC2. Three days after infection, RFP and GFP cells were mixed at a 1:1 ratio and treated with DMSO, 1 μM Compound D, or 10 μM Compound D. The change of RFP+/GFP+ ratio was monitored by flow cytometry every 2-3 days thereafter. Similarly, OCI-AML2 cells stably expressing Cas9 were infected with lentiviral vector constitutively expressing GFP and non-targeting sgRNA (sgNT), or with lentiviral vectors constitutively expressing RFP and non-targeting sgRNA (sgNT) or sgRNAs targeting TSC1 or TSC2. Three days after infection, RFP and GFP cells were mixed at a 1:1 ratio and treated with DMSO, 0.1 μM Compound D, or 1 M Compound D. The change of RFP+/GFP+ ratio was monitored by flow cytometry every 2-3 days thereafter.

Immunoblot analysis was also performed. One million U937 cells per sample were washed with ice-cold 1×PBS and lysed with 100 μl 2×LDS buffer containing 2-mercaptoethanol two weeks after infection. Lysates were boiled for 10 minutes at 95° C. Whole cell extracts were resolved by SDS-PAGE gel electrophoresis, transferred onto a nitrocellulose membrane using the Turboblot system (Bio-Rad), and probed with the indicated primary antibodies. Bound antibodies were detected with IRDye-680 or -800 conjugated secondary antibodies using a LI-COR scanner. Exemplary results are shown in FIG. 7E.

In the CRISPR screen, nearly every sgRNA targeting genes encoding subunits of the TSC complex, the GATORI complex, or the KICSTOR complex was significantly enriched by Compound D (FIG. 7K). Knockout of TSC1 or TSC2 resulted in enhancement of S6K1 phosphorylation, indicative of mTOR hyperactivation (FIGS. 7B and 7E). Consistent with the CRISPR screen, TSC1 or TSC2 deficiency conferred a growth advantage in the presence of, but not in the absence of, Compound D in U937 and OCI-AML2, when tested in the CRISPR competition assay (FIGS. 7H-7J). TSC1 or TSC2 loss partially blocked GSPT1 degradation induced by Compound D, providing an explanation for the attenuated Compound D response upon mTOR activation (FIGS. 8A and 8B).

The results of the CRISPR competition assays (see FIGS. 7F and 7G) also show that depletion of TSC1 or TSC2 increased resistance to Compound D in U937 and OCI-AML2 cells.

Knockout of TSC1 and TSC2 Attenuated Compound D Induced GSPT1 Degradation

To understand whether the effect of mTOR activation on Compound D-induced GSPT1 degradation can be ascribed to an increased rate of GSPT1 protein synthesis and/or a decreased rate of GSPT1 degradation, the change of GSPT1 protein half-life in response to TSC1 or TSC2 loss in the presence or absence of Compound D was determined. Cotreatment with Compound D and cycloheximide, a protein synthesis inhibitor, downregulated GSPT1 expression and TSC1 or TSC2 loss largely abrogated this effect, whereas cycloheximide treatment alone did not show much effect on the protein level of GSPT1 in U937 parental, TSC1−/− and TSC2−/− cells (FIG. 8C). Hyperactivation of mTOR also exhibited the same effect on the degradation of HA-tagged GSPT1 induced by Compound D, as compared to that of endogenous GSPT1 (FIG. 8D). Because TSC1 or TSC2 deficiency did not affect the degradation of Ikaros by pomalidomide (FIG. 8E), it was reasoned that mTOR activation might limit the accessibility of GSPT1 by cereblon without affecting the activity of the cereblon E3 ligase complex or the 26S proteasome. Consistent with this hypothesis, TSC1 loss significantly reduced the interaction between HA-tagged GSPT1 and endogenous cereblon induced by Compound D (FIG. 8F).

Knockout of TSC1 and TSC2 was accomplished in AML cell lines, U937 and OCI-AML2, by CRISPR-mediated gene editing tool as previously described. Western blot analysis confirmed knockout of these genes in U937 and OCI-AML2 cells, respectively (see FIGS. 8A and 8B). Furthermore, FIGS. 8A and 8B show that TSC1 or TSC2 knockout. Attenuated Compound D induced GSPT1 degradation relative to Compound D induced GSPT1 degradation in parental, non-targeting (sgNT-1), and non-coding (sgNC-8) controls.

Knockout of GCN2 and Other Negative Regulators of mTOR Conferred Resistance to Compound D

A CRISPR competition assay was performed as shown in the upper panel of FIG. 5A, and similarly to the assay described above. U937 cells stably expressing Cas9 were infected with a lentiviral vector constitutively expressing GFP and non-targeting sgRNA (sgNT), or with lentiviral vectors constitutively expressing RFP and non-targeting sgRNA (sgNT) or sgRNAs targeting GCN2. On day 2, RFP and GFP cells were mixed at a 1:1 ratio and treated with DMSO or 100 nM Compound D. The change of RFP+/GFP+ ratio was monitored by flow cytometry every 2-3 days thereafter.

To confirm knockout of the target genes, one million cells per sample were washed in ice-cold 1×PBS before harvest in Buffer A [50 mM Tris.Cl (pH 7.6), 150 mM NaCl, 1% Triton X-100, 1 mM EDTA, 1 mM EGTA, 1 mM P-glycerophosphate, 2.5 mM sodium pyrophosphate, 1 mM Na3VO4, 1 μg/mL leupeptin, one tablet of Complete ULTRA protease inhibitor cocktail (Roche), and one tablet of PhosSTOP phosphatase inhibitor cocktail (Roche)]. Whole cell extracts were collected after centrifugation at top speed for 10 minutes, resolved by SDS-PAGE gel electrophoresis, transferred onto a nitrocellulose membrane using the Turboblot system (Bio-Rad), and probed with the indicated primary antibodies. Bound antibodies were detected with IRDye-680 or -800 conjugated secondary antibodies using a LI-COR scanner. Western blot analysis confirmed GCN2 knockout (see FIG. 9A).

The results of the CRISPR competition assay is shown in FIG. 9B, which demonstrates that U937 cells lacking GCN2 were resistant to Compound D.

Next, the roles of additional negative regulators of mTOR were studied. To knockout the studied genes, an electroporation-based method was used where a ribonucleoprotein (RNP) consisting of the Cas9 protein and gene-targeting gRNA was directly delivered to the U937 cells. A crRNA-tracrRNA duplex was first prepared consisting of the crRNA with a 20 nt sequence specific to each target gene and the universal tracrRNA to guide the Cas9 nuclease to the genomic locus. The sgRNA sequences targeting CRBN, ATF4, GCN1, GCN2, DDIT4, TSC1, TSC2, or non-targeting (NT-1) and non-coding (NC-8) controls were selected from the Cellecta sgRNA library. After pre-coupling the Cas9 enzyme to the crRNA-tracrRNA duplex, the complex was mixed with one million U937 cells per sample in nucleofection solution (4D-Nucleofector X kit SE; Lonza). Cells were electroporated in a 4D nucleofector according to the manufacturer's instructions and plated into complete cell culture media.

To confirm knockout of the target genes, one million U937 cells per sample were washed with ice-cold 1×PBS and lysed with 100 μl 2×LDS buffer containing 2-mercaptoethanol five days after electroporation. Lysates were boiled for 10 minutes at 95° C. Whole cell extracts were resolved by SDS-PAGE gel electrophoresis, transferred onto a nitrocellulose membrane using the Turboblot system (Bio-Rad), and probed with the indicated primary antibodies. Bound antibodies were detected with IRDye-680 or -800 conjugated secondary antibodies using a LI-COR scanner (see FIG. 9C).

Then a cell proliferation assay was performed. Compound D dissolved in DMSO was injected into 96-well cell culture plates using a HP D300 digital dispenser in triplicate for each final Compound D concentration. One week after electroporation to knockout the indicated genes, U937 Cas9 cell lines were plated in triplicate with 5000 cells in 100 μL complete culture media per well in the 96-well plates. After 5 days, cell proliferation was assessed using CTG (CellTiter-Glo) according to the manufacturer's instructions to assess the effects of Compound D on proliferation of each cell line.

Proliferation assays show that Compound D exhibited dose-dependent anti-proliferative effects in parental and non-coding (NC-8) controls (see FIGS. 9D and 9E). However, depletion of GCN2, ATF4, GCN1, CRBN, DDIT4, TSC1, and TSC2 diminished this effect (see FIGS. 9D and 9E).

In sum, enhanced sensitivity to Compound D was observed when mTOR or its components was knocked out. In addition, when suppression of mTOR is reduced by the depletion of various upstream and downstream negative regulators of mTOR, cells developed resistance to Compound D. Taken together, these data suggest that Compound D resistance in AML cells is driven at least in part by the mTOR signaling pathway. 6.6. Compound D Resistance Mediated by ILF2/ILF3

In addition to the genes involved in the mTOR pathway found to be implicated in Compound D resistance, ILF2 and ILF3 were shown to contribute to Compound D resistance (see FIG. 5). ILF2 and ILF3 form a heterodimeric complex, acting as a transcription factor that regulates IL-2 expression during T-cell activation. To determine the underlying mechanism of ILF2/3-mediated Compound D resistance, ILF2 and ILF3 were knocked out.

Next, CRISPR competition assays were conducted in U937 and OCI-AML2 cells as described above. Specifically, U937 and OCI-AML2 cell lines were transduced with plenti-EF1á-Cas9-P2A-Blast, followed by Blasticidin selection to establish Cas9-expressing cell lines. U937-Cas9 and OCI-AML2-Cas9 cells were then transduced with pRSG17-U6-sgNT-1-UbiC-TagGFP2-2A-Puro, or pRSG16-U6-UbiC-TagRFP-2A-Puro vectors expressing sgNT-1, sgNC-1, sgNC-8 or gene-specific sgRNAs. Transduced cells were washed with 1×PBS 72 hours post-transduction. Cells were assessed for transduction via RFP or GFP fluorescence reporter expression. Once fluorescence reporter expression was confirmed, RFP- and GFP-transduced cells were mixed at a 1:1 ratio and seeded in 2 ml culture media per well in 12-well tissue culture plates at a cell density of 200,000 cells per mL, and treated with DMSO or an appropriate dose of Compound. Remaining cells underwent puromycin selection for 3-7 days and were harvested for immunoblot analysis to confirm gene knock-out. After seeding cells for culture in 12-well plates, 100 iL cell culture per well was removed for flow cytometric analysis as the baseline “Day 0” RFP- and GFP-positive percentages in each well. This was repeated every 2-4 days for flow cytometric analysis of the RFP- and GFP-positive percentages over the indicated timecourse for each experiment. The RFP⁺/GFP⁺ ratios at each timepoint were normalized to their respective RFP+/GFP+ ratio on “Day 0.”

Knockout of ILF2 and ILF3 Conferred Compound D Resistance

Knockout of ILF2 and ILF3 in U937 and OCI-AML2 cells was accomplished by CRISPR-mediated gene editing tools as described above. Western blot analysis as shown in FIGS. 10A and 10C confirmed knockout of these genes in U937 cells, and the analysis shown in FIG. 10E confirms knockout of these genes in OCI-AML2 cells. The CRISPR competition analysis shows that ILF2 or ILF3 knockout in U937 and OCI-AML2 cells led to Compound D resistance (see FIGS. 10B, 10D, 10F). In FIGS. 10A, 10B, 10G and 10H, U937 Cas9 cells had been transduced with inducible vectors that had their sgRNA expression induced for several days prior to each assay with 1 μg/ml doxycycline. Constitutively-expressing sgRNAs were used in the remaining ILF2/3 assays shown (FIGS. 10C, 10D, 10E, and 10F).

Knockout of ILF3 Led to CRBN Downregulation and GSPT1 Accumulation

To ascertain if inactivation of ILF3 abrogates the response to Compound D, a flow cytometry-based CRISPR competition assay was utilized to discern the effect of ILF3 knockout on cell fitness in the presence or absence of Compound D (FIG. 10I). Specifically, U937-Cas9 cells were transduced with plenti-H1TO-EF1a-HTLV-TetR-P2A-RFP-P2A-Puro vectors expressing sgNT-1, sgNC-1, sgILF3-2, or sgILF3-4 to generate stable, doxycycline-inducible sgRNA cell lines. U937-Cas9 cells inducibly expressing sgNT-1, sgNC-1, sgILF3-2 or sgILF3-4 were treated with 1 μg/ml of doxycycline, and on the same day, U937-Cas9 cells were transduced with pRSG17-U6-sgNT-1-UbiC-TagGFP2-2A-Puro. After 3 days, RFP- and GFP-expressing cells were mixed at a 1:1 ratio and treated with DMSO or Compound D, followed by cell viability assessment by flow cytometry as described below.

For cell apoptosis assays, the ability of Compound D to induce apoptosis was assessed in selected AML cell lines at the time points and compound concentrations indicated. For Annexin V/7AAD readout by flow cytometry, AML cell lines were plated into flat bottom 96-well plates (BD Falcon) at a seeding density of 0.1-0.3×10⁶ cells per mL in 200 μL complete media. Compound D was dispensed onto the plates and the cells were incubated for 24-48 hrs. At the end of the incubation period, 100 μL of cells were transferred into a 96-well U-bottom plate (BD Falcon), centrifuged at 1200 rpm for 5 minutes, and the media was removed. 2.5 μL of Annexin V—AF647 (Biolegend) and 5 μL 7AAD (Biolegend) were diluted into 100 μL of 1×Annexin binding buffer (BD Biosciences). Fifteen minutes after addition of 100 μL of the Annexin V/7AAD buffer into each well, cells were analyzed using the Attune Flow Cytometer (Invitrogen). The apoptosis induction curve was processed and graphed using GraphPad Prism Version 7 (P<0.05, unpaired two-sided t-test, is considered as significant).

Doxycycline-induced expression of sgILF3, but not a control sgRNA, sgNT or sgNC, triggered effective knockout of ILF3 in U937 cells, leading to significant enrichment of ILF3-depleted cells over control cells, consistent with the CRISPR screen result (FIGS. 10B and 10J). ILF3 loss drastically reduced cereblon protein levels, and hence Compound D-induced GSPT1 degradation, likely contributing to the attenuated activity of Compound D upon ILF3 loss (FIGS. 10G and 10J). ILF2/NF45 and ILF3/NF90 form a heterodimeric complex, which is known to regulate gene expression at multiple levels including RNA transcription, alternative splicing, translation and microRNA biogenesis (Marchesini et al., Cancer Cell, 2017, 32:88-100; Pfeifer et al., Proc Natl Acad Sci USA 2008; Reichman et al., 2002; Sakamoto et al., 2009; Masuda et al., 2013). ILF2 knockout displayed the same effect as did ILF3 knockout in U937 (FIGS. 10C, 10D and 10K). Additionally, the effects of ILF2 or ILF3 ablation on cereblon expression and Compound D response can also be observed in OCI-AML2 (FIGS. 10E and 10F).

The results shown in FIGS. 10A, 10C, 10E, 10G indicate that knockout of TLF2 or ILF3 led to CRBN downregulation and attenuated Compound D induced GSPT1 degradation relative to Compound D induced GSPT1 degradation in parental, non-targeting (sgNT-1), and non-coding (sgNC-8) controls.

To investigate the mechanism by which ILF2/ILF3 complex regulates cereblon expression, mRNA-Seq was performed in U937-Cas9 cells expressing sgNT and sgILF3. Specifically, U937-Cas9 cells expressing inducible sgNT-1 or sgILF3-2 were treated with or without 1 μg/ml of doxycycline in triplicate for 5 days. Poly-A selected mRNA libraries were prepared using the TruSeq mRNA kit (Illumina) according to the manufacturer's protocol. Briefly, total RNA was extracted from cell pellets using the RNeasy Mini Kit, followed by mRNA isolation via poly-A selection. Purified mRNAs were fragmented and converted to 1st strand cDNAs with reverse transcriptase. The resulting 1st strand cDNAs were converted to double stranded cDNAs, and subjected to end-repair, A-tailing, and adapter ligation. The libraries were amplified and sequenced using Illumina's HiSeq 4000 (2×150 bp configuration, single index, per lane). Fastq converted sequence files were adapter and quality trimmed using Cutadapt v1.15 (Martin et al., EMBnetjournal, 2011, 17:10-12) and aligned to genome version hg38 using STAR v2.5 (Dobin et al., Bioinformatics, 2013, 29:15-21). A flattened exon file based on Gencode v24 annotation was prepared using the python script, “dexseq_prepare_annotation.py” provided with R package DEXSeq (Anders et al., Genome Res., 2012, 22:2008-17). This script generates unique exon bins by collapsing overlapping exons or exon regions as well as representing regions of unique exon boundaries from different transcripts. The “featureCounts” function from Subread v1.6.2 package was used to generate exon bin counts for each STAR aligned bam file (Liao et al., Bioinformatics, 2014, 30:923-30). Processed read counts were assessed for differential gene expression and alternative splicing between the two experimental conditions, ILF3 KO versus non-targeting (NT) controls, using R package edgeR (Robinson et al., Bioinformatics, 2010, 26:139-40). The “diffSpliceDGE” function applied to normalized exon bin counts fitted by a generalized linear model, tests for differential exon usage (DEU). DEU is a measure comparing the log 2 fold change (log FC) of an exon bin relative to the overall gene's log FC. Significant DEU thus signifies significant differences in relative abundance of an exon or exon region compared to abundance across the entire gene between the two experimental conditions. A false discovery rate (FDR)<0.05 was applied to define statistically significant gene and exon abundance.

Pathway enrichment analysis was performed using a hypergeometric model to identify Reactome pathways associated with a greater than expected number of differentially expressed genes between sgILF3-2 and sgNT-1 transfected cells. Genes with evidence of differential exon usage were similarly interrogated for Reactome pathway enrichment, each using the R package ReactomePA (Yu and He, Mol. Biosyst., 2016, 12:477-79).

ILF3 loss significantly (FDR<0.05) affected the expression of 645 genes, many of which are related to influenza infection and replication and, less significantly, translation elongation (FIGS. 11A and 11B). In alignment with the role of ILF2/ILF3 in pre-mRNA splicing, ILF3 loss drastically changed the level of alternatively spliced transcripts of 967 genes involved in several cellular functions including processing of pre-mRNA and rRNA, chromatin modification, and non-sense mediated mRNA decay (FIGS. 11A and 11B). This transcriptomic analysis revealed a drastic change of exon usage of CRBN, but not its total mRNA level, in response to ILF3 loss (FIGS. 7K, 11A and 11B). Human CRBN has 15 splicing variants, two of which (CRBN-201 and CRBN-203) produce a full-length functional protein (FIG. 11C). ILF3 knockout reduced the mRNA level of CRBN-201 and CRBN-203, and increased the level of splicing variant CRBN-213, which is composed of exons 1-4 and a cryptic exon 5 containing a premature stop codon (FIGS. 7K and 11C). CRBN-213 encodes a truncated cereblon protein which lacks the critical domain involved in binding of all cereblon modulators.

Then, quantitative PCR was used to analyze CRBN splicing isoforms. U937 Cas9 cells expressing doxycycline-inducible sgRNA vectors targeting ILF3 or a non-targeting (sgNT) control were induced with 1 μg/ml doxycycline for three days. After the induction, one million U937 cells per sample were washed with ice-cold 1×PBS and total RNA was isolated from each sample using the RNeasy Plus Mini kit (Qiagen). After reverse transcription to synthesize cDNA using the AffinityScript Reverse Transcription kit with random primers, SYBR Green qPCR was performed using primers specific to the full-length CRBN transcript, the truncated CRBN-213 transcript, or a GAPDH transcript as a loading control. The expression levels of the full length and truncated CRBN isoforms were compared between the ILF3 knock-out and sgNT cell lines. As shown in FIG. 611, loss of ILF3 resulted in decreased levels of full-length isoform 1 and 2 of CRBN and increased the expression of alternatively spliced CRBN-213. This suggests that ILF3 regulates the expression of CRBN post-transcriptionally. When ILF3 is depleted, alternative transcripts of CRBN are unable to regulate its downstream targets like GSPT1.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

6.7. Compound D Anti-Proliferative Activity Mediated by ISR

The integrated stress response (ISR) is an evolutionarily conserved homeostatic pathway. The activation of the ISR is initiated by the phosphorylation of the translation initiation factor eIF2a by one of four homologous stress-sensing kinases, PERK, GCN2, HRI or PKR, resulting in the inhibition of global protein translation and preferential translation of the ISR effector ATF4 and other genes possessing upstream open reading frames (Barid and Wek, Advances in Nutrition, 2012, 3:307-21; Donnelly et al., CMLS, 2013, 3493-511). GCN2 forms a complex with GCN1 on translating ribosomes and activates the ATF4 pathway when sensing cellular stresses including amino acid deprivation, protein translation stalling, proteasome inhibition, UV irradiation, and oxidative stress (Anda et al., PLoS One, 2017, 12: e0182143).

In the CRISPR screen, nearly all sgRNAs targeting GCN1, GCN2, as well as ATF4 and its downstream transcriptional target gene DDIT4 were significantly enriched by Compound D (FIG. 411), while enrichment of sgRNAs targeting other eIF2a kinases including PERK (EIF2AK3), HRI(EIF2AK1) and PKR (EIF2AK2) was not observed. Using the CRISPR competition assay we confirmed that loss of GCN1, GCN2, ATF4 or DDIT4 protected the cells against Compound D-induced growth inhibition in U937 and OCI-AML2 cells (FIGS. 12A-12K), suggesting that activation of the GCN2-mediated ISR plays a critical role in the anti-AML effect of Compound D. Indeed, Compound D treatment triggered the rapid phosphorylation of eIF2a, accumulation of ATF4 and its transcriptional targets DDIT4, CHOP and ATF3, and subsequent induction of apoptosis (FIGS. 13A and 13B).

Quantitative RT-PCR analysis confirmed the marked induction of ATF4 target genes ATF3, CHOP and DDIT4 at the mRNA level upon treatment with Compound D (FIGS. 13C and 13D). Specifically, following incubation with DMSO or Compound D, cells were collected via centrifugation at 2000 rpm for 2 minutes. Cell pellets were then washed once in ice-cold PBS and snap-frozen in liquid nitrogen. Total RNA was extracted using the RNeasy Mini Kit according to the manufacturer's instructions, and reverse-transcribed into first-strand cDNA using the AffinityScript QPCR cDNA Synthesis Kit with random primers. The cDNA transcripts of GADPH, DDIT3 (CHOP), DDIT4 (REDD), and ATF3 were quantified by the ViiA™ 7 Real-Time PCR System using TaqMan Gene Expression Assay probes (Invitrogen). The cDNA level of various CRBN transcripts was quantified using RT² SYBR Green qPCR Mastermixes (Qiagen). Each reaction was performed in triplicates or quadruplicates, and values were averaged to calculate the relative expression level. Primer sequences for detected alternatively transcribed CRBN transcripts are listed in Table 1 below:

TABLE 1
qRT-PCR Primer Sequences

| Probe | Primer Name | Sequence |
|---|---|---|
| Probe 1 | CRBN.201/202-1 For | GCAGTGCTATCCAGCGACTT |
| | CRBN.201/202-1 Rev | GCCGGCCTATCAGATTCAA |
| Probe 2 | CRBN.201/202-2 For | AGCGACTTCGCTGTGAATTA |
| | CRBN.201/202-2 Rev | AGGCATACCCAGGAAACCAG |
| Probe 3 | CRBN.213-1 For | AAAGGGAAGCACAGTTTGGA |
| | CRBN.213-1 Rev | GATGGGAGATGAGGTTGGAA |
| Probe 4 | CRBN.213-2For | AGAACCTTTGCTGTTCTTGCA |
| | CRBN.213-2 Rev | AGCTTCCCAGGTGATTCTGA |
| Control | GADPH For | GAAGGTGAAGGTCGGAGTCA |
| | GADPH Rev | GACAAGCTTCCCGTTCTCAG |

To determine if the activation of the ISR by Compound D is solely mediated by GCN2, the effect of GCN2 ablation on Compound D response was evaluated. GCN2 knockout inhibited the phosphorylation of eIF2a, induction of ATF4 and its target gene DDIT4, and the cleavage of caspase-3 in U937 cells treated with Compound D (FIGS. 13A and 13C). GCN2 loss largely, but not completely, blocked the induction of other ATF4 target genes ATF3 and CHOP (FIG. 13C), suggesting the involvement of additional signaling pathways capable of inducing these two genes. Reintroduction of GCN2 wild-type, but not any of its enzymatically dead mutants T899A/T904A, K619R and F1143L/R1144L, significantly restored the response to Compound D in U937 GCN2−/− cells (FIGS. 13E and 13F). Collectively, these findings suggest that activation of the ISR pathway in response to GSPT1 degradation at least partially modulates the anti-AML activity of Compound D.

What is claimed is:

1. A method of identifying a subject having cancer who is likely to be responsive to a treatment comprising a compound or predicting the responsiveness of a subject having or suspected of having cancer to a treatment comprising the compound, comprising:
i. providing a sample from the subject;
ii. measuring gene expression level of one or more genes in the sample; and
iii. identifying the subject as being likely to be responsive to the treatment comprising the compound if the expression level of the gene is different from a reference level,
wherein the compound is 2-(4-chlorophenyl)-N-((2-(2,6-dioxopiperidin-3-yl)-1-oxoisoindolin-5-yl)methyl)-2,2-difluoroacetamide (Compound D), which has the following structure: 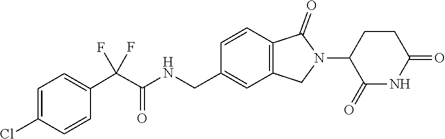 [SMILES: O=C1CCC(N2Cc3cc(CNC(=O)C(F)(F)c4ccc(Cl)cc4)ccc3C2=O)C(=O)N1]
or a stereoisomer or mixture of stereoisomers, isotopologue, pharmaceutically acceptable salt, tautomer, solvate, hydrate, co-crystal, clathrate, or polymorph thereof, and
wherein the gene is a gene involved in mTOR signaling, or the gene is ILF2 or ILF3.

2. A method of treating a subject having cancer with a compound, comprising:
(a) identifying the subject having cancer that may be responsive to the treatment comprising the compound, comprising:
i. providing a sample from the subject;
ii. measuring gene expression level of one or more genes in the sample; and
iii. identifying the subject as being likely to be responsive to the treatment comprising the compound if the expression level of the gene is different from a reference level,
(b) administering the subject a therapeutically effective amount of the compound if the subject is identified as being likely to be responsive to the treatment comprising the compound,
wherein the compound is Compound D, or a stereoisomer or mixture of stereoisomers, isotopologue, pharmaceutically acceptable salt, tautomer, solvate, hydrate, co-crystal, clathrate, or polymorph thereof and
wherein the gene is a gene involved in mTOR signaling, or the gene is ILF2 or ILF3.

3. The method of claim 1 or claim 2, wherein the gene is a gene involved in mTOR signaling.

4. The method of claim 3, wherein the gene is a positive regulator of mTOR signaling.

5. The method of claim 3, wherein the gene is mTOR.

6. The method of claim 3, wherein the gene is Raptor.

7. The method of claim 3, wherein the gene is Rictor.

8. The method of any one of claims 4-7, wherein the method comprises identifying the subject as being likely to be responsive to the treatment comprising the compound if the expression level of the gene is lower than a reference level.

9. The method of claim 8, wherein the reference level is the expression level of the gene in a subject resistant to Compound D.

10. The method of claim 8, wherein the reference level is the expression level of the gene in a subject without the cancer.

11. The method of claim 8, wherein the reference level is a pre-determined level.

12. The method of claim 3, wherein the gene is a negative regulator of mTOR signaling.

13. The method of claim 3, wherein the gene is TSC1

14. The method of claim 3, wherein the gene is TSC2.

15. The method of claim 3, wherein the gene is GCN1.

16. The method of claim 3, wherein the gene is GCN2.

17. The method of claim 3, wherein the gene is DDIT4.

18. The method of claim 3, wherein the gene is ATF4.

19. The method of any one of claims 12-18, wherein the method comprises identifying the subject as being likely to be responsive to the treatment comprising the compound if the expression level of the gene is higher than a reference level.

20. The method of claim 19, wherein the reference level is the expression level of the gene in a subject responsive to Compound D.

21. The method of claim 19, wherein the reference level is the expression level of the gene in a subject without the cancer.

22. The method of claim 19, wherein the reference level is a pre-determined level.

23. The method of claim 1 or claim 2, wherein the gene is ILF2.

24. The method of claim 1 or claim 2, wherein the gene is ILF3.

25. The method of claim 23 or claim 24, wherein the method comprises identifying the subject as being likely to be responsive to the treatment comprising the compound if the expression level of the gene is higher than a reference level.

26. The method of claim 25, wherein the reference level is the expression level of the gene in a subject responsive to Compound D.

27. The method of claim 25, wherein the reference level is the expression level of the gene in a subject without the cancer.

28. The method of claim 25, wherein the reference level is a pre-determined level.

29. The method of any one of claims 1 to 28, wherein the cancer is a hematological cancer.

30. The method of any one of claims 1 to 28, wherein the cancer is a lymphoma.

31. The method of any one of claim 1 to 28, wherein the cancer is a leukemia.

32. The method of claim 31, wherein the cancer is AML.

33. A method of identifying a subject having cancer who is likely to be responsive to a treatment comprising a compound or predicting the responsiveness of a subject having or suspected of having cancer to a treatment comprising the compound, comprising:
i. providing a sample from the subject;
ii. determining a sequence of a biomarker in the sample; and
iii. identifying the subject as being unlikely to be responsive to the treatment comprising the compound if a mutation is identified in the biomarker, and/or identifying the subject as being likely to be responsive to the treatment comprising the compound if the mutation is not identified in the biomarker;
wherein the compound is Compound D, or a stereoisomer or mixture of stereoisomers, isotopologue, pharmaceutically acceptable salt, tautomer, solvate, hydrate, co-crystal, clathrate, or polymorph thereof, and
wherein the biomarker is CRBN or GSPT1.

34. The method of claim 33, wherein the biomarker is GSPT1, and wherein the mutation is a mutation of amino acid residue C568, L569, V570, D571, K572, K573, S574, G575, or E576 of GSPT1.

35. The method of claim 34, wherein the mutation is selected from a group consisting of K572, K573, S574, G575, and combinations thereof.

36. The method of claim 35, wherein the mutation comprises G575N.

37. The method of claim 33, wherein the biomarker is CRBN, and wherein the mutation is a mutation of amino acid residue N351, H357, W380, Y384, W386, or W400.

38. The method of claim 37, wherein the mutation is Y384A or W386A.

39. A method of treating a subject having cancer comprising administering to the subject a compound, wherein the subject has been determined to be likely to be responsive to the compound according a method comprising:
i. providing a sample from the subject;
ii. determining a sequence of a biomarker in the sample; and
iii. identifying the subject as being likely to be responsive to the compound if a mutation is not identified in the biomarker;
wherein the compound is Compound D, or a stereoisomer or mixture of stereoisomers, isotopologue, pharmaceutically acceptable salt, tautomer, solvate, hydrate, co-crystal, clathrate, or polymorph thereof, and
wherein the biomarker is CRBN or GSPT1.

40. A method of treating a subject having cancer comprising administering to the subject a second compound, wherein the subject has been determined to be unlikely to be responsive to a first compound according a method comprising:
i. providing a sample from the subject;
ii. determining a sequence of a biomarker in the sample; and
iii. identifying the subject as being unlikely to be responsive to the compound if a mutation is identified in the biomarker;
wherein the first compound is Compound D, or a stereoisomer or mixture of stereoisomers, isotopologue, pharmaceutically acceptable salt, tautomer, solvate, hydrate, co-crystal, clathrate, or polymorph thereof,
wherein the second compound is not Compound D, or a stereoisomer or mixture of stereoisomers, isotopologue, pharmaceutically acceptable salt, tautomer, solvate, hydrate, co-crystal, clathrate, or polymorph thereof, and
wherein the biomarker is CRBN or GSPT1.

41. The method of claim 39 or claim 40, wherein the biomarker is GSPT1, and wherein the mutation is a mutation of amino acid residue C568, L569, V570, D571, K572, K573, S574, G575, or E576 of GSPT1.

42. The method of claim 41, wherein the mutation is selected from a group consisting of K572, K573, S574, G575, and combinations thereof.

43. The method of claim 42, wherein the mutation comprises G575N.

44. The method of claim 39 or claim 40, wherein the biomarker is CRBN, and wherein the mutation is a mutation of amino acid residue N351, H357, W380, Y384, W386, or W400.

45. The method of claim 44, wherein the mutation is Y384A or W386A.

46. The method of any one of claims 33 to 45, wherein the cancer is a hematological cancer.

47. The method of any one of claims 33 to 45, wherein the cancer is a lymphoma.

48. The method of any one of claim 33 to 45, wherein the cancer is a leukemia.

49. The method of any one of claims 33 to 45, wherein the cancer is AML.


SEQUENCE LISTING (10 sequences)

SEQ ID NO: 1 - DNA, 20 bp, Artificial Sequence
  qRT-PCR Primer-CRBN.201/202-1 For - Probe 1
gcagtgctatccagcgactt

SEQ ID NO: 2 - DNA, 19 bp, Artificial Sequence
  qRT-PCR Primer-CRBN.201/202-1 Rev - Probe 1
gccggcctatcagattcaa

SEQ ID NO: 3 - DNA, 20 bp, Artificial Sequence
  qRT-PCR Primer-CRBN.201/202-2 For - Probe 2
agcgacttcgctgtgaatta

SEQ ID NO: 4 - DNA, 20 bp, Artificial Sequence
  qRT-PCR Primer-CRBN.201/202-2 Rev - Probe 2
aggcatacccaggaaaccag

SEQ ID NO: 5 - DNA, 20 bp, Artificial Sequence
  qRT-PCR Primer-CRBN.213-1 For - Probe 3
aaagggaagcacagtttgga

SEQ ID NO: 6 - DNA, 20 bp, Artificial Sequence
  qRT-PCR Primer-CRBN.213-1 Rev - Probe 3
gatgggagatgaggttggaa

SEQ ID NO: 7 - DNA, 21 bp, Artificial Sequence
  qRT-PCR Primer-CRBN.213-2 For - Probe 4
agaacctttgctgttcttgca

SEQ ID NO: 8 - DNA, 20 bp, Artificial Sequence
  qRT-PCR Primer-CRBN.213-2 Rev - Probe 4
agcttcccaggtgattctga

SEQ ID NO: 9 - DNA, 20 bp, Artificial Sequence
  qRT-PCR Primer-GADPH For - Control
gaaggtgaaggtcggagtca

SEQ ID NO: 10 - DNA, 20 bp, Artificial Sequence
  qRT-PCR Primer-GADPH Rev - Control
gacaagcttcccgttctcag